(19) United States
(12) Patent Application Publication
(10) Pub. No.: US 2025/0177433 A1
(43) Pub. Date: Jun. 5, 2025

(54) COMPOUNDS AND METHODS FOR REDUCING DMPK EXPRESSION

(71) Applicant: Ionis Pharmaceuticals, Inc., Carlsbad, CA (US)

(72) Inventors: Frank Rigo, Carlsbad, CA (US); Eric E. Swayze, Encinitas, CA (US); Paymaan Jafer-Nejad, Carlsbad, CA (US); Michael Oestergaard, Carlsbad, CA (US); Kar Yun Karen Ling, Carlsbad, CA (US); Susan M. Freier, San Diego, CA (US); Huynh-Hoa Bui, San Diego, CA (US)

(73) Assignee: Ionis Pharmaceuticals, Inc., Carlsbad, CA (US)

(21) Appl. No.: 18/687,221

(22) PCT Filed: Aug. 31, 2022
(86) PCT No.: PCT/US2022/075768

§ 371 Date: Feb. 27, 2024

Related U.S. Application Data
(60) Provisional application No. 63/239,741, filed on Sep. 1, 2021.

(51) Int. Cl. A61K 31/7125; A61P 21/00; C12N 15/11

(52) U.S. Cl. CPC A61K 31/7125; A61P 21/00; C12N 15/11; C12N 2310/314; C12N 2310/315; C12N 2310/32; C12N 2310/3341

(57) ABSTRACT

Provided are oligomeric compounds, methods, and pharmaceutical compositions for DMPK the amount or activity of DMPK RNA in a cell or animal, and in certain instances reducing the amount of DMPK protein in a cell or animal. Such oligomeric compounds, methods, and pharmaceutical compositions are useful to treat type 1 myotonic dystrophy.

### SEQUENCE LISTING

The present application is being filed along with a Sequence Listing in electronic format. The Sequence Listing is provided as a file entitled BIOL0430WOSEQ.xml, created on Aug. 25, 2022, which is 2,065 KB in size. The information in the electronic format of the sequence listing is incorporated herein by reference in its entirety.

### FIELD

Provided are oligomeric compounds, methods, and pharmaceutical compositions for reducing the amount or activity of DMPK RNA in a cell or animal, and in certain instances reducing the amount of DMPK protein in a cell or animal. Such oligomeric compounds, methods, and pharmaceutical compositions are useful to treat type 1 myotonic dystrophy (DM1) in an animal.

### BACKGROUND

Myotonic dystrophy type 1 (DM1) is the most common form of muscular dystrophy in adults with an estimated frequency of 1 in 7,500 (Harper P S., Myotonic Dystrophy. London: W. B. Saunders Company; 2001). DM1 is an autosomal dominant disorder caused by expansion of a non-coding CTG repeat in DMPK1. DMPK1 is a gene encoding a cytosolic serine/threonine kinase (Brook J D, et al., Cell., 1992, 68(4):799-808). The physiologic functions and substrates of this kinase have not been fully determined. The expanded CTG repeat is located in the 3′ untranslated region (UTR) of DMPK1. This mutation leads to RNA dominance, a process in which expression of RNA containing an expanded CUG repeat (CUGexp) induces cell dysfunction (Osborne R J and Thornton C A., Human Molecular Genetics., 2006, 15(2): R162-R169).

The DMPK gene normally has 5-37 CTG repeats in the 3′ untranslated region. In type 1 myotonic dystrophy, this number is significantly expanded and is, for example, in the range of 50 to greater than 3,500 (Harper, Myotonic Dystrophy (Saunders, London, ed. 3, 2001); Annu. Rev. Neurosci. 29: 259, 2006; EMBO J. 19: 4439, 2000; Curr Opin Neurol. 20: 572, 2007).

The CUGexp tract interacts with RNA binding proteins including muscleblind-like (MBNL) protein, a splicing factor, and causes the mutant transcript to be retained in nuclear foci. The toxicity of this RNA stems from sequestration of RNA binding proteins and activation of signaling pathways. Studies in animal models have shown that phenotypes of DM1 can be reversed if toxicity of CUGexp RNA is reduced (Wheeler™, et al., Science., 2009, 325(5938):336-339; Mulders S A, et al., Proc Natl Acad Sci USA., 2009, 106(33):13915-13920).

In DM1, skeletal muscle is the most severely affected tissue, but the disease also has important effects on cardiac and smooth muscle, ocular lens, and brain. The cranial, distal limb, and diaphragm muscles are preferentially affected. Manual dexterity is compromised early, which causes several decades of severe disability. The median age at death is 55 years, usually from respiratory failure (de Die-Smulders C E, et al., Brain., 1998, 121 (Pt 8):1557-1563).

Antisense technology is emerging as an effective means for modulating expression of certain gene products and may therefore prove to be uniquely useful in a number of therapeutic, diagnostic, and research applications for the modulation of DMPK1.

Presently there is no treatment that can modify the course of DM1. The burden of disease, therefore, is significant. It is, therefore, an object herein to provide compounds, compositions, and methods for treating DM1.

### SUMMARY

Oligomeric compounds, methods, and pharmaceutical compositions of certain embodiments described herein are useful for reducing or inhibiting DMPK expression in a cell or animal. In certain embodiments, DMPK RNA or protein levels can be reduced in a cell or animal. In certain embodiments, the subject has type 1 myotonic dystrophy (DM1). In certain embodiments, the subject has a disease or disorder associated with a mutation in DMPK.

Also provided are methods of treating an animal having type 1 myotonic dystrophy.

### DETAILED DESCRIPTION

It is to be understood that both the foregoing general description and the following detailed description are exemplary and explanatory only and are not restrictive. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated-by-reference for the portions of the document discussed herein, as well as in their entirety.

### Definitions

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Where permitted, all patents, applications, published applications and other publications and other data referred to throughout in the disclosure are incorporated by reference herein in their entirety.

Unless otherwise indicated, the following terms have the following meanings:

As used herein, “2′-deoxynucleoside” means a nucleoside comprising a 2′-H(H) deoxyfuranosyl sugar moiety. In certain embodiments, a 2′-deoxynucleoside is a 2′-β-D-deoxynucleoside and comprises a 2′-β-D-deoxyribosyl sugar moiety, which has the β-D ribosyl configuration as found in naturally occurring deoxyribonucleic acids (DNA). In certain embodiments, a 2′-deoxynucleoside may comprise a modified nucleobase or may comprise an RNA nucleobase (uracil).

As used herein, “2′-MOE” means a 2′-OCH₂CH₂OCH₃ group in place of the 2′-OH group of a furanosyl sugar moiety. A “2′-MOE sugar moiety” means a sugar moiety with a 2′-OCH₂CH₂OCH₃ group in place of the 2′-OH group of a furanosyl sugar moiety. Unless otherwise indicated, a 2′-MOE sugar moiety is in the β-D-ribosyl configuration. “MOE” means O-methoxyethyl.

As used herein, “2′-MOE nucleoside” means a nucleoside comprising a 2′-MOE sugar moiety.

As used herein, “2′-OMe” means a 2′-OCH₃ group in place of the 2′-OH group of a furanosyl sugar moiety. A “2′-O-methyl sugar moiety” or “2′-OMe sugar moiety” means a sugar moiety with a 2′-OCH₃ group in place of the 2′-OH group of a furanosyl sugar moiety. Unless otherwise indicated, a 2′-OMe sugar moiety is in the β-D-ribosyl configuration.

As used herein, “2′-OMe nucleoside” means a nucleoside comprising a 2′-OMe sugar moiety.

As used herein, “5-methylcytosine” means a cytosine modified with a methyl group attached to the 5 position. A 5-methylcytosine is a modified nucleobase.

As used herein, “ameliorate” in reference to a treatment means improvement in at least one symptom or hallmark relative to the same symptom or hallmark in the absence of the treatment. In certain embodiments, amelioration is the reduction in the severity or frequency of a symptom or hallmark or the delayed onset or slowing of progression in the severity or frequency of a symptom or hallmark. In certain embodiments, the symptom or hallmark is one or more of muscle stiffness, myotonia, disabling distal weakness, weakness in face and jaw muscles, difficulty in swallowing, drooping of the eyelids (ptosis), weakness of neck muscles, weakness in arm and leg muscles, persistent muscle pain, hypersomnia, muscle wasting, dysphagia, respiratory insufficiency, irregular heartbeat, heart muscle damage, apathy, insulin resistance, and cataracts.

As used herein, “antisense agent” means an antisense compound and optionally one or more additional features, such as a sense compound.

As used herein, “cerebrospinal fluid” or “CSF” means the fluid filling the space around the brain and spinal cord. “Artificial cerebrospinal fluid” or “aCSF” means a prepared or manufactured fluid that has certain properties (e.g., osmolarity, pH, and/or electrolytes) of cerebrospinal fluid and is biocompatible with CSF.

As used herein, “conjugate group” means a group of atoms that is directly attached to an oligonucleotide. Conjugate groups include a conjugate moiety and a conjugate linker that attaches the conjugate moiety to the oligonucleotide.

As used herein, “conjugate linker” means a single bond or a group of atoms comprising at least one bond that connects a conjugate moiety to an oligonucleotide.

As used herein, “conjugate moiety” means a group of atoms that modifies one or more properties of a molecule compared to the identical molecule lacking the conjugate moiety, including but not limited to pharmacodynamics, pharmacokinetics, stability, binding, absorption, tissue distribution, cellular distribution, cellular uptake, charge and clearance.

As used herein, “constrained ethyl” or “cEt” or “cEt sugar moiety” means a β-D ribosyl bicyclic sugar moiety wherein the second ring of the bicyclic sugar is formed via a bridge connecting the 4′-carbon and the 2′-carbon of the β-D ribosyl sugar moiety, wherein the bridge has the formula 4′-CH(CH₃)—O-2′, and wherein the methyl group of the bridge is in the S configuration.

As used herein, “cEt nucleoside” means a nucleoside comprising a cEt sugar moiety.

As used herein, “deoxy region” means a region of 5-12 contiguous nucleotides, wherein at least 70% of the nucleosides comprise a β-D-2′-deoxyribosyl sugar moiety. In certain embodiments, a deoxy region is the gap of a gapmer.

As used herein, “internucleoside linkage” is the covalent linkage between adjacent nucleosides in an oligonucleotide. As used herein “modified internucleoside linkage” means any internucleoside linkage other than a phosphodiester internucleoside linkage.

As used herein, “linked nucleosides” are nucleosides that are connected in a contiguous sequence (i.e., no additional nucleosides are presented between those that are linked).

As used herein, “motif” means the pattern of unmodified and/or modified sugar moieties, nucleobases, and/or internucleoside linkages, in an oligonucleotide.

As used herein, “modified nucleoside” means a nucleoside comprising a modified nucleobase and/or a modified sugar moiety.

As used herein, “non-bicyclic modified sugar moiety” means a modified sugar moiety that comprises a modification, such as a substituent, that does not form a bridge between two atoms of the sugar to form a second ring.

As used herein, “nucleobase” means an unmodified nucleobase or a modified nucleobase. A nucleobase is a heterocyclic moiety. As used herein an “unmodified nucleobase” is adenine (A), thymine (T), cytosine (C), uracil (U), or guanine (G). As used herein, a “modified nucleobase” is a group of atoms other than unmodified A, T, C, U, or G capable of pairing with at least one other nucleobase. A “5-methylcytosine” is a modified nucleobase. A universal base is a modified nucleobase that can pair with any one of the five unmodified nucleobases.

As used herein, “nucleobase sequence” means the order of contiguous nucleobases in a nucleic acid or oligonucleotide independent of any sugar or internucleoside linkage modification.

As used herein, “nucleoside” means a compound or fragment of a compound comprising a nucleobase and a sugar moiety. The nucleobase and sugar moiety are each, independently, unmodified or modified.

As used herein, “oligomeric compound” means an oligonucleotide and optionally one or more additional features, such as a conjugate group or terminal group. An oligomeric compound may be paired with a second oligomeric compound that is complementary to the first oligomeric compound or may be unpaired. A “singled-stranded oligomeric compound” is an unpaired oligomeric compound.

As used herein, “oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “pharmaceutically acceptable carrier or diluent” means any substance suitable for use in administering to an animal. Certain such carriers enable pharmaceutical compositions to be formulated as, for example, tablets, pills, dragees, capsules, liquids, gels, syrups, slurries, suspension and lozenges for the oral ingestion by a subject. In certain embodiments, a pharmaceutically acceptable carrier or diluent is sterile water, sterile saline, sterile buffer solution, or sterile artificial cerebrospinal fluid.

As used herein “pharmaceutically acceptable salts” means physiologically and pharmaceutically acceptable salts of compounds. Pharmaceutically acceptable salts retain the desired biological activity of the parent compound and do not impart undesired toxicological effects thereto.

As used herein “pharmaceutical composition” means a mixture of substances suitable for administering to a subject. For example, a pharmaceutical composition may comprise an oligomeric compound and a sterile aqueous solution. In certain embodiments, a pharmaceutical composition shows activity in free uptake assay in certain cell lines.

As used herein, “prodrug” means an inactive or less active form of a compound which, when administered to a subject, is metabolized to form the active, or more active, compound. In certain embodiments, a prodrug comprises a cell-targeting moiety and at least one active compound.

As used herein, “stereorandom” or “stereorandom chiral center” in the context of a population of molecules of identical molecular formula means a chiral center that is not controlled during synthesis, or enriched following synthesis, for a particular absolute stereochemical configuration. The stereochemical configuration of a chiral center is random when it is the result of a synthetic method that is not designed to control the stereochemical configuration. For example, in a population of molecules comprising a stereorandom chiral center, the number of molecules having the (S) configuration of the stereorandom chiral center may be but is not necessarily the same as the number of molecules having the (R) configuration of the stereorandom chiral center (“racemic”). In certain embodiments, the stereorandom chiral center is not racemic because one absolute configuration predominates following synthesis, e.g., due to the action of non-chiral reagents near the enriched stereochemistry of an adjacent sugar moiety. In certain embodiments, the stereorandom chiral center is at the phosphorous atom of a stereorandom phosphorothioate internucleoside linkage.

As used herein, “sugar moiety” means an unmodified sugar moiety or a modified sugar moiety. As used herein, “unmodified sugar moiety” means a 2′-OH(H) ribosyl moiety, as found in RNA (an “unmodified RNA sugar moiety”), or a 2′-H(H) deoxyribosyl sugar moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. As used herein, “modified sugar moiety” or “modified sugar” means a modified furanosyl sugar moiety or a sugar surrogate.

As used herein, “symptom or hallmark” means any physical feature or test result that indicates the existence or extent of a disease or disorder. In certain embodiments, a symptom is apparent to a subject or to a medical professional examining or testing said subject. In certain embodiments, a hallmark is apparent upon invasive diagnostic testing, including, but not limited to, post-mortem tests.

As used herein, “target nucleic acid” and “target RNA” mean a nucleic acid that an oligomeric compound is designed to affect. Target RNA means an RNA transcript and includes pre-mRNA and mRNA unless otherwise specified.

As used herein, “target region” means a portion of a target nucleic acid to which an oligomeric compound is designed to hybridize.

As used herein, “terminal group” means a chemical group or group of atoms that is covalently linked to a terminus of an oligonucleotide.

As used herein, “antisense activity” means any detectable and/or measurable change attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound.

As used herein, “gapmer” means a modified oligonucleotide comprising an internal region positioned between external regions having one or more nucleosides, wherein the nucleosides comprising the internal region are chemically distinct from the nucleoside or nucleosides comprising the external regions, and wherein the modified oligonucleotide supports RNAse H cleavage. The internal region may be referred to as the “gap” and the external regions may be referred to as the “wings.” In certain embodiments, the internal region is a deoxy region. The positions of the internal region or gap refer to the order of the nucleosides of the internal region and are counted starting from the 5′-end of the internal region. Unless otherwise indicated, “gapmer” refers to a sugar motif. In certain embodiments, each nucleoside of the gap is a 2′-β-D-deoxynucleoside. As used herein, the term “MOE gapmer” indicates a gapmer having a gap comprising 2′-β-D-deoxynucleosides and wings comprising 2′-MOE nucleosides. As used herein, the term “cEt gapmer” indicates a gapmer having a gap comprising 2′-β-D-deoxynucleosides and wings comprising cEt nucleosides. Unless otherwise indicated, a gapmer may comprise one or more modified internucleoside linkages and/or modified nucleobases and such modifications do not necessarily follow the gapmer pattern of the sugar modifications.

As used herein, “hybridization” means the annealing of oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases. In certain embodiments, complementary nucleic acid molecules include, but are not limited to, an antisense compound and a nucleic acid target. In certain embodiments, complementary nucleic acid molecules include, but are not limited to, an oligonucleotide and a nucleic acid target.

As used herein, “RNAi agent” means an antisense agent that acts, at least in part, through RISC or Ago2 to modulate a target nucleic acid and/or protein encoded by a target nucleic acid. RNAi agents include, but are not limited to double-stranded siRNA, single-stranded RNAi (ssRNAi), and microRNA, including microRNA mimics. RNAi agents may comprise conjugate groups and/or terminal groups. In certain embodiments, an RNAi agent modulates the amount and/or activity, of a target nucleic acid. The term RNAi agent excludes antisense agents that act through RNase H.

As used herein, “RNase H agent” means an antisense agent that acts through RNase H to modulate a target nucleic acid and/or protein encoded by a target nucleic acid. In certain embodiments, RNase H agents are single-stranded. In certain embodiments, RNase H agents are double-stranded. RNase H compounds may comprise conjugate groups and/or terminal groups. In certain embodiments, an RNase H agent modulates the amount and/or activity of a target nucleic acid. The term RNase H agent excludes antisense agents that act principally through RISC/Ago2.

As used herein, “standard cell assay” means the assays described in Examples 1-3, and reasonable variations thereof.

As used herein, “treating” means improving a subject's disease or condition by administering an oligomeric compound described herein. In certain embodiments, treating a subject improves a symptom relative to the same symptom in the absence of the treatment. In certain embodiments, treatment reduces in the severity or frequency of a symptom, or delays the onset of a symptom, slows the progression of a symptom, or slows the severity or frequency of a symptom.

As used herein, “therapeutically effective amount” means an amount of a pharmaceutical agent or composition that provides a therapeutic benefit to an animal. For example, a therapeutically effective amount improves a symptom of a disease.

### Certain Embodiments

Embodiment 1. An oligomeric compound comprising a modified oligonucleotide consisting of 12 to 30 linked nucleosides, wherein the nucleobase sequence of the modified oligonucleotide is at least 80% complementary to an equal length portion of a DMPK nucleic acid, and wherein the modified oligonucleotide has at least one modification selected from a modified sugar moiety and a modified internucleoside linkage.

Embodiment 2. The oligomeric compound of embodiment 1, wherein the DMPK nucleic acid has the nucleobase sequence of SEQ ID NO: 1 or SEQ ID NO: 2.

Embodiment 3. The oligomeric compound of embodiment 1 or embodiment 2, wherein the nucleobase sequence of the modified oligonucleotide is at least 80% complementary to an equal length portion within:
nucleobases 9052-9103 of SEQ ID NO: 1;
nucleobases 9228-9256 of SEQ ID NO: 1;
nucleobases 9574-9610 of SEQ ID NO: 1;
nucleobases 10010-10043 of SEQ ID NO: 1;
nucleobases 10271-10298 of SEQ ID NO: 1;
nucleobases 10364-10391 of SEQ ID NO: 1;
nucleobases 10683-10707 of SEQ ID NO: 1;
nucleobases 10709-10734 of SEQ ID NO: 1;
nucleobases 10812-10857 of SEQ ID NO: 1;
nucleobases 11853-11879 of SEQ ID NO: 1;
nucleobases 13310-13350 of SEQ ID NO: 1;
nucleobases 13999-14046 of SEQ ID NO: 1;
nucleobases 14090-14118 of SEQ ID NO: 1;
nucleobases 14232-14258 of SEQ ID NO: 1;
nucleobases 17565-17594 of SEQ ID NO: 1;
nucleobases 17731-17761 of SEQ ID NO: 1;
nucleobases 19719-19753 of SEQ ID NO: 1;
nucleobases 19795-19869 of SEQ ID NO: 1;
nucleobases 19888-19942 of SEQ ID NO: 1;
nucleobases 19915-19942 of SEQ ID NO: 1;
nucleobases 20871-20905 of SEQ ID NO: 1;
nucleobases 21117-21153 of SEQ ID NO: 1; or
nucleobases 22118-22143 of SEQ ID NO: 1.

Embodiment 4. The oligomeric compound of any of embodiments 1-3, wherein the modified oligonucleotide has a nucleobase sequence comprising at least 12, 13, 14, 15, or 16 contiguous nucleobases of a nucleobase sequence selected from:
SEQ ID Nos: 132, 186, 256, 327, 446, 1374, 1596, 1667, 1747, 1818, 1895, 1964, 2038, 2121, 2191;
SEQ ID NOs: 510, 1173, 1668, 1748, 1819, 1896;
SEQ ID NOs: 1376, 1448, 1526, 1599, 1670;
SEQ ID NOs: 1823, 1900, 1969, 2043;
SEQ ID NOs: 1380, 1452, 1530, 1901, 1970, 2044, 2127, 2197;
SEQ ID NOs: 1206, 1381, 1453, 1531, 1604, 1971, 2045, 2128, 2198;
SEQ ID NOs: 640, 714, 821, 1172, 1677, 1757, 1828;
SEQ ID NOs: 43, 115, 202, 900, 960, 1027, 1195, 1905;
SEQ ID NOs: 1384, 1456, 1534, 1607, 1678, 1758;
SEQ ID NOs: 1387, 1977, 2051, 2134, 2204;
SEQ ID NOs: 1296, 1351, 1425, 1501, 1793, 1867, 1979, 2052, 2083, 2092, 2206;
SEQ ID NOs: 49, 159, 208, 293, 402, 471, 556, 618, 676, 692, 754, 817, 901, 971, 1038, 1744, 1791, 1863, 1960, 2016, 2119, 2163;
SEQ ID NOs: 1718, 1814, 1891, 1941;
SEQ ID NOs: 41, 140, 888, 981, 1033, 2081, 2154;
SEQ ID NOs: 444, 508, 573, 1874, 1949, 2060, 2103;
SEQ ID NOs: 274, 337, 410, 526, 575, 665, 712, 829, 897, 1397, 1467, 2138, 2210, 2270;
SEQ ID NOs: 1432, 1509, 1580, 1654, 1729, 1801;
SEQ ID NOs: 355, 412, 506, 567, 673, 747, 832, 904, 956, 1399, 1469, 1545, 1581, 1655, 1730, 1841, 1916, 1988, 1989, 2027, 2106, 2177;
SEQ ID Nos: 160, 249, 313, 371, 424, 503, 588, 647, 755, 789, 882, 1248-1254, 1263-1264, 1266-1273, 1284-1285, 1332, 1400, 1489, 1619, 1637, 1638, 1639, 1656, 1709, 2006, 2079, 2082, 2085, 2153, 2303;
SEQ ID NOs: 503, 588, 647, 755, 789, 882, 1263, 1264, 1332, 1400, 1619, 1637, 1638, 1639, 1656, 1709, 2006, 2079, 2082, 2085, 2153, 2303;
SEQ ID NOs: 144, 233, 291, 328, 435, 482, 564, 642, 748, 808, 874, 955, 1339, 1340, 1341, 1492, 1732, 1803, 2321, 2322, 2323;
SEQ ID NOs: 576, 652, 724, 811, 870, 1359, 1433, 1510, 1583, 1692;
SEQ ID NOs: 696, 1255-1259, 1265, 1274-1277, 1283, 1330, and 1331.

Embodiment 5. The oligomeric compound of any of embodiments 1-4, wherein the nucleobase sequence of the modified oligonucleotide is at least 85%, at least 90%, at least 95%, or 100% complementary to an equal length portion of the DMPK nucleic acid.

Embodiment 6. An oligomeric compound, wherein the oligomeric compound comprises a modified oligonucleotide consisting of 12 to 30 linked nucleosides, wherein the nucleobase sequence of the modified oligonucleotide comprises at least 8, at least 9, at least 10, at least 11, at least 12, at least 13, at least 14, at least 15, or at least 16 contiguous nucleobases of the nucleobase sequences of any of SEQ ID NOs: 18-2334, and wherein the modified oligonucleotide has at least one modification selected from a modified sugar moiety and a modified internucleoside linkage.

Embodiment 7. An oligomeric compound, wherein the oligomeric compound comprises a modified oligonucleotide consisting of 12 to 30 linked nucleosides, wherein the nucleobase sequence of the modified oligonucleotide comprises at least 8, at least 9, at least 10, at least 11, at least 12, at least 13, at least 14, at least 15, at least 16, at least 17, at least 18, at least 19, or 20 contiguous nucleobases of the nucleobase sequences of any of SEQ ID NOs: 18-1264 or 1278-1329, and wherein the modified oligonucleotide has at least one modification selected from a modified sugar moiety and a modified internucleoside linkage.

Embodiment 8. The oligomeric compound of embodiment 6 or embodiment 7, wherein the modified oligonucleotide has a nucleobase sequence comprising the nucleobase sequence of any of SEQ ID NOs: 18-2334.

Embodiment 9. The oligomeric compound of embodiment 8, wherein the modified oligonucleotide has a nucleobase sequence consisting of the nucleobase sequence of any of SEQ ID NOs: 18-2334.

Embodiment 10. The oligomeric compound of any of embodiments 6-9, wherein the nucleobase sequence of the modified oligonucleotide is at least 85%, at least 90%, at least 95%, or 100% complementary to an equal length portion of a DMPK nucleic acid, wherein the DMPK nucleic acid has the nucleobase sequence of SEQ ID NO: 1 or SEQ ID NO: 2.

Embodiment 11. The oligomeric compound of any of embodiments 1-10, wherein the modified oligonucleotide consists of 12 to 20, 14 to 20, 15 to 20, 16 to 18, 16 to 20, 17 to 20, 18 to 20, or 18 to 22 linked nucleosides.

Embodiment 12. The oligomeric compound of any of embodiments 1-10, wherein the modified oligonucleotide consists of 16 linked nucleosides.

Embodiment 13. The oligomeric compound of any of embodiments 1-10, wherein the modified oligonucleotide consists of 18 linked nucleosides.

Embodiment 14. The oligomeric compound of any of embodiments 1-10, wherein the modified oligonucleotide consists of 20 linked nucleosides.

Embodiment 15. The oligomeric compound of any of embodiments 1-14, wherein at least one nucleoside of the modified oligonucleotide comprises a modified sugar moiety.

Embodiment 16. The oligomeric compound of embodiment 15, wherein the modified sugar moiety comprises a bicyclic sugar moiety.

Embodiment 17. The oligomeric compound of embodiment 16, wherein the bicyclic sugar moiety comprises a 2′-4′ bridge selected from —O—CH₂—; and —O—CH(CH₃)—.

Embodiment 18. The oligomeric compound of embodiment 15, wherein the modified sugar moiety comprises a non-bicyclic modified sugar moiety.

Embodiment 19. The oligomeric compound of embodiment 18, wherein the non-bicyclic modified sugar moiety is a 2′-MOE sugar moiety or 2′-OMe sugar moiety.

Embodiment 20. The oligomeric compound of any of embodiments 1-19, wherein at least one nucleoside of the modified oligonucleotide compound comprises a sugar surrogate.

Embodiment 21. The oligomeric compound of any of embodiments 1-20, wherein the modified oligonucleotide comprises at least one modified internucleoside linkage.

Embodiment 22. The oligomeric compound of embodiment 21, wherein at least one modified internucleoside linkage is a phosphorothioate internucleoside linkage.

Embodiment 23. The oligomeric compound of embodiment 21, wherein at least one modified internucleoside linkage is a mesyl phosphoramidate internucleoside linkage.

Embodiment 24. The oligomeric compound of any of embodiments 21-23, wherein each internucleoside linkage is a modified internucleoside linkage.

Embodiment 25. The oligomeric compound of embodiment 24, wherein each internucleoside linkage is a phosphorothioate internucleoside linkage.

Embodiment 26. The oligomeric compound of any of embodiments 21-23, wherein at least one internucleoside linkage of the modified oligonucleotide is a phosphodiester internucleoside linkage.

Embodiment 27. The oligomeric compound of any of embodiments 1-23 or 25-26, wherein each internucleoside linkage of the modified oligonucleotide is independently selected from a phosphodiester or a phosphorothioate internucleoside linkage.

Embodiment 28. The oligomeric compound of any of embodiments 1-23 or 25-26, wherein each internucleoside linkage of the modified oligonucleotide is independently selected from a phosphodiester internucleoside linkage, a phosphorothioate internucleoside linkage, or a mesyl phosphoramidate internucleoside linkage.

Embodiment 29. The oligomeric compound of any of embodiments 1-23 or 26-28, wherein at least 10, at least 11, at least 12, at least 13, at least 14, at least 15, at least 16, at least 17, or at least 18 internucleoside linkages of the modified oligonucleotide are phosphorothioate internucleoside linkages.

Embodiment 30. The oligomeric compound of any of embodiments 1-23 or 26-29, wherein at least 1, at least 2, at least 3, at least 4, or at least 5 internucleoside linkages of the modified oligonucleotide are mesyl phosphoramidate internucleoside linkages.

Embodiment 31. The oligomeric compound of embodiment 21, wherein the internucleoside linkage motif of the modified oligonucleotide is selected from soooossssssssssooss, sssssssssssssss, sooossssssssssoooss, soosssssssssoooss, sooosssssssssooss, sooooossssssssssoss, soooosssssssssoss, ssssxssssssssss, sssssssssssss, soossssssssssos, sosssssssssssos, soosxssssssssos, ooooxoooooooooo, sssssxsssssssss, soossxsssssssos, wherein each “s” represents a phosphorothioate internucleoside linkage, each “o” represents a phosphodiester internucleoside linkage, and each “x” represents a methoxypropyl phosphonate internucleoside linkage.

Embodiment 32. The oligomeric compound of any of embodiments 1-31, wherein the modified oligonucleotide comprises at least one modified nucleobase.

Embodiment 33. The oligomeric compound of embodiment 32, wherein the modified nucleobase is 5-methylcytosine.

Embodiment 34. The oligomeric compound of embodiment 33, wherein each cytosine is a 5-methylcytosine.

Embodiment 35. The oligomeric compound of any of embodiments 1-34, wherein the modified oligonucleotide comprises a deoxy region.

Embodiment 36. The oligomeric compound of embodiment 35, wherein each nucleoside of the deoxy region is a 2′-β-D-deoxynucleoside.

Embodiment 37. The oligomeric compound of embodiment 35 or embodiment 36, wherein the deoxy region consists of 6, 7, 8, 9, 10, or 6-10 linked nucleosides.

Embodiment 38. The oligomeric compound of any of embodiments 35-37, wherein each nucleoside immediately adjacent to the deoxy region comprises a modified sugar moiety.

Embodiment 39. The oligomeric compound of any of embodiments 35-37, wherein the deoxy region is flanked on the 5′-side by a 5′-external region consisting of 1-6 linked 5′-external region nucleosides and on the 3′-side by a 3′-external region consisting of 1-6 linked 3′-external region nucleosides; wherein
the 3′-most nucleoside of the 5′ external region comprises a modified sugar moiety; and
the 5′-most nucleoside of the 3′ external region comprises a modified sugar moiety.

Embodiment 40. The oligomeric compound of embodiment 39, wherein each nucleoside of the 3′ external region comprises a modified sugar moiety.

Embodiment 41. The oligomeric compound of embodiment 39 or embodiment 40, wherein each nucleoside of the 5′ external region comprises a modified sugar moiety.

Embodiment 42. The oligomeric compound of any of embodiments 39-41, wherein the modified oligonucleotide has:
a 5′ external region consisting of 5 linked nucleosides;
a deoxy region consisting of 10 linked nucleosides; and
a 3′ external region consisting of 5 linked nucleosides;
   wherein each of the 5′ external region nucleosides and each of the 3′ external region nucleosides is a 2′-MOE nucleoside.

Embodiment 43. The oligomeric compound of any of embodiments 39-41, wherein the modified oligonucleotide has:
a 5′ external region consisting of 6 linked nucleosides;
a deoxy region consisting of 10 linked nucleosides; and
a 3′ external region consisting of 4 linked nucleosides;
   wherein each of the 5′ external region nucleosides and each of the 3′ external region nucleosides is a 2′-MOE nucleoside.

Embodiment 44. The oligomeric compound of any of embodiments 39-41, wherein the modified oligonucleotide has:
a 5′ external region consisting of 4 linked nucleosides;
a deoxy region consisting of 10 linked nucleosides; and
a 3′ external region consisting of 6 linked nucleosides;
   wherein each of the 5′ external region nucleosides and each of the 3′ external region nucleosides is a 2′-MOE nucleoside.

Embodiment 45. The oligomeric compound of any of embodiments 39-41, wherein the modified oligonucleotide has:
a 5′ external region consisting of 3 linked nucleosides;
a deoxy region consisting of 10 linked nucleosides; and
a 3′ external region consisting of 3 linked nucleosides;
   wherein each of the 5′ external region nucleosides and each of the 3′ external region nucleosides is a cEt nucleoside.

Embodiment 46. The oligomeric compound of any of embodiments 39-41, wherein the modified oligonucleotide has:
a 5′ external region consisting of 1-6 linked nucleosides;
a deoxy region consisting of 6-10 linked nucleosides; and
a 3′ external region consisting of 1-6 linked nucleosides;
   wherein each of the 5′ external region nucleosides and each of the 3′ external region nucleosides is a cEt nucleoside or a 2′-MOE nucleoside; and each of the deoxy region nucleosides is a 2′-β-D-deoxynucleoside.

Embodiment 47. The oligomeric compound of any of embodiments 39-41, wherein the modified oligonucleotide has a sugar motif comprising:
a 5′ external region consisting of 3-6 linked nucleosides;
a deoxy region consisting of 7-8 linked nucleosides; and
a 3′ external region consisting of 3-6 linked nucleosides; wherein
each of the 3′ external region nucleosides is selected from a 2′-MOE nucleoside and a cEt nucleoside, and the 5′ external region has the following formula:

(Nk)n(Nd)(Nx)
wherein each Nk is a bicyclic nucleoside, Nx 2′-OMe nucleoside and Nd is a 2′-β-D-deoxynucleoside; and n is from 1-4.

Embodiment 48. An oligomeric compound of any of embodiments 1-38, wherein the modified oligonucleotide has a sugar motif (5′ to 3′) selected from: eeeeeddddddddddeeeee, kkkddddddddddkkk, eekkddddddddkkee, eeeeddddddddddeeeeee, eeeeddddddddeeeeee, eeeeeddddddddeeeee, eeeeeeddddddddddeeee, eeeeeeddddddddeeee, kkkedddddddddkkk, kkkdyddddddddkkk, kkeddddddddddkkk, kekddddddddddkkk, ekkddddddddddkke, kkddddddddddkk, ekkkddddddddkkke, ekkddddddddddkkk, kkkddddddddddkke, kkkdd[5′-(S)-Me-d]dddddddkkk, kkkdd[5′-(R)-Me-d]dddddddkkk, kkkdd[5′-(R)-allyl-d]dddddddkkk, kkkddd[5′-(R)-Me-d]ddddddkkk, wherein each “d” represents a 2′-β-D-deoxyribosyl sugar moiety, each “e” represents a 2′-MOE sugar moiety, each “y” represents a 2′-OMe sugar moiety, each “[5′-(S)-Me-d]” represents a 5′-(S)-methyl-β-D-2′-deoxyribosyl sugar moiety, each “[5′-(R)-Me-d]” represents a 5′-(R)-methyl-β-D-2′-deoxyribosyl sugar moiety, and each “[5′-(R)-allyl-d]” represents a 5′-(R)-allyl-β-D-2′-deoxyribosyl sugar moiety, and each “k” represents a cEt sugar moiety.

Embodiment 49. The oligomeric compound of any of embodiments 1-48, wherein the oligomeric compound comprises a conjugate group.

Embodiment 50. The oligomeric compound of embodiment 49, wherein the conjugate group comprises a conjugate linker and a conjugate moiety.

Embodiment 51. The oligomeric compound of embodiment 50, wherein the conjugate moiety is a lipophilic group.

Embodiment 52. The oligomeric compound of embodiment 50, wherein the conjugate moiety is selected from a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl.

Embodiment 53. The oligomeric compound of embodiment 50, wherein the conjugate moiety is a 6-palmitamidohexyl conjugate moiety.

Embodiment 54. The oligomeric compound of any of embodiments 50-53 wherein the conjugate linker is a phosphodiester linker.

Embodiment 55. The oligomeric compound of any one of embodiments 49-54, wherein the conjugate group has the following structure:

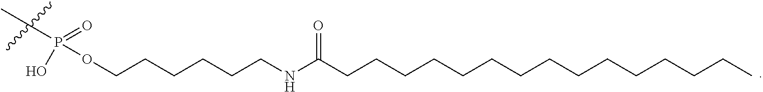 [SMILES: *P(=O)(O)OCCCCCCNC(=O)CCCCCCCCCCCCCCC]

Embodiment 56. The oligomeric compound of any of embodiments 50-54, wherein the conjugate linker consists of a single bond.

Embodiment 57. The oligomeric compound of any of embodiments 50-56, wherein the conjugate linker is cleavable.

Embodiment 58. The oligomeric compound of any of embodiments 50-57, wherein the conjugate linker comprises 1-3 linker-nucleosides.

Embodiment 59. The oligomeric compound of any of embodiments 50-58, wherein the conjugate linker does not comprise any linker nucleosides.

Embodiment 60. The oligomeric compound of any of embodiments 49-59, wherein the conjugate group is attached to the modified oligonucleotide at the 5′-end of the modified oligonucleotide.

Embodiment 61. The oligomeric compound of any of embodiments 49-59, wherein the conjugate group is attached to the modified oligonucleotide at the 3′-end of the modified oligonucleotide.

Embodiment 62. The oligomeric compound of any of embodiments 49-61, wherein the conjugate group comprises a cell-targeting moiety.

Embodiment 63. A population of oligomeric compounds of any of embodiments 1-62, wherein the population is chirally enriched for modified oligonucleotides comprising at least one particular phosphorothioate internucleoside linkage having a particular stereochemical configuration.

Embodiment 64. The population of embodiment 63, wherein the population is chirally enriched for modified oligonucleotides comprising at least one particular phosphorothioate internucleoside linkage having the (Sp) or (Rp) configuration.

Embodiment 65. The population of embodiment 63, wherein the population is chirally enriched for modified oligonucleotides having a particular, independently selected stereochemical configuration at each phosphorothioate internucleoside linkage.

Embodiment 66. The population of embodiment 63, wherein the population is chirally enriched for modified oligonucleotides having the (Rp) configuration at one particular phosphorothioate internucleoside linkage and the (Sp) configuration at each of the remaining phosphorothioate internucleoside linkages.

Embodiment 67. The population of embodiment 63, wherein the population is chirally enriched for modified oligonucleotides having at least 3 contiguous phosphorothioate internucleoside linkages in the Sp, Sp, and Rp configurations, in the 5′ to 3′ direction.

Embodiment 68. A population of oligomeric compounds of any of embodiments 1-63, wherein all of the phosphorothioate internucleoside linkages of the modified oligonucleotide are stereorandom.

Embodiment 69. An oligomeric duplex, comprising a first oligomeric compound and a second oligomeric compound comprising a second modified oligonucleotide, wherein the first oligomeric compound is an oligomeric compound of any of embodiments 1-63.

Embodiment 70. The oligomeric duplex of embodiment 69, wherein the second modified oligonucleotide consists of 8 to 80 linked nucleosides, and wherein the nucleobase sequence of the second modified oligonucleotide comprises a complementary region of at least 8 nucleobases that is at least 90% complementary to an equal length portion of the first modified oligonucleotide.

Embodiment 71. An antisense agent comprising an antisense compound, wherein the antisense compound is the oligomeric compound of any of embodiments 1-62.

Embodiment 72. The antisense agent of embodiment 71, wherein the antisense agent is an RNase H agent capable of reducing the amount of DMPK nucleic acid through activation of RNase H.

Embodiment 73. The antisense agent of any of embodiments 71-72, wherein the antisense agent comprises a conjugate group, wherein the conjugate group comprises a cell-targeting moiety.

Embodiment 74. A pharmaceutical composition comprising an oligomeric compound of any of embodiments 1-62, a population of any of embodiments 63-68, an oligomeric duplex of any of embodiments 69-70, or an antisense agent of any of embodiments 71-73, and a pharmaceutically acceptable diluent or carrier.

Embodiment 75. The pharmaceutical composition of embodiment 74, wherein the pharmaceutically acceptable diluent is phosphate-buffered saline or artificial cerebrospinal fluid.

Embodiment 76. The pharmaceutical composition of embodiment 75, wherein the pharmaceutical composition consists essentially of the oligomeric compound, the population, the oligomeric duplex, or the antisense agent, and phosphate-buffered saline or artificial cerebrospinal fluid.

Embodiment 77. A method comprising administering to a subject an oligomeric compound of any of embodiments 1-62, a population of any of embodiments 63-68, an oligomeric duplex of any of embodiments 69-70, an antisense agent of any of embodiments 71-73, or a pharmaceutical composition of any of embodiments 74-76.

Embodiment 78. A method of treating a disease associated with DMPK, comprising administering to a subject having a disease associated with DMPK a therapeutically effective amount of an oligomeric compound of any of embodiments 1-62, a population of any of embodiments 63-68, an oligomeric duplex of any of embodiments 69-70, an antisense agent of any of embodiments 71-73, or a pharmaceutical composition of any of embodiments 74-76; thereby treating the disease associated with DMPK.

Embodiment 79. The method of embodiment 78, wherein the disease associated with DMPK is type 1 myotonic dystrophy.

Embodiment 80. The method of any of embodiments 77-79, wherein administering the oligomeric compound of any of embodiments 1-62, population of any of embodiments 63-68, oligomeric duplex of any of embodiments 69-70, antisense agent of any of embodiments 71-73, or a pharmaceutical composition of any of embodiments 74-76 reduces one or more of muscle stiffness, myotonia, disabling distal weakness, weakness in face and jaw muscles, difficulty in swallowing, drooping of the eyelids (ptosis), weakness of neck muscles, weakness in arm and leg muscles, persistent muscle pain, hypersomnia, muscle wasting, dysphagia, respiratory insufficiency, irregular heartbeat, heart muscle damage, apathy, insulin resistance, and cataracts.

Embodiment 81. The method of any of embodiments 78-80, wherein the subject is human.

Embodiment 82. A method of reducing expression of DMPK in a cell comprising contacting the cell with an oligomeric compound of any of embodiments 1-62, a population of any of embodiments 63-68, an oligomeric duplex of any of embodiments 69-70, an antisense agent of any of embodiments 71-73, or a pharmaceutical composition of any of embodiments 74-76.

Embodiment 83. The method of embodiment 82, wherein the cell is a muscle cell or a neuron.

Embodiment 84. The method of embodiment 82 or embodiment 83, wherein the cell is a human cell.

Embodiment 85. Use of an oligomeric compound of any of embodiments 1-62, a population of any of embodiments 63-68, an oligomeric duplex of any of embodiments 69-70, an antisense agent of any of embodiments 71-73, or a pharmaceutical composition of any of embodiments 74-76 for treating a disease associated with DMPK.

Embodiment 86. Use of an oligomeric compound of any of embodiments 1-62, a population of any of embodiments 63-68, an oligomeric duplex of any of embodiments 69-70, an antisense agent of any of embodiments 71-73, or a pharmaceutical composition of any of embodiments 74-76 in the manufacture of a medicament for treating a disease associated with DMPK.

Embodiment 87. The use of embodiment 85 or embodiment 86, wherein the disease associated with DMPK is type 1 myotonic dystrophy.

### I. Certain Oligonucleotides

In certain embodiments, provided herein are oligomeric compounds comprising oligonucleotides, which consist of linked nucleosides. Oligonucleotides may be unmodified oligonucleotides (RNA or DNA) or may be modified oligonucleotides. Modified oligonucleotides comprise at least one modification relative to unmodified RNA or DNA. That is, modified oligonucleotides comprise at least one modified nucleoside (comprising a modified sugar moiety and/or a modified nucleobase) and/or at least one modified internucleoside linkage.

### A. Certain Modified Nucleosides

Modified nucleosides comprise a modified sugar moiety or a modified nucleobase or both a modified sugar moiety and a modified nucleobase. In certain embodiments, modified nucleosides comprising the following modified sugar moieties and/or the following modified nucleobases are incorporated into modified oligonucleotides.

### 1. Certain Sugar Moieties

In certain embodiments, modified sugar moieties are non-bicyclic modified sugar moieties comprising a furanosyl ring with one or more substituent groups none of which bridges two atoms of the furanosyl ring to form a bicyclic structure. Such non-bridging substituents may be at any position of the furanosyl, including but not limited to substituents at the 2′, 3′, 4′, and/or 5′ positions. Examples of 2′-substituent groups suitable for non-bicyclic modified sugar moieties include but are not limited to 2′-O(CH₂)₂OCH₃ (“MOE” or “O-methoxyethyl”).

In certain embodiments, modified furanosyl sugar moieties and nucleosides incorporating such modified furanosyl sugar moieties are further defined by isomeric configuration. For example, a 2′-deoxyfuranosyl sugar moiety may be in seven isomeric configurations other than the naturally occurring β-D-deoxyribosyl configuration. Such modified sugar moieties are described in, e.g., WO 2019/157531, incorporated by reference herein. A 2′-modified sugar moiety has an additional stereocenter at the 2′-position relative to a 2′-deoxyfuranosyl sugar moiety; therefore, such sugar moieties have a total of sixteen possible isomeric configurations. 2′-modified sugar moieties described herein are in the β-D-ribosyl isomeric configuration unless otherwise specified.

Certain modified sugar moieties comprise a substituent that bridges two atoms of the furanosyl ring to form a second ring, resulting in a bicyclic sugar moiety. In certain such embodiments, the bicyclic sugar moiety comprises a bridge between the 4′ and the 2′ furanose ring atoms. Examples of such 4′ to 2′ bridging sugar substituents include but are not limited to: 4′-CH2-2′, 4′-(CH2)2-2′, 4′-(CH2)3-2′, 4′-CH2-O-2′ (“LNA”), 4′-CH2-S-2′, 4′-(CH2)2-O-2′ (“ENA”), 4′-CH(CH3)—O-2′ (referred to as “constrained ethyl” or “cEt”), 4′-CH2-O—CH2-2′, 4′-CH2-N(R)-2′, 4′-C—H(CH2OCH3)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH3)(CH3)—O-2′ and analogs thereof (see, e.g., Seth et al., U.S. Pat. No. 8,278,283), 4′-CH2-N(OCH3)-2′ and analogs thereof (see, e.g., Prakash et al., U.S. Pat. No. 8,278,425), 4′-CH2-O—N(CH3)-2′ (see, e.g., Allerson et al., U.S. Pat. No. 7,696,345 and Allerson et al., U.S. Pat. No. 8,124,745), 4′-CH2-C—(H)(CH3)-2′ (see, e.g., Zhou, et al., J. Org. Chem., 2009, 74, 118-134), 4′-CH2-C—(═CH2)-2′ and analogs thereof (see e.g., Seth et al., U.S. Pat. No. 8,278,426), 4′ C(RaRb)—N(R)—O-2′, 4′-C(RaRb)—O—N(R)-2′, 4′-CH2-O—N(R)-2′, and 4′-CH2-N(R)—O-2′, wherein each R, Ra, and Rb is, independently, H, a protecting group, or C₁-C₁₂ alkyl (see, e.g. Imanishi et al., U.S. Pat. No. 7,427,672).

### 2. Certain Modified Nucleobases

In certain embodiments, modified oligonucleotides comprise one or more nucleosides comprising an unmodified nucleobase. In certain embodiments, modified oligonucleotides comprise one or more nucleosides comprising a modified nucleobase. Examples of modified nucleobases include 5-methylcytosine.

Publications that teach the preparation of certain modified nucleobases include without limitation, Manoharan et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., U.S. Pat. No. 6,166,199; and Matteucci et al., U.S. Pat. No. 6,005,096.

### 3. Certain Modified Internucleoside Linkages

The naturally occurring internucleoside linkage of RNA and DNA is a 3′ to 5′ phosphodiester linkage. In certain embodiments, nucleosides of modified oligonucleotides may be linked together using one or more modified internucleoside linkages. The two main classes of internucleoside linking groups are defined by the presence or absence of a phosphorus atom. Representative phosphorus-containing internucleoside linkages include but are not limited to phosphates, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS—P═S”). Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. In certain embodiments, internucleoside linkages having a chiral atom can be prepared as a racemic mixture, or as separate enantiomers. Methods of preparation of phosphorous-containing and non-phosphorous-containing internucleoside linkages are well known to those skilled in the art.

In certain embodiments, a modified internucleoside linkage is any of those described in WO/2021/030778, incorporated by reference herein. In certain embodiments, a modified internucleoside linkage comprises the formula:

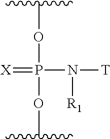

wherein independently for each internucleoside linking group of the modified oligonucleotide:
X is selected from O or S;
R₁ is selected from H, C₁-C₆ alkyl, and substituted C₁-C₆ alkyl; and
T is selected from SO₂R₂, C(═O)R₃, and P(═O)R₄R₅, wherein:
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group;
R₃ is selected from an aryl, a substituted aryl, CH₃, N(CH₃)₂, OCH₃ and a conjugate group;
R₄ is selected from OCH₃, OH, C₁-C₆ alkyl, substituted C₁-C₆ alkyl and a conjugate group; and
R₅ is selected from OCH₃, OH, C₁-C₆ alkyl, and substituted C₁-C₆ alkyl.

In certain embodiments, a modified internucleoside linkage comprises a mesyl phosphoramidate linking group having a formula:

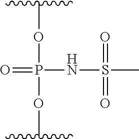 [SMILES: *OP(=O)(NS(C)(=O)=O)O*]

In certain embodiments, a mesyl phosphoramidate internucleoside linkage may comprise a chiral center. In certain embodiments, modified oligonucleotides comprising (Rp) and/or (Sp) mesyl phosphoramidates comprise one or more of the following formulas, respectively, wherein “B” indicates a nucleobase:

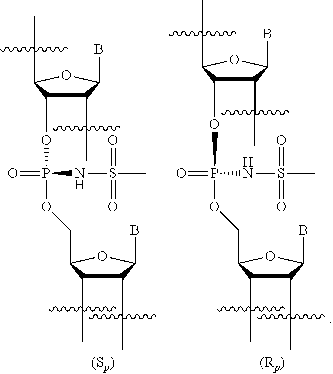

Representative internucleoside linkages having a chiral center include but are not limited to alkylphosphonates, mesyl phosphoramidates, and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate or other linkages containing chiral centers in particular stereochemical configurations. In certain embodiments, populations of modified oligonucleotides comprise phosphorothioate internucleoside linkages wherein all of the phosphorothioate internucleoside linkages are stereorandom. In certain embodiments, populations of modified oligonucleotides comprise mesyl phosphoramidate internucleoside linkages wherein all of the mesyl phosphoramidate internucleoside linkages are stereorandom. Such modified oligonucleotides can be generated using synthetic methods that result in random selection of the stereochemical configuration of each phosphorothioate or mesyl phosphoramidate linkage. Nonetheless, each individual phosphorothioate or mesyl phosphoramidate of each individual oligonucleotide molecule has a defined stereoconfiguration. In certain embodiments, populations of modified oligonucleotides are enriched for modified oligonucleotides comprising one or more particular phosphorothioate or mesyl phosphoramidate internucleoside linkages in a particular, independently selected stereochemical configuration. In certain embodiments, the particular configuration of the particular phosphorothioate or mesyl phosphoramidate linkage is present in at least 65% of the molecules in the population. In certain embodiments, the particular configuration of the particular phosphorothioate or mesyl phosphoramidate linkage is present in at least 70% of the molecules in the population. In certain embodiments, the particular configuration of the particular phosphorothioate or mesyl phosphoramidate linkage is present in at least 80% of the molecules in the population. In certain embodiments, the particular configuration of the particular phosphorothioate or mesyl phosphoramidate linkage is present in at least 90% of the molecules in the population. In certain embodiments, the particular configuration of the particular phosphorothioate or mesyl phosphoramidate linkage is present in at least 99% of the molecules in the population. Such chirally enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate or mesyl phosphoramidate in the (Sp) configuration. In certain embodiments, a population of modified oligonucleotides is enriched for modified oligonucleotides having at least one phosphorothioate or mesyl phosphoramidate in the (Rp) configuration. In certain embodiments, modified oligonucleotides comprising (Rp) and/or (Sp) phosphorothioates comprise one or more of the following formulas, respectively, wherein “B” indicates a nucleobase:

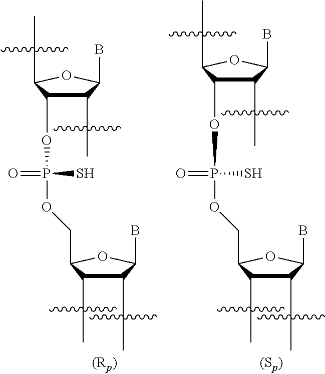

Unless otherwise indicated, chiral internucleoside linkages of modified oligonucleotides described herein can be stereorandom or in a particular stereochemical configuration.

Neutral internucleoside linkages include, without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), methoxypropyl (MOP), and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

In certain embodiments, modified oligonucleotides comprise one or more inverted nucleoside, as shown below:

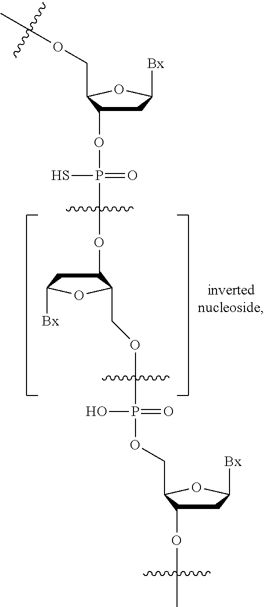

wherein each Bx independently represents any nucleobase.

In certain embodiments, an inverted nucleoside is terminal (i.e., the last nucleoside on one end of an oligonucleotide) and so only one internucleoside linkage depicted above will be present. In certain such embodiments, additional features (such as a conjugate group) may be attached to the inverted nucleoside. Such terminal inverted nucleosides can be attached to either or both ends of an oligonucleotide.

In certain embodiments, such groups lack a nucleobase and are referred to herein as inverted sugar moieties. In certain embodiments, an inverted sugar moiety is terminal (i.e., attached to the last nucleoside on one end of an oligonucleotide) and so only one internucleoside linkage above will be present. In certain such embodiments, additional features (such as a conjugate group) may be attached to the inverted sugar moiety. Such terminal inverted sugar moieties can be attached to either or both ends of an oligonucleotide.

In certain embodiments, nucleic acids can be linked 2′ to 5′ rather than the standard 3′ to 5′ linkage. Such a linkage is illustrated below.

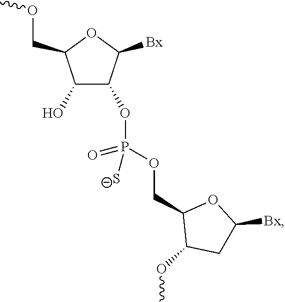

wherein each Bx represents any nucleobase.

### B. Certain Motifs

In certain embodiments, modified oligonucleotides comprise one or more modified nucleosides comprising a modified sugar moiety. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

### 1. Certain Sugar Motifs

In certain embodiments, oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

### Gapmer Oligonucleotides

In certain embodiments, modified oligonucleotides comprise or consist of a region having a gapmer motif, which is defined by two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer comprise 1-6 nucleosides. In certain embodiments, each nucleoside of each wing of a gapmer comprises a modified sugar moiety. In certain embodiments, at least one nucleoside of each wing of a gapmer comprises a modified sugar moiety. In certain embodiments, at least two nucleosides of each wing of a gapmer comprises a modified sugar moiety. In certain embodiments, at least three nucleosides of each wing of a gapmer comprises a modified sugar moiety. In certain embodiments, at least four nucleosides of each wing of a gapmer comprises a modified sugar moiety.

In certain embodiments, the gap of a gapmer comprises 7-12 nucleosides. In certain embodiments, each nucleoside of the gap of a gapmer comprises a 2′-β-D-deoxyribosyl sugar moiety. In certain embodiments, at least one nucleoside of the gap of a gapmer comprises a modified sugar moiety.

In certain embodiments, the gapmer is a deoxy gapmer. In certain embodiments, the nucleosides on the gap side of each wing/gap junction comprise 2′-deoxyribosyl sugar moieties and the nucleosides on the wing sides of each wing/gap junction comprise modified sugar moieties. In certain embodiments, each nucleoside of the gap comprises a 2′-β-D-deoxyribosyl sugar moiety. In certain embodiments, each nucleoside of each wing of a gapmer comprises a modified sugar moiety. In certain embodiments, at least one nucleoside of the gap of a gapmer comprises a modified sugar moiety. In certain embodiments, one nucleoside of the gap comprises a modified sugar moiety and each remaining nucleoside of the gap comprises a 2′-deoxyribosyl sugar moiety. In certain embodiments, at least one nucleoside of the gap of a gapmer comprises a 2′-OMe sugar moiety.

Herein, the lengths (number of nucleosides) of the three regions of a gapmer may be provided using the notation [# of nucleosides in the 5′-wing]−[# of nucleosides in the gap]−[# of nucleosides in the 3′-wing]. Thus, a 3-10-3 gapmer consists of 3 linked nucleosides in each wing and 10 linked nucleosides in the gap. Where such nomenclature is followed by a specific modification, that modification is the modification in each sugar moiety of each wing and the gap nucleosides comprise 2′-β-D-deoxyribosyl sugar moieties. Thus, a 5-10-5 MOE gapmer consists of 5 linked 2′-MOE nucleosides in the 5′-wing, 10 linked 2′-β-D-deoxynucleosides in the gap, and 5 linked 2′-MOE nucleosides in the 3′-wing. A 6-10-4 MOE gapmer consists of 6 linked 2′-MOE nucleosides in the 5′-wing, 10 linked 2′-β-D-deoxynucleosides in the gap, and 4 linked 2′-MOE nucleosides in the 3′-wing. A 3-10-3 cEt gapmer consists of 3 linked cEt nucleosides in the 5′-wing, 10 linked 2′-β-D-deoxynucleosides in the gap, and 3 linked cEt nucleosides in the 3′-wing. In certain embodiments, modified oligonucleotides are 5-10-5 MOE gapmers. In certain embodiments, modified oligonucleotides are 6-10-4 MOE gapmers. In certain embodiments, modified oligonucleotides are 5-10-5 cEt gapmers.

In certain embodiments, the modified oligonucleotide has a sugar motif (5′ to 3′) selected from: eeeeeddddddddddeeeee, kkkddddddddddkkk, eekkddddddddkkee, eeeeddddddddddeeeeee, eeeeddddddddeeeeee, eeeeeddddddddeeeee, eeeeeeddddddddddeeee, eeeeeeddddddddeeee, kkkedddddddddkkk, kkkdyddddddddkkk, kkeddddddddddkkk, kekddddddddddkkk, ekkddddddddddkke, kkddddddddddkk, ekkkddddddddkkke, ekkddddddddddkkk, kkkddddddddddkke, kkkdd[5′-(S)-Me-d]dddddddkkk, kkkdd[5′-(R)-Me-d]dddddddkkk, kkkdd[5′-(R)-allyl-d]dddddddkkk, kkkddd[5′-(R)-Me-d]ddddddkkk, wherein each “d” represents a 2′-β-D-deoxyribosyl sugar moiety, each “e” represents a 2′-MOE sugar moiety, each “y” represents a 2′-OMe sugar moiety, each “[5′-(S)-Me-d]” represents a 5′-(S)-methyl-β-D-2′-deoxyribosyl sugar moiety, each “[5′-(R)-Me-d]” represents a 5′-(R)-methyl-β-D-2′-deoxyribosyl sugar moiety, and each “[5′-(R)-allyl-d]” represents a 5′-(R)-allyl-β-D-2′-deoxyribosyl sugar moiety, and each “k” represents a cEt sugar moiety.

In certain embodiments, modified oligonucleotides have a sugar motif selected from 5′ to 3′: eeeeeddddddddddeeeee; wherein each “d” represents a 2′-β-D-deoxyribosyl sugar moiety, and each “e” represents a 2′-MOE sugar moiety.

In certain embodiments, modified oligonucleotides have a sugar motif selected from 5′ to 3′: eeeeeeddddddddddeeee; wherein each “d” represents a 2′-β-D-deoxyribosyl sugar moiety, and each “e” represents a 2′-MOE sugar moiety.

In certain embodiments, modified oligonucleotides have the sugar motif from 5′ to 3′: kkkddddddddddkkk; wherein each “d” represents a 2′-β-D-deoxyribosyl sugar moiety, and each “k” represents a cEt sugar moiety.

### 2. Certain Nucleobase Motifs

In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each cytosine is modified. In certain embodiments, some or all of the cytosine nucleobases in a modified oligonucleotide are 5-methylcytosines. In certain embodiments, all of the cytosine nucleobases are 5-methylcytosines and all of the other nucleobases of the modified oligonucleotide are unmodified nucleobases.

In certain embodiments, oligonucleotides having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of said nucleoside is a 2′-deoxyribosyl sugar moiety.

### 3. Certain Internucleoside Linkage Motifs

In certain embodiments, oligonucleotides comprise modified and/or unmodified internucleoside linkages arranged along the oligonucleotide or region thereof in a defined pattern or motif. In certain embodiments, each internucleoside linking group is a phosphodiester internucleoside linkage (P═O). In certain embodiments, each internucleoside linking group of a modified oligonucleotide is a phosphorothioate internucleoside linkage (P═S). In certain embodiments, each internucleoside linkage of a modified oligonucleotide is independently selected from a phosphorothioate internucleoside linkage and phosphodiester internucleoside linkage. In certain embodiments, each phosphorothioate internucleoside linkage is independently selected from a stereorandom phosphorothioate, a (Sp) phosphorothioate, and a (Rp) phosphorothioate.

In certain embodiments, the sugar motif of a modified oligonucleotide is a gapmer and the internucleoside linkages within the gap are all modified. In certain such embodiments, some, or all of the internucleoside linkages in the wings are unmodified phosphodiester internucleoside linkages. In certain embodiments, the terminal internucleoside linkages are modified. In certain embodiments, the sugar motif of a modified oligonucleotide is a gapmer, and the internucleoside linkage motif comprises at least one phosphodiester internucleoside linkage in at least one wing, wherein the at least one phosphodiester linkage is not a terminal internucleoside linkage, and the remaining internucleoside linkages are phosphorothioate internucleoside linkages. In certain such embodiments, all of the phosphorothioate linkages are stereorandom. In certain embodiments, all of the phosphorothioate linkages in the wings are (Sp) phosphorothioates, and the gap comprises at least one Sp, Sp, Rp motif. In certain embodiments, populations of modified oligonucleotides are enriched for modified oligonucleotides comprising such internucleoside linkage motifs.

In certain embodiments, the internucleoside linkage motif of the modified oligonucleotide is selected from soooossssssssssooss, sssssssssssssss, sooossssssssssoooss, soosssssssssoooss, sooosssssssssooss, sooooossssssssssoss, soooosssssssssoss, ssssxssssssssss, sssssssssssss, soossssssssssos, sosssssssssssos, soosxssssssssos, ooooxoooooooooo, sssssxsssssssss, soossxsssssssos, wherein each “s” represents a phosphorothioate internucleoside linkage, each “o” represents a phosphodiester internucleoside linkage, and each “x” represents a methoxypropyl phosphonate internucleoside linkage. In certain embodiments, modified oligonucleotides have an internucleoside linkage motif of (5′ to 3′): sooosssssssssssooss phosphorothioate internucleoside linkage. In certain embodiments, modified oligonucleotides have an internucleoside linkage motif of (5′ to 3′): sooooossssssssssoss, wherein each “s” represents a phosphorothioate internucleoside linkage and each “o” represents a phosphodiester internucleoside linkage.

### C. Certain Lengths

It is possible to increase or decrease the length of an oligonucleotide without eliminating activity. For example, in Woolf et al., Proc. Natl. Acad. Sci. USA, 1992, 89, 7305-7309, 1992), a series of oligonucleotides 13-25 nucleobases in length were tested for their ability to induce cleavage of a target nucleic acid in an oocyte injection model. Oligonucleotides 25 nucleobases in length with 8 or 11 mismatch bases near the ends of the oligonucleotides were able to direct specific cleavage of the target nucleic acid, albeit to a lesser extent than the oligonucleotides that contained no mismatches. Similarly, target specific cleavage was achieved using 13 nucleobase oligonucleotides, including those with 1 or 3 mismatches.

In certain embodiments, oligonucleotides (including modified oligonucleotides) can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 27, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

In certain embodiments, oligonucleotides consist of 16 linked nucleosides. In certain embodiments, oligonucleotides consist of 17 linked nucleosides. In certain embodiments, oligonucleotides consist of 18 linked nucleosides. In certain embodiments, oligonucleotides consist of 19 linked nucleosides. In certain embodiments, oligonucleotides consist of 20 linked nucleosides.

### D. Certain Modified Oligonucleotides

In certain embodiments, the above modifications (sugar, nucleobase, internucleoside linkage) are incorporated into a modified oligonucleotide. In certain embodiments, modified oligonucleotides are characterized by their modification motifs and overall lengths. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. For example, the internucleoside linkages within the wing regions of a sugar gapmer may be the same or different from one another and may be the same or different from the internucleoside linkages of the gap region of the sugar motif. Likewise, such sugar gapmer oligonucleotides may comprise one or more modified nucleobase independent of the gapmer pattern of the sugar modifications. Unless otherwise indicated, all modifications are independent of nucleobase sequence.

### E. Certain Populations of Modified Oligonucleotides

Populations of modified oligonucleotides in which all of the modified oligonucleotides of the population have the same molecular formula can be stereorandom populations or chirally enriched populations. All of the chiral centers of all of the modified oligonucleotides are stereorandom in a stereorandom population. In a chirally enriched population, at least one particular chiral center is not stereorandom in the modified oligonucleotides of the population. In certain embodiments, the modified oligonucleotides of a chirally enriched population are enriched for β-D ribosyl sugar moieties, and all of the phosphorothioate internucleoside linkages are stereorandom. In certain embodiments, the modified oligonucleotides of a chirally enriched population are enriched for both β-D ribosyl sugar moieties and at least one, particular phosphorothioate internucleoside linkage in a particular stereochemical configuration.

### F. Nucleobase Sequence

In certain embodiments, oligonucleotides (unmodified or modified oligonucleotides) are further described by their nucleobase sequence. In certain embodiments oligonucleotides have a nucleobase sequence that is complementary to a second oligonucleotide or an identified reference nucleic acid, such as a target nucleic acid. In certain such embodiments, a portion of an oligonucleotide has a nucleobase sequence that is complementary to a second oligonucleotide or an identified reference nucleic acid, such as a target nucleic acid. In certain embodiments, the nucleobase sequence of a portion or entire length of an oligonucleotide is at least 50%, at least 60%, at least 70%, at least 80%, at least 85%, at least 90%, at least 95%, or 100% complementary to the second oligonucleotide or nucleic acid, such as a target nucleic acid.

### II. Certain Oligomeric Compounds

In certain embodiments, provided herein are oligomeric compounds, which consist of an oligonucleotide (modified or unmodified) and optionally one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, modified or unmodified nucleosides, and two or more nucleosides that are independently modified or unmodified.

### A. Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance.

In certain embodiments, conjugation of one or more carbohydrate moieties to a modified oligonucleotide can optimize one or more properties of the modified oligonucleotide. In certain embodiments, the carbohydrate moiety is attached to a modified subunit of the modified oligonucleotide. For example, the ribose sugar of one or more ribonucleotide subunits of a modified oligonucleotide can be replaced with another moiety, e.g. a non-carbohydrate (preferably cyclic) carrier to which is attached a carbohydrate ligand. A ribonucleotide subunit in which the ribose sugar of the subunit has been so replaced is referred to herein as a ribose replacement modification subunit (RRMS), which is a modified sugar moiety. A cyclic carrier may be a carbocyclic ring system, i.e., one or more ring atoms may be a heteroatom, e.g., nitrogen, oxygen, sulphur. The cyclic carrier may be a monocyclic ring system, or may contain two or more rings, e.g. fused rings. The cyclic carrier may be a fully saturated ring system, or it may contain one or more double bonds. In certain embodiments, the modified oligonucleotide is a gapmer.

In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N. Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

In certain embodiments, the conjugate group may comprise a conjugate moiety selected from any of a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl.

In certain embodiments, the conjugate group may comprise a conjugate moiety selected from any of a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, or C5 alkyl, where the alkyl chain has one or more unsaturated bonds.

In certain embodiments, a conjugate group is a lipid having the following structure:

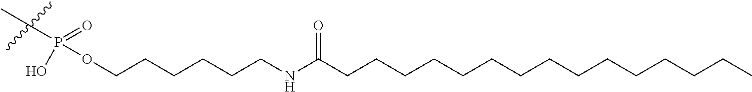 [SMILES: *P(=O)(O)OCCCCCCNC(=O)CCCCCCCCCCCCCCC]

### 1. Conjugate Moieties

Conjugate moieties include, without limitation, intercalators, reporter molecules, polyamines, polyamides, peptides, carbohydrates (e.g., GalNAc), vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

### 2. Coniugate Linkers

Conjugate moieties are attached to oligonucleotides through conjugate linkers. In certain oligomeric compounds, the conjugate linker is a single chemical bond (i.e., the conjugate moiety is attached directly to an oligonucleotide through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises pyrrolidine.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate moieties to compounds, such as the oligonucleotides provided herein. In general, a bifunctional linking moiety comprises at least two functional groups. One of the functional groups is selected to react with a particular site on a compound and the other is selected to react with a conjugate moiety. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise exactly 3 linker-nucleosides. In certain embodiments, conjugate linkers comprise the TCA motif. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such an oligomeric compound is more than 30. Alternatively, an oligomeric compound may comprise a modified oligonucleotide consisting of 8-30 nucleosides and no conjugate group. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxynucleoside that is attached to either the 3′ or 5-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxyadenosine.

### 3. Cell-Targeting Moieties

In certain embodiments, a conjugate group comprises a cell-targeting moiety. In certain embodiments, a conjugate group has the general formula:

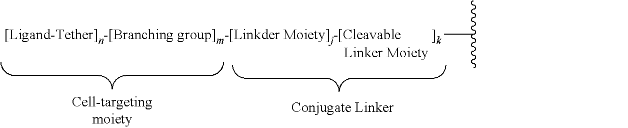

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group.

In certain embodiments, each ligand of a cell-targeting moiety has an affinity for at least one type of receptor on a target cell. In certain embodiments, each ligand has an affinity for at least one type of receptor on the surface of a mammalian liver cell. In certain embodiments, each ligand has an affinity for the hepatic asialoglycoprotein receptor (ASGP-R). In certain embodiments, each ligand is a carbohydrate.

In certain embodiments, a conjugate group comprises a cell-targeting conjugate moiety. In certain embodiments, a conjugate group has the general formula:

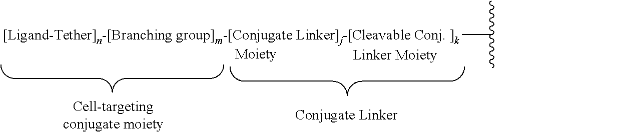

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group. In certain embodiments, cell-targeting moieties comprise three tethered ligands covalently attached to a branching group.

### B. Certain Terminal Groups

In certain embodiments, oligomeric compounds comprise one or more terminal groups. In certain such embodiments, oligomeric compounds comprise a stabilized 5′-phosphate. Stabilized 5′-phosphates include, but are not limited to 5′-phosphonates, including, but not limited to 5′-vinylphosphonates. In certain embodiments, terminal groups comprise one or more abasic sugar moieties and/or inverted nucleosides. In certain embodiments, terminal groups comprise one or more 2′-linked nucleosides or sugar moieties. In certain such embodiments, the 2′-linked group is an abasic sugar moiety.

### III. Antisense Activity

In certain embodiments, oligomeric compounds and oligomeric duplexes are capable of hybridizing to a target nucleic acid, resulting in at least one antisense activity; such oligomeric compounds and oligomeric duplexes are antisense compounds. In certain embodiments, antisense compounds have antisense activity when they reduce or inhibit the amount or activity of a target nucleic acid by 25% or more in the standard cell assay. In certain embodiments, antisense compounds selectively affect one or more target nucleic acid. Such antisense compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in significant undesired antisense activity.

In certain antisense activities, hybridization of an antisense compound to a target nucleic acid results in recruitment of a protein that cleaves the target nucleic acid. For example, certain antisense compounds result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, described herein are antisense compounds that are sufficiently “DNA-like” to elicit RNase H activity. In certain embodiments, one or more non-DNA-like nucleoside in the gap of a gapmer is tolerated.

In certain antisense activities, an antisense compound or a portion of an antisense compound is loaded into an RNA-induced silencing complex (RISC), ultimately resulting in cleavage of the target nucleic acid. For example, certain antisense compounds result in cleavage of the target nucleic acid by Argonaute. Antisense compounds that are loaded into RISC are RNAi compounds. RNAi compounds may be double-stranded (siRNA or dsRNAi) or single-stranded (ssRNA).

In certain embodiments, hybridization of an antisense compound to a target nucleic acid does not result in recruitment of a protein that cleaves that target nucleic acid. In certain embodiments, hybridization of the antisense compound to the target nucleic acid results in alteration of splicing of the target nucleic acid. In certain embodiments, hybridization of an antisense compound to a target nucleic acid results in inhibition of a binding interaction between the target nucleic acid and a protein or other nucleic acid. In certain embodiments, hybridization of an antisense compound to a target nucleic acid results in alteration of translation of the target nucleic acid.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein and/or a phenotypic change in a cell or animal.

### IV. Certain Target Nucleic Acids

In certain embodiments, oligomeric compounds comprise or consist of an oligonucleotide comprising a region that is complementary to a target nucleic acid. In certain embodiments, the target nucleic acid is an endogenous RNA molecule. In certain embodiments, the target nucleic acid encodes a protein. In certain such embodiments, the target nucleic acid is selected from: a mature mRNA and a pre-mRNA, including intronic, exonic and untranslated regions. In certain embodiments, the target RNA is a mature mRNA. In certain embodiments, the target nucleic acid is a pre-mRNA. In certain embodiments, the target region is entirely within an intron. In certain embodiments, the target region spans an intron/exon junction. In certain embodiments, the target region is at least 50% within an intron.

### A. Complementarity/Mismatches to the Target Nucleic Acid

In certain embodiments, oligonucleotides are complementary to the target nucleic acid over the entire length of the oligonucleotide. In certain embodiments, oligonucleotides are 99%, 95%, 90%, 85%, or 80% complementary to the target nucleic acid. In certain embodiments, oligonucleotides are at least 80% complementary to the target nucleic acid over the entire length of the oligonucleotide and comprise a region that is 100% or fully complementary to a target nucleic acid. In certain embodiments, the region of full complementarity is from 6 to 20, 10 to 18, or 18 to 20 nucleobases in length.

It is possible to introduce mismatch bases without eliminating activity. For example, Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo. Maher and Dolnick (Nuc. Acid. Res. 16:3341-3358, 1988) tested a series of tandem 14 nucleobase oligonucleotides, and a 28 and 42 nucleobase oligonucleotides comprised of the sequence of two or three of the tandem oligonucleotides, respectively, for their ability to arrest translation of human DHFR in a rabbit reticulocyte assay. Each of the three 14 nucleobase oligonucleotides alone was able to inhibit translation, albeit at a more modest level than the 28 or 42 nucleobase oligonucleotides.

In certain embodiments, oligonucleotides are complementary to the target nucleic acid over the entire length of the oligonucleotide. In certain embodiments, oligonucleotides are 99%, 95%, 90%, 85%, or 80% complementary to the target nucleic acid. In certain embodiments, oligonucleotides are at least 80% complementary to the target nucleic acid over the entire length of the oligonucleotide and comprise a portion that is 100% or fully complementary to a target nucleic acid. In certain embodiments, the portion of full complementarity is 6, 7, 8, 9, 10, 11, 12, 13, 14, 15, 16, 17, 18, 19, 20, 21, 22, 23, or 24 nucleobases in length.

### B. DMPK

In certain embodiments, oligomeric compounds comprise or consist of an oligonucleotide comprising a region that is complementary to a target nucleic acid, wherein the target nucleic acid is a DMPK nucleic acid. In certain embodiments, a DMPK nucleic acid has the nucleobase sequence set forth in SEQ ID NO: 1 (the complement of GENBANK Accession No. NT_011109.16, truncated from nucleotides 18539000 to 18566000), SEQ ID NO: 2 (GENBANK Accession No. NM_004409.4). In certain embodiments, a DMPK nucleic acid has the nucleobase sequence set forth in SEQ ID NO: 3 (the complement of GENBANK Accession No. NC_000019.10, truncated from nucleosides 45767001 to 45786000), SEQ ID NO: 4 (GENBANK Accession No. NM_001288764.1), and/or SEQ ID NO: 5 (GENBANK Accession No. NM_001081560.2).

In certain embodiments, contacting a cell with an oligomeric compound complementary to SEQ ID NO: 1 or SEQ ID NO: 2 reduces the amount of DMPK RNA, and in certain embodiments reduces the amount of DMPK protein. In certain embodiments, contacting a cell with an oligomeric compound complementary to SEQ ID NO: 3, SEQ ID NO: 4, and/or SEQ ID NO: 5 reduces the amount of DMPK RNA, and in certain embodiments reduces the amount of DMPK protein. In certain embodiments, the oligomeric compound consists of a modified oligonucleotide. In certain embodiments, the oligomeric compound consists of a modified oligonucleotide and a conjugate group.

In certain embodiments, an oligomeric compound complementary to any one of SEQ ID NOs: 1-5 is capable of reducing the amount of DMPK RNA in vitro by at least 10%, at least 20%, at least 30%, at least 40%, at least 50%, at least 60%, at least 70%, at least 80%, or at least 90% when administered according to the standard cell assay. In certain embodiments, an oligomeric compound complementary to any one of SEQ ID NOs: 1-5 is capable of reducing the amount of DMPK protein in vitro by at least 10%, at least 20%, at least 30%, at least 40%, at least 50%, at least 60%, at least 70%, at least 80%, or at least 90% when administered according to the standard cell assay. In certain embodiments, an oligomeric compound complementary to any one of SEQ ID NOs: 1-5 is capable of reducing the amount of DMPK in the CSF of a subject by at least 10%, at least 20%, at least 30%, at least 40%, at least 50%, at least 60%, at least 70%, at least 80%, or at least 90%. In certain embodiments, an oligomeric compound complementary to any one of SEQ ID NOs: 1-5 is capable of reducing the amount of DMPK protein in the CSF of a subject by at least 10%, at least 20%, at least 30%, at least 40%, at least 50%, at least 60%, at least 70%, at least 80%, or at least 90%. In certain embodiments, an oligomeric compound complementary to any one of SEQ ID NOs: 1-5 is capable of reducing the amount of DMPK in the muscle tissue of a subject by at least 10%, at least 20%, at least 30%, at least 40%, at least 50%, at least 60%, at least 70%, at least 80%, or at least 90%. In certain embodiments, an oligomeric compound complementary to any one of SEQ ID NOs: 1-5 is capable of reducing the amount of DMPK protein in the muscle tissue of a subject by at least 10%, at least 20%, at least 30%, at least 40%, at least 50%, at least 60%, at least 70%, at least 80%, or at least 90%.

### C. Certain Target Nucleic Acids in Certain Tissues

In certain embodiments, oligomeric compounds comprise or consist of an oligonucleotide comprising a region that is complementary to a target nucleic acid, wherein the target nucleic acid is expressed in a pharmacologically relevant tissue. In certain embodiments, the pharmacologically relevant tissues are muscle tissues, such as heart, diaphragm, tibialis anterior, gastrocnemius, and quadriceps muscles. In certain embodiments, the target nucleic acid is expressed in a pharmacologically relevant cell. In certain embodiments the pharmacologically relevant cell is a muscle cell. In some embodiments the muscle cell is a skeletal muscle cell. In some embodiments, the skeletal muscle cell is tibialis anterior, gastrocnemius, or quadriceps.

In certain embodiments, the pharmacologically relevant tissues are tissues of the CNS. In some embodiments, the tissue is selected from cortex and hippocampus.

### V. Certain Pharmaceutical Compositions

In certain embodiments, described herein are pharmaceutical compositions comprising one or more oligomeric compounds. In certain embodiments, the one or more oligomeric compounds each consists of a modified oligonucleotide. In certain embodiments, the pharmaceutical composition comprises a pharmaceutically acceptable diluent or carrier. In certain embodiments, a pharmaceutical composition comprises or consists of a sterile saline solution and one or more oligomeric compound. In certain embodiments, the sterile saline is pharmaceutical grade saline. In certain embodiments, a pharmaceutical composition comprises or consists of one or more oligomeric compound and sterile water. In certain embodiments, the sterile water is pharmaceutical grade water. In certain embodiments, a pharmaceutical composition comprises or consists of one or more oligomeric compound and phosphate-buffered saline (PBS). In certain embodiments, the sterile PBS is pharmaceutical grade PBS. In certain embodiments, a pharmaceutical composition comprises or consists of one or more oligomeric compound and artificial cerebrospinal fluid (“artificial CSF” or “aCSF”). In certain embodiments, the artificial cerebrospinal fluid is pharmaceutical grade artificial cerebrospinal fluid.

In certain embodiments, a pharmaceutical composition comprises a modified oligonucleotide and PBS. In certain embodiments, a pharmaceutical composition consists of a modified oligonucleotide and PBS. In certain embodiments, a pharmaceutical composition consists essentially of a modified oligonucleotide and PBS. In certain embodiments, the PBS is pharmaceutical grade.

In certain embodiments, a pharmaceutical composition comprises a modified oligonucleotide and artificial cerebrospinal fluid. In certain embodiments, a pharmaceutical composition consists of a modified oligonucleotide and artificial cerebrospinal fluid. In certain embodiments, a pharmaceutical composition consists essentially of a modified oligonucleotide and artificial cerebrospinal fluid. In certain embodiments, the artificial cerebrospinal fluid is pharmaceutical grade. In certain embodiments, aCSF comprises sodium chloride, potassium chloride, sodium dihydrogen phosphate dihydrate, sodium phosphate dibasic anhydrous, calcium chloride dihydrate, and magnesium chloride hexahydrate. In certain embodiments, the pH of an aCSF solution is modulated with a suitable pH-adjusting agent, for example, with acids such as hydrochloric acid and alkalis such as sodium hydroxide, to a range of from about 7.1-7.3, or to about 7.2.

In certain embodiments, pharmaceutical compositions comprise one or more oligomeric compound and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, oligomeric compounds may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions comprising an oligomeric compound encompass any pharmaceutically acceptable salts of the oligomeric compound, esters of the oligomeric compound, or salts of such esters. In certain embodiments, pharmaceutical compositions comprising oligomeric compounds comprising one or more oligonucleotide, upon administration to an animal, including a human, are capable of providing (directly or indirectly) the biologically active metabolite or residue thereof. Accordingly, for example, the disclosure is also drawn to pharmaceutically acceptable salts of oligomeric compounds, prodrugs, pharmaceutically acceptable salts of such prodrugs, and other bioequivalents. Suitable pharmaceutically acceptable salts include, but are not limited to, sodium and potassium salts. In certain embodiments, prodrugs comprise one or more conjugate group attached to an oligonucleotide, wherein the conjugate group is cleaved by endogenous nucleases within the body.

Lipid moieties have been used in nucleic acid therapies in a variety of methods. In certain such methods, the nucleic acid, such as an oligomeric compound, is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions comprise a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver the one or more pharmaceutical agents of the present invention to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, intrathecal (IT), intracerebroventricular (ICV), etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

Under certain conditions, certain compounds disclosed herein act as acids. Although such compounds may be drawn or described in protonated (free acid) form, or ionized and in association with a cation (salt) form, aqueous solutions of such compounds exist in equilibrium among such forms. For example, a phosphate linkage of an oligonucleotide in aqueous solution exists in equilibrium among free acid, anion and salt forms. Unless otherwise indicated, compounds described herein are intended to include all such forms. Moreover, certain oligonucleotides have several such linkages, each of which is in equilibrium. Thus, oligonucleotides in solution exist in an ensemble of forms at multiple positions all at equilibrium. The term “oligonucleotide” is intended to include all such forms. Drawn structures necessarily depict a single form. Nevertheless, unless otherwise indicated, such drawings are likewise intended to include corresponding forms. Herein, a structure depicting the free acid of a compound followed by the term “or a salt thereof” expressly includes all such forms that may be fully or partially protonated/de-protonated/in association with a cation. In certain instances, one or more specific cation is identified.

In certain embodiments, modified oligonucleotides or oligomeric compounds are in aqueous solution with sodium. In certain embodiments, modified oligonucleotides or oligomeric compounds are in aqueous solution with potassium. In certain embodiments, modified oligonucleotides or oligomeric compounds are in PBS. In certain embodiments, modified oligonucleotides or oligomeric compounds are in water. In certain such embodiments, the pH of the solution is adjusted with NaOH and/or HCl to achieve a desired pH.

Herein, certain specific doses are described. A dose may be in the form of a dosage unit. For clarity, a dose (or dosage unit) of a modified oligonucleotide or an oligomeric compound in milligrams indicates the mass of the free acid form of the modified oligonucleotide or oligomeric compound. As described above, in aqueous solution, the free acid is in equilibrium with anionic and salt forms. However, for the purpose of calculating dose, it is assumed that the modified oligonucleotide or oligomeric compound exists as a solvent-free, sodium-acetate free, anhydrous, free acid. For example, where a modified oligonucleotide or an oligomeric compound is in solution comprising sodium (e.g., saline), the modified oligonucleotide or oligomeric compound may be partially or fully de-protonated and in association with Na+ ions. However, the mass of the protons is nevertheless counted toward the weight of the dose, and the mass of the Na+ ions is not counted toward the weight of the dose. Thus, for example, a dose, or dosage unit, of 10 mg of Compound No. 598769, equals the number of fully protonated molecules that weighs 10 mg. This would be equivalent to 10.59 mg of solvent-free, sodium acetate-free, anhydrous sodiated Compound No. 598769. When an oligomeric compound comprises a conjugate group, the mass of the conjugate group is included in calculating the dose of such oligomeric compound. If the conjugate group also has an acid, the conjugate group is likewise assumed to be fully protonated for the purpose of calculating dose.

### IV. Certain Hotspot Regions

In certain embodiments, nucleobases in the ranges specified below comprise a hotspot region of DMPK nucleic acid.

### 1. Nucleobases 19888-19942 of SEO ID NO:1

In certain embodiments, nucleobases 19888-19942 of SEQ ID NO: 1 comprise a hotspot region. In certain embodiments, modified oligonucleotides are complementary within nucleobases 19888-19942 of SEQ ID NO: 1. In certain embodiments, modified oligonucleotides are 20 nucleobases in length. In certain embodiments, modified oligonucleotides are 18 nucleobases in length. In certain embodiments, modified oligonucleotides are 16 nucleobases in length. In certain embodiments, modified oligonucleotides are gapmers. In certain embodiments, modified oligonucleotides are mixed wing gapmers.

In certain embodiments, the gapmers are 5-10-5 MOE gapmers. In certain embodiments, the gapmers are 6-10-4 MOE gapmers. In certain embodiments, the gapmers are 6-8-4 MOE gapmers, 4-10-6 MOE gapmers, 4-8-6 MOE gapmers, 5-8-5 MOE gapmers, or 4-9-3 or 3-10-3 mixed MOE/cEt gapmers. In certain embodiments, the mixed wing gapmers have the sugar motif in order from 5′ to 3′: ekkddddddddddkke, ekkkddddddddkkke, kekddddddddddkkk, kkeddddddddddkkk, or kkkedddddddddkkk; wherein ‘d’ represents a 2′-β-D-deoxyribosyl sugar moiety, ‘k’ represents a cEt sugar moiety, and ‘e’ represents a 2′-MOE sugar moiety. In certain embodiments, the gapmers comprise a 2′-substituted nucleoside in the gap. In certain embodiments, the 2′-substituted nucleoside comprises a 2′-OMe sugar moiety. In certain embodiments, the 2′-substituted nucleoside is at position 2 of the gap (5′ to 3′). In certain embodiments, the gapmers have the sugar motif in order from 5′ to 3′: kkkdyddddddddkkk; wherein ‘d’ represents a 2′-β-D-deoxyribosyl sugar moiety, ‘k’ represents a cEt sugar moiety, ‘e’ represents a 2′-MOE sugar moiety, and “y” represents a 2′-OMe sugar moiety.

In certain embodiments, the internucleoside linkages of the modified oligonucleotides are phosphorothioate internucleoside linkages and phosphodiester internucleoside linkages. In certain embodiments, the phosphodiester (“o”) and phosphorothioate (“s”) internucleoside linkages are arranged in order from 5′ to 3′: In certain embodiments, modified nucleotides have an internucleoside linkage motif of sooooossssssssssoss, soooosssssssssoss, soooossssssssssooss, sooosssssssssooss, sooossssssssssoooss, or soosssssssssoooss wherein each “s” represents a phosphorothioate internucleoside linkage and each “o” represents a phosphodiester internucleoside linkage.

The nucleobase sequences of SEQ ID NOs: 160, 249, 313, 503, 588, 647, 755, 789, 882, 972, 1248-1254, 1263-1264, 1284-1285, 1332, 1400 are complementary within nucleobases 19888-19942 of SEQ ID NO: 1.

The nucleobase sequence of Compound Nos.: 1003033, 1017049, 1338115, 1380289, 1380457, 1380460, 1380571, 1380679, 1380748, 1380870, 1381153, 1400769, 1400772, 1459315, 1459345, 1459346, 1459348, 1459349, 1459351-1459356, 1459367-1459369,1459372-1459375, 1459377-1459379,1459393-1459396, 1459398-1459402, 1459422-1459429, 1459439-1459446, 1459456-1459459, 1459461, 1459463, 1459464, 1459980-1459983, and 1459988-1549991 are complementary within nucleobases 19888-19942 of SEQ ID NO: 1.

In certain embodiments, modified oligonucleotides complementary within nucleobases 19888-19942 of SEQ ID NO: 1 achieve at least 41% reduction of DMPK RNA in vitro in the standard cell assay. In certain embodiments, modified oligonucleotides complementary within nucleobases 19888-19942 of SEQ ID NO: 1 achieve an average of 81% reduction of DMPK RNA in vitro in the standard cell assay. In certain embodiments, modified oligonucleotides complementary within nucleobases 19888-19942 of SEQ ID NO: 1 achieve a maximum of 98% reduction of DMPK RNA in vitro in the standard cell assay.

### 2. Additional Hotspot Regions

In certain embodiments, the ranges described in the Table below comprise hotspot regions. Each hotspot region begins with the nucleobase of SEQ ID NO:1 identified in the “Start Site SEQ ID NO: 1” column and ends with the nucleobase of SEQ ID NO: 1 identified in the “Stop Site SEQ ID NO: 1” column. In certain embodiments, modified oligonucleotides are complementary within any of the hotspot regions 1-23, as defined in the table below. In certain embodiments, modified oligonucleotides are 20 nucleobases in length. In certain embodiments, modified oligonucleotides are 18 nucleobases in length. In certain embodiments, modified oligonucleotides are 16 nucleobases in length. In certain embodiments, modified oligonucleotides are gapmers. In certain embodiments, modified oligonucleotides are mixed wing gapmers.

In certain embodiments, the gapmers are 5-10-5 MOE gapmers. In certain embodiments, the gapmers are 6-10-4 MOE gapmers. In certain embodiments, the gapmers are 6-8-4 MOE gapmers, 4-10-6 MOE gapmers, 4-8-6 MOE gapmers, 5-8-5 MOE gapmers, or 4-9-3 or 3-10-3 mixed MOE/cEt gapmers. In certain embodiments, the mixed wing gapmers have the sugar motif in order from 5′ to 3′: ekkddddddddddkke, ekkkddddddddkkke, kekddddddddddkkk, kkeddddddddddkkk, or kkkedddddddddkkk; wherein ‘d’ represents a 2′-β-D-deoxyribosyl sugar moiety, ‘k’ represents a cEt sugar moiety, and ‘e’ represents a 2′-MOE sugar moiety. In certain embodiments, the gapmers comprise a 2′-substituted nucleoside in the gap. In certain embodiments, the 2′-substituted nucleoside comprises a 2′-OMe sugar moiety. In certain embodiments, the 2′-substituted nucleoside is at position 2 of the gap (5′ to 3′). In certain embodiments, the gapmers have the sugar motif in order from 5′ to 3′: kkkdyddddddddkkk; wherein ‘d’ represents a 2′-β-D-deoxyribosyl sugar moiety, ‘k’ represents a cEt sugar moiety, ‘e’ represents a 2′-MOE sugar moiety, and “y” represents a 2′-OMe sugar moiety.

In certain embodiments, the internucleoside linkages of the modified oligonucleotides are phosphorothioate internucleoside linkages and phosphodiester internucleoside linkages. In certain embodiments, the phosphodiester (“o”) and phosphorothioate (“s”) internucleoside linkages are arranged in order from 5′ to 3′: In certain embodiments, modified nucleotides have an internucleoside linkage motif of sooooossssssssssoss, soooosssssssssoss, soooossssssssssooss, sooosssssssssooss, sooossssssssssoooss, or soosssssssssoooss wherein each “s” represents a phosphorothioate internucleoside linkage and each “o” represents a phosphodiester internucleoside linkage.

The nucleobase sequences of compounds listed in the “Compound No. in range” column in the table below are complementary to SEQ ID NO: 2 within the specified hotspot region. The nucleobase sequences of the oligonucleotides listed in the “SEQ ID NO: in range” column in the table below are complementary to the target sequence, SEQ ID NO: 2, within the specified hotspot region.

In certain embodiments, modified oligonucleotides complementary to nucleobases within the hotspot region achieve at least “Min. % Red. in vitro” (minimum % reduction, relative to untreated control cells) of DPMK RNA in vitro in the standard cell assay, as indicated in the table below. In certain embodiments, modified oligonucleotides complementary to nucleobases within the hotspot region achieve an average of “Avg. % Red. in vitro” (average % reduction, relative to untreated control cells) of DMPK RNA in vitro in the standard cell assay, as indicated in the table below. In certain embodiments, modified oligonucleotides complementary to nucleobases within the hotspot region achieve a maximum of “Max. % Red. in vitro” (maximum % reduction, relative to untreated control cells) of DMPK RNA in vitro in the standard cell assay, as indicated in the table below.

TABLE 1
DMPK Hotspots

| | SEQ ID | SEQ ID | | | | | |
|---|---|---|---|---|---|---|---|
| | NO: 1 | NO: 1 | Avg. % | Min. % | Max. % | | |
| Hotspot | Start | Stop | Red. in | Red. in | Red. in | | |
| ID | Site | Site | vitro | vitro | vitro | Compound No. in range | SEQ ID NO in range |
| 1 | 9052 | 9103 | 68 | 32 | 94 | 1016769-1016778, 1060884, | 132, 186, 256, 327, |
| | | | | | | 1380292, 1380370, 1380716, | 446, 1374, 1596, 1667, |
| | | | | | | 1381101, 1381134 | 1747, 1818, 1895, |
| | | | | | | | 1964, 2038, 2121, 2191 |
| 2 | 9228 | 9256 | 81 | 71 | 92 | 1016782-1016785, 1380835, | 510, 1173, 1668, 1748, |
| | | | | | | 1380875 | 1819, 1896 |
| 3 | 9574 | 9610 | 86 | 83 | 89 | 1016802-1016806 | 1376, 1448, 1526, |
| | | | | | | | 1599, 1670 |
| 4 | 10010 | 10043 | 84 | 75 | 90 | 1016832-1016835 | 1823, 1900, 1969, 2043 |
| 5 | 10271 | 10298 | 78 | 54 | 89 | 1016845-1016852 | 1380, 1452, 1530, |
| | | | | | | | 1901, 1970, 2044, |
| | | | | | | | 2127, 2197 |
| 6 | 10364 | 10391 | 83 | 73 | 91 | 1016858-1016865, 1382695 | 1206, 1381, 1453, |
| | | | | | | | 1531, 1604, 1971, |
| | | | | | | | 2045, 2128, 2198 |
| 7 | 10683 | 10707 | 79 | 62 | 91 | 1016890-1016892, 1060880, | 640, 714, 821, 1172, |
| | | | | | | 1380295, 1380373, | 1677, 1757, 1828 |
| | | | | | | 1380516, 1380995, 1381015 | |
| 8 | 10709 | 10734 | 79 | 67 | 93 | 1060886, 1380431, 1380656, | 43, 115, 202, 900, 960, |
| | | | | | | 1380700, 1380721, 1381015, | 1027, 1195, 1905 |
| | | | | | | 1381069, 1382680 | |
| 9 | 10812 | 10857 | 81 | 77 | 87 | 1016898-1016903 | 1384, 1456, 1534, |
| | | | | | | | 1607, 1678, 1758 |
| 10 | 11853 | 11879 | 82 | 75 | 86 | 1016930-1016934 | 1387, 1977, 2051, |
| | | | | | | | 2134, 2204 |
| 11 | 13310 | 13350 | 83 | 70 | 94 | 570428, 1002722, 1002723, | 1296, 1351, 1425, |
| | | | | | | 1002725, 1002727-1002729, | 1501, 1793, 1867, |
| | | | | | | 1016947-1016949, 1460184 | 1979, 2052, 2083, |
| | | | | | | | 2092, 2206 |
| 12 | 13999 | 14046 | 70 | 35 | 90 | 1002583-1002586, 1016726- | 49, 159, 208, 293, 402, |
| | | | | | | 1016728, 1060873, 1380598, | 471, 556, 618, 676, |
| | | | | | | 1380842, 1380864, 1380944, | 692, 754, 817, 901, |
| | | | | | | 1380978, 1381079, 1381080, | 971, 1038, 1744, 1791, |
| | | | | | | 1381108, 1381255, 1381363, | 1863, 1960, 2016, |
| | | | | | | 1381470, 1381501, 1381507, | 2119, 2163 |
| | | | | | | 1381657, 1381677, 1459343, | |
| | | | | | | 1459344, 1459364, 1459365, | |
| | | | | | | 1459366, 1459392, 1459420 | |
| 13 | 14090 | 14118 | 87 | 84 | 89 | 1002591, 1016733, 1016734, | 1718, 1814, 1891, 1941 |
| | | | | | | 1002593 | |
| 14 | 14232 | 14258 | 70 | 56 | 88 | 570263, 570266, 1380496, | 41, 140, 888, 981, |
| | | | | | | 1380688, 1380778, 1381212, | 1033, 2081, 2154 |
| | | | | | | 1381287 | |
| 15 | 17565 | 17594 | 79 | 48 | 98 | 1002947, 1002948, 1002953, | 444, 508, 573, 1874, |
| | | | | | | 1017016, 1060901, 1380588, | 1949, 2060, 2103 |
| | | | | | | 1380785, 1381123 | |
| 16 | 17731 | 17761 | 80 | 57 | 99 | 570725, 1017023-1017026, | 274, 337, 410, 526, |
| | | | | | | 1309473, 1309469, 1380286, | 575, 665, 712, 829, |
| | | | | | | 1380432, 1380469, 1380647, | 897, 1397, 1467, 2138, |
| | | | | | | 1380690, 1380847, 1381021, | 2210, 2270, |
| | | | | | | 1381132, 1381214 | |
| 17 | 19719 | 19753 | 86 | 70 | 94 | 1003014-1003020 | 1432, 1509, 1580, |
| | | | | | | | 1654, 1729, 1801 |
| 18 | 19795 | 19869 | 84 | 29 | 100 | 1003025-1003030, 1017040- | 355, 412, 506, 567, |
| | | | | | | 1017046, 1060864, 1060883, | 673, 747, 832, 904, |
| | | | | | | 1060889, 1060891, 1380454, | 956, 1399, 1469, 1545, |
| | | | | | | 1380508, 1380630, 1380777, | 1581, 1655, 1730, |
| | | | | | | 1380803, 1380830, 1380963, | 1841, 1916, 1988, |
| | | | | | | 1381176, 1381183 | 1989, 2027, 2106, 2177 |
| 19 | 19888 | 19942 | 81 | 41 | 98 | 1003033, 1003034, 1017049, | 160, 249, 313, 371, |
| | | | | | | 1017051, 1060860, 1060870, | 424, 503, 588, 647, |
| | | | | | | 1207018, 1207019, 1207020, | 755, 789, 882, 1248- |
| | | | | | | 1207021, 1207024, 1207025, | 1254, 1263-1264, |
| | | | | | | 1207072, 1207074, 1207075, | 1266-1273, 1284-1285, |
| | | | | | | 1207130, 1213273, 1213275, | 1332, 1400, 1489, |
| | | | | | | 1213276, 1213282, 1215869, | 1619, 1637, 1638, |
| | | | | | | 1215870, 1273291, 1273292, | 1639, 1656, 1709, |
| | | | | | | 1273297, 1273301, 1338115, | 2006, 2079, 2082, |
| | | | | | | 1380289, 1380457, 1380460, | 2085, 2153, 2303 |
| | | | | | | 1380571, 1380679, 1380748, | |
| | | | | | | 1380870, 1381153, 1381230, | |
| | | | | | | 1381513, 1381524, 1381668, | |
| | | | | | | 1400769, 1400772, 1459345, | |
| | | | | | | 1459346, 1459348, 1459349, | |
| | | | | | | 1459351-1459356, 1459367- | |
| | | | | | | 1459369, 1459372-1459375, | |
| | | | | | | 1459377-1459379, 1459393- | |
| | | | | | | 1459396, 1459398-1459402, | |
| | | | | | | 1459422-1459429, 1459439- | |
| | | | | | | 1459446, 1459456-1459459, | |
| | | | | | | 1459461, 1459463, 1459464, | |
| | | | | | | 1459980-1459983, 1459988- | |
| | | | | | | 1549991 | |
| 20 | 19915 | 19942 | 85 | 61 | 98 | 1003033, 1003034, 1017049, | 503, 588, 647, 755, |
| | | | | | | 1380289, 1380457, 1380460, | 789, 882, 1263, 1264, |
| | | | | | | 1380571, 1380679, 1380748, | 1332, 1400, 1619, |
| | | | | | | 1017051, 1060860, 1060870, | 1637, 1638, 1639, |
| | | | | | | 1207018, 1207019, 1207020, | 1656, 1709, 2006, |
| | | | | | | 1207021, 1207024, 1207025, | 2079, 2082, 2085, |
| | | | | | | 1207072, 1207074, 1207075, | 2153, 2303 |
| | | | | | | 1207130, 1213275, 1213276, | |
| | | | | | | 1213282, 1215869, 1215870, | |
| | | | | | | 1273291, 1273292, 1273297, | |
| | | | | | | 1273301, 1380870, 1338115, | |
| | | | | | | 1381153, 1400769, 1400772, | |
| | | | | | | 1459980-1459983, 1459988- | |
| | | | | | | 1459991 | |
| 21 | 20871 | 20905 | 79 | 35 | 100 | 570784-570787, 1003047, 1003048, | 144, 233, 291, 328, |
| | | | | | | 1059892, 1059894, 1059903, | 435, 482, 564, 642, |
| | | | | | | 1380294, 1380304, 1380382, | 748, 808, 874, 955, |
| | | | | | | 1380403, 1380453, 1380597, | 1339, 1340, 1341, |
| | | | | | | 1380678, 1380975, 1380977, | 1492, 1732, 1803, |
| | | | | | | 1381178, 1381332, 1381435 | 2321, 2322, 2323 |
| 22 | 21117 | 21153 | 82 | 56 | 98 | 1003078-1003081, 1017069, | 576, 652, 724, 811, |
| | | | | | | 1060875, 1060898, 1380355, | 870, 1359, 1433, 1510, |
| | | | | | | 1380489, 1380502, 1380693, | 1583, 1692 |
| | | | | | | 1380758 | |
| 23 | 22118 | 22143 | 84* | 84* | 84* | 1381456, 1459383, 1459386, | 696, 1255-1259, 1265, |
| | | | | | | 1459387, 1459407-1459409, | 1274-1277, 1283, |
| | | | | | | 1459412, 1459413, 1459415, | 1330, 1331 |
| | | | | | | 1459421, 1459433-1459438, | |
| | | | | | | 1459449, 1459450-1459453, | |
| | | | | | | 1459455, 1459462, 1459465 | |

*Only a single compound was tested in vitro; average in vivo reduction in cortex is 46.7% with the RTS38096 primer probe set.

### Certain Comparator Compositions

In certain embodiments, ISIS-DMPK_{Rx} (generic name baliforsen; Compound No. 598769), entered into clinical trials for treatment of DM1, is a comparator compound (see, e.g., Thornton, et al., Neurology, 86 (16 supplement): P3.163, 2016). ISIS-DMV1PK_{Rx}, 598769 was previously described in WO2015/021457, incorporated herein by reference, and has a nucleobase sequence (from 5′ to 3′) of TCCCGAATGTCCGACA (SEQ ID NO: 1337). The sugar motif for Compound No. 598769 is (from 5′ to 3′): eekkddddddddkkee; wherein each “e” represents a 2′-MOE sugar moiety, each “d” represents a 2′-β-D-deoxyribosyl sugar moiety, and each “k” represents a cEt sugar moiety. The internucleoside linkage motif for Compound No. 598769 is (from 5′ to 3′): sssssssssssssss; wherein each “s” represents a phosphorothioate internucleoside linkage. Each cytosine nucleobase in Compound No. 598769 is a 5-methylcytosine.

In certain embodiments, Compound No. 486178, although not entered into clinical trials, is a comparator compound (see, e.g., Yadava, et al., Hum. Mol. Genetics, 29(9): 1440-1453, 2020; Pandey, et al., J. Pharamacol. Expt. Therapy, 355(2):329-340, 2015). Compound No. 486178 was previously described in WO 2015/021457 A2, WO 2017/053995 A1, and WO 2019/118916 A1, each of which is incorporated herein by reference, and consists of the nucleobase sequence (from 5′ to 3′): ACAATAAATACCGAGG (SEQ ID NO: 1336). The sugar motif for Compound No. 486178 is (from 5′ to 3′): kkkddddddddddkkk; wherein each “d” represents a 2′-β-D-deoxyribosyl sugar moiety, and each “k” represents a cEt sugar moiety. The internucleoside linkage motif for Compound No. 486178 is (from 5′ to 3′): sssssssssssssss; wherein each “s” represents a phosphorothioate internucleoside linkage. Each cytosine nucleobase in Compound No. 486178 is a 5-methylcytosine.

In certain embodiments, compounds described herein are superior relative to compounds described WO2015/021457, because they demonstrate one or more improved properties, such as activity, potency, and/or tolerability.

### Nonlimiting Disclosure and Incorporation by Reference

Each of the literature and patent publications listed herein is incorporated by reference in its entirety.

While certain compounds, compositions and methods described herein have been described with specificity in accordance with certain embodiments, the following examples serve only to illustrate the compounds described herein and are not intended to limit the same. Each of the references, GenBank accession numbers, ENSEMBL identifiers, and the like recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH in place of one 2′-H of DNA) or as an RNA having a modified base (thymine (methylated uracil) in place of an uracil of RNA). Accordingly, nucleic acid sequences provided herein, including, but not limited to those in the sequence listing, are intended to encompass nucleic acids containing any combination of natural or modified RNA and/or DNA, unless otherwise stated, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “AT^{m}CGAUCG,” wherein ^{m}C indicates a cytosine base comprising a methyl group at the 5-position.

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as a or β such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their stereorandom and optically pure forms, unless specified otherwise. Likewise, tautomeric forms of the compounds herein are also included unless otherwise indicated. Unless otherwise indicated, compounds described herein are intended to include corresponding salt forms.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O, and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### EXAMPLES

The following examples illustrate certain embodiments of the present disclosure and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif. And, for example, where a particular high-affinity modification appears at a particular position, other high-affinity modifications at the same position are considered suitable, unless otherwise indicated.

### Example 1: Effect of 5-10-5 MOE Modified Oligonucleotides with Mixed PO/PS Backbone Internucleoside Linkages on Human DMPK In Vitro, Single Dose

Modified oligonucleotides complementary to human DMPK nucleic acid were synthesized and tested for their effect on DMPK RNA levels in vitro. The modified oligonucleotides were tested in a series of experiments using the same culture conditions. The results are presented in the table below.

The modified oligonucleotides in the table below are 5-10-5 MOE modified oligonucleotides with mixed PO/PS backbone internucleoside linkages. The modified oligonucleotides are 20 nucleosides in length. The sugar motif for the modified oligonucleotides is (from 5′ to 3′): eeeeeddddddddddeeeee; wherein each ‘d’ represents a 2′-β-D-deoxyribosyl sugar moiety, and each ‘e’ represents a 2′-MOE sugar moiety. The internucleoside linkage motif for the modified oligonucleotides is (from 5′ to 3′): soooossssssssssooss wherein each ‘s’ represents a phosphorothioate internucleoside linkage, and each ‘o’ represents a phosphodiester internucleoside linkage. All cytosine nucleobases are 5-methylcytosines.

“Start site” indicates the 5′-most nucleoside of the target sequence to which the modified oligonucleotide is complementary. “Stop site” indicates the 3′-most nucleoside of the target sequence to which the modified oligonucleotide is complementary. As shown in the tables below, the modified oligonucleotides are complementary to SEQ ID NO: 1 (the complement of GENBANK Accession No. NT_011109.16, truncated from nucleotides 18539000 to 18566000), SEQ ID NO: 2 (GENBANK Accession No. NM_004409.4). ‘N/A’ indicates that the modified oligonucleotide is not 100% complementary to that particular target sequence.

Cultured A431 cells at a density of 10,000 cells per well were treated with 4,000 nM of modified oligonucleotide by free uptake. After a treatment period of approximately 48 hours, RNA was isolated from the cells and DMPK RNA levels were measured by quantitative real-time RTPCR. HumanDMPK primer probe set RTS38095 (forward nucleobase sequence CTGAGCCGGGAGATGGA, designated herein as SEQ ID NO: 6; reverse nucleobase sequence GGACGTGTGCCTCTAGGT, designated herein as SEQ ID NO: 7; probe nucleobase sequence TGACTGGCGAAGTTCTGGTTGTCC, designated herein as SEQ ID NO: 8) was used to measure DMPK RNA levels. DMPK RNA levels were normalized to total RNA content, as measured by RIBOGREEN®. Results are presented as percent of DMPK RNA, relative to untreated control cells (% UTC). The values marked by the symbol “†” indicate that the modified oligonucleotide is complementary to the amplicon region of the primer probe set. Additional assays may be used to measure the activity of the modified oligonucleotides complementary to the amplicon region.

Each separate experiment described in this example is identified by an Assay Identification letter in the table column labeled “AID”.

TABLE 2
Reduction of DMPK RNA by 5-10-5 MOE modified oligonucleotides with mixed PS/PO backbone
internucleoside linkages

| | SEQ ID | SEQ ID | SEQ ID | SEQ ID | | | | |
|---|---|---|---|---|---|---|---|---|
| | NO: 1 | NO: 1 | NO: 2 | NO: 2 | | DMPK | | SEQ |
| Compound | Start | Stop | Start | Stop | Nucleobase Sequence | (% | | ID |
| No. | Site | Site | Site | Site | (5′ to 3′) | UTC) | AID | NO |
| 1052866 | 24728 | 24747 | 2786 | 2805 | AGACAATAAATACCGAGGAA | 38 | A | 18 |
| 1052873 | 19496 | 19515 | 1357 | 1376 | CTTCCCGAATGTCCGACAGT | 62 | A | 19 |
| 1052874 | 19497 | 19516 | 1358 | 1377 | CCTTCCCGAATGTCCGACAG | 78 | A | 20 |
| 1052881 | 21094 | 21113 | N/A | N/A | ATGTGTAATGTTGTCCAGTA | 13 | A | 21 |
| 1380291 | 14533 | 14552 | N/A | N/A | CAGCTTCACCCTAGGACTGT | 60 | A | 22 |
| 1380335 | 19360 | 19379 | N/A | N/A | AAGTGGCCCCTCCAGCATTT | 50 | A | 23 |
| 1380384 | 17699 | 17718 | N/A | N/A | ACACTCTCCATAATTCTCTA | 36 | A | 24 |
| 1380388 | 22196 | 22215 | N/A | N/A | AAATCAGGATTCCCACCTGC | 90 | A | 25 |
| 1380412 | 24633 | 24652 | 2691 | 2710 | CGAGGTCAATAAATATCCAA | 49 | A | 26 |
| 1380425 | 20815 | 20834 | N/A | N/A | CCTAGGCTGGAATCTATCAT | 117 | A | 27 |
| 1380445 | 22294 | 22313 | N/A | N/A | CCTAATGCCCTCACGACAAA | 88 | A | 28 |
| 1380447 | 14186 | 14205 | N/A | N/A | TACCTCTAGATTCAGATGCA | 30 | A | 29 |
| 1380462 | 22051 | 22070 | N/A | N/A | TTACTTAATGCCCCACTGTA | 94 | A | 30 |
| 1380463 | 9687 | 9706 | N/A | N/A | CAGGACTCTACGATTCCAAA | 66 | A | 31 |
| 1380486 | 17607 | 17626 | N/A | N/A | TCAGCAAAAGGGCACCCAGA | 125 | A | 32 |
| 1380505 | 19666 | 19685 | N/A | N/A | CAGTAAGGTTCCAAGACTGA | 86 | A | 33 |
| 1380530 | 24458 | 24477 | 2516 | 2535 | ACAAGAAAGCTTTGCACTTT | 99 | A | 34 |
| 1380533 | 21977 | 21996 | N/A | N/A | TGCTTCTGTTCAGGAAGTCC | 85 | A | 35 |
| 1380537 | 15919 | 15938 | 673 | 692 | ACTTGCTCAGCAGTGTCAGC | 65 | A | 36 |
| 1380591 | 15635 | 15654 | N/A | N/A | GGCAGATTCACTCCCCCTGA | 38 | A | 37 |
| 1380602 | 19926 | 19945 | N/A | N/A | TGGCCTTATTGTTATATGGC | 115 | A | 38 |
| 1380607 | 17251 | 17270 | N/A | N/A | TGTCCTTACTCCAACTTTAT | 53 | A | 39 |
| 1380634 | 14050 | 14069 | N/A | N/A | CCATCTCTCAGTCCTCCAGG | 29 | A | 40 |
| 1380688 | 14238 | 14257 | N/A | N/A | GTTCTCATGTAGAATGTCCT | 37 | A | 41 |
| 1380696 | 22143 | 22162 | N/A | N/A | CACTTGGCACCTTTCCTTCC | 86 | A | 42 |
| 1380700 | 10713 | 10732 | N/A | N/A | CTTCCATAATTTAACACTCT | 23 | A | 43 |
| 1380751 | 22341 | 22360 | N/A | N/A | CACTAACACAACCTATGTCC | 95 | A | 44 |
| 1380767 | 15502 | 15521 | N/A | N/A | GGTAAGAGACCCCCCGCAAC | 71 | A | 45 |
| 1380776 | 16714 | 16733 | 943 | 962 | CTGTCCCAGGCCCACCGCCC | 163 | A | 46 |
| 1380808 | 22311 | 22330 | N/A | N/A | CAAAATCCCTCCAGCTCCCT | 97 | A | 47 |
| 1380816 | 20954 | 20973 | N/A | N/A | GCCAGGGACCACTGCCATCT | 70 | A | 48 |
| 1380842 | 14020 | 14039 | N/A | N/A | TAGTCCTACCCCTTATTTAC | 37 | A | 49 |
| 1380874 | 13978 | 13997 | N/A | N/A | AAGTCAAGGTCCTATGACTA | 139 | A | 50 |
| 1380895 | 9051 | 9070 | N/A | N/A | CACTAAGATTTCCCTGGCTT | 70 | A | 51 |
| 1380908 | 21467 | 21486 | N/A | N/A | CCTCCCTTGACATGTGACCG | 70 | A | 52 |
| 1380910 | 16699 | 16718 | 928 | 947 | CGCCCACAGCCTGCAGGATC | 73 | A | 53 |
| 1380967 | 24045 | 24064 | 2103 | 2122 | CCCCGGAGTCGAAGACAGTT | 128 | A | 54 |
| 1380973 | 19715 | 19734 | N/A | N/A | AATTTAAGGTCCTCCAACTC | 109 | A | 55 |
| 1381019 | 22325 | 22344 | N/A | N/A | GTCCCTCTGCTGCTCAAAAT | 79 | A | 56 |
| 1381038 | 17283 | 17302 | N/A | N/A | ATGAGTGATTCAGGACCCCA | 47 | A | 57 |
| 1381128 | 20888 | 20907 | N/A | N/A | TAATTTACTTGTGATAAGCA | 58 | A | 58 |
| 1381136 | 21366 | 21385 | N/A | N/A | ATGTGGTCCTAAGACTGGGC | 95 | A | 59 |
| 1381151 | 21053 | 21072 | N/A | N/A | AACTGTTCTCTTAGACAAAG | 83 | A | 60 |
| 1381218 | 24442 | 24461 | 2500 | 2519 | CTTTGCGAACCAACGATAGG | 120 | A | 61 |
| 1381229 | 17724 | 17743 | N/A | N/A | GTTGCTTCCCTTCAGGGCAC | 83 | A | 62 |
| 1381231 | 12150 | 12169 | 183 | 202 | GGACAGGCAGCACCATGGCC | 86 | A | 63 |
| 1381234 | 22262 | 22281 | N/A | N/A | GTTTTGTTTCCTGCTGGCCT | 48 | A | 64 |
| 1381256 | 17118 | 17137 | 1271 | 1290 | GTGGCACCTTCGAAATCCGG | 85 | A | 65 |
| 1381259 | 19859 | 19878 | N/A | N/A | ACTTCATATTTTCCAAGTTC | 45 | A | 66 |
| 1381267 | 24341 | 24360 | 2399 | 2418 | CTCAGCCTGGCCGAAAGAAA | 71 | A | 67 |
| 1381274 | 12772 | 12791 | N/A | N/A | CCTGCCTGTCGGCTGCGCCC | 63 | A | 68 |
| 1381290 | 24200 | 24219 | 2258 | 2277 | CGCGGACCCGGCCCCTCCCT | 166 | A | 69 |
| 1381293 | 13688 | 13707 | N/A | N/A | TGAGCCCTTTTAAGGCAGCA | 88 | A | 70 |
| 1381317 | 15193 | 15212 | 569 | 588 | CGGTCCCCATTCACCAACAC | 125 | A | 71 |
| 1381322 | 17152 | 17171 | 1305 | 1324 | CCCGTCCTCCACCAAGTCGA | 71 | A | 72 |
| 1381346 | 24394 | 24413 | 2452 | 2471 | CGGCCCGGCTTGCTGCCTTC | 94 | A | 73 |
| 1381369 | 19566 | 19585 | N/A | N/A | AGTGCTTACCTGAGGGCCAT | 100 | A | 74 |
| 1381373 | 24224 | 24243 | 2282 | 2301 | ACCCTTCGAGCCCCGTTCGC | 107 | A | 75 |
| 1381383 | 15979 | 15998 | 733 | 752 | TGGCCATGACAATCTCCGCC | 78 | A | 76 |
| 1381393 | 24512 | 24531 | 2570 | 2589 | CCGAGTAAGCAGGCAGAGAT | 63 | A | 77 |
| 1381398 | 17368 | 17387 | N/A | N/A | GCTCTGTGTTCCCCCACTGG | 62 | A | 78 |
| 1381407 | 12523 | 12542 | N/A | N/A | GCTGACCTTACTCTGCCCCT | 31 | A | 79 |
| 1381443 | 15938 | 15957 | 692 | 711 | GCCGGAATCCGCTCCCCAAA | 81 | A | 80 |
| 1381459 | 24083 | 24102 | 2141 | 2160 | TCTGTGCCGTGCCCCGGGCA | 87 | A | 81 |
| 1381475 | 17017 | 17036 | 1170 | 1189 | ACCCCGGCCCAGCCGTGTCT | 73 | A | 82 |
| 1381500 | 24617 | 24636 | 2675 | 2694 | CCAAACCGCCGAAGCGGGCG | 131 | A | 83 |
| 1381523 | 15075 | 15094 | N/A | N/A | CCCCTCACCTCGCCCCTCTT | 58 | A | 84 |
| 1381525 | 24241 | 24260 | 2299 | 2318 | CATTCCCGGCTACAAGGACC | 79 | A | 85 |
| 1381526 | 24376 | 24395 | 2434 | 2453 | TCCCAGGCCTGCAGTTTGCC | 126 | A | 86 |
| 1381535 | 22628 | 22647 | 1581 | 1600 | CTCGGCCTCAGCCTCTGCCG | 114 | A | 87 |
| 1381538 | 24137 | 24156 | 2195 | 2214 | GGGCGGAGACCCACGCTCGG | 100 | A | 88 |
| 1381541 | 17494 | 17513 | N/A | N/A | TCAGCTCAGATAGCTCCCCA | 113 | A | 89 |
| 1381549 | 14666 | 14685 | 376 | 395 | CCTCCTTAAGCCTCACCACG | 77 | A | 90 |
| 1381556 | 23676 | 23695 | N/A | N/A | GCGGCCTGTGTTGATTGGCT | 104 | A | 91 |
| 1381583 | 22885 | 22904 | 1733 | 1752 | TGTGCCTCTAGGTCCCGGTT | 16† | A | 92 |
| 1381656 | 15017 | 15036 | 472 | 491 | CCTGGCCCGTCTGCTTCATC | 85 | A | 93 |
| 1381666 | 13012 | 13031 | N/A | N/A | GCTGACCCACACGGCTCATA | 42 | A | 94 |
| 1381684 | 24532 | 24551 | 2590 | 2609 | GTTTGGCAAAAGCAAATTTC | 91 | A | 95 |
| 1052867 | 24729 | 24748 | 2787 | 2806 | CAGACAATAAATACCGAGGA | 20 | B | 96 |
| 1052873 | 19496 | 19515 | 1357 | 1376 | CTTCCCGAATGTCCGACAGT | 118 | B | 19 |
| 1052875 | 19498 | 19517 | 1359 | 1378 | ACCTTCCCGAATGTCCGACA | 54 | B | 97 |
| 1380281 | 15636 | 15655 | N/A | N/A | GGGCAGATTCACTCCCCCTG | 72 | B | 98 |
| 1380348 | 21370 | 21389 | N/A | N/A | ACAGATGTGGTCCTAAGACT | 60 | B | 99 |
| 1380353 | 17252 | 17271 | N/A | N/A | CTGTCCTTACTCCAACTTTA | 57 | B | 100 |
| 1380414 | 22328 | 22347 | N/A | N/A | TATGTCCCTCTGCTGCTCAA | 47 | B | 101 |
| 1380464 | 20890 | 20909 | N/A | N/A | TCTAATTTACTTGTGATAAG | 54 | B | 102 |
| 1380475 | 9688 | 9707 | N/A | N/A | TCAGGACTCTACGATTCCAA | 118 | B | 103 |
| 1380518 | 14534 | 14553 | N/A | N/A | TCAGCTTCACCCTAGGACTG | 45 | B | 104 |
| 1380521 | 21978 | 21997 | N/A | N/A | CTGCTTCTGTTCAGGAAGTC | 125 | B | 105 |
| 1380557 | 13979 | 13998 | N/A | N/A | GAAGTCAAGGTCCTATGACT | 90 | B | 106 |
| 1380564 | 14189 | 14208 | N/A | N/A | CAGTACCTCTAGATTCAGAT | 16 | B | 107 |
| 1380570 | 21469 | 21488 | N/A | N/A | CTCCTCCCTTGACATGTGAC | 49 | B | 108 |
| 1380595 | 22052 | 22071 | N/A | N/A | CTTACTTAATGCCCCACTGT | 82 | B | 109 |
| 1380618 | 16715 | 16734 | 944 | 963 | CCTGTCCCAGGCCCACCGCC | 79 | B | 110 |
| 1380623 | 19716 | 19735 | N/A | N/A | AAATTTAAGGTCCTCCAACT | 59 | B | 111 |
| 1380627 | 15921 | 15940 | 675 | 694 | AAACTTGCTCAGCAGTGTCA | 104 | B | 112 |
| 1380638 | 14051 | 14070 | N/A | N/A | CCCATCTCTCAGTCCTCCAG | 33 | B | 113 |
| 1380644 | 17119 | 17138 | 1272 | 1291 | GGTGGCACCTTCGAAATCCG | 130 | B | 114 |
| 1380656 | 10714 | 10733 | N/A | N/A | GCTTCCATAATTTAACACTC | 25 | B | 115 |
| 1380659 | 21097 | 21116 | N/A | N/A | AGTATGTGTAATGTTGTCCA | 1 | B | 116 |
| 1380670 | 19668 | 19687 | N/A | N/A | AACAGTAAGGTTCCAAGACT | 55 | B | 117 |
| 1380671 | 17700 | 17719 | N/A | N/A | CACACTCTCCATAATTCTCT | 106 | B | 118 |
| 1380680 | 22906 | 22925 | 1754 | 1773 | CGCTCCTGCAACTGCCGGAC | 87† | B | 119 |
| 1380691 | 17729 | 17748 | N/A | N/A | ATCCTGTTGCTTCCCTTCAG | 49 | B | 120 |
| 1380711 | 22144 | 22163 | N/A | N/A | CCACTTGGCACCTTTCCTTC | 61 | B | 121 |
| 1380713 | 21054 | 21073 | N/A | N/A | CAACTGTTCTCTTAGACAAA | 47 | B | 122 |
| 1380752 | 17284 | 17303 | N/A | N/A | AATGAGTGATTCAGGACCCC | 52 | B | 123 |
| 1380763 | 15504 | 15523 | N/A | N/A | CAGGTAAGAGACCCCCCGCA | 85 | B | 124 |
| 1380764 | 22312 | 22331 | N/A | N/A | TCAAAATCCCTCCAGCTCCC | 90 | B | 125 |
| 1380807 | 17609 | 17628 | N/A | N/A | GTTCAGCAAAAGGGCACCCA | 35 | B | 126 |
| 1380839 | 16700 | 16719 | 929 | 948 | CCGCCCACAGCCTGCAGGAT | 125 | B | 127 |
| 1380926 | 20955 | 20974 | N/A | N/A | AGCCAGGGACCACTGCCATC | 81 | B | 128 |
| 1380941 | 24459 | 24478 | 2517 | 2536 | CACAAGAAAGCTTTGCACTT | 82 | B | 129 |
| 1380979 | 19927 | 19946 | N/A | N/A | ATGGCCTTATTGTTATATGG | 73 | B | 130 |
| 1381082 | 13957 | 13976 | N/A | N/A | GAATGTTAAACTGGGCAGCC | 102 | B | 131 |
| 1381101 | 9053 | 9072 | N/A | N/A | GACACTAAGATTTCCCTGGC | 38 | B | 132 |
| 1381107 | 22343 | 22362 | N/A | N/A | AACACTAACACAACCTATGT | 95 | B | 133 |
| 1381138 | 19361 | 19380 | N/A | N/A | AAAGTGGCCCCTCCAGCATT | 47 | B | 134 |
| 1381158 | 22295 | 22314 | N/A | N/A | CCCTAATGCCCTCACGACAA | 106 | B | 135 |
| 1381167 | 24634 | 24653 | 2692 | 2711 | ACGAGGTCAATAAATATCCA | 54 | B | 136 |
| 1381224 | 22198 | 22217 | N/A | N/A | ACAAATCAGGATTCCCACCT | 89 | B | 137 |
| 1381243 | 24226 | 24245 | 2284 | 2303 | GGACCCTTCGAGCCCCGTTC | 80 | B | 138 |
| 1381265 | 12775 | 12794 | N/A | N/A | ACACCTGCCTGTCGGCTGCG | 66 | B | 139 |
| 1381287 | 14239 | 14258 | N/A | N/A | CGTTCTCATGTAGAATGTCC | 43 | B | 140 |
| 1381297 | 24517 | 24536 | 2575 | 2594 | ATTTCCCGAGTAAGCAGGCA | 74 | B | 141 |
| 1381313 | 24066 | 24085 | 2124 | 2143 | GCACTCAGTCTTCCAACGGG | 85 | B | 142 |
| 1381321 | 24242 | 24261 | 2300 | 2319 | GCATTCCCGGCTACAAGGAC | 90 | B | 143 |
| 1381332 | 20871 | 20890 | N/A | N/A | GCAATGCATTATTTGTGTAA | 8 | B | 144 |
| 1381347 | 24618 | 24637 | 2676 | 2695 | TCCAAACCGCCGAAGCGGGC | 86 | B | 145 |
| 1381350 | 13014 | 13033 | N/A | N/A | TGGCTGACCCACACGGCTCA | 133 | B | 146 |
| 1381360 | 17370 | 17389 | N/A | N/A | CTGCTCTGTGTTCCCCCACT | 69 | B | 147 |
| 1381370 | 24533 | 24552 | 2591 | 2610 | GGTTTGGCAAAAGCAAATTT | 113 | B | 148 |
| 1381379 | 17153 | 17172 | 1306 | 1325 | GCCCGTCCTCCACCAAGTCG | 77 | B | 149 |
| 1381411 | 15939 | 15958 | 693 | 712 | GGCCGGAATCCGCTCCCCAA | 72 | B | 150 |
| 1381412 | 15195 | 15214 | 571 | 590 | GCCGGTCCCCATTCACCAAC | 83 | B | 151 |
| 1381421 | 24138 | 24157 | 2196 | 2215 | TGGGCGGAGACCCACGCTCG | 105 | B | 152 |
| 1381424 | 19567 | 19586 | N/A | N/A | CAGTGCTTACCTGAGGGCCA | 102 | B | 153 |
| 1381434 | 15980 | 15999 | 734 | 753 | ATGGCCATGACAATCTCCGC | 94 | B | 154 |
| 1381445 | 22264 | 22283 | N/A | N/A | GGGTTTTGTTTCCTGCTGGC | 102 | B | 155 |
| 1381447 | 24343 | 24362 | 2401 | 2420 | GCCTCAGCCTGGCCGAAAGA | 93 | B | 156 |
| 1381467 | 24395 | 24414 | 2453 | 2472 | ACGGCCCGGCTTGCTGCCTT | 101 | B | 157 |
| 1381486 | 17496 | 17515 | N/A | N/A | TCTCAGCTCAGATAGCTCCC | 59 | B | 158 |
| 1381501 | 14021 | 14040 | N/A | N/A | TTAGTCCTACCCCTTATTTA | 43 | B | 159 |
| 1381513 | 19891 | 19910 | N/A | N/A | GCATTCTTTTACAACTGATT | 2 | B | 160 |
| 1381528 | 12151 | 12170 | 184 | 203 | TGGACAGGCAGCACCATGGC | 65 | B | 161 |
| 1381561 | 24201 | 24220 | 2259 | 2278 | CCGCGGACCCGGCCCCTCCC | 134 | B | 162 |
| 1381562 | 24379 | 24398 | 2437 | 2456 | CCTTCCCAGGCCTGCAGTTT | 103 | B | 163 |
| 1381567 | 14667 | 14686 | 377 | 396 | ACCTCCTTAAGCCTCACCAC | 93 | B | 164 |
| 1381595 | 15019 | 15038 | 474 | 493 | CACCTGGCCCGTCTGCTTCA | 79 | B | 165 |
| 1381607 | 22634 | 22653 | 1587 | 1606 | CGTCACCTCGGCCTCAGCCT | 179 | B | 166 |
| 1381610 | 24084 | 24103 | 2142 | 2161 | TTCTGTGCCGTGCCCCGGGC | 89 | B | 167 |
| 1381617 | 15076 | 15095 | N/A | N/A | GCCCCTCACCTCGCCCCTCT | 67 | B | 168 |
| 1381620 | 24443 | 24462 | 2501 | 2520 | ACTTTGCGAACCAACGATAG | 85 | B | 169 |
| 1381626 | 17021 | 17040 | 1174 | 1193 | CTCCACCCCGGCCCAGCCGT | 74 | B | 170 |
| 1381664 | 12524 | 12543 | N/A | N/A | TGCTGACCTTACTCTGCCCC | 39 | B | 171 |
| 1381672 | 23823 | 23842 | N/A | N/A | ATTGGCTCCTGGGACTCGCC | 99 | B | 172 |
| 1052868 | 24730 | 24749 | 2788 | 2807 | ACAGACAATAAATACCGAGG | 30 | C | 173 |
| 1052873 | 19496 | 19515 | 1357 | 1376 | CTTCCCGAATGTCCGACAGT | 61 | C | 19 |
| 1052876 | 19499 | 19518 | 1360 | 1379 | CACCTTCCCGAATGTCCGAC | 52 | C | 174 |
| 1380318 | 21055 | 21074 | N/A | N/A | CCAACTGTTCTCTTAGACAA | 44 | C | 175 |
| 1380319 | 22145 | 22164 | N/A | N/A | ACCACTTGGCACCTTTCCTT | 55 | C | 176 |
| 1380321 | 16716 | 16735 | 945 | 964 | GCCTGTCCCAGGCCCACCGC | 98 | C | 177 |
| 1380328 | 22296 | 22315 | N/A | N/A | TCCCTAATGCCCTCACGACA | 102 | C | 178 |
| 1380398 | 17701 | 17720 | N/A | N/A | CCACACTCTCCATAATTCTC | 44 | C | 179 |
| 1380430 | 20956 | 20975 | N/A | N/A | GAGCCAGGGACCACTGCCAT | 82 | C | 180 |
| 1380578 | 24380 | 24399 | 2438 | 2457 | GCCTTCCCAGGCCTGCAGTT | 82 | C | 181 |
| 1380586 | 17286 | 17305 | N/A | N/A | GGAATGAGTGATTCAGGACC | 20 | C | 182 |
| 1380631 | 14052 | 14071 | N/A | N/A | GCCCATCTCTCAGTCCTCCA | 39 | C | 183 |
| 1380651 | 22329 | 22348 | N/A | N/A | CTATGTCCCTCTGCTGCTCA | 40 | C | 184 |
| 1380655 | 22313 | 22332 | N/A | N/A | CTCAAAATCCCTCCAGCTCC | 117 | C | 185 |
| 1380716 | 9054 | 9073 | N/A | N/A | AGACACTAAGATTTCCCTGG | 33 | C | 186 |
| 1380724 | 17253 | 17272 | N/A | N/A | ACTGTCCTTACTCCAACTTT | 43 | C | 187 |
| 1380740 | 14190 | 14209 | N/A | N/A | CCAGTACCTCTAGATTCAGA | 16 | C | 188 |
| 1380775 | 19929 | 19948 | N/A | N/A | AAATGGCCTTATTGTTATAT | 61 | C | 189 |
| 1380780 | 15922 | 15941 | 676 | 695 | CAAACTTGCTCAGCAGTGTC | 88 | C | 190 |
| 1380782 | 14535 | 14554 | N/A | N/A | CTCAGCTTCACCCTAGGACT | 28 | C | 191 |
| 1380809 | 19669 | 19688 | N/A | N/A | AAACAGTAAGGTTCCAAGAC | 64 | C | 192 |
| 1380820 | 21979 | 21998 | N/A | N/A | TCTGCTTCTGTTCAGGAAGT | 134 | C | 193 |
| 1380845 | 17614 | 17633 | N/A | N/A | GTATTGTTCAGCAAAAGGGC | 43 | C | 194 |
| 1380855 | 9689 | 9708 | N/A | N/A | CTCAGGACTCTACGATTCCA | 45 | C | 195 |
| 1380873 | 17120 | 17139 | 1273 | 1292 | CGGTGGCACCTTCGAAATCC | 85 | C | 196 |
| 1380884 | 21376 | 21395 | N/A | N/A | ACCTCCACAGATGTGGTCCT | 77 | C | 197 |
| 1380951 | 22907 | 22926 | 1755 | 1774 | CCGCTCCTGCAACTGCCGGA | 90† | C | 198 |
| 1380957 | 21099 | 21118 | N/A | N/A | ATAGTATGTGTAATGTTGTC | 35 | C | 199 |
| 1380982 | 16701 | 16720 | 930 | 949 | ACCGCCCACAGCCTGCAGGA | 89 | C | 200 |
| 1381006 | 22053 | 22072 | N/A | N/A | CCTTACTTAATGCCCCACTG | 57 | C | 201 |
| 1381015 | 10715 | 10734 | N/A | N/A | GGCTTCCATAATTTAACACT | 21 | C | 202 |
| 1381022 | 19362 | 19381 | N/A | N/A | TAAAGTGGCCCCTCCAGCAT | 51 | C | 203 |
| 1381034 | 13980 | 13999 | N/A | N/A | AGAAGTCAAGGTCCTATGAC | 34 | C | 204 |
| 1381057 | 17730 | 17749 | N/A | N/A | TATCCTGTTGCTTCCCTTCA | 60 | C | 205 |
| 1381100 | 20891 | 20910 | N/A | N/A | TTCTAATTTACTTGTGATAA | 75 | C | 206 |
| 1381103 | 15637 | 15656 | N/A | N/A | TGGGCAGATTCACTCCCCCT | 67 | C | 207 |
| 1381108 | 14022 | 14041 | N/A | N/A | TTTAGTCCTACCCCTTATTT | 56 | C | 208 |
| 1381127 | 19718 | 19737 | N/A | N/A | GAAAATTTAAGGTCCTCCAA | 46 | C | 209 |
| 1381137 | 22199 | 22218 | N/A | N/A | AACAAATCAGGATTCCCACC | 95 | C | 210 |
| 1381189 | 15505 | 15524 | N/A | N/A | CCAGGTAAGAGACCCCCCGC | 90 | C | 211 |
| 1381203 | 13958 | 13977 | N/A | N/A | GGAATGTTAAACTGGGCAGC | 18 | C | 212 |
| 1381207 | 24444 | 24463 | 2502 | 2521 | CACTTTGCGAACCAACGATA | 54 | C | 213 |
| 1381245 | 12155 | 12174 | 188 | 207 | ATGTTGGACAGGCAGCACCA | 67 | C | 214 |
| 1381251 | 22266 | 22285 | N/A | N/A | CTGGGTTTTGTTTCCTGCTG | 43 | C | 215 |
| 1381254 | 12776 | 12795 | N/A | N/A | AACACCTGCCTGTCGGCTGC | 66 | C | 216 |
| 1381263 | 17154 | 17173 | 1307 | 1326 | AGCCCGTCCTCCACCAAGTC | 137 | C | 217 |
| 1381291 | 24520 | 24539 | 2578 | 2597 | CAAATTTCCCGAGTAAGCAG | 97 | C | 218 |
| 1381292 | 23890 | 23909 | 1948 | 1967 | CCTCCGATAGGCCAGGCCTA | 71 | C | 219 |
| 1381296 | 24243 | 24262 | 2301 | 2320 | AGCATTCCCGGCTACAAGGA | 64 | C | 220 |
| 1381311 | 15077 | 15096 | N/A | N/A | AGCCCCTCACCTCGCCCCTC | 122 | C | 221 |
| 1381324 | 22589 | 22608 | N/A | N/A | TGCCACTTCAGCCTGTGTAT | 201 | C | 222 |
| 1381331 | 24139 | 24158 | 2197 | 2216 | CTGGGCGGAGACCCACGCTC | 147 | C | 223 |
| 1381357 | 21588 | 21607 | N/A | N/A | CCTCCTCTGCTTAGGAAAAG | 80 | C | 224 |
| 1381361 | 15982 | 16001 | 736 | 755 | CTATGGCCATGACAATCTCC | 77 | C | 225 |
| 1381382 | 24345 | 24364 | 2403 | 2422 | GGGCCTCAGCCTGGCCGAAA | 84 | C | 226 |
| 1381388 | 24460 | 24479 | 2518 | 2537 | GCACAAGAAAGCTTTGCACT | 56 | C | 227 |
| 1381404 | 15021 | 15040 | 476 | 495 | TACACCTGGCCCGTCTGCTT | 96 | C | 228 |
| 1381414 | 17023 | 17042 | 1176 | 1195 | TGCTCCACCCCGGCCCAGCC | 145 | C | 229 |
| 1381415 | 14310 | 14329 | N/A | N/A | AAGACCCAGTTCTTCCACCT | 38 | C | 230 |
| 1381426 | 24635 | 24654 | 2693 | 2712 | GACGAGGTCAATAAATATCC | 57 | C | 231 |
| 1381429 | 15952 | 15971 | 706 | 725 | AGCGCGCCATCTCGGCCGGA | 95 | C | 232 |
| 1381435 | 20872 | 20891 | N/A | N/A | AGCAATGCATTATTTGTGTA | 13 | C | 233 |
| 1381454 | 24089 | 24108 | 2147 | 2166 | GCGGCTTCTGTGCCGTGCCC | 147 | C | 234 |
| 1381462 | 22675 | 22694 | 1628 | 1647 | GTGAGCACCTCCTCCTCCAG | 210 | C | 235 |
| 1381472 | 17371 | 17390 | N/A | N/A | ACTGCTCTGTGTTCCCCCAC | 49 | C | 236 |
| 1381494 | 13057 | 13076 | N/A | N/A | CAGGAGAACTAAAGGACGCA | 38 | C | 237 |
| 1381495 | 19568 | 19587 | N/A | N/A | GCAGTGCTTACCTGAGGGCC | 82 | C | 238 |
| 1381502 | 12736 | 12755 | N/A | N/A | GCTGGCCCTCCTGGCTTGCC | 158 | C | 239 |
| 1381510 | 14668 | 14687 | 378 | 397 | GACCTCCTTAAGCCTCACCA | 75 | C | 240 |
| 1381514 | 17509 | 17528 | N/A | N/A | TCATCCCTCCAAGTCTCAGC | 55 | C | 241 |
| 1381571 | 24396 | 24415 | 2454 | 2473 | GACGGCCCGGCTTGCTGCCT | 80 | C | 242 |
| 1381585 | 24227 | 24246 | 2285 | 2304 | AGGACCCTTCGAGCCCCGTT | 91 | C | 243 |
| 1381597 | 24202 | 24221 | 2260 | 2279 | GCCGCGGACCCGGCCCCTCC | 156 | C | 244 |
| 1381618 | 24620 | 24639 | 2678 | 2697 | TATCCAAACCGCCGAAGCGG | 88 | C | 245 |
| 1381629 | 24534 | 24553 | 2592 | 2611 | GGGTTTGGCAAAAGCAAATT | 88 | C | 246 |
| 1381640 | 15245 | 15264 | N/A | N/A | CACCAGGTAGTTCTCATCCT | 70 | C | 247 |
| 1381645 | 24068 | 24087 | 2126 | 2145 | GGGCACTCAGTCTTCCAACG | 68 | C | 248 |
| 1381668 | 19893 | 19912 | N/A | N/A | TTGCATTCTTTTACAACTGA | 5 | C | 249 |
| 1052869 | 24731 | 24750 | 2789 | 2808 | GACAGACAATAAATACCGAG | 43 | D | 250 |
| 1052873 | 19496 | 19515 | 1357 | 1376 | CTTCCCGAATGTCCGACAGT | 74 | D | 19 |
| 1380308 | 17156 | 17175 | 1309 | 1328 | TGAGCCCGTCCTCCACCAAG | 106 | D | 251 |
| 1380316 | 24636 | 24655 | 2694 | 2713 | GGACGAGGTCAATAAATATC | 71 | D | 252 |
| 1380331 | 15506 | 15525 | N/A | N/A | TCCAGGTAAGAGACCCCCCG | 88 | D | 253 |
| 1380343 | 19793 | 19812 | N/A | N/A | ACAAGATTCTGGGAAGCCCA | 41 | D | 254 |
| 1380356 | 20957 | 20976 | N/A | N/A | TGAGCCAGGGACCACTGCCA | 68 | D | 255 |
| 1380370 | 9055 | 9074 | N/A | N/A | GAGACACTAAGATTTCCCTG | 31 | D | 256 |
| 1380378 | 19500 | 19519 | 1361 | 1380 | GCACCTTCCCGAATGTCCGA | 35 | D | 257 |
| 1380389 | 17617 | 17636 | N/A | N/A | CCAGTATTGTTCAGCAAAAG | 41 | D | 258 |
| 1380416 | 17322 | 17341 | N/A | N/A | CCTCAGTAGTAGATGGGCAC | 51 | D | 259 |
| 1380485 | 13984 | 14003 | N/A | N/A | TCTCAGAAGTCAAGGTCCTA | 32 | D | 260 |
| 1380528 | 14053 | 14072 | N/A | N/A | AGCCCATCTCTCAGTCCTCC | 48 | D | 261 |
| 1380585 | 16720 | 16739 | 949 | 968 | AGCTGCCTGTCCCAGGCCCA | 91 | D | 262 |
| 1380601 | 17702 | 17721 | N/A | N/A | GCCACACTCTCCATAATTCT | 72 | D | 263 |
| 1380614 | 15638 | 15657 | N/A | N/A | CTGGGCAGATTCACTCCCCC | 79 | D | 264 |
| 1380615 | 19673 | 19692 | N/A | N/A | CCCAAAACAGTAAGGTTCCA | 52 | D | 265 |
| 1380643 | 22146 | 22165 | N/A | N/A | GACCACTTGGCACCTTTCCT | 48 | D | 266 |
| 1380662 | 20892 | 20911 | N/A | N/A | TTTCTAATTTACTTGTGATA | 66 | D | 267 |
| 1380683 | 14536 | 14555 | N/A | N/A | TCTCAGCTTCACCCTAGGAC | 23 | D | 268 |
| 1380725 | 21056 | 21075 | N/A | N/A | ACCAACTGTTCTCTTAGACA | 13 | D | 269 |
| 1380769 | 15923 | 15942 | 677 | 696 | CCAAACTTGCTCAGCAGTGT | 63 | D | 270 |
| 1380805 | N/A | N/A | 779 | 798 | GGTTTGATGTCCCTGTGCAC | 72 | D | 271 |
| 1380827 | 21981 | 22000 | N/A | N/A | TGTCTGCTTCTGTTCAGGAA | 74 | D | 272 |
| 1380828 | 13960 | 13979 | N/A | N/A | TAGGAATGTTAAACTGGGCA | 18 | D | 273 |
| 1380847 | 17731 | 17750 | N/A | N/A | GTATCCTGTTGCTTCCCTTC | 20 | D | 274 |
| 1380872 | 14191 | 14210 | N/A | N/A | TCCAGTACCTCTAGATTCAG | 25 | D | 275 |
| 1380881 | 17254 | 17273 | N/A | N/A | CACTGTCCTTACTCCAACTT | 56 | D | 276 |
| 1380890 | 19363 | 19382 | N/A | N/A | CTAAAGTGGCCCCTCCAGCA | 36 | D | 277 |
| 1380909 | 24445 | 24464 | 2503 | 2522 | GCACTTTGCGAACCAACGAT | 40 | D | 278 |
| 1380948 | 16702 | 16721 | 931 | 950 | CACCGCCCACAGCCTGCAGG | 87 | D | 279 |
| 1380981 | 22314 | 22333 | N/A | N/A | GCTCAAAATCCCTCCAGCTC | 64 | D | 280 |
| 1380990 | 21377 | 21396 | N/A | N/A | CACCTCCACAGATGTGGTCC | 91 | D | 281 |
| 1381027 | 22908 | 22927 | 1756 | 1775 | TCCGCTCCTGCAACTGCCGG | 96† | D | 282 |
| 1381028 | 22297 | 22316 | N/A | N/A | CTCCCTAATGCCCTCACGAC | 89 | D | 283 |
| 1381052 | 17121 | 17140 | 1274 | 1293 | TCGGTGGCACCTTCGAAATC | 81 | D | 284 |
| 1381076 | 24461 | 24480 | 2519 | 2538 | TGCACAAGAAAGCTTTGCAC | 76 | D | 285 |
| 1381090 | 22054 | 22073 | N/A | N/A | CCCTTACTTAATGCCCCACT | 87 | D | 286 |
| 1381097 | 9691 | 9710 | N/A | N/A | GACTCAGGACTCTACGATTC | 89 | D | 287 |
| 1381104 | 22201 | 22220 | N/A | N/A | GGAACAAATCAGGATTCCCA | 74† | D | 288 |
| 1381146 | 22330 | 22349 | N/A | N/A | CCTATGTCCCTCTGCTGCTC | 73 | D | 289 |
| 1381165 | 11701 | 11720 | N/A | N/A | CCGACAAGCTCCAGAACTGG | 47 | D | 290 |
| 1381178 | 20873 | 20892 | N/A | N/A | AAGCAATGCATTATTTGTGT | 16 | D | 291 |
| 1381217 | 21107 | 21126 | N/A | N/A | GATAAGGTATAGTATGTGTA | 15 | D | 292 |
| 1381255 | 14023 | 14042 | N/A | N/A | CTTTAGTCCTACCCCTTATT | 65 | D | 293 |
| 1381266 | 17530 | 17549 | N/A | N/A | GGAGGAGTCCTCTCCTGCTT | 78 | D | 294 |
| 1381285 | 20703 | 20722 | N/A | N/A | GCACGATTTTTTCAATTTTT | 15 | D | 295 |
| 1381305 | 24346 | 24365 | 2404 | 2423 | AGGGCCTCAGCCTGGCCGAA | 45 | D | 296 |
| 1381333 | 24228 | 24247 | 2286 | 2305 | AAGGACCCTTCGAGCCCCGT | 93 | D | 297 |
| 1381337 | 14313 | 14332 | N/A | N/A | AATAAGACCCAGTTCTTCCA | 43 | D | 298 |
| 1381354 | 24381 | 24400 | 2439 | 2458 | TGCCTTCCCAGGCCTGCAGT | 106 | D | 299 |
| 1381375 | 24535 | 24554 | 2593 | 2612 | CGGGTTTGGCAAAAGCAAAT | 70 | D | 300 |
| 1381378 | 12745 | 12764 | N/A | N/A | CTGCCCCATGCTGGCCCTCC | 61 | D | 301 |
| 1381395 | 12161 | 12180 | 194 | 213 | GCTGACATGTTGGACAGGCA | 140 | D | 302 |
| 1381413 | 24246 | 24265 | 2304 | 2323 | AGCAGCATTCCCGGCTACAA | 54 | D | 303 |
| 1381417 | 24090 | 24109 | 2148 | 2167 | CGCGGCTTCTGTGCCGTGCC | 80 | D | 304 |
| 1381432 | 22676 | 22695 | 1629 | 1648 | GGTGAGCACCTCCTCCTCCA | 100 | D | 305 |
| 1381437 | 24069 | 24088 | 2127 | 2146 | CGGGCACTCAGTCTTCCAAC | 88 | D | 306 |
| 1381438 | 19642 | 19661 | N/A | N/A | GCAACTCCATTGGCTGCCAA | 148 | D | 307 |
| 1381442 | 14669 | 14688 | 379 | 398 | GGACCTCCTTAAGCCTCACC | 82 | D | 308 |
| 1381457 | 13058 | 13077 | N/A | N/A | CCAGGAGAACTAAAGGACGC | 45 | D | 309 |
| 1381465 | 17053 | 17072 | 1206 | 1225 | AAAGAAGAAGGGATGTGTCC | 125 | D | 310 |
| 1381487 | 22590 | 22609 | N/A | N/A | CTGCCACTTCAGCCTGTGTA | 81 | D | 311 |
| 1381509 | 23892 | 23911 | 1950 | 1969 | CGCCTCCGATAGGCCAGGCC | 109 | D | 312 |
| 1381524 | 19894 | 19913 | N/A | N/A | TTTGCATTCTTTTACAACTG | 13 | D | 313 |
| 1381543 | 21666 | 21685 | N/A | N/A | GCTGCTTCCAAGACCTCCTG | 83 | D | 314 |
| 1381546 | 24140 | 24159 | 2198 | 2217 | GCTGGGCGGAGACCCACGCT | 70 | D | 315 |
| 1381566 | 15953 | 15972 | 707 | 726 | AAGCGCGCCATCTCGGCCGG | 77 | D | 316 |
| 1381574 | 24521 | 24540 | 2579 | 2598 | GCAAATTTCCCGAGTAAGCA | 61 | D | 317 |
| 1381600 | 24203 | 24222 | 2261 | 2280 | GGCCGCGGACCCGGCCCCTC | 114 | D | 318 |
| 1381614 | 15246 | 15265 | N/A | N/A | TCACCAGGTAGTTCTCATCC | 31 | D | 319 |
| 1381621 | 22267 | 22286 | N/A | N/A | GCTGGGTTTTGTTTCCTGCT | 88 | D | 320 |
| 1381638 | 17372 | 17391 | N/A | N/A | GACTGCTCTGTGTTCCCCCA | 38 | D | 321 |
| 1381643 | 24622 | 24641 | 2680 | 2699 | AATATCCAAACCGCCGAAGC | 67 | D | 322 |
| 1381651 | 15078 | 15097 | N/A | N/A | CAGCCCCTCACCTCGCCCCT | 91 | D | 323 |
| 1381674 | 15060 | 15079 | 515 | 534 | CTCTTCAGCATGTCCCACTT | 76 | D | 324 |
| 1381686 | 12777 | 12796 | N/A | N/A | GAACACCTGCCTGTCGGCTG | 152 | D | 325 |
| 1381687 | 24397 | 24416 | 2455 | 2474 | GGACGGCCCGGCTTGCTGCC | 78 | D | 326 |
| 1052873 | 19496 | 19515 | 1357 | 1376 | CTTCCCGAATGTCCGACAGT | 127 | E | 19 |
| 1380292 | 9056 | 9075 | N/A | N/A | TGAGACACTAAGATTTCCCT | 51 | E | 327 |
| 1380304 | 20875 | 20894 | N/A | N/A | ATAAGCAATGCATTATTTGT | 28 | E | 328 |
| 1380305 | 19674 | 19693 | N/A | N/A | GCCCAAAACAGTAAGGTTCC | 73 | E | 329 |
| 1380352 | 22298 | 22317 | N/A | N/A | GCTCCCTAATGCCCTCACGA | 84 | E | 330 |
| 1380364 | 17157 | 17176 | 1310 | 1329 | GTGAGCCCGTCCTCCACCAA | 99 | F | 331 |
| 1380371 | 22055 | 22074 | N/A | N/A | ACCCTTACTTAATGCCCCAC | 62 | E | 332 |
| 1380402 | 11703 | 11722 | N/A | N/A | TCCCGACAAGCTCCAGAACT | 64 | E | 333 |
| 1380436 | N/A | N/A | 780 | 799 | GGGTTTGATGTCCCTGTGCA | 77 | E | 334 |
| 1380438 | 21113 | 21132 | N/A | N/A | ATTAATGATAAGGTATAGTA | 94 | E | 335 |
| 1380448 | 17323 | 17342 | N/A | N/A | TCCTCAGTAGTAGATGGGCA | 68 | E | 336 |
| 1380469 | 17732 | 17751 | N/A | N/A | TGTATCCTGTTGCTTCCCTT | 31 | E | 337 |
| 1380490 | 17122 | 17141 | 1275 | 1294 | GTCGGTGGCACCTTCGAAAT | 59 | F | 338 |
| 1380498 | 22147 | 22166 | N/A | N/A | TGACCACTTGGCACCTTTCC | 105 | E | 339 |
| 1380520 | 21057 | 21076 | N/A | N/A | AACCAACTGTTCTCTTAGAC | 47 | E | 340 |
| 1380525 | 19364 | 19383 | N/A | N/A | TCTAAAGTGGCCCCTCCAGC | 64 | E | 341 |
| 1380580 | 15925 | 15944 | 679 | 698 | CCCCAAACTTGCTCAGCAGT | 113 | E | 342 |
| 1380590 | 16721 | 16740 | 950 | 969 | TAGCTGCCTGTCCCAGGCCC | 94 | E | 343 |
| 1380622 | 15507 | 15526 | N/A | N/A | TTCCAGGTAAGAGACCCCCC | 70 | E | 344 |
| 1380652 | 17255 | 17274 | N/A | N/A | GCACTGTCCTTACTCCAACT | 26 | E | 345 |
| 1380654 | 24153 | 24172 | 2211 | 2230 | TCACAGGACTGGAGCTGGGC | 77 | E | 346 |
| 1380669 | 20958 | 20977 | N/A | N/A | GTGAGCCAGGGACCACTGCC | 98 | E | 347 |
| 1380705 | 17703 | 17722 | N/A | N/A | TGCCACACTCTCCATAATTC | 98 | E | 348 |
| 1380717 | 16704 | 16723 | 933 | 952 | CCCACCGCCCACAGCCTGCA | 99 | E | 349 |
| 1380736 | 22315 | 22334 | N/A | N/A | TGCTCAAAATCCCTCCAGCT | 125 | E | 350 |
| 1380770 | 22331 | 22350 | N/A | N/A | ACCTATGTCCCTCTGCTGCT | 63 | E | 351 |
| 1380779 | 24637 | 24656 | 2695 | 2714 | AGGACGAGGTCAATAAATAT | 76 | E | 352 |
| 1380790 | 14522 | 14541 | N/A | N/A | TAGGACTGTCTGCTTCCCAG | 65 | E | 353 |
| 1380796 | 24446 | 24465 | 2504 | 2523 | TGCACTTTGCGAACCAACGA | 42 | E | 354 |
| 1380803 | 19795 | 19814 | N/A | N/A | TTACAAGATTCTGGGAAGCC | 7 | E | 355 |
| 1380837 | 17618 | 17637 | N/A | N/A | CCCAGTATTGTTCAGCAAAA | 85 | E | 356 |
| 1380843 | 10409 | 10428 | N/A | N/A | CCCCCAAATTTTGTGCAGGT | 53 | E | 357 |
| 1380850 | 19501 | 19520 | 1362 | 1381 | CGCACCTTCCCGAATGTCCG | 71 | E | 358 |
| 1380853 | 15640 | 15659 | N/A | N/A | CACTGGGCAGATTCACTCCC | 86 | E | 359 |
| 1380871 | 22227 | 22246 | N/A | N/A | GAATGATTCAGCCAAACTAC | 78 | E | 360 |
| 1380896 | 14054 | 14073 | N/A | N/A | CAGCCCATCTCTCAGTCCTC | 77 | E | 361 |
| 1380950 | 20922 | 20941 | N/A | N/A | AGATATCAACTTCCTTTTCC | 57 | E | 362 |
| 1380965 | 21982 | 22001 | N/A | N/A | CTGTCTGCTTCTGTTCAGGA | 56 | E | 363 |
| 1380968 | 13985 | 14004 | N/A | N/A | CTCTCAGAAGTCAAGGTCCT | 44 | E | 364 |
| 1380983 | 22909 | 22928 | 1757 | 1776 | ATCCGCTCCTGCAACTGCCG | 124† | E | 365 |
| 1380987 | 24732 | 24751 | 2790 | 2809 | GGACAGACAATAAATACCGA | 69 | E | 366 |
| 1381061 | 14192 | 14211 | N/A | N/A | ATCCAGTACCTCTAGATTCA | 24 | E | 367 |
| 1381078 | 13961 | 13980 | N/A | N/A | CTAGGAATGTTAAACTGGGC | 54 | E | 368 |
| 1381119 | 24382 | 24401 | 2440 | 2459 | CTGCCTTCCCAGGCCTGCAG | 109 | E | 369 |
| 1381168 | 21379 | 21398 | N/A | N/A | GCCACCTCCACAGATGTGGT | 118 | E | 370 |
| 1381230 | 19909 | 19928 | N/A | N/A | GGCTGATTCAAAGAATTTGC | 59 | E | 371 |
| 1381303 | 22591 | 22610 | N/A | N/A | ACTGCCACTTCAGCCTGTGT | 125 | E | 372 |
| 1381310 | 24070 | 24089 | 2128 | 2147 | CCGGGCACTCAGTCTTCCAA | 92 | E | 373 |
| 1381323 | 15248 | 15267 | N/A | N/A | GCTCACCAGGTAGTTCTCAT | 98 | E | 374 |
| 1381342 | 24247 | 24266 | 2305 | 2324 | CAGCAGCATTCCCGGCTACA | 67 | E | 375 |
| 1381351 | 15961 | 15980 | 715 | 734 | CCAGGTAGAAGCGCGCCATC | 83 | E | 376 |
| 1381376 | 21810 | 21829 | 1475 | 1494 | TCAAGCAGCTGCTCGGCCTC | 104 | E | 377 |
| 1381425 | 12164 | 12183 | 197 | 216 | TCGGCTGACATGTTGGACAG | 123 | E | 378 |
| 1381428 | 24091 | 24110 | 2149 | 2168 | GCGCGGCTTCTGTGCCGTGC | 112 | E | 379 |
| 1381433 | 14654 | 14673 | N/A | N/A | TCACCACGATGGGCTCCGCT | 93 | E | 380 |
| 1381444 | 24536 | 24555 | 2594 | 2613 | GCGGGTTTGGCAAAAGCAAA | 88 | E | 381 |
| 1381446 | 24204 | 24223 | 2262 | 2281 | CGGCCGCGGACCCGGCCCCT | 100 | E | 382 |
| 1381450 | 24462 | 24481 | 2520 | 2539 | ATGCACAAGAAAGCTTTGCA | 68 | E | 383 |
| 1381464 | 17059 | 17078 | 1212 | 1231 | GAGGCCAAAGAAGAAGGGAT | 69 | E | 384 |
| 1381482 | 20705 | 20724 | N/A | N/A | TGGCACGATTTTTTCAATTT | 61 | E | 385 |
| 1381493 | 13060 | 13079 | N/A | N/A | GGCCAGGAGAACTAAAGGAC | 91 | F | 386 |
| 1381499 | 15061 | 15080 | 516 | 535 | CCTCTTCAGCATGTCCCACT | 78 | E | 387 |
| 1381512 | 24522 | 24541 | 2580 | 2599 | AGCAAATTTCCCGAGTAAGC | 69 | E | 388 |
| 1381515 | 15082 | 15101 | N/A | N/A | CGCCCAGCCCCTCACCTCGC | 170 | E | 389 |
| 1381518 | 24623 | 24642 | 2681 | 2700 | AAATATCCAAACCGCCGAAG | 57 | E | 390 |
| 1381531 | 14670 | 14689 | 380 | 399 | CGGACCTCCTTAAGCCTCAC | 84 | E | 391 |
| 1381533 | 19644 | 19663 | N/A | N/A | CTGCAACTCCATTGGCTGCC | 92 | E | 392 |
| 1381581 | 12746 | 12765 | N/A | N/A | GCTGCCCCATGCTGGCCCTC | 66 | E | 393 |
| 1381602 | 17373 | 17392 | N/A | N/A | AGACTGCTCTGTGTTCCCCC | 62 | F | 394 |
| 1381605 | 24229 | 24248 | 2287 | 2306 | CAAGGACCCTTCGAGCCCCG | 127 | E | 395 |
| 1381612 | 22677 | 22696 | 1630 | 1649 | GGGTGAGCACCTCCTCCTCC | 141 | E | 396 |
| 1381634 | 23893 | 23912 | 1951 | 1970 | GCGCCTCCGATAGGCCAGGC | 77 | E | 397 |
| 1381647 | 17532 | 17551 | N/A | N/A | GAGGAGGAGTCCTCTCCTGC | 108 | E | 398 |
| 1381655 | 24398 | 24417 | 2456 | 2475 | CGGACGGCCCGGCTTGCTGC | 118 | E | 399 |
| 1381661 | 12779 | 12798 | N/A | N/A | CCGAACACCTGCCTGTCGGC | 141 | E | 400 |
| 1381665 | 22278 | 22297 | N/A | N/A | CAAAAGGCCTTGCTGGGTTT | 57 | E | 401 |
| 1381677 | 14024 | 14043 | N/A | N/A | GCTTTAGTCCTACCCCTTAT | 19 | E | 402 |
| 1381679 | 24347 | 24366 | 2405 | 2424 | CAGGGCCTCAGCCTGGCCGA | 72 | F | 403 |
| 1052873 | 19496 | 19515 | 1357 | 1376 | CTTCCCGAATGTCCGACAGT | 110 | F | 19 |
| 1380290 | 21115 | 21134 | N/A | N/A | TCATTAATGATAAGGTATAG | 56 | F | 404 |
| 1380334 | 21058 | 21077 | N/A | N/A | AAACCAACTGTTCTCTTAGA | 58 | F | 405 |
| 1380340 | 20959 | 20978 | N/A | N/A | TGTGAGCCAGGGACCACTGC | 94 | F | 406 |
| 1380347 | 15329 | 15348 | N/A | N/A | ATCCTAGAGCTTCCTCTCCC | 78 | F | 407 |
| 1380420 | 17619 | 17638 | N/A | N/A | CCCCAGTATTGTTCAGCAAA | 107 | F | 408 |
| 1380427 | 19676 | 19695 | N/A | N/A | GGGCCCAAAACAGTAAGGTT | 160 | F | 409 |
| 1380432 | 17734 | 17753 | N/A | N/A | CTTGTATCCTGTTGCTTCCC | 30 | F | 410 |
| 1380440 | 22148 | 22167 | N/A | N/A | CTGACCACTTGGCACCTTTC | 85 | F | 411 |
| 1380454 | 19796 | 19815 | N/A | N/A | GTTACAAGATTCTGGGAAGC | 22 | F | 412 |
| 1380466 | 13986 | 14005 | N/A | N/A | CCTCTCAGAAGTCAAGGTCC | 66 | F | 413 |
| 1380543 | 17704 | 17723 | N/A | N/A | CTGCCACACTCTCCATAATT | 95 | F | 414 |
| 1380547 | 21986 | 22005 | N/A | N/A | GTTTCTGTCTGCTTCTGTTC | 33 | F | 415 |
| 1380593 | 17256 | 17275 | N/A | N/A | GGCACTGTCCTTACTCCAAC | 39 | F | 416 |
| 1380609 | 14523 | 14542 | N/A | N/A | CTAGGACTGTCTGCTTCCCA | 63 | F | 417 |
| 1380621 | 13965 | 13984 | N/A | N/A | ATGACTAGGAATGTTAAACT | 53 | F | 418 |
| 1380645 | 24383 | 24402 | 2441 | 2460 | GCTGCCTTCCCAGGCCTGCA | 212 | F | 419 |
| 1380706 | 11704 | 11723 | N/A | N/A | CTCCCGACAAGCTCCAGAAC | 75 | F | 420 |
| 1380708 | 22910 | 22929 | 1758 | 1777 | CATCCGCTCCTGCAACTGCC | 169 | F | 421 |
| 1380723 | 19656 | 19675 | N/A | N/A | CCAAGACTGATCCTGCAACT | 41 | F | 422 |
| 1380731 | 10410 | 10429 | N/A | N/A | GCCCCCAAATTTTGTGCAGG | 65 | F | 423 |
| 1380750 | 19913 | 19932 | N/A | N/A | ATATGGCTGATTCAAAGAAT | 57 | F | 424 |
| 1380756 | 15508 | 15527 | N/A | N/A | ATTCCAGGTAAGAGACCCCC | 47 | F | 425 |
| 1380781 | 22316 | 22335 | N/A | N/A | CTGCTCAAAATCCCTCCAGC | 74 | F | 426 |
| 1380798 | 20923 | 20942 | N/A | N/A | GAGATATCAACTTCCTTTTC | 27 | F | 427 |
| 1380799 | 17123 | 17142 | 1276 | 1295 | TGTCGGTGGCACCTTCGAAA | 85 | F | 428 |
| 1380815 | 22056 | 22075 | N/A | N/A | CACCCTTACTTAATGCCCCA | 128 | F | 429 |
| 1380856 | 16705 | 16724 | 934 | 953 | GCCCACCGCCCACAGCCTGC | 83 | F | 430 |
| 1380867 | 22279 | 22298 | N/A | N/A | ACAAAAGGCCTTGCTGGGTT | 63 | F | 431 |
| 1380888 | 14055 | 14074 | N/A | N/A | CCAGCCCATCTCTCAGTCCT | 56 | F | 432 |
| 1380923 | 22299 | 22318 | N/A | N/A | AGCTCCCTAATGCCCTCACG | 91 | F | 433 |
| 1380940 | 19502 | 19521 | 1363 | 1382 | GCGCACCTTCCCGAATGTCC | 92 | F | 434 |
| 1380975 | 20877 | 20896 | N/A | N/A | TGATAAGCAATGCATTATTT | 65 | F | 435 |
| 1381031 | 15926 | 15945 | 680 | 699 | TCCCCAAACTTGCTCAGCAG | 89 | F | 436 |
| 1381037 | 24733 | 24752 | 2791 | 2810 | GGGACAGACAATAAATACCG | 57 | F | 437 |
| 1381048 | 22228 | 22247 | N/A | N/A | GGAATGATTCAGCCAAACTA | 47 | F | 438 |
| 1381050 | N/A | N/A | 781 | 800 | CGGGTTTGATGTCCCTGTGC | 90 | F | 439 |
| 1381056 | 17158 | 17177 | 1311 | 1330 | AGTGAGCCCGTCCTCCACCA | 67 | F | 440 |
| 1381085 | 22332 | 22351 | N/A | N/A | AACCTATGTCCCTCTGCTGC | 78 | F | 441 |
| 1381095 | 16722 | 16741 | 951 | 970 | GTAGCTGCCTGTCCCAGGCC | 124 | F | 442 |
| 1381099 | 17334 | 17353 | N/A | N/A | TCACTGCTGGGTCCTCAGTA | 88 | F | 443 |
| 1381123 | 17566 | 17585 | N/A | N/A | AGCTTGTTACACGGTGAAGA | 52 | F | 444 |
| 1381131 | 14193 | 14212 | N/A | N/A | TATCCAGTACCTCTAGATTC | 62 | F | 445 |
| 1381134 | 9057 | 9076 | N/A | N/A | CTGAGACACTAAGATTTCCC | 47 | F | 446 |
| 1381200 | 24447 | 24466 | 2505 | 2524 | TTGCACTTTGCGAACCAACG | 28 | F | 447 |
| 1381215 | 13470 | 13489 | N/A | N/A | AGGTTTTTCCAGAGGCTGAA | 13 | F | 448 |
| 1381219 | 19367 | 19386 | N/A | N/A | TTATCTAAAGTGGCCCCTCC | 85 | F | 449 |
| 1381238 | 24523 | 24542 | 2581 | 2600 | AAGCAAATTTCCCGAGTAAG | 62 | F | 450 |
| 1381276 | 17060 | 17079 | 1213 | 1232 | CGAGGCCAAAGAAGAAGGGA | 70 | F | 451 |
| 1381280 | 24403 | 24422 | 2461 | 2480 | GAACACGGACGGCCCGGCTT | 104 | F | 452 |
| 1381308 | 14656 | 14675 | 366 | 385 | CCTCACCACGATGGGCTCCG | 60 | F | 453 |
| 1381314 | 22592 | 22611 | N/A | N/A | AACTGCCACTTCAGCCTGTG | 175 | F | 454 |
| 1381325 | 24071 | 24090 | 2129 | 2148 | CCCGGGCACTCAGTCTTCCA | 110 | F | 455 |
| 1381328 | 24248 | 24267 | 2306 | 2325 | GCAGCAGCATTCCCGGCTAC | 78 | F | 456 |
| 1381330 | 23914 | 23933 | 1972 | 1991 | CGGCGAACAGGAGCAGGGAA | 121 | F | 457 |
| 1381336 | 24638 | 24657 | 2696 | 2715 | GAGGACGAGGTCAATAAATA | 56 | F | 458 |
| 1381338 | 24624 | 24643 | 2682 | 2701 | TAAATATCCAAACCGCCGAA | 99 | F | 459 |
| 1381340 | 17454 | 17473 | N/A | N/A | CCTTCCTTGCTGAGTCAGGA | 73 | F | 460 |
| 1381343 | 15752 | 15771 | N/A | N/A | CCCCAGCCCAGAGATAACCA | 127 | F | 461 |
| 1381389 | 15962 | 15981 | 716 | 735 | GCCAGGTAGAAGCGCGCCAT | 84 | F | 462 |
| 1381396 | 15083 | 15102 | N/A | N/A | CCGCCCAGCCCCTCACCTCG | 92 | F | 463 |
| 1381402 | 24463 | 24482 | 2521 | 2540 | CATGCACAAGAAAGCTTTGC | 97 | F | 464 |
| 1381418 | 14671 | 14690 | 381 | 400 | TCGGACCTCCTTAAGCCTCA | 44 | F | 465 |
| 1381478 | 12781 | 12800 | N/A | N/A | CGCCGAACACCTGCCTGTCG | 141 | F | 466 |
| 1381479 | 20717 | 20736 | N/A | N/A | GACCACCATGCCTGGCACGA | 100 | F | 467 |
| 1381480 | 21811 | 21830 | 1476 | 1495 | CTCAAGCAGCTGCTCGGCCT | 131 | F | 468 |
| 1381491 | 12165 | 12184 | 198 | 217 | CTCGGCTGACATGTTGGACA | 97 | F | 469 |
| 1381504 | 15062 | 15081 | 517 | 536 | CCCTCTTCAGCATGTCCCAC | 93 | F | 470 |
| 1381507 | 14026 | 14045 | N/A | N/A | GTGCTTTAGTCCTACCCCTT | 17 | F | 471 |
| 1381527 | 24538 | 24557 | 2596 | 2615 | AAGCGGGTTTGGCAAAAGCA | 89 | F | 472 |
| 1381572 | 21390 | 21409 | N/A | N/A | GCTTGGCTCTGGCCACCTCC | 121 | F | 473 |
| 1381577 | 24348 | 24367 | 2406 | 2425 | TCAGGGCCTCAGCCTGGCCG | 132 | F | 474 |
| 1381579 | 12747 | 12766 | N/A | N/A | AGCTGCCCCATGCTGGCCCT | 148 | F | 475 |
| 1381590 | 24230 | 24249 | 2288 | 2307 | ACAAGGACCCTTCGAGCCCC | 89 | F | 476 |
| 1381604 | 24093 | 24112 | 2151 | 2170 | GGGCGCGGCTTCTGTGCCGT | 97 | F | 477 |
| 1381670 | 24157 | 24176 | 2215 | 2234 | CGGATCACAGGACTGGAGCT | 46 | F | 478 |
| 1381675 | 22678 | 22697 | 1631 | 1650 | CGGGTGAGCACCTCCTCCTC | 249 | F | 479 |
| 1381685 | 24208 | 24227 | 2266 | 2285 | TCGCCGGCCGCGGACCCGGC | 114 | F | 480 |
| 1052873 | 19496 | 19515 | 1357 | 1376 | CTTCCCGAATGTCCGACAGT | 77 | G | 19 |
| 1380279 | 22284 | 22303 | N/A | N/A | TCACGACAAAAGGCCTTGCT | 61 | G | 481 |
| 1380294 | 20878 | 20897 | N/A | N/A | GTGATAAGCAATGCATTATT | 18 | G | 482 |
| 1380302 | 13987 | 14006 | N/A | N/A | GCCTCTCAGAAGTCAAGGTC | 71 | G | 483 |
| 1380310 | 17462 | 17481 | N/A | N/A | CTCCAGGACCTTCCTTGCTG | 85 | G | 484 |
| 1380314 | 21060 | 21079 | N/A | N/A | GAAAACCAACTGTTCTCTTA | 75 | G | 485 |
| 1380392 | 16706 | 16725 | 935 | 954 | GGCCCACCGCCCACAGCCTG | 80 | G | 486 |
| 1380401 | 22149 | 22168 | N/A | N/A | CCTGACCACTTGGCACCTTT | 78 | G | 487 |
| 1380404 | 17159 | 17178 | 1312 | 1331 | CAGTGAGCCCGTCCTCCACC | 110 | G | 488 |
| 1380417 | 17339 | 17358 | N/A | N/A | CTAGGTCACTGCTGGGTCCT | 85 | G | 489 |
| 1380421 | 13966 | 13985 | N/A | N/A | TATGACTAGGAATGTTAAAC | 63 | G | 490 |
| 1380519 | 15927 | 15946 | 681 | 700 | CTCCCCAAACTTGCTCAGCA | 65 | G | 491 |
| 1380545 | 20924 | 20943 | N/A | N/A | CGAGATATCAACTTCCTTTT | 29 | G | 492 |
| 1380568 | 22911 | 22930 | 1759 | 1778 | CCATCCGCTCCTGCAACTGC | 130 | G | 493 |
| 1380569 | 24659 | 24678 | 2717 | 2736 | CTGTAGCCTGTCAGCGAGTC | 71 | G | 494 |
| 1380573 | 14056 | 14075 | N/A | N/A | TCCAGCCCATCTCTCAGTCC | 58 | G | 495 |
| 1380600 | 10411 | 10430 | N/A | N/A | GGCCCCCAAATTTTGTGCAG | 69 | G | 496 |
| 1380608 | 17124 | 17143 | 1277 | 1296 | GTGTCGGTGGCACCTTCGAA | 51 | G | 497 |
| 1380625 | 17257 | 17276 | N/A | N/A | AGGCACTGTCCTTACTCCAA | 65 | G | 498 |
| 1380686 | 21987 | 22006 | N/A | N/A | GGTTTCTGTCTGCTTCTGTT | 8 | G | 499 |
| 1380697 | 11705 | 11724 | N/A | N/A | GCTCCCGACAAGCTCCAGAA | 42 | G | 500 |
| 1380703 | 22333 | 22352 | N/A | N/A | CAACCTATGTCCCTCTGCTG | 104 | G | 501 |
| 1380712 | 17705 | 17724 | N/A | N/A | CCTGCCACACTCTCCATAAT | 61 | G | 502 |
| 1380748 | 19915 | 19934 | N/A | N/A | TTATATGGCTGATTCAAAGA | 30 | G | 503 |
| 1380749 | 22058 | 22077 | N/A | N/A | CACACCCTTACTTAATGCCC | 97 | G | 504 |
| 1380753 | 24158 | 24177 | 2216 | 2235 | CCGGATCACAGGACTGGAGC | 69 | G | 505 |
| 1380777 | 19800 | 19819 | N/A | N/A | AATGGTTACAAGATTCTGGG | 11 | G | 506 |
| 1380783 | 20805 | 20824 | N/A | N/A | AATCTATCATGGCTCACTGA | 77 | G | 507 |
| 1380785 | 17568 | 17587 | N/A | N/A | ACAGCTTGTTACACGGTGAA | 25 | G | 508 |
| 1380868 | 14194 | 14213 | N/A | N/A | GTATCCAGTACCTCTAGATT | 12 | G | 509 |
| 1380875 | 9240 | 9259 | N/A | N/A | AGAAATAGATTCTGGTTCGA | 29 | G | 510 |
| 1380886 | 15331 | 15350 | N/A | N/A | CAATCCTAGAGCTTCCTCTC | 87 | G | 511 |
| 1380917 | 16723 | 16742 | 952 | 971 | CGTAGCTGCCTGTCCCAGGC | 86 | G | 512 |
| 1380934 | 22300 | 22319 | N/A | N/A | CAGCTCCCTAATGCCCTCAC | 130 | G | 513 |
| 1380954 | 21116 | 21135 | N/A | N/A | TTCATTAATGATAAGGTATA | 54 | G | 514 |
| 1381000 | 22229 | 22248 | N/A | N/A | AGGAATGATTCAGCCAAACT | 58 | G | 515 |
| 1381009 | 17620 | 17639 | N/A | N/A | TCCCCAGTATTGTTCAGCAA | 45 | G | 516 |
| 1381011 | 20982 | 21001 | N/A | N/A | ATTTAAACATGTGTCAGTAC | 36 | G | 517 |
| 1381121 | 15510 | 15529 | N/A | N/A | CCATTCCAGGTAAGAGACCC | 77 | G | 518 |
| 1381125 | 22317 | 22336 | N/A | N/A | GCTGCTCAAAATCCCTCCAG | 53 | G | 519 |
| 1381143 | 19503 | 19522 | 1364 | 1383 | GGCGCACCTTCCCGAATGTC | 74 | G | 520 |
| 1381156 | 13471 | 13490 | N/A | N/A | AAGGTTTTTCCAGAGGCTGA | 20 | G | 521 |
| 1381162 | 24384 | 24403 | 2442 | 2461 | TGCTGCCTTCCCAGGCCTGC | 94 | G | 522 |
| 1381175 | 12869 | 12888 | N/A | N/A | GTCTGCAAAGCTGGTTCTCC | 51 | G | 523 |
| 1381180 | 14524 | 14543 | N/A | N/A | CCTAGGACTGTCTGCTTCCC | 85 | G | 524 |
| 1381213 | 19368 | 19387 | N/A | N/A | CTTATCTAAAGTGGCCCCTC | 93 | G | 525 |
| 1381214 | 17735 | 17754 | N/A | N/A | TCTTGTATCCTGTTGCTTCC | 26 | G | 526 |
| 1381223 | 24448 | 24467 | 2506 | 2525 | TTTGCACTTTGCGAACCAAC | 28 | G | 527 |
| 1381228 | 12748 | 12767 | N/A | N/A | CAGCTGCCCCATGCTGGCCC | 100 | G | 528 |
| 1381235 | 19657 | 19676 | N/A | N/A | TCCAAGACTGATCCTGCAAC | 44 | G | 529 |
| 1381236 | 24350 | 24369 | 2408 | 2427 | CGTCAGGGCCTCAGCCTGGC | 102 | G | 530 |
| 1381239 | 21812 | 21831 | 1477 | 1496 | GCTCAAGCAGCTGCTCGGCC | 76 | G | 531 |
| 1381295 | 21391 | 21410 | N/A | N/A | TGCTTGGCTCTGGCCACCTC | 75 | G | 532 |
| 1381298 | 24249 | 24268 | 2307 | 2326 | AGCAGCAGCATTCCCGGCTA | 50 | G | 533 |
| 1381307 | 24231 | 24250 | 2289 | 2308 | TACAAGGACCCTTCGAGCCC | 80 | G | 534 |
| 1381319 | 24407 | 24426 | 2465 | 2484 | GATGGAACACGGACGGCCCG | 81 | G | 535 |
| 1381334 | 24524 | 24543 | 2582 | 2601 | AAAGCAAATTTCCCGAGTAA | 63 | G | 536 |
| 1381341 | 24212 | 24231 | 2270 | 2289 | CCGTTCGCCGGCCGCGGACC | 64 | G | 537 |
| 1381356 | 23948 | 23967 | 2006 | 2025 | ATGCAGCCCAGGGCGGCGGC | 105 | G | 538 |
| 1381368 | 24625 | 24644 | 2683 | 2702 | ATAAATATCCAAACCGCCGA | 79 | G | 539 |
| 1381377 | 24072 | 24091 | 2130 | 2149 | CCCCGGGCACTCAGTCTTCC | 91 | G | 540 |
| 1381399 | 22679 | 22698 | 1632 | 1651 | CCGGGTGAGCACCTCCTCCT | 148 | G | 541 |
| 1381431 | 15162 | 15181 | N/A | N/A | CACGGAAGCACGACACCTGC | 74 | G | 542 |
| 1381469 | 24548 | 24567 | 2606 | 2625 | ATCCCCGAAAAAGCGGGTTT | 70 | G | 543 |
| 1381483 | 22596 | 22615 | N/A | N/A | CTGGAACTGCCACTTCAGCC | 127 | G | 544 |
| 1381496 | 16300 | 16319 | N/A | N/A | TCGGGTTTGATGTCCCTGCA | 119 | G | 545 |
| 1381521 | 24773 | 24792 | 2831 | 2850 | GCCTTTTATTCGCGAGGGTC | 46 | G | 546 |
| 1381560 | 14672 | 14691 | 382 | 401 | GTCGGACCTCCTTAAGCCTC | 45 | G | 547 |
| 1381613 | 24464 | 24483 | 2522 | 2541 | TCATGCACAAGAAAGCTTTG | 62 | G | 548 |
| 1381616 | 19696 | 19715 | N/A | N/A | CTGGCCTCTTAGGAGTCTTT | 104 | G | 549 |
| 1381625 | 15963 | 15982 | 717 | 736 | CGCCAGGTAGAAGCGCGCCA | 84 | G | 550 |
| 1381627 | 17070 | 17089 | 1223 | 1242 | CCATCCCAGTCGAGGCCAAA | 71 | G | 551 |
| 1381632 | 15063 | 15082 | 518 | 537 | CCCCTCTTCAGCATGTCCCA | 71 | G | 552 |
| 1381639 | 14657 | 14676 | 367 | 386 | GCCTCACCACGATGGGCTCC | 80 | G | 553 |
| 1381644 | 15768 | 15787 | N/A | N/A | GGGCAGAGACCTGCAGCCCC | 107 | G | 554 |
| 1381646 | 12166 | 12185 | 199 | 218 | CCTCGGCTGACATGTTGGAC | 90 | G | 555 |
| 1381657 | 14027 | 14046 | N/A | N/A | AGTGCTTTAGTCCTACCCCT | 24 | G | 556 |
| 1381658 | 24095 | 24114 | 2153 | 2172 | GTGGGCGCGGCTTCTGTGCC | 102 | G | 557 |
| 1052873 | 19496 | 19515 | 1357 | 1376 | CTTCCCGAATGTCCGACAGT | 74 | H | 19 |
| 1380282 | 17706 | 17725 | N/A | N/A | ACCTGCCACACTCTCCATAA | 66 | H | 558 |
| 1380322 | 13472 | 13491 | N/A | N/A | GAAGGTTTTTCCAGAGGCTG | 21 | H | 559 |
| 1380324 | 22285 | 22304 | N/A | N/A | CTCACGACAAAAGGCCTTGC | 98 | H | 560 |
| 1380357 | 20984 | 21003 | N/A | N/A | CTATTTAAACATGTGTCAGT | 37 | H | 561 |
| 1380363 | 17464 | 17483 | N/A | N/A | CCCTCCAGGACCTTCCTTGC | 84 | H | 562 |
| 1380381 | 15928 | 15947 | 682 | 701 | GCTCCCCAAACTTGCTCAGC | 109 | H | 563 |
| 1380403 | 20880 | 20899 | N/A | N/A | TTGTGATAAGCAATGCATTA | 37 | H | 564 |
| 1380467 | 16707 | 16726 | 936 | 955 | AGGCCCACCGCCCACAGCCT | 90 | H | 565 |
| 1380500 | 11706 | 11725 | N/A | N/A | TGCTCCCGACAAGCTCCAGA | 49 | H | 566 |
| 1380508 | 19803 | 19822 | N/A | N/A | GTTAATGGTTACAAGATTCT | 36 | H | 567 |
| 1380526 | 13967 | 13986 | N/A | N/A | CTATGACTAGGAATGTTAAA | 61 | H | 568 |
| 1380527 | 22912 | 22931 | 1760 | 1779 | TCCATCCGCTCCTGCAACTG | 90 | H | 569 |
| 1380529 | 14525 | 14544 | N/A | N/A | CCCTAGGACTGTCTGCTTCC | 81 | H | 570 |
| 1380548 | 15166 | 15185 | 542 | 561 | TCCTCACGGAAGCACGACAC | 73 | H | 571 |
| 1380549 | 10412 | 10431 | N/A | N/A | GGGCCCCCAAATTTTGTGCA | 97 | H | 572 |
| 1380588 | 17569 | 17588 | N/A | N/A | GACAGCTTGTTACACGGTGA | 32 | H | 573 |
| 1380641 | 12871 | 12890 | N/A | N/A | CTGTCTGCAAAGCTGGTTCT | 80 | H | 574 |
| 1380647 | 17736 | 17755 | N/A | N/A | TTCTTGTATCCTGTTGCTTC | 26 | H | 575 |
| 1380693 | 21117 | 21136 | N/A | N/A | GTTCATTAATGATAAGGTAT | 4 | H | 576 |
| 1380709 | 24385 | 24404 | 2443 | 2462 | TTGCTGCCTTCCCAGGCCTG | 86 | H | 577 |
| 1380722 | 21062 | 21081 | N/A | N/A | TAGAAAACCAACTGTTCTCT | 65 | H | 578 |
| 1380746 | 19658 | 19677 | N/A | N/A | TTCCAAGACTGATCCTGCAA | 79 | H | 579 |
| 1380760 | 16725 | 16744 | 954 | 973 | CCCGTAGCTGCCTGTCCCAG | 131 | H | 580 |
| 1380761 | 22301 | 22320 | N/A | N/A | CCAGCTCCCTAATGCCCTCA | 66 | H | 581 |
| 1380802 | 17258 | 17277 | N/A | N/A | TAGGCACTGTCCTTACTCCA | 74 | H | 582 |
| 1380812 | 24626 | 24645 | 2684 | 2703 | AATAAATATCCAAACCGCCG | 69 | H | 583 |
| 1380824 | 22318 | 22337 | N/A | N/A | TGCTGCTCAAAATCCCTCCA | 71 | H | 584 |
| 1380846 | 19504 | 19523 | 1365 | 1384 | CGGCGCACCTTCCCGAATGT | 84 | H | 585 |
| 1380857 | 20925 | 20944 | N/A | N/A | ACGAGATATCAACTTCCTTT | 24 | H | 586 |
| 1380862 | 21990 | 22009 | N/A | N/A | CGTGGTTTCTGTCTGCTTCT | 31 | H | 587 |
| 1380870 | 19919 | 19938 | N/A | N/A | ATTGTTATATGGCTGATTCA | 26 | H | 588 |
| 1380876 | 17160 | 17179 | 1313 | 1332 | GCAGTGAGCCCGTCCTCCAC | 104 | H | 589 |
| 1380892 | 20807 | 20826 | N/A | N/A | GGAATCTATCATGGCTCACT | 12 | H | 590 |
| 1380920 | 9242 | 9261 | N/A | N/A | ACAGAAATAGATTCTGGTTC | 72 | H | 591 |
| 1380925 | 15512 | 15531 | N/A | N/A | TCCCATTCCAGGTAAGAGAC | 106 | H | 592 |
| 1380959 | 22230 | 22249 | N/A | N/A | CAGGAATGATTCAGCCAAAC | 48 | H | 593 |
| 1380971 | 19369 | 19388 | N/A | N/A | GCTTATCTAAAGTGGCCCCT | 102 | H | 594 |
| 1380989 | 22334 | 22353 | N/A | N/A | ACAACCTATGTCCCTCTGCT | 73 | H | 595 |
| 1381039 | 13988 | 14007 | N/A | N/A | GGCCTCTCAGAAGTCAAGGT | 79 | H | 596 |
| 1381046 | 15332 | 15351 | N/A | N/A | CCAATCCTAGAGCTTCCTCT | 72 | H | 597 |
| 1381054 | 24035 | 24054 | 2093 | 2112 | GAAGACAGTTCTAGGGTTCA | 53 | H | 598 |
| 1381068 | 24449 | 24468 | 2507 | 2526 | CTTTGCACTTTGCGAACCAA | 48 | H | 599 |
| 1381133 | 17340 | 17359 | N/A | N/A | TCTAGGTCACTGCTGGGTCC | 74 | H | 600 |
| 1381145 | 24660 | 24679 | 2718 | 2737 | CCTGTAGCCTGTCAGCGAGT | 63 | H | 601 |
| 1381159 | 17621 | 17640 | N/A | N/A | ATCCCCAGTATTGTTCAGCA | 27 | H | 602 |
| 1381170 | 14196 | 14215 | N/A | N/A | TTGTATCCAGTACCTCTAGA | 29 | H | 603 |
| 1381196 | 24113 | 24132 | 2171 | 2190 | GTTGTGAACTGGCAGGCGGT | 72 | H | 604 |
| 1381232 | 15005 | 15024 | 460 | 479 | GCTTCATCTTCACTACCGCT | 53 | H | 605 |
| 1381288 | 24213 | 24232 | 2271 | 2290 | CCCGTTCGCCGGCCGCGGAC | 119 | H | 606 |
| 1381306 | 24250 | 24269 | 2308 | 2327 | CAGCAGCAGCATTCCCGGCT | 58 | H | 607 |
| 1381316 | 21392 | 21411 | N/A | N/A | CTGCTTGGCTCTGGCCACCT | 90 | H | 608 |
| 1381326 | 24368 | 24387 | 2426 | 2445 | CTGCAGTTTGCCCATCCACG | 62 | H | 609 |
| 1381359 | 24465 | 24484 | 2523 | 2542 | GTCATGCACAAGAAAGCTTT | 93 | H | 610 |
| 1381362 | 12167 | 12186 | 200 | 219 | ACCTCGGCTGACATGTTGGA | 84 | H | 611 |
| 1381397 | 24549 | 24568 | 2607 | 2626 | GATCCCCGAAAAAGCGGGTT | 86 | H | 612 |
| 1381401 | 24774 | 24793 | 2832 | 2851 | GGCCTTTTATTCGCGAGGGT | 82 | H | 613 |
| 1381408 | 17071 | 17090 | 1224 | 1243 | ACCATCCCAGTCGAGGCCAA | 118 | H | 614 |
| 1381420 | 24160 | 24179 | 2218 | 2237 | GCCCGGATCACAGGACTGGA | 108 | H | 615 |
| 1381451 | 15769 | 15788 | N/A | N/A | TGGGCAGAGACCTGCAGCCC | 88 | H | 616 |
| 1381458 | 17140 | 17159 | 1293 | 1312 | CAAGTCGAAGTTGCATGTGT | 59 | H | 617 |
| 1381470 | 14028 | 14047 | N/A | N/A | GAGTGCTTTAGTCCTACCCC | 51 | H | 618 |
| 1381476 | 15964 | 15983 | 718 | 737 | CCGCCAGGTAGAAGCGCGCC | 73 | H | 619 |
| 1381492 | 22597 | 22616 | 1550 | 1569 | GCTGGAACTGCCACTTCAGC | 100 | H | 620 |
| 1381497 | 14057 | 14076 | N/A | N/A | GTCCAGCCCATCTCTCAGTC | 63 | H | 621 |
| 1381506 | 14658 | 14677 | 368 | 387 | AGCCTCACCACGATGGGCTC | 124 | H | 622 |
| 1381530 | 22085 | 22104 | N/A | N/A | TCGACTTCTCAGATCCCCAG | 56 | H | 623 |
| 1381537 | 19700 | 19719 | N/A | N/A | AACTCTGGCCTCTTAGGAGT | 92 | H | 624 |
| 1381553 | 24232 | 24251 | 2290 | 2309 | CTACAAGGACCCTTCGAGCC | 95 | H | 625 |
| 1381555 | 21816 | 21835 | 1481 | 1500 | TGTGGCTCAAGCAGCTGCTC | 109 | H | 626 |
| 1381558 | 24073 | 24092 | 2131 | 2150 | GCCCCGGGCACTCAGTCTTC | 74 | H | 627 |
| 1381565 | 16324 | 16343 | 806 | 825 | CCACAGCGGTCCAGCAGGAT | 90 | H | 628 |
| 1381568 | 24409 | 24428 | 2467 | 2486 | AGGATGGAACACGGACGGCC | 98 | H | 629 |
| 1381578 | 24525 | 24544 | 2583 | 2602 | AAAAGCAAATTTCCCGAGTA | 52 | H | 630 |
| 1381608 | 22681 | 22700 | 1634 | 1653 | TGCCGGGTGAGCACCTCCTC | 213 | H | 631 |
| 1381676 | 15064 | 15083 | 519 | 538 | GCCCCTCTTCAGCATGTCCC | 79 | H | 632 |
| 1381678 | 22150 | 22169 | N/A | N/A | GCCTGACCACTTGGCACCTT | 87 | H | 633 |
| 1381680 | 12749 | 12768 | N/A | N/A | GCAGCTGCCCCATGCTGGCC | 91 | H | 634 |
| 1052873 | 19496 | 19515 | 1357 | 1376 | CTTCCCGAATGTCCGACAGT | 91 | I | 19 |
| 1052886 | 24119 | 24138 | 2177 | 2196 | GGAGCGGTTGTGAACTGGCA | 37 | I | 635 |
| 1380285 | 13969 | 13988 | N/A | N/A | TCCTATGACTAGGAATGTTA | 61 | I | 636 |
| 1380345 | 14058 | 14077 | N/A | N/A | GGTCCAGCCCATCTCTCAGT | 23 | I | 637 |
| 1380351 | 17707 | 17726 | N/A | N/A | CACCTGCCACACTCTCCATA | 68 | I | 638 |
| 1380361 | 17599 | 17618 | N/A | N/A | AGGGCACCCAGAGCCGAGCA | 67 | I | 639 |
| 1380373 | 10684 | 10703 | N/A | N/A | CAGACAATAGCAAGGGCAGC | 38 | I | 640 |
| 1380376 | 15168 | 15187 | 544 | 563 | TCTCCTCACGGAAGCACGAC | 91 | I | 641 |
| 1380382 | 20881 | 20900 | N/A | N/A | CTTGTGATAAGCAATGCATT | 62 | I | 642 |
| 1380399 | 17465 | 17484 | N/A | N/A | GCCCTCCAGGACCTTCCTTG | 69 | I | 643 |
| 1380405 | 20926 | 20945 | N/A | N/A | CACGAGATATCAACTTCCTT | 14 | I | 644 |
| 1380426 | 9244 | 9263 | N/A | N/A | CAACAGAAATAGATTCTGGT | 63 | I | 645 |
| 1380439 | 17259 | 17278 | N/A | N/A | GTAGGCACTGTCCTTACTCC | 64 | I | 646 |
| 1380460 | 19920 | 19939 | N/A | N/A | TATTGTTATATGGCTGATTC | 21 | I | 647 |
| 1380473 | 22302 | 22321 | N/A | N/A | TCCAGCTCCCTAATGCCCTC | 88 | I | 648 |
| 1380495 | 17161 | 17180 | 1314 | 1333 | GGCAGTGAGCCCGTCCTCCA | 80 | I | 649 |
| 1380497 | 24163 | 24182 | 2221 | 2240 | CGGGCCCGGATCACAGGACT | 71 | I | 650 |
| 1380499 | 17341 | 17360 | N/A | N/A | GTCTAGGTCACTGCTGGGTC | 40 | I | 651 |
| 1380502 | 21118 | 21137 | N/A | N/A | AGTTCATTAATGATAAGGTA | 20 | I | 652 |
| 1380504 | 19370 | 19389 | N/A | N/A | GGCTTATCTAAAGTGGCCCC | 101 | I | 653 |
| 1380513 | 15333 | 15352 | N/A | N/A | CCCAATCCTAGAGCTTCCTC | 77 | I | 654 |
| 1380522 | 24036 | 24055 | 2094 | 2113 | CGAAGACAGTTCTAGGGTTC | 24 | I | 655 |
| 1380539 | 20808 | 20827 | N/A | N/A | TGGAATCTATCATGGCTCAC | 36 | I | 656 |
| 1380550 | 21991 | 22010 | N/A | N/A | CCGTGGTTTCTGTCTGCTTC | 29 | I | 657 |
| 1380555 | 22913 | 22932 | 1761 | 1780 | CTCCATCCGCTCCTGCAACT | 67 | I | 658 |
| 1380558 | 24662 | 24681 | 2720 | 2739 | GTCCTGTAGCCTGTCAGCGA | 64 | I | 659 |
| 1380575 | 22286 | 22305 | N/A | N/A | CCTCACGACAAAAGGCCTTG | 46 | I | 660 |
| 1380599 | 22151 | 22170 | N/A | N/A | CGCCTGACCACTTGGCACCT | 88 | I | 661 |
| 1380648 | 15929 | 15948 | 683 | 702 | CGCTCCCCAAACTTGCTCAG | 107 | I | 662 |
| 1380664 | 16726 | 16745 | 955 | 974 | GCCCGTAGCTGCCTGTCCCA | 63 | I | 663 |
| 1380668 | 22231 | 22250 | N/A | N/A | TCAGGAATGATTCAGCCAAA | 30 | I | 664 |
| 1380690 | 17737 | 17756 | N/A | N/A | TTTCTTGTATCCTGTTGCTT | 43 | I | 665 |
| 1380710 | 14526 | 14545 | N/A | N/A | ACCCTAGGACTGTCTGCTTC | 69 | I | 666 |
| 1380718 | 24450 | 24469 | 2508 | 2527 | GCTTTGCACTTTGCGAACCA | 60 | I | 667 |
| 1380737 | 22335 | 22354 | N/A | N/A | CACAACCTATGTCCCTCTGC | 65 | I | 668 |
| 1380741 | 16708 | 16727 | 937 | 956 | CAGGCCCACCGCCCACAGCC | 63 | I | 669 |
| 1380765 | 13473 | 13492 | N/A | N/A | AGAAGGTTTTTCCAGAGGCT | 4 | I | 670 |
| 1380766 | 24386 | 24405 | 2444 | 2463 | CTTGCTGCCTTCCCAGGCCT | 87 | I | 671 |
| 1380774 | 19505 | 19524 | 1366 | 1385 | GCGGCGCACCTTCCCGAATG | 68 | I | 672 |
| 1380830 | 19804 | 19823 | N/A | N/A | AGTTAATGGTTACAAGATTC | 31 | I | 673 |
| 1380900 | 15621 | 15640 | N/A | N/A | CCCTGAGATGTTCTGGGAAA | 86 | I | 674 |
| 1380907 | 15882 | 15901 | N/A | N/A | ATACTCCATGACCAGGTACT | 86 | I | 675 |
| 1380944 | 14012 | 14031 | N/A | N/A | CCCCTTATTTACAGATGACA | 50 | I | 676 |
| 1380969 | 19659 | 19678 | N/A | N/A | GTTCCAAGACTGATCCTGCA | 78 | I | 677 |
| 1380986 | 12872 | 12891 | N/A | N/A | CCTGTCTGCAAAGCTGGTTC | 83 | I | 678 |
| 1381010 | 21063 | 21082 | N/A | N/A | CTAGAAAACCAACTGTTCTC | 90 | I | 679 |
| 1381111 | 21045 | 21064 | N/A | N/A | TCTTAGACAAAGTAGCATGA | 53 | I | 680 |
| 1381112 | 11708 | 11727 | N/A | N/A | CTTGCTCCCGACAAGCTCCA | 44 | I | 681 |
| 1381192 | 17624 | 17643 | N/A | N/A | TGGATCCCCAGTATTGTTCA | 46 | I | 682 |
| 1381209 | 22319 | 22338 | N/A | N/A | CTGCTGCTCAAAATCCCTCC | 60 | I | 683 |
| 1381252 | 24466 | 24485 | 2524 | 2543 | CGTCATGCACAAGAAAGCTT | 52 | I | 684 |
| 1381257 | 15972 | 15991 | 726 | 745 | GACAATCTCCGCCAGGTAGA | 46 | I | 685 |
| 1381260 | 12750 | 12769 | N/A | N/A | GGCAGCTGCCCCATGCTGGC | 96 | I | 686 |
| 1381271 | 21817 | 21836 | 1482 | 1501 | GTGTGGCTCAAGCAGCTGCT | 36 | I | 687 |
| 1381277 | 24369 | 24388 | 2427 | 2446 | CCTGCAGTTTGCCCATCCAC | 82 | I | 688 |
| 1381286 | 15065 | 15084 | 520 | 539 | CGCCCCTCTTCAGCATGTCC | 78 | I | 689 |
| 1381304 | 12170 | 12189 | 203 | 222 | CGCACCTCGGCTGACATGTT | 89 | I | 690 |
| 1381320 | 14197 | 14216 | N/A | N/A | GTTGTATCCAGTACCTCTAG | 17 | I | 691 |
| 1381363 | 14029 | 14048 | N/A | N/A | GGAGTGCTTTAGTCCTACCC | 39 | I | 692 |
| 1381372 | 24074 | 24093 | 2132 | 2151 | TGCCCCGGGCACTCAGTCTT | 112 | I | 693 |
| 1381427 | 22870 | 22889 | 1718 | 1737 | CGGTTCCGAGCCTCTGCCTC | 12† | I | 694 |
| 1381448 | 24233 | 24252 | 2291 | 2310 | GCTACAAGGACCCTTCGAGC | 118 | I | 695 |
| 1381456 | 22121 | 22140 | N/A | N/A | GGCTTGTTTCTCCTTCACCA | 16 | I | 696 |
| 1381461 | 15008 | 15027 | 463 | 482 | TCTGCTTCATCTTCACTACC | 94 | I | 697 |
| 1381471 | 24217 | 24236 | 2275 | 2294 | GAGCCCCGTTCGCCGGCCGC | 111 | I | 698 |
| 1381544 | 24410 | 24429 | 2468 | 2487 | GAGGATGGAACACGGACGGC | 79 | I | 699 |
| 1381548 | 16332 | 16351 | 814 | 833 | GGATGTGGCCACAGCGGTCC | 102 | I | 700 |
| 1381552 | 24550 | 24569 | 2608 | 2627 | GGATCCCCGAAAAAGCGGGT | 110 | I | 701 |
| 1381580 | 14660 | 14679 | 370 | 389 | TAAGCCTCACCACGATGGGC | 89 | I | 702 |
| 1381592 | 24776 | 24795 | 2834 | 2853 | AGGGCCTTTTATTCGCGAGG | 47 | I | 703 |
| 1381596 | 24627 | 24646 | 2685 | 2704 | CAATAAATATCCAAACCGCC | 70 | I | 704 |
| 1381624 | 24251 | 24270 | 2309 | 2328 | GCAGCAGCAGCATTCCCGGC | 40 | I | 705 |
| 1381637 | 19705 | 19724 | N/A | N/A | CCTCCAACTCTGGCCTCTTA | 69 | I | 706 |
| 1381642 | 17144 | 17163 | 1297 | 1316 | CCACCAAGTCGAAGTTGCAT | 62 | I | 707 |
| 1381650 | 22601 | 22620 | 1554 | 1573 | AGCCGCTGGAACTGCCACTT | 43 | I | 708 |
| 1381654 | 21396 | 21415 | N/A | N/A | GAGACTGCTTGGCTCTGGCC | 74 | I | 709 |
| 1381669 | 24526 | 24545 | 2584 | 2603 | CAAAAGCAAATTTCCCGAGT | 68 | I | 710 |
| 1381673 | 17073 | 17092 | 1226 | 1245 | AGACCATCCCAGTCGAGGCC | 85 | I | 711 |
| 1052873 | 19496 | 19515 | 1357 | 1376 | CTTCCCGAATGTCCGACAGT | 88 | J | 19 |
| 1380286 | 17738 | 17757 | N/A | N/A | TTTTCTTGTATCCTGTTGCT | 24 | J | 712 |
| 1380288 | 17601 | 17620 | N/A | N/A | AAAGGGCACCCAGAGCCGAG | 96 | J | 713 |
| 1380295 | 10686 | 10705 | N/A | N/A | CACAGACAATAGCAAGGGCA | 29 | J | 714 |
| 1380313 | 16709 | 16728 | 938 | 957 | CCAGGCCCACCGCCCACAGC | 78 | J | 715 |
| 1380320 | 20809 | 20828 | N/A | N/A | CTGGAATCTATCATGGCTCA | 19 | J | 716 |
| 1380337 | 22336 | 22355 | N/A | N/A | ACACAACCTATGTCCCTCTG | 48 | J | 717 |
| 1380349 | 17162 | 17181 | 1315 | 1334 | TGGCAGTGAGCCCGTCCTCC | 114 | J | 718 |
| 1380350 | 24387 | 24406 | 2445 | 2464 | GCTTGCTGCCTTCCCAGGCC | 113 | J | 719 |
| 1380368 | 15622 | 15641 | N/A | N/A | CCCCTGAGATGTTCTGGGAA | 94 | J | 720 |
| 1380446 | 17342 | 17361 | N/A | N/A | AGTCTAGGTCACTGCTGGGT | 30 | J | 721 |
| 1380478 | 15334 | 15353 | N/A | N/A | CCCCAATCCTAGAGCTTCCT | 111 | J | 722 |
| 1380484 | 19506 | 19525 | 1367 | 1386 | AGCGGCGCACCTTCCCGAAT | 73 | J | 723 |
| 1380489 | 21119 | 21138 | N/A | N/A | GAGTTCATTAATGATAAGGT | 2 | J | 724 |
| 1380541 | 24451 | 24470 | 2509 | 2528 | AGCTTTGCACTTTGCGAACC | 53 | J | 725 |
| 1380552 | 22232 | 22251 | N/A | N/A | CTCAGGAATGATTCAGCCAA | 55 | J | 726 |
| 1380605 | 21459 | 21478 | N/A | N/A | GACATGTGACCGCTGCAGAC | 29 | J | 727 |
| 1380674 | 21992 | 22011 | N/A | N/A | TCCGTGGTTTCTGTCTGCTT | 33 | J | 728 |
| 1380684 | 24037 | 24056 | 2095 | 2114 | TCGAAGACAGTTCTAGGGTT | 36 | J | 729 |
| 1380689 | 22303 | 22322 | N/A | N/A | CTCCAGCTCCCTAATGCCCT | 79 | J | 730 |
| 1380754 | 17260 | 17279 | N/A | N/A | GGTAGGCACTGTCCTTACTC | 80 | J | 731 |
| 1380755 | 14527 | 14546 | N/A | N/A | CACCCTAGGACTGTCTGCTT | 69 | J | 732 |
| 1380757 | 22287 | 22306 | N/A | N/A | CCCTCACGACAAAAGGCCTT | 96 | J | 733 |
| 1380762 | 21064 | 21083 | N/A | N/A | ACTAGAAAACCAACTGTTCT | 89 | J | 734 |
| 1380772 | 12873 | 12892 | N/A | N/A | GCCTGTCTGCAAAGCTGGTT | 82 | J | 735 |
| 1380789 | 15930 | 15949 | 684 | 703 | CCGCTCCCCAAACTTGCTCA | 96 | J | 736 |
| 1380791 | 13474 | 13493 | N/A | N/A | GAGAAGGTTTTTCCAGAGGC | 12 | J | 737 |
| 1380814 | 13970 | 13989 | N/A | N/A | GTCCTATGACTAGGAATGTT | 52 | J | 738 |
| 1380829 | 19371 | 19390 | N/A | N/A | GGGCTTATCTAAAGTGGCCC | 120 | J | 739 |
| 1380883 | 22320 | 22339 | N/A | N/A | TCTGCTGCTCAAAATCCCTC | 74 | J | 740 |
| 1380937 | 19660 | 19679 | N/A | N/A | GGTTCCAAGACTGATCCTGC | 64 | J | 741 |
| 1380946 | 11709 | 11728 | N/A | N/A | CCTTGCTCCCGACAAGCTCC | 85 | J | 742 |
| 1380949 | 22914 | 22933 | 1762 | 1781 | ACTCCATCCGCTCCTGCAAC | 65 | J | 743 |
| 1380958 | 9681 | 9700 | N/A | N/A | TCTACGATTCCAAAACTGAG | 85 | J | 744 |
| 1380960 | 24702 | 24721 | 2760 | 2779 | GGTCTCAGTGCATCCAAAAC | 64 | J | 745 |
| 1380961 | 17708 | 17727 | N/A | N/A | GCACCTGCCACACTCTCCAT | 95 | J | 746 |
| 1380963 | 19805 | 19824 | N/A | N/A | AAGTTAATGGTTACAAGATT | 45 | J | 747 |
| 1380977 | 20882 | 20901 | N/A | N/A | ACTTGTGATAAGCAATGCAT | 33 | J | 748 |
| 1381007 | 19709 | 19728 | N/A | N/A | AGGTCCTCCAACTCTGGCCT | 74 | J | 749 |
| 1381012 | 15883 | 15902 | 637 | 656 | AATACTCCATGACCAGGTAC | 61 | J | 750 |
| 1381045 | 20927 | 20946 | N/A | N/A | TCACGAGATATCAACTTCCT | 58 | J | 751 |
| 1381047 | 21047 | 21066 | N/A | N/A | TCTCTTAGACAAAGTAGCAT | 39 | J | 752 |
| 1381077 | 17466 | 17485 | N/A | N/A | AGCCCTCCAGGACCTTCCTT | 119 | J | 753 |
| 1381080 | 14015 | 14034 | N/A | N/A | CTACCCCTTATTTACAGATG | 36 | J | 754 |
| 1381153 | 19921 | 19940 | N/A | N/A | TTATTGTTATATGGCTGATT | 11 | J | 755 |
| 1381174 | 15169 | 15188 | 545 | 564 | CTCTCCTCACGGAAGCACGA | 96 | J | 756 |
| 1381211 | 24628 | 24647 | 2686 | 2705 | TCAATAAATATCCAAACCGC | 72 | J | 757 |
| 1381242 | 24235 | 24254 | 2293 | 2312 | CGGCTACAAGGACCCTTCGA | 85 | J | 758 |
| 1381244 | 14198 | 14217 | N/A | N/A | GGTTGTATCCAGTACCTCTA | 52 | J | 759 |
| 1381250 | 24413 | 24432 | 2471 | 2490 | GTGGAGGATGGAACACGGAC | 56 | J | 760 |
| 1381284 | 21875 | 21894 | N/A | N/A | ACCAACTTACTGTTTCATCC | 92 | J | 761 |
| 1381301 | 22158 | 22177 | N/A | N/A | AGGCTCTCGCCTGACCACTT | 92 | J | 762 |
| 1381315 | 24130 | 24149 | 2188 | 2207 | GACCCACGCTCGGAGCGGTT | 103 | J | 763 |
| 1381348 | 15974 | 15993 | 728 | 747 | ATGACAATCTCCGCCAGGTA | 83 | J | 764 |
| 1381355 | 12171 | 12190 | 204 | 223 | CCGCACCTCGGCTGACATGT | 96 | J | 765 |
| 1381365 | 14030 | 14049 | N/A | N/A | AGGAGTGCTTTAGTCCTACC | 54 | J | 766 |
| 1381366 | 24331 | 24350 | 2389 | 2408 | CCGAAAGAAAGAAATGGTCT | 59 | J | 767 |
| 1381403 | 16333 | 16352 | 815 | 834 | CGGATGTGGCCACAGCGGTC | 96 | J | 768 |
| 1381410 | 12753 | 12772 | N/A | N/A | CCTGGCAGCTGCCCCATGCT | 116 | J | 769 |
| 1381436 | 24527 | 24546 | 2585 | 2604 | GCAAAAGCAAATTTCCCGAG | 59 | J | 770 |
| 1381441 | 24219 | 24238 | 2277 | 2296 | TCGAGCCCCGTTCGCCGGCC | 112 | J | 771 |
| 1381455 | 24370 | 24389 | 2428 | 2447 | GCCTGCAGTTTGCCCATCCA | 93 | J | 772 |
| 1381466 | 24552 | 24571 | 2610 | 2629 | CGGGATCCCCGAAAAAGCGG | 89 | J | 773 |
| 1381481 | 15010 | 15029 | 465 | 484 | CGTCTGCTTCATCTTCACTA | 87 | J | 774 |
| 1381498 | 17074 | 17093 | 1227 | 1246 | GAGACCATCCCAGTCGAGGC | 63 | J | 775 |
| 1381517 | 24164 | 24183 | 2222 | 2241 | GCGGGCCCGGATCACAGGAC | 98 | J | 776 |
| 1381536 | 22125 | 22144 | N/A | N/A | CCATGGCTTGTTTCTCCTTC | 96 | J | 777 |
| 1381542 | 22610 | 22629 | 1563 | 1582 | CGCAGGGACAGCCGCTGGAA | 95 | J | 778 |
| 1381545 | 22871 | 22890 | 1719 | 1738 | CCGGTTCCGAGCCTCTGCCT | 9† | J | 779 |
| 1381547 | 24777 | 24796 | 2835 | 2854 | GAGGGCCTTTTATTCGCGAG | 93 | J | 780 |
| 1381575 | 17146 | 17165 | 1299 | 1318 | CTCCACCAAGTCGAAGTTGC | 81 | J | 781 |
| 1381576 | 14661 | 14680 | 371 | 390 | TTAAGCCTCACCACGATGGG | 72 | J | 782 |
| 1381609 | 14165 | 14184 | N/A | N/A | GTGGTTCTTGAACCACACTT | 86 | J | 783 |
| 1381615 | 16845 | 16864 | N/A | N/A | GCTCACCTTGTAGTGGACGA | 104 | J | 784 |
| 1381636 | 24075 | 24094 | 2133 | 2152 | GTGCCCCGGGCACTCAGTCT | 50 | J | 785 |
| 1381653 | 24484 | 24503 | 2542 | 2561 | ACGCTCCCCAGAGCAGGGCG | 112 | J | 786 |
| 1381667 | 15066 | 15085 | 521 | 540 | TCGCCCCTCTTCAGCATGTC | 62 | J | 787 |
| 1381681 | 17693 | 17712 | N/A | N/A | TCCATAATTCTCTAATTCTC | 60 | J | 788 |
| 1052873 | 19496 | 19515 | 1357 | 1376 | CTTCCCGAATGTCCGACAGT | 73 | K | 19 |
| 1380289 | 19922 | 19941 | N/A | N/A | CTTATTGTTATATGGCTGAT | 5 | K | 789 |
| 1380300 | 24388 | 24407 | 2446 | 2465 | GGCTTGCTGCCTTCCCAGGC | 148 | K | 790 |
| 1380311 | 17350 | 17369 | N/A | N/A | GGACTGTAAGTCTAGGTCAC | 138 | K | 791 |
| 1380342 | 20810 | 20829 | N/A | N/A | GCTGGAATCTATCATGGCTC | 80 | K | 792 |
| 1380346 | 15884 | 15903 | 638 | 657 | TAATACTCCATGACCAGGTA | 41 | K | 793 |
| 1380362 | 17602 | 17621 | N/A | N/A | AAAAGGGCACCCAGAGCCGA | 103 | K | 794 |
| 1380369 | 12874 | 12893 | N/A | N/A | CGCCTGTCTGCAAAGCTGGT | 65 | K | 795 |
| 1380380 | 24038 | 24057 | 2096 | 2115 | GTCGAAGACAGTTCTAGGGT | 17 | K | 796 |
| 1380424 | 22191 | 22210 | N/A | N/A | AGGATTCCCACCTGCCCAAG | 58 | K | 797 |
| 1380442 | 21066 | 21085 | N/A | N/A | TGACTAGAAAACCAACTGTT | 81 | K | 798 |
| 1380483 | 22321 | 22340 | N/A | N/A | CTCTGCTGCTCAAAATCCCT | 87 | K | 799 |
| 1380493 | 24452 | 24471 | 2510 | 2529 | AAGCTTTGCACTTTGCGAAC | 47 | K | 800 |
| 1380494 | 22233 | 22252 | N/A | N/A | TCTCAGGAATGATTCAGCCA | 54 | K | 801 |
| 1380532 | 21462 | 21481 | N/A | N/A | CTTGACATGTGACCGCTGCA | 63 | K | 802 |
| 1380603 | 15171 | 15190 | 547 | 566 | CCCTCTCCTCACGGAAGCAC | 51 | K | 803 |
| 1380613 | 9683 | 9702 | N/A | N/A | ACTCTACGATTCCAAAACTG | 62 | K | 804 |
| 1380616 | 19373 | 19392 | N/A | N/A | CAGGGCTTATCTAAAGTGGC | 76 | K | 805 |
| 1380629 | 15630 | 15649 | N/A | N/A | ATTCACTCCCCCTGAGATGT | 59 | K | 806 |
| 1380632 | 17163 | 17182 | 1316 | 1335 | ATGGCAGTGAGCCCGTCCTC | 91 | K | 807 |
| 1380678 | 20883 | 20902 | N/A | N/A | TACTTGTGATAAGCAATGCA | 32 | K | 808 |
| 1380687 | 22337 | 22356 | N/A | N/A | AACACAACCTATGTCCCTCT | 54 | K | 809 |
| 1380702 | 22915 | 22934 | 1763 | 1782 | AACTCCATCCGCTCCTGCAA | 103 | K | 810 |
| 1380758 | 21121 | 21140 | N/A | N/A | TGGAGTTCATTAATGATAAG | 42 | K | 811 |
| 1380800 | 13971 | 13990 | N/A | N/A | GGTCCTATGACTAGGAATGT | 63 | K | 812 |
| 1380801 | 14528 | 14547 | N/A | N/A | TCACCCTAGGACTGTCTGCT | 41 | K | 813 |
| 1380811 | 24629 | 24648 | 2687 | 2706 | GTCAATAAATATCCAAACCG | 85 | K | 814 |
| 1380818 | 17261 | 17280 | N/A | N/A | AGGTAGGCACTGTCCTTACT | 117 | K | 815 |
| 1380844 | 15335 | 15354 | N/A | N/A | CCCCCAATCCTAGAGCTTCC | 35 | K | 816 |
| 1380864 | 14016 | 14035 | N/A | N/A | CCTACCCCTTATTTACAGAT | 22 | K | 817 |
| 1380912 | 22304 | 22323 | N/A | N/A | CCTCCAGCTCCCTAATGCCC | 87 | K | 818 |
| 1380939 | 17468 | 17487 | N/A | N/A | AAAGCCCTCCAGGACCTTCC | 73 | K | 819 |
| 1380972 | 19710 | 19729 | N/A | N/A | AAGGTCCTCCAACTCTGGCC | 58 | K | 820 |
| 1380995 | 10688 | 10707 | N/A | N/A | AGCACAGACAATAGCAAGGG | 9 | K | 821 |
| 1381020 | 21048 | 21067 | N/A | N/A | TTCTCTTAGACAAAGTAGCA | 47 | K | 822 |
| 1381042 | 20928 | 20947 | N/A | N/A | CTCACGAGATATCAACTTCC | 78 | K | 823 |
| 1381051 | 11711 | 11730 | N/A | N/A | CCCCTTGCTCCCGACAAGCT | 88 | K | 824 |
| 1381070 | 13477 | 13496 | N/A | N/A | TGGGAGAAGGTTTTTCCAGA | 62 | K | 825 |
| 1381087 | 21993 | 22012 | N/A | N/A | CTCCGTGGTTTCTGTCTGCT | 42 | K | 826 |
| 1381102 | 24165 | 24184 | 2223 | 2242 | GGCGGGCCCGGATCACAGGA | 100 | K | 827 |
| 1381126 | 22288 | 22307 | N/A | N/A | GCCCTCACGACAAAAGGCCT | 84 | K | 828 |
| 1381132 | 17739 | 17758 | N/A | N/A | TTTTTCTTGTATCCTGTTGC | 24 | K | 829 |
| 1381140 | 16710 | 16729 | 939 | 958 | CCCAGGCCCACCGCCCACAG | 77 | K | 830 |
| 1381177 | 19507 | 19526 | 1368 | 1387 | TAGCGGCGCACCTTCCCGAA | 89 | K | 831 |
| 1381183 | 19844 | 19863 | N/A | N/A | AGTTCTGAAGTCCTGTGGCT | 27 | K | 832 |
| 1381201 | 19662 | 19681 | N/A | N/A | AAGGTTCCAAGACTGATCCT | 94 | K | 833 |
| 1381233 | 17710 | 17729 | N/A | N/A | GGGCACCTGCCACACTCTCC | 79 | K | 834 |
| 1381240 | 15975 | 15994 | 729 | 748 | CATGACAATCTCCGCCAGGT | 67 | K | 835 |
| 1381249 | 12177 | 12196 | 210 | 229 | CCTCAGCCGCACCTCGGCTG | 77 | K | 836 |
| 1381258 | 24237 | 24256 | 2295 | 2314 | CCCGGCTACAAGGACCCTTC | 62 | K | 837 |
| 1381268 | 17076 | 17095 | 1229 | 1248 | CGGAGACCATCCCAGTCGAG | 74 | K | 838 |
| 1381273 | 14031 | 14050 | N/A | N/A | GAGGAGTGCTTTAGTCCTAC | 67 | K | 839 |
| 1381275 | 22127 | 22146 | N/A | N/A | TTCCATGGCTTGTTTCTCCT | 56 | K | 840 |
| 1381279 | 15011 | 15030 | 466 | 485 | CCGTCTGCTTCATCTTCACT | 59 | K | 841 |
| 1381294 | 15071 | 15090 | N/A | N/A | TCACCTCGCCCCTCTTCAGC | 63 | K | 842 |
| 1381345 | 21878 | 21897 | N/A | N/A | TCCACCAACTTACTGTTTCA | 124 | K | 843 |
| 1381349 | 24553 | 24572 | 2611 | 2630 | GCGGGATCCCCGAAAAAGCG | 72 | K | 844 |
| 1381374 | 24500 | 24519 | 2558 | 2577 | GCAGAGATCGCGCCAGACGC | 63 | K | 845 |
| 1381380 | 24778 | 24797 | 2836 | 2855 | GGAGGGCCTTTTATTCGCGA | 94 | K | 846 |
| 1381387 | 22621 | 22640 | 1574 | 1593 | TCAGCCTCTGCCGCAGGGAC | 211 | K | 847 |
| 1381390 | 17694 | 17713 | N/A | N/A | CTCCATAATTCTCTAATTCT | 46 | K | 848 |
| 1381439 | 24528 | 24547 | 2586 | 2605 | GGCAAAAGCAAATTTCCCGA | 70 | K | 849 |
| 1381484 | 24438 | 24457 | 2496 | 2515 | GCGAACCAACGATAGGTGGG | 61 | K | 850 |
| 1381490 | 24332 | 24351 | 2390 | 2409 | GCCGAAAGAAAGAAATGGTC | 22 | K | 851 |
| 1381522 | 14199 | 14218 | N/A | N/A | GGGTTGTATCCAGTACCTCT | 83 | K | 852 |
| 1381529 | 15931 | 15950 | 685 | 704 | TCCGCTCCCCAAACTTGCTC | 67 | K | 853 |
| 1381534 | 22872 | 22891 | 1720 | 1739 | CCCGGTTCCGAGCCTCTGCC | 4† | K | 854 |
| 1381540 | 24724 | 24743 | 2782 | 2801 | AATAAATACCGAGGAATGTC | 64 | K | 855 |
| 1381569 | 24076 | 24095 | 2134 | 2153 | CGTGCCCCGGGCACTCAGTC | 82 | K | 856 |
| 1381570 | 14166 | 14185 | N/A | N/A | GGTGGTTCTTGAACCACACT | 113 | K | 857 |
| 1381588 | 17148 | 17167 | 1301 | 1320 | TCCTCCACCAAGTCGAAGTT | 103 | K | 858 |
| 1381589 | 24220 | 24239 | 2278 | 2297 | TTCGAGCCCCGTTCGCCGGC | 84 | K | 859 |
| 1381591 | 14662 | 14681 | 372 | 391 | CTTAAGCCTCACCACGATGG | 74 | K | 860 |
| 1381619 | 24372 | 24391 | 2430 | 2449 | AGGCCTGCAGTTTGCCCATC | 16 | K | 861 |
| 1381633 | 16848 | 16867 | N/A | N/A | CGTGCTCACCTTGTAGTGGA | 43 | K | 862 |
| 1381662 | 24133 | 24152 | 2191 | 2210 | GGAGACCCACGCTCGGAGCG | 76 | K | 863 |
| 1381682 | 12754 | 12773 | N/A | N/A | CCCTGGCAGCTGCCCCATGC | 134 | K | 864 |
| 1381688 | 16334 | 16353 | 816 | 835 | GCGGATGTGGCCACAGCGGT | 83 | K | 865 |
| 1052863 | 24725 | 24744 | 2783 | 2802 | CAATAAATACCGAGGAATGT | 43 | L | 866 |
| 1052873 | 19496 | 19515 | 1357 | 1376 | CTTCCCGAATGTCCGACAGT | 67 | L | 19 |
| 1380296 | 15336 | 15355 | N/A | N/A | ACCCCCAATCCTAGAGCTTC | 119 | L | 867 |
| 1380329 | 22234 | 22253 | N/A | N/A | TTCTCAGGAATGATTCAGCC | 26 | L | 868 |
| 1380330 | 17711 | 17730 | N/A | N/A | AGGGCACCTGCCACACTCTC | 3 | L | 869 |
| 1380355 | 21123 | 21142 | N/A | N/A | GCTGGAGTTCATTAATGATA | 26 | L | 870 |
| 1380360 | 24390 | 24409 | 2448 | 2467 | CCGGCTTGCTGCCTTCCCAG | 62 | L | 871 |
| 1380418 | 15932 | 15951 | 686 | 705 | ATCCGCTCCCCAAACTTGCT | 46 | L | 872 |
| 1380419 | 22193 | 22212 | N/A | N/A | TCAGGATTCCCACCTGCCCA | 50 | L | 873 |
| 1380453 | 20884 | 20903 | N/A | N/A | TTACTTGTGATAAGCAATGC | 22 | L | 874 |
| 1380492 | 17351 | 17370 | N/A | N/A | TGGACTGTAAGTCTAGGTCA | 79 | L | 875 |
| 1380509 | 21088 | 21107 | N/A | N/A | AATGTTGTCCAGTAATAAAA | 51 | L | 876 |
| 1380515 | 17164 | 17183 | 1317 | 1336 | CATGGCAGTGAGCCCGTCCT | 61 | L | 877 |
| 1380517 | 24453 | 24472 | 2511 | 2530 | AAAGCTTTGCACTTTGCGAA | 24 | L | 878 |
| 1380562 | 22322 | 22341 | N/A | N/A | CCTCTGCTGCTCAAAATCCC | 95 | L | 879 |
| 1380574 | 13973 | 13992 | N/A | N/A | AAGGTCCTATGACTAGGAAT | 23 | L | 880 |
| 1380624 | 17603 | 17622 | N/A | N/A | CAAAAGGGCACCCAGAGCCG | 52 | L | 881 |
| 1380679 | 19923 | 19942 | N/A | N/A | CCTTATTGTTATATGGCTGA | 3 | L | 882 |
| 1380681 | 22338 | 22357 | N/A | N/A | TAACACAACCTATGTCCCTC | 99 | L | 883 |
| 1380699 | 12875 | 12894 | N/A | N/A | GCGCCTGTCTGCAAAGCTGG | 54 | L | 884 |
| 1380701 | 21049 | 21068 | N/A | N/A | GTTCTCTTAGACAAAGTAGC | 16 | L | 885 |
| 1380732 | 19491 | 19510 | 1352 | 1371 | CGAATGTCCGACAGTGTCTC | 77 | L | 886 |
| 1380743 | 17469 | 17488 | N/A | N/A | GAAAGCCCTCCAGGACCTTC | 74 | L | 887 |
| 1380778 | 14232 | 14251 | N/A | N/A | ATGTAGAATGTCCTGGGTAA | 25 | L | 888 |
| 1380859 | 22917 | 22936 | 1765 | 1784 | GCAACTCCATCCGCTCCTGC | 84 | L | 889 |
| 1380860 | 17262 | 17281 | N/A | N/A | AAGGTAGGCACTGTCCTTAC | 121 | L | 890 |
| 1380914 | 22289 | 22308 | N/A | N/A | TGCCCTCACGACAAAAGGCC | 81 | L | 891 |
| 1380918 | 19711 | 19730 | N/A | N/A | TAAGGTCCTCCAACTCTGGC | 32 | L | 892 |
| 1380928 | 24630 | 24649 | 2688 | 2707 | GGTCAATAAATATCCAAACC | 48 | L | 893 |
| 1380931 | 9684 | 9703 | N/A | N/A | GACTCTACGATTCCAAAACT | 68 | L | 894 |
| 1380933 | 21463 | 21482 | N/A | N/A | CCTTGACATGTGACCGCTGC | 42 | L | 895 |
| 1380942 | 14529 | 14548 | N/A | N/A | TTCACCCTAGGACTGTCTGC | 39 | L | 896 |
| 1381021 | 17742 | 17761 | N/A | N/A | CATTTTTTCTTGTATCCTGT | 18 | L | 897 |
| 1381062 | 15885 | 15904 | 639 | 658 | GTAATACTCCATGACCAGGT | 33 | L | 898 |
| 1381065 | 15632 | 15651 | N/A | N/A | AGATTCACTCCCCCTGAGAT | 68 | L | 899 |
| 1381069 | 10709 | 10728 | N/A | N/A | CATAATTTAACACTCTTCAA | 33 | L | 900 |
| 1381079 | 14017 | 14036 | N/A | N/A | TCCTACCCCTTATTTACAGA | 26 | L | 901 |
| 1381106 | 22305 | 22324 | N/A | N/A | CCCTCCAGCTCCCTAATGCC | 109 | L | 902 |
| 1381144 | 24042 | 24061 | 2100 | 2119 | CGGAGTCGAAGACAGTTCTA | 64 | L | 903 |
| 1381176 | 19848 | 19867 | N/A | N/A | TCCAAGTTCTGAAGTCCTGT | 7 | L | 904 |
| 1381188 | 19663 | 19682 | N/A | N/A | TAAGGTTCCAAGACTGATCC | 60 | L | 905 |
| 1381193 | 20811 | 20830 | N/A | N/A | GGCTGGAATCTATCATGGCT | 134 | L | 906 |
| 1381206 | 15172 | 15191 | 548 | 567 | TCCCTCTCCTCACGGAAGCA | 56 | L | 907 |
| 1381226 | 16711 | 16730 | 940 | 959 | TCCCAGGCCCACCGCCCACA | 69 | L | 908 |
| 1381237 | 13605 | 13624 | N/A | N/A | GCCACAAAAGGAGTGCTCCT | 82 | L | 909 |
| 1381246 | 15012 | 15031 | 467 | 486 | CCCGTCTGCTTCATCTTCAC | 92 | L | 910 |
| 1381253 | 24779 | 24798 | 2837 | 2856 | TGGAGGGCCTTTTATTCGCG | 30 | L | 911 |
| 1381269 | 12178 | 12197 | 211 | 230 | GCCTCAGCCGCACCTCGGCT | 79 | L | 912 |
| 1381302 | 16850 | 16869 | N/A | N/A | GCCGTGCTCACCTTGTAGTG | 66 | L | 913 |
| 1381318 | 15072 | 15091 | N/A | N/A | CTCACCTCGCCCCTCTTCAG | 42 | L | 914 |
| 1381352 | 20929 | 20948 | N/A | N/A | GCTCACGAGATATCAACTTC | 45 | L | 915 |
| 1381353 | 17077 | 17096 | 1230 | 1249 | CCGGAGACCATCCCAGTCGA | 53 | L | 916 |
| 1381381 | 17696 | 17715 | N/A | N/A | CTCTCCATAATTCTCTAATT | 56 | L | 917 |
| 1381385 | 24188 | 24207 | 2246 | 2265 | CCCTCCCTCCCCGGCCGCTA | 142 | L | 918 |
| 1381405 | 14663 | 14682 | 373 | 392 | CCTTAAGCCTCACCACGATG | 83 | L | 919 |
| 1381409 | 24554 | 24573 | 2612 | 2631 | CGCGGGATCCCCGAAAAAGC | 67 | L | 920 |
| 1381419 | 17149 | 17168 | 1302 | 1321 | GTCCTCCACCAAGTCGAAGT | 63 | L | 921 |
| 1381422 | 22622 | 22641 | 1575 | 1594 | CTCAGCCTCTGCCGCAGGGA | 144 | L | 922 |
| 1381423 | 22040 | 22059 | N/A | N/A | CCCACTGTAACTACAGAGAC | 94 | L | 923 |
| 1381440 | 21880 | 21899 | N/A | N/A | CCTCCACCAACTTACTGTTT | 104 | L | 924 |
| 1381449 | 14167 | 14186 | N/A | N/A | AGGTGGTTCTTGAACCACAC | 64 | L | 925 |
| 1381463 | 24503 | 24522 | 2561 | 2580 | CAGGCAGAGATCGCGCCAGA | 67 | L | 926 |
| 1381473 | 22137 | 22156 | N/A | N/A | GCACCTTTCCTTCCATGGCT | 39 | L | 927 |
| 1381489 | 24221 | 24240 | 2279 | 2298 | CTTCGAGCCCCGTTCGCCGG | 101 | L | 928 |
| 1381503 | 24529 | 24548 | 2587 | 2606 | TGGCAAAAGCAAATTTCCCG | 50 | L | 929 |
| 1381505 | 14045 | 14064 | N/A | N/A | TCTCAGTCCTCCAGGAGGAG | 60 | L | 930 |
| 1381508 | 16378 | 16397 | 860 | 879 | GTTCCATCTGCCCGCAGCTT | 41 | L | 931 |
| 1381550 | 12145 | 12164 | 178 | 197 | GGCAGCACCATGGCCCCTCC | 102 | L | 932 |
| 1381559 | 24077 | 24096 | 2135 | 2154 | CCGTGCCCCGGGCACTCAGT | 121 | L | 933 |
| 1381563 | 22878 | 22897 | 1726 | 1745 | CTAGGTCCCGGTTCCGAGCC | 5† | L | 934 |
| 1381573 | 19530 | 19549 | 1391 | 1410 | CCCACAAAAGGCAGGTGGAC | 129 | L | 935 |
| 1381586 | 24333 | 24352 | 2391 | 2410 | GGCCGAAAGAAAGAAATGGT | 72 | L | 936 |
| 1381599 | 24373 | 24392 | 2431 | 2450 | CAGGCCTGCAGTTTGCCCAT | 36 | L | 937 |
| 1381611 | 24439 | 24458 | 2497 | 2516 | TGCGAACCAACGATAGGTGG | 54 | L | 938 |
| 1381623 | 12760 | 12779 | N/A | N/A | CTGCGCCCCTGGCAGCTGCC | 100 | L | 939 |
| 1381648 | 15976 | 15995 | 730 | 749 | CCATGACAATCTCCGCCAGG | 44 | L | 940 |
| 1381663 | 24238 | 24257 | 2296 | 2315 | TCCCGGCTACAAGGACCCTT | 70 | L | 941 |
| 1381671 | 24134 | 24153 | 2192 | 2211 | CGGAGACCCACGCTCGGAGC | 24 | L | 942 |
| 1052864 | 24726 | 24745 | 2784 | 2803 | ACAATAAATACCGAGGAATG | 56 | M | 943 |
| 1052873 | 19496 | 19515 | 1357 | 1376 | CTTCCCGAATGTCCGACAGT | 93 | M | 19 |
| 1380359 | 24043 | 24062 | 2101 | 2120 | CCGGAGTCGAAGACAGTTCT | 82 | M | 944 |
| 1380372 | 21089 | 21108 | N/A | N/A | TAATGTTGTCCAGTAATAAA | 57 | M | 945 |
| 1380379 | 24454 | 24473 | 2512 | 2531 | GAAAGCTTTGCACTTTGCGA | 62 | M | 946 |
| 1380411 | 15173 | 15192 | 549 | 568 | GTCCCTCTCCTCACGGAAGC | 48 | M | 947 |
| 1380434 | 19357 | 19376 | N/A | N/A | TGGCCCCTCCAGCATTTTTT | 88 | M | 948 |
| 1380444 | 20812 | 20831 | N/A | N/A | AGGCTGGAATCTATCATGGC | 72 | M | 949 |
| 1380450 | 22048 | 22067 | N/A | N/A | CTTAATGCCCCACTGTAACT | 93 | M | 950 |
| 1380481 | 22339 | 22358 | N/A | N/A | CTAACACAACCTATGTCCCT | 93 | M | 951 |
| 1380553 | 19492 | 19511 | 1353 | 1372 | CCGAATGTCCGACAGTGTCT | 62 | M | 952 |
| 1380577 | 16697 | 16716 | 926 | 945 | CCCACAGCCTGCAGGATCTC | 68 | M | 953 |
| 1380583 | 17166 | 17185 | 1319 | 1338 | ACCATGGCAGTGAGCCCGTC | 72 | M | 954 |
| 1380597 | 20886 | 20905 | N/A | N/A | ATTTACTTGTGATAAGCAAT | 30 | M | 955 |
| 1380630 | 19850 | 19869 | N/A | N/A | TTTCCAAGTTCTGAAGTCCT | 28 | M | 956 |
| 1380650 | 17713 | 17732 | N/A | N/A | TCAGGGCACCTGCCACACTC | 99 | M | 957 |
| 1380719 | 24631 | 24650 | 2689 | 2708 | AGGTCAATAAATATCCAAAC | 56 | M | 958 |
| 1380720 | 21975 | 21994 | N/A | N/A | CTTCTGTTCAGGAAGTCCCT | 75 | M | 959 |
| 1380721 | 10710 | 10729 | N/A | N/A | CCATAATTTAACACTCTTCA | 17 | M | 960 |
| 1380739 | 17276 | 17295 | N/A | N/A | ATTCAGGACCCCAGAAGGTA | 66 | M | 961 |
| 1380817 | 21465 | 21484 | N/A | N/A | TCCCTTGACATGTGACCGCT | 81 | M | 962 |
| 1380821 | 14531 | 14550 | N/A | N/A | GCTTCACCCTAGGACTGTCT | 26 | M | 963 |
| 1380885 | 15633 | 15652 | N/A | N/A | CAGATTCACTCCCCCTGAGA | 90 | M | 964 |
| 1380889 | 17604 | 17623 | N/A | N/A | GCAAAAGGGCACCCAGAGCC | 67 | M | 965 |
| 1380904 | 17697 | 17716 | N/A | N/A | ACTCTCCATAATTCTCTAAT | 109 | M | 966 |
| 1380905 | 13974 | 13993 | N/A | N/A | CAAGGTCCTATGACTAGGAA | 35 | M | 967 |
| 1380930 | 17470 | 17489 | N/A | N/A | AGAAAGCCCTCCAGGACCTT | 82 | M | 968 |
| 1380953 | 9685 | 9704 | N/A | N/A | GGACTCTACGATTCCAAAAC | 57 | M | 969 |
| 1380955 | 22237 | 22256 | N/A | N/A | GCCTTCTCAGGAATGATTCA | 78 | M | 970 |
| 1380978 | 14018 | 14037 | N/A | N/A | GTCCTACCCCTTATTTACAG | 39 | M | 971 |
| 1380993 | 19924 | 19943 | N/A | N/A | GCCTTATTGTTATATGGCTG | 102 | M | 972 |
| 1381016 | 15337 | 15356 | N/A | N/A | CACCCCCAATCCTAGAGCTT | 77 | M | 973 |
| 1381043 | 22194 | 22213 | N/A | N/A | ATCAGGATTCCCACCTGCCC | 137 | M | 974 |
| 1381083 | 19664 | 19683 | N/A | N/A | GTAAGGTTCCAAGACTGATC | 28 | M | 975 |
| 1381084 | 20930 | 20949 | N/A | N/A | AGCTCACGAGATATCAACTT | 124 | M | 976 |
| 1381094 | 16712 | 16731 | 941 | 960 | GTCCCAGGCCCACCGCCCAC | 75 | M | 977 |
| 1381124 | 22323 | 22342 | N/A | N/A | CCCTCTGCTGCTCAAAATCC | 55 | M | 978 |
| 1381142 | 22291 | 22310 | N/A | N/A | AATGCCCTCACGACAAAAGG | 60 | M | 979 |
| 1381161 | 22306 | 22325 | N/A | N/A | TCCCTCCAGCTCCCTAATGC | 101 | M | 980 |
| 1381212 | 14233 | 14252 | N/A | N/A | CATGTAGAATGTCCTGGGTA | 44 | M | 981 |
| 1381216 | 19712 | 19731 | N/A | N/A | TTAAGGTCCTCCAACTCTGG | 91 | M | 982 |
| 1381221 | 21050 | 21069 | N/A | N/A | TGTTCTCTTAGACAAAGTAG | 50 | M | 983 |
| 1381247 | 13606 | 13625 | N/A | N/A | GGCCACAAAAGGAGTGCTCC | 109 | M | 984 |
| 1381261 | 21170 | 21189 | N/A | N/A | GTTCAATCCTGACCCACCGT | 63 | M | 985 |
| 1381272 | 12766 | 12785 | N/A | N/A | TGTCGGCTGCGCCCCTGGCA | 136 | M | 986 |
| 1381281 | 24391 | 24410 | 2449 | 2468 | CCCGGCTTGCTGCCTTCCCA | 82 | M | 987 |
| 1381282 | 15977 | 15996 | 731 | 750 | GCCATGACAATCTCCGCCAG | 62 | M | 988 |
| 1381283 | 12521 | 12540 | N/A | N/A | TGACCTTACTCTGCCCCTCC | 40 | M | 989 |
| 1381299 | 15910 | 15929 | 664 | 683 | GCAGTGTCAGCAGGTCCCCG | 78 | M | 990 |
| 1381312 | 14047 | 14066 | N/A | N/A | TCTCTCAGTCCTCCAGGAGG | 125 | M | 991 |
| 1381327 | 22883 | 22902 | 1731 | 1750 | TGCCTCTAGGTCCCGGTTCC | 10† | M | 992 |
| 1381329 | 19531 | 19550 | 1392 | 1411 | GCCCACAAAAGGCAGGTGGA | 103 | M | 993 |
| 1381358 | 12147 | 12166 | 180 | 199 | CAGGCAGCACCATGGCCCCT | 115 | M | 994 |
| 1381364 | 15073 | 15092 | N/A | N/A | CCTCACCTCGCCCCTCTTCA | 64 | M | 995 |
| 1381371 | 17015 | 17034 | 1168 | 1187 | CCCGGCCCAGCCGTGTCTCC | 123 | M | 996 |
| 1381384 | 24374 | 24393 | 2432 | 2451 | CCAGGCCTGCAGTTTGCCCA | 104 | M | 997 |
| 1381391 | 17150 | 17169 | 1303 | 1322 | CGTCCTCCACCAAGTCGAAG | 68 | M | 998 |
| 1381392 | 24555 | 24574 | 2613 | 2632 | GCGCGGGATCCCCGAAAAAG | 108 | M | 999 |
| 1381406 | 24195 | 24214 | 2253 | 2272 | ACCCGGCCCCTCCCTCCCCG | 65 | M | 1000 |
| 1381416 | 22138 | 22157 | N/A | N/A | GGCACCTTTCCTTCCATGGC | 46 | M | 1001 |
| 1381452 | 24530 | 24549 | 2588 | 2607 | TTGGCAAAAGCAAATTTCCC | 87 | M | 1002 |
| 1381460 | 15013 | 15032 | 468 | 487 | GCCCGTCTGCTTCATCTTCA | 74 | M | 1003 |
| 1381468 | 14664 | 14683 | 374 | 393 | TCCTTAAGCCTCACCACGAT | 115 | M | 1004 |
| 1381474 | 17352 | 17371 | N/A | N/A | CTGGACTGTAAGTCTAGGTC | 67 | M | 1005 |
| 1381485 | 24336 | 24355 | 2394 | 2413 | CCTGGCCGAAAGAAAGAAAT | 104 | M | 1006 |
| 1381511 | 24440 | 24459 | 2498 | 2517 | TTGCGAACCAACGATAGGTG | 58 | M | 1007 |
| 1381520 | 17078 | 17097 | 1231 | 1250 | CCCGGAGACCATCCCAGTCG | 128 | M | 1008 |
| 1381532 | 22626 | 22645 | 1579 | 1598 | CGGCCTCAGCCTCTGCCGCA | 136 | M | 1009 |
| 1381539 | 12919 | 12938 | N/A | N/A | CCCAAAGTTGTCCCTCCTGG | 49 | M | 1010 |
| 1381557 | 24222 | 24241 | 2280 | 2299 | CCTTCGAGCCCCGTTCGCCG | 83 | M | 1011 |
| 1381582 | 24506 | 24525 | 2564 | 2583 | AAGCAGGCAGAGATCGCGCC | 98 | M | 1012 |
| 1381584 | 24135 | 24154 | 2193 | 2212 | GCGGAGACCCACGCTCGGAG | 66 | M | 1013 |
| 1381603 | 24780 | 24799 | 2838 | 2857 | ATGGAGGGCCTTTTATTCGC | 20 | M | 1014 |
| 1381631 | 22918 | 22937 | 1766 | 1785 | AGCAACTCCATCCGCTCCTG | 160 | M | 1015 |
| 1381641 | 24080 | 24099 | 2138 | 2157 | GTGCCGTGCCCCGGGCACTC | 99 | M | 1016 |
| 1381649 | 14169 | 14188 | N/A | N/A | GCAGGTGGTTCTTGAACCAC | 95 | M | 1017 |
| 1381660 | 15936 | 15955 | 690 | 709 | CGGAATCCGCTCCCCAAACT | 59 | M | 1018 |
| 1381683 | 24239 | 24258 | 2297 | 2316 | TTCCCGGCTACAAGGACCCT | 92 | M | 1019 |
| 1052865 | 24727 | 24746 | 2785 | 2804 | GACAATAAATACCGAGGAAT | 31 | N | 1020 |
| 1052870 | 19493 | 19512 | 1354 | 1373 | CCCGAATGTCCGACAGTGTC | 39 | N | 1021 |
| 1052873 | 19496 | 19515 | 1357 | 1376 | CTTCCCGAATGTCCGACAGT | 86 | N | 19 |
| 1052879 | 21092 | 21111 | N/A | N/A | GTGTAATGTTGTCCAGTAAT | 4 | N | 1022 |
| 1380293 | 20887 | 20906 | N/A | N/A | AATTTACTTGTGATAAGCAA | 42 | N | 1023 |
| 1380326 | 17353 | 17372 | N/A | N/A | ACTGGACTGTAAGTCTAGGT | 145 | N | 1024 |
| 1380358 | 17280 | 17299 | N/A | N/A | AGTGATTCAGGACCCCAGAA | 21 | N | 1025 |
| 1380377 | 21052 | 21071 | N/A | N/A | ACTGTTCTCTTAGACAAAGT | 64 | N | 1026 |
| 1380431 | 10711 | 10730 | N/A | N/A | TCCATAATTTAACACTCTTC | 17 | N | 1027 |
| 1380433 | 19359 | 19378 | N/A | N/A | AGTGGCCCCTCCAGCATTTT | 59 | N | 1028 |
| 1380437 | 21976 | 21995 | N/A | N/A | GCTTCTGTTCAGGAAGTCCC | 64 | N | 1029 |
| 1380456 | 19925 | 19944 | N/A | N/A | GGCCTTATTGTTATATGGCT | 83 | N | 1030 |
| 1380458 | 14048 | 14067 | N/A | N/A | ATCTCTCAGTCCTCCAGGAG | 67 | N | 1031 |
| 1380461 | 14532 | 14551 | N/A | N/A | AGCTTCACCCTAGGACTGTC | 47 | N | 1032 |
| 1380496 | 14234 | 14253 | N/A | N/A | TCATGTAGAATGTCCTGGGT | 26 | N | 1033 |
| 1380511 | 24044 | 24063 | 2102 | 2121 | CCCGGAGTCGAAGACAGTTC | 133 | N | 1034 |
| 1380514 | 22340 | 22359 | N/A | N/A | ACTAACACAACCTATGTCCC | 77 | N | 1035 |
| 1380559 | 17605 | 17624 | N/A | N/A | AGCAAAAGGGCACCCAGAGC | 62 | N | 1036 |
| 1380587 | 24456 | 24475 | 2514 | 2533 | AAGAAAGCTTTGCACTTTGC | 46 | N | 1037 |
| 1380598 | 14019 | 14038 | N/A | N/A | AGTCCTACCCCTTATTTACA | 17 | N | 1038 |
| 1380637 | 19665 | 19684 | N/A | N/A | AGTAAGGTTCCAAGACTGAT | 38 | N | 1039 |
| 1380653 | 17117 | 17136 | 1270 | 1289 | TGGCACCTTCGAAATCCGGT | 77 | N | 1040 |
| 1380685 | 20813 | 20832 | N/A | N/A | TAGGCTGGAATCTATCATGG | 58 | N | 1041 |
| 1380692 | 24632 | 24651 | 2690 | 2709 | GAGGTCAATAAATATCCAAA | 54 | N | 1042 |
| 1380695 | 22309 | 22328 | N/A | N/A | AAATCCCTCCAGCTCCCTAA | 70 | N | 1043 |
| 1380734 | 21466 | 21485 | N/A | N/A | CTCCCTTGACATGTGACCGC | 90 | N | 1044 |
| 1380806 | 22050 | 22069 | N/A | N/A | TACTTAATGCCCCACTGTAA | 126 | N | 1045 |
| 1380831 | 16698 | 16717 | 927 | 946 | GCCCACAGCCTGCAGGATCT | 64 | N | 1046 |
| 1380849 | 15174 | 15193 | 550 | 569 | CGTCCCTCTCCTCACGGAAG | 48 | N | 1047 |
| 1380915 | 9686 | 9705 | N/A | N/A | AGGACTCTACGATTCCAAAA | 55 | N | 1048 |
| 1380991 | 19852 | 19871 | N/A | N/A | ATTTTCCAAGTTCTGAAGTC | 70 | N | 1049 |
| 1381003 | 15634 | 15653 | N/A | N/A | GCAGATTCACTCCCCCTGAG | 61 | N | 1050 |
| 1381014 | 22141 | 22160 | N/A | N/A | CTTGGCACCTTTCCTTCCAT | 54 | N | 1051 |
| 1381026 | 22195 | 22214 | N/A | N/A | AATCAGGATTCCCACCTGCC | 87 | N | 1052 |
| 1381059 | 13975 | 13994 | N/A | N/A | TCAAGGTCCTATGACTAGGA | 20 | N | 1053 |
| 1381093 | 22324 | 22343 | N/A | N/A | TCCCTCTGCTGCTCAAAATC | 112 | N | 1054 |
| 1381120 | 16713 | 16732 | 942 | 961 | TGTCCCAGGCCCACCGCCCA | 112 | N | 1055 |
| 1381147 | 22238 | 22257 | N/A | N/A | AGCCTTCTCAGGAATGATTC | 63 | N | 1056 |
| 1381169 | 22293 | 22312 | N/A | N/A | CTAATGCCCTCACGACAAAA | 81 | N | 1057 |
| 1381202 | 19714 | 19733 | N/A | N/A | ATTTAAGGTCCTCCAACTCT | 73 | N | 1058 |
| 1381205 | 15339 | 15358 | N/A | N/A | AGCACCCCCAATCCTAGAGC | 59 | N | 1059 |
| 1381241 | 24375 | 24394 | 2433 | 2452 | CCCAGGCCTGCAGTTTGCCC | 105 | N | 1060 |
| 1381248 | 24392 | 24411 | 2450 | 2469 | GCCCGGCTTGCTGCCTTCCC | 83 | N | 1061 |
| 1381262 | 22884 | 22903 | 1732 | 1751 | GTGCCTCTAGGTCCCGGTTC | 13† | N | 1062 |
| 1381264 | 24782 | 24801 | 2840 | 2859 | AGATGGAGGGCCTTTTATTC | 47 | N | 1063 |
| 1381270 | 17016 | 17035 | 1169 | 1188 | CCCCGGCCCAGCCGTGTCTC | 84 | N | 1064 |
| 1381278 | 24337 | 24356 | 2395 | 2414 | GCCTGGCCGAAAGAAAGAAA | 124 | N | 1065 |
| 1381289 | 17173 | 17192 | 1326 | 1345 | CCCGCTCACCATGGCAGTGA | 130 | N | 1066 |
| 1381300 | 13685 | 13704 | N/A | N/A | GCCCTTTTAAGGCAGCAGGA | 77 | N | 1067 |
| 1381309 | 17491 | 17510 | N/A | N/A | GCTCAGATAGCTCCCCACTC | 102 | N | 1068 |
| 1381335 | 15978 | 15997 | 732 | 751 | GGCCATGACAATCTCCGCCA | 97 | N | 1069 |
| 1381339 | 12767 | 12786 | N/A | N/A | CTGTCGGCTGCGCCCCTGGC | 86 | N | 1070 |
| 1381344 | 19534 | 19553 | 1395 | 1414 | GTAGCCCACAAAAGGCAGGT | 93 | N | 1071 |
| 1381367 | 24441 | 24460 | 2499 | 2518 | TTTGCGAACCAACGATAGGT | 58 | N | 1072 |
| 1381386 | 15016 | 15035 | 471 | 490 | CTGGCCCGTCTGCTTCATCT | 122 | N | 1073 |
| 1381394 | 15914 | 15933 | 668 | 687 | CTCAGCAGTGTCAGCAGGTC | 53 | N | 1074 |
| 1381400 | 15937 | 15956 | 691 | 710 | CCGGAATCCGCTCCCCAAAC | 64 | N | 1075 |
| 1381430 | 24507 | 24526 | 2565 | 2584 | TAAGCAGGCAGAGATCGCGC | 54 | N | 1076 |
| 1381453 | 20946 | 20965 | N/A | N/A | CCACTGCCATCTGGTGAGCT | 60 | N | 1077 |
| 1381477 | 24240 | 24259 | 2298 | 2317 | ATTCCCGGCTACAAGGACCC | 57 | N | 1078 |
| 1381488 | 23026 | 23045 | N/A | N/A | CCTCCTCCAGGTGTCTATAC | 120 | N | 1079 |
| 1381516 | 14184 | 14203 | N/A | N/A | CCTCTAGATTCAGATGCAGG | 70 | N | 1080 |
| 1381519 | 24556 | 24575 | 2614 | 2633 | GGCGCGGGATCCCCGAAAAA | 89 | N | 1081 |
| 1381551 | 12148 | 12167 | 181 | 200 | ACAGGCAGCACCATGGCCCC | 119 | N | 1082 |
| 1381554 | 12522 | 12541 | N/A | N/A | CTGACCTTACTCTGCCCCTC | 19 | N | 1083 |
| 1381564 | 14665 | 14684 | 375 | 394 | CTCCTTAAGCCTCACCACGA | 69 | N | 1084 |
| 1381587 | 24223 | 24242 | 2281 | 2300 | CCCTTCGAGCCCCGTTCGCC | 105 | N | 1085 |
| 1381593 | 21210 | 21229 | N/A | N/A | AGCTATCCTGGACATGCGCC | 53 | N | 1086 |
| 1381594 | 24136 | 24155 | 2194 | 2213 | GGCGGAGACCCACGCTCGGA | 79 | N | 1087 |
| 1381598 | 24082 | 24101 | 2140 | 2159 | CTGTGCCGTGCCCCGGGCAC | 104 | N | 1088 |
| 1381601 | 24531 | 24550 | 2589 | 2608 | TTTGGCAAAAGCAAATTTCC | 82 | N | 1089 |
| 1381606 | 17722 | 17741 | N/A | N/A | TGCTTCCCTTCAGGGCACCT | 63 | N | 1090 |
| 1381622 | 17698 | 17717 | N/A | N/A | CACTCTCCATAATTCTCTAA | 77 | N | 1091 |
| 1381628 | 12999 | 13018 | N/A | N/A | GCTCATAGGAACCGAGACTT | 75 | N | 1092 |
| 1381630 | 15074 | 15093 | N/A | N/A | CCCTCACCTCGCCCCTCTTC | 93 | N | 1093 |
| 1381635 | 17151 | 17170 | 1304 | 1323 | CCGTCCTCCACCAAGTCGAA | 63 | N | 1094 |
| 1381652 | 24199 | 24218 | 2257 | 2276 | GCGGACCCGGCCCCTCCCTC | 85 | N | 1095 |
| 1381659 | 22627 | 22646 | 1580 | 1599 | TCGGCCTCAGCCTCTGCCGC | 111 | N | 1096 |
| 1052873 | 19496 | 19515 | 1357 | 1376 | CTTCCCGAATGTCCGACAGT | 83 | O | 19 |
| 1380292 | 9056 | 9075 | N/A | N/A | TGAGACACTAAGATTTCCCT | 68 | O | 327 |
| 1380373 | 10684 | 10703 | N/A | N/A | CAGACAATAGCAAGGGCAGC | 38 | O | 640 |
| 1380565 | 10405 | 10424 | N/A | N/A | CAAATTTTGTGCAGGTGGGG | 59 | O | 1097 |
| 1380665 | 10407 | 10426 | N/A | N/A | CCCAAATTTTGTGCAGGTGG | 21 | O | 1098 |
| 1382635 | 819 | 838 | N/A | N/A | ATCCTACAATGGTTCGGGCA | 96 | O | 1099 |
| 1382636 | 1127 | 1146 | N/A | N/A | TCATATTTTCCACCAACCTC | 122 | O | 1100 |
| 1382637 | 5046 | 5065 | N/A | N/A | CTCCATGTATGATTCTACAA | 58 | O | 1101 |
| 1382639 | 7676 | 7695 | N/A | N/A | CAGCCACTTCAGATATGTCA | 51 | O | 1102 |
| 1382641 | 5911 | 5930 | N/A | N/A | GTGCAATCCTGCATTTGTCC | 76 | O | 1103 |
| 1382642 | 6319 | 6338 | N/A | N/A | GGGCCTGTCTGTCCTCTGCA | 117 | O | 1104 |
| 1382643 | 3672 | 3691 | N/A | N/A | CTGCATCCTAATGGCATATA | 72 | O | 1105 |
| 1382644 | 1784 | 1803 | N/A | N/A | GGGCCCGAGCCGCCCTCCGC | 139 | O | 1106 |
| 1382645 | 910 | 929 | N/A | N/A | CCATCATAGATCTTTCTAGT | 80 | O | 1107 |
| 1382647 | 3437 | 3456 | N/A | N/A | GCTTGTTGAGGTCAATGGAC | 74 | O | 1108 |
| 1382649 | 2190 | 2209 | N/A | N/A | CCGTTGGCTCCCACGACGAC | 83 | O | 1109 |
| 1382651 | 6786 | 6805 | N/A | N/A | CCTCTTTTCCTCATCCAATC | 86 | O | 1110 |
| 1382653 | 1808 | 1827 | N/A | N/A | GCGCAGTCCCCCATGGCGGC | 94 | O | 1111 |
| 1382655 | 10234 | 10253 | N/A | N/A | CTGGTTGGGAGGAGATGCCC | 88 | O | 1112 |
| 1382657 | 7653 | 7672 | N/A | N/A | TGGTCTTGTCGATCAACCGC | 84 | O | 1113 |
| 1382658 | 4825 | 4844 | N/A | N/A | CTGCAAAATGGGCAGCACAC | 103 | O | 1114 |
| 1382659 | 6084 | 6103 | N/A | N/A | CAGGGCTCAGGTGAGCCCCA | 138 | O | 1115 |
| 1382662 | 6234 | 6253 | N/A | N/A | GCAGCCTCTCTCAAGCAGGG | 45 | O | 1116 |
| 1382665 | 7673 | 7692 | N/A | N/A | CCACTTCAGATATGTCACCT | 31 | O | 1117 |
| 1382671 | 3601 | 3620 | N/A | N/A | CTCCTCATTGAACAGCTTGC | 98 | O | 1118 |
| 1382672 | 7354 | 7373 | N/A | N/A | GCCACAGAATTGTCCTGTAT | 47 | O | 1119 |
| 1382673 | 2835 | 2854 | N/A | N/A | GGGTTCTCACCACATCGCTT | 42 | O | 1120 |
| 1382675 | 2546 | 2565 | N/A | N/A | CTTGCATCACCTCATGATTC | 79 | O | 1121 |
| 1382677 | 3455 | 3474 | N/A | N/A | AGATCCGCTTGTCAATTGGC | 51 | O | 1122 |
| 1382679 | 3676 | 3695 | N/A | N/A | GCAGCTGCATCCTAATGGCA | 111 | O | 1123 |
| 1382683 | 5221 | 5240 | N/A | N/A | ATCCACTGACTGTTCCGTTC | 29 | O | 1124 |
| 1382688 | 3577 | 3596 | N/A | N/A | GTCCTTTTTGATGAGATCCA | 67 | O | 1125 |
| 1382689 | 3451 | 3470 | N/A | N/A | CCGCTTGTCAATTGGCTTGT | 108 | O | 1126 |
| 1382694 | 6919 | 6938 | N/A | N/A | GAAGCGATCTTTGTTTGGTT | 40 | O | 1127 |
| 1382700 | 2463 | 2482 | N/A | N/A | CCATCATGTCCAATAGTCAC | 93 | O | 1128 |
| 1382702 | 8213 | 8232 | N/A | N/A | CTCCTCTTCGCCGCTCCGCT | 102 | O | 1129 |
| 1382703 | 7668 | 7687 | N/A | N/A | TCAGATATGTCACCTTGGTC | 24 | O | 1130 |
| 1382705 | 2549 | 2568 | N/A | N/A | GGGCTTGCATCACCTCATGA | 120 | O | 1131 |
| 1382707 | 961 | 980 | N/A | N/A | CAGCTCCTACTCATAGCCAG | 136 | O | 1132 |
| 1382709 | 1780 | 1799 | N/A | N/A | CCGAGCCGCCCTCCGCGCCG | 71 | O | 1133 |
| 1382712 | 892 | 911 | N/A | N/A | GTCTCATGTAGTCAATGGCC | 118 | O | 1134 |
| 1382713 | 3453 | 3472 | N/A | N/A | ATCCGCTTGTCAATTGGCTT | 60 | O | 1135 |
| 1382716 | 3600 | 3619 | N/A | N/A | TCCTCATTGAACAGCTTGCT | 102 | O | 1136 |
| 1382718 | 5933 | 5952 | N/A | N/A | CTGCACTGGCCACACGGCTT | 87 | O | 1137 |
| 1382719 | 5920 | 5939 | N/A | N/A | ACGGCTTCTGTGCAATCCTG | 93 | O | 1138 |
| 1382722 | 9680 | 9699 | N/A | N/A | CTACGATTCCAAAACTGAGG | 40 | O | 1139 |
| 1382723 | 6286 | 6305 | N/A | N/A | TGCTCTGTGAAGTATCAGCC | 85 | O | 1140 |
| 1382724 | 6788 | 6807 | N/A | N/A | CCCCTCTTTTCCTCATCCAA | 85 | O | 1141 |
| 1382727 | 1766 | 1785 | N/A | N/A | GCGCCGCCCGCCGCCATCTT | 78 | O | 1142 |
| 1382728 | 5903 | 5922 | N/A | N/A | CTGCATTTGTCCTAGCTGGC | 90 | O | 1143 |
| 1382733 | 2236 | 2255 | N/A | N/A | CCTCCATCCTTTCAGCACCC | 70 | O | 1144 |
| 1382734 | 6914 | 6933 | N/A | N/A | GATCTTTGTTTGGTTCACTA | 20 | O | 1145 |
| 1382735 | 1071 | 1090 | N/A | N/A | CCTCACTTGTCCTCCAAAAC | 104 | O | 1146 |
| 1382736 | 2456 | 2475 | N/A | N/A | GTCCAATAGTCACCATCCCA | 35 | O | 1147 |
| 1382739 | 8202 | 8221 | N/A | N/A | CGCTCCGCTCCCCATCAGCA | 101 | O | 1148 |
| 1382740 | 2612 | 2631 | N/A | N/A | GCTCAATCTCAAGACCCCTC | 81 | O | 1149 |
| 1382741 | 11304 | 11323 | N/A | N/A | GAAACAGAAACATTTCGGGG | 27 | O | 1150 |
| 1382746 | 7358 | 7377 | N/A | N/A | GCTTGCCACAGAATTGTCCT | 125 | O | 1151 |
| 1382747 | 4809 | 4828 | N/A | N/A | ACACTTCTGTATCCACTGGT | 31 | O | 1152 |
| 1382748 | 5869 | 5888 | N/A | N/A | TGTCCTTCTTGGCTGGGTGT | 64 | O | 1153 |
| 1382752 | 9476 | 9495 | N/A | N/A | CTCTGTTCTAAGAGGTGATG | 77 | O | 1154 |
| 1382755 | 10496 | 10515 | N/A | N/A | GAGGTCATTGTACTTGGCAG | 19 | O | 1155 |
| 1382757 | 10009 | 10028 | N/A | N/A | CACGCAATAATCAAAGTCCT | 77 | O | 1156 |
| 1382758 | 10757 | 10776 | N/A | N/A | AGTATAAATAAGAGGTCCTG | 39 | O | 1157 |
| 1382760 | 5053 | 5072 | N/A | N/A | GAGCAATCTCCATGTATGAT | 20 | O | 1158 |
| 1382761 | 4813 | 4832 | N/A | N/A | CAGCACACTTCTGTATCCAC | 27 | O | 1159 |
| 1382762 | 8060 | 8079 | N/A | N/A | GACCAGGTCATCTTCGCCAC | 95 | O | 1160 |
| 1382764 | 2554 | 2573 | N/A | N/A | CCTCTGGGCTTGCATCACCT | 116 | O | 1161 |
| 1382766 | 6916 | 6935 | N/A | N/A | GCGATCTTTGTTTGGTTCAC | 42 | O | 1162 |
| 1382767 | 7307 | 7326 | N/A | N/A | GAACAGTAGTGGTCCGTGGC | 52 | O | 1163 |
| 1382770 | 8196 | 8215 | N/A | N/A | GCTCCCCATCAGCACCGGCT | 98 | O | 1164 |
| 1382771 | 7689 | 7708 | N/A | N/A | TCTCCGACTCAGGCAGCCAC | 82 | 0 | 1165 |
| 1382772 | 5121 | 5140 | N/A | N/A | GAGCAGTCCCTGCCCGCGGA | 88 | O | 1166 |
| 1382773 | 5208 | 5227 | N/A | N/A | TCCGTTCACTCCCTTCAGCC | 54 | O | 1167 |
| 1382774 | 8054 | 8073 | N/A | N/A | GTCATCTTCGCCACCCGTCA | 40 | O | 1168 |
| 1382775 | 1131 | 1150 | N/A | N/A | TCCTTCATATTTTCCACCAA | 99 | O | 1169 |
| 1382779 | 7392 | 7411 | N/A | N/A | CTCGATGTCCCATCTTTGGC | 103 | O | 1170 |
| 1382780 | 1133 | 1152 | N/A | N/A | CATCCTTCATATTTTCCACC | 145 | O | 1171 |
| 1052873 | 19496 | 19515 | 1357 | 1376 | CTTCCCGAATGTCCGACAGT | 100 | P | 19 |
| 1380516 | 10683 | 10702 | N/A | N/A | AGACAATAGCAAGGGCAGCT | 14 | P | 1172 |
| 1380835 | 9239 | 9258 | N/A | N/A | GAAATAGATTCTGGTTCGAG | 24 | P | 1173 |
| 1380898 | 10406 | 10425 | N/A | N/A | CCAAATTTTGTGCAGGTGGG | 41 | P | 1174 |
| 1380915 | 9686 | 9705 | N/A | N/A | AGGACTCTACGATTCCAAAA | 88 | P | 1048 |
| 1382638 | 3430 | 3449 | N/A | N/A | GAGGTCAATGGACTTGAGGG | 36 | P | 1175 |
| 1382640 | 3449 | 3468 | N/A | N/A | GCTTGTCAATTGGCTTGTTG | 113 | P | 1176 |
| 1382646 | 7206 | 7225 | N/A | N/A | CAGCGCACCATTCAATCCTC | 62 | P | 1177 |
| 1382648 | 3681 | 3700 | N/A | N/A | CGCAGGCAGCTGCATCCTAA | 87 | P | 1178 |
| 1382650 | 8199 | 8218 | N/A | N/A | TCCGCTCCCCATCAGCACCG | 85 | P | 1179 |
| 1382652 | 5906 | 5925 | N/A | N/A | ATCCTGCATTTGTCCTAGCT | 55 | P | 1180 |
| 1382654 | 10233 | 10252 | N/A | N/A | TGGTTGGGAGGAGATGCCCT | 103 | P | 1181 |
| 1382656 | 4814 | 4833 | N/A | N/A | GCAGCACACTTCTGTATCCA | 47 | P | 1182 |
| 1382660 | 7356 | 7375 | N/A | N/A | TTGCCACAGAATTGTCCTGT | 78 | P | 1183 |
| 1382661 | 1130 | 1149 | N/A | N/A | CCTTCATATTTTCCACCAAC | 87 | P | 1184 |
| 1382663 | 2458 | 2477 | N/A | N/A | ATGTCCAATAGTCACCATCC | 38 | P | 1185 |
| 1382664 | 1779 | 1798 | N/A | N/A | CGAGCCGCCCTCCGCGCCGC | 84 | P | 1186 |
| 1382666 | 10495 | 10514 | N/A | N/A | AGGTCATTGTACTTGGCAGT | 17 | P | 1187 |
| 1382667 | 9550 | 9569 | N/A | N/A | CGCCTACTATGACCTTCCCA | 63 | P | 1188 |
| 1382668 | 6917 | 6936 | N/A | N/A | AGCGATCTTTGTTTGGTTCA | 46 | P | 1189 |
| 1382669 | 2484 | 2503 | N/A | N/A | TCCCACCTCATCTCAGATAC | 77 | P | 1190 |
| 1382670 | 7692 | 7711 | N/A | N/A | GGCTCTCCGACTCAGGCAGC | 63 | P | 1191 |
| 1382674 | 2563 | 2582 | N/A | N/A | TCTCCATCACCTCTGGGCTT | 75 | P | 1192 |
| 1382676 | 5192 | 5211 | N/A | N/A | AGCCATTCACTCATTCTGAG | 33 | P | 1193 |
| 1382678 | 6331 | 6350 | N/A | N/A | GCTCCAATGCCTGGGCCTGT | 109 | P | 1194 |
| 1382680 | 10712 | 10731 | N/A | N/A | TTCCATAATTTAACACTCTT | 29 | P | 1195 |
| 1382681 | 6885 | 6904 | N/A | N/A | GCAGTGCACACAGGAGGCAG | 67 | P | 1196 |
| 1382682 | 3452 | 3471 | N/A | N/A | TCCGCTTGTCAATTGGCTTG | 80 | P | 1197 |
| 1382684 | 4811 | 4830 | N/A | N/A | GCACACTTCTGTATCCACTG | 16 | P | 1198 |
| 1382685 | 1110 | 1129 | N/A | N/A | CTCCAATGTATATTTCCATG | 82 | P | 1199 |
| 1382686 | 956 | 975 | N/A | N/A | CCTACTCATAGCCAGTGCTC | 94 | P | 1200 |
| 1382687 | 2308 | 2327 | N/A | N/A | GTCCATCACATTTAGAATGC | 41 | P | 1201 |
| 1382690 | 7398 | 7417 | N/A | N/A | AGGAGCCTCGATGTCCCATC | 82 | P | 1202 |
| 1382691 | 6250 | 6269 | N/A | N/A | CTGCTCTGAACACTTCGCAG | 108 | P | 1203 |
| 1382692 | 1145 | 1164 | N/A | N/A | GCTAACTCCAAACATCCTTC | 111 | P | 1204 |
| 1382693 | 8912 | 8931 | N/A | N/A | CGCCTTGCCCGGCCGGGCCC | 111 | P | 1205 |
| 1382695 | 10365 | 10384 | N/A | N/A | TCATGGTTAGTCTTGTTAAT | 25 | P | 1206 |
| 1382696 | 2547 | 2566 | N/A | N/A | GCTTGCATCACCTCATGATT | 103 | P | 1207 |
| 1382697 | 5917 | 5936 | N/A | N/A | GCTTCTGTGCAATCCTGCAT | 89 | P | 1208 |
| 1382698 | 7661 | 7680 | N/A | N/A | TGTCACCTTGGTCTTGTCGA | 53 | P | 1209 |
| 1382699 | 2981 | 3000 | N/A | N/A | TCCTAGCATTCTCTTCACTT | 74 | P | 1210 |
| 1382701 | 11537 | 11556 | N/A | N/A | CTGCTTTATACCAGCTTTTT | 28 | P | 1211 |
| 1382704 | 5376 | 5395 | N/A | N/A | AACTGTCTGTCTCCCCAGCT | 78 | P | 1212 |
| 1382706 | 1807 | 1826 | N/A | N/A | CGCAGTCCCCCATGGCGGCG | 114 | P | 1213 |
| 1382708 | 893 | 912 | N/A | N/A | AGTCTCATGTAGTCAATGGC | 89 | P | 1214 |
| 1382710 | 6165 | 6184 | N/A | N/A | AAGGAGCTGCTCTCCCAGGA | 92 | P | 1215 |
| 1382711 | 3673 | 3692 | N/A | N/A | GCTGCATCCTAATGGCATAT | 104 | P | 1216 |
| 1382714 | 3463 | 3482 | N/A | N/A | GCCCTTGTAGATCCGCTTGT | 98 | P | 1217 |
| 1382715 | 5047 | 5066 | N/A | N/A | TCTCCATGTATGATTCTACA | 26 | P | 1218 |
| 1382717 | 8061 | 8080 | N/A | N/A | TGACCAGGTCATCTTCGCCA | 76 | P | 1219 |
| 1382720 | 10957 | 10976 | N/A | N/A | TTGTGTGACTCGCAGGTCCG | 54 | P | 1220 |
| 1382721 | 7670 | 7689 | N/A | N/A | CTTCAGATATGTCACCTTGG | 47 | P | 1221 |
| 1382725 | 5875 | 5894 | N/A | N/A | TCAGAATGTCCTTCTTGGCT | 56 | P | 1222 |
| 1382726 | 1869 | 1888 | N/A | N/A | GCCGTCGCCCGGGAGTAGCT | 54 | P | 1223 |
| 1382729 | 6055 | 6074 | N/A | N/A | GCTCCTCCACAGGCTTCCAA | 93 | P | 1224 |
| 1382730 | 7687 | 7706 | N/A | N/A | TCCGACTCAGGCAGCCACTT | 65 | P | 1225 |
| 1382731 | 7652 | 7671 | N/A | N/A | GGTCTTGTCGATCAACCGCT | 93 | P | 1226 |
| 1382732 | 3662 | 3681 | N/A | N/A | ATGGCATATAGTGGGTAGGT | 20 | P | 1227 |
| 1382737 | 7674 | 7693 | N/A | N/A | GCCACTTCAGATATGTCACC | 55 | P | 1228 |
| 1382738 | 3454 | 3473 | N/A | N/A | GATCCGCTTGTCAATTGGCT | 55 | P | 1229 |
| 1382742 | 2550 | 2569 | N/A | N/A | TGGGCTTGCATCACCTCATG | 107 | P | 1230 |
| 1382743 | 4856 | 4875 | N/A | N/A | CCGACTTATTTACTAGCCTC | 81 | P | 1231 |
| 1382744 | 6787 | 6806 | N/A | N/A | CCCTCTTTTCCTCATCCAAT | 72 | P | 1232 |
| 1382745 | 962 | 981 | N/A | N/A | ACAGCTCCTACTCATAGCCA | 138 | P | 1233 |
| 1382749 | 5211 | 5230 | N/A | N/A | TGTTCCGTTCACTCCCTTCA | 43 | P | 1234 |
| 1382750 | 7391 | 7410 | N/A | N/A | TCGATGTCCCATCTTTGGCG | 99 | P | 1235 |
| 1382751 | 5095 | 5114 | N/A | N/A | CGTTTACTGGTATCTCCTCG | 21 | P | 1236 |
| 1382753 | 6915 | 6934 | N/A | N/A | CGATCTTTGTTTGGTTCACT | 20 | P | 1237 |
| 1382754 | 859 | 878 | N/A | N/A | CCTAGAGTGTCCCAAGGGCA | 110 | P | 1238 |
| 1382756 | 2834 | 2853 | N/A | N/A | GGTTCTCACCACATCGCTTT | 58 | P | 1239 |
| 1382759 | 8056 | 8075 | N/A | N/A | AGGTCATCTTCGCCACCCGT | 77 | P | 1240 |
| 1382763 | 5925 | 5944 | N/A | N/A | GCCACACGGCTTCTGTGCAA | 111 | P | 1241 |
| 1382765 | 8205 | 8224 | N/A | N/A | CGCCGCTCCGCTCCCCATCA | 90 | P | 1242 |
| 1382768 | 7351 | 7370 | N/A | N/A | ACAGAATTGTCCTGTATCTC | 55 | P | 1243 |
| 1382769 | 6317 | 6336 | N/A | N/A | GCCTGTCTGTCCTCTGCAGC | 101 | P | 1244 |
| 1382776 | 1781 | 1800 | N/A | N/A | CCCGAGCCGCCCTCCGCGCC | 86 | P | 1245 |
| 1382777 | 3578 | 3597 | N/A | N/A | TGTCCTTTTTGATGAGATCC | 65 | P | 1246 |
| 1382778 | 1132 | 1151 | N/A | N/A | ATCCTTCATATTTTCCACCA | 108 | P | 1247 |

### Example 2: Effect of 3-10-3 cET Modified Oligonucleotides with Uniform Phosphorothioate Internucleoside Linkages on Human DMPK In Vitro, Single Dose

Modified oligonucleotides complementary to human DMPK nucleic acid were synthesized and tested for their effect on DMPK RNA levels in vitro. The results are presented in the tables below.

The modified oligonucleotides in the tables below are 3-10-3 cEt modified oligonucleotides with uniform phosphorothioate internucleoside linkages. The modified oligonucleotides are 16 nucleosides in length. The sugar motif for the modified oligonucleotides is (from 5′ to 3′): kkkddddddddddkkk; wherein each ‘d’ represents a 2′-β-D-deoxyribosyl sugar moiety, and each ‘k’ represents a cEt sugar moiety. The internucleoside linkage motif for the modified oligonucleotides is (from 5′ to 3′): sssssssssssssss wherein each ‘s’ represents a phosphorothioate internucleoside linkage. All cytosine nucleobases are 5-methylcytosines.

“Start site” indicates the 5′-most nucleoside of the target sequence to which the modified oligonucleotide is complementary. “Stop site” indicates the 3′-most nucleoside of the target sequence to which the modified oligonucleotide is complementary. As shown in the tables below, the modified oligonucleotides are complementary to SEQ TD NO: 1 (the complement of GENBANK Accession No. NT_011109.16, truncated from nucleotides 18539000 to 18566000), SEQ ID NO: 2 (GENBANK Accession No. NM_004409.4), SEQ ID NO: 3 (the complement of GENBANK Accession No. NC_000019.10, truncated from nucleosides 45767001 to 45786000), SEQ ID NO: 4 (GENBANK Accession No. NM_001288764.1), and/or SEQ ID NO: 5 (GENBANK Accession No. NM_001081560.2). ‘N/A’ indicates that the modified oligonucleotide is not 100% complementary to that particular target sequence.

Cultured A431 cells at a density of 10,000 cells per well were treated with 2,000 nM of modified oligonucleotide by free uptake as indicated in the tables below. After a treatment period of approximately 48 hours. RNA was isolated from the cells and DMPK RNA levels were measured by quantitative real-time RTPCR. Human DMPK primer probe set RTS38095 (forward nucleobase sequence CTGAGCCGGGAGATGGA, designated herein as SEQ TD NO: 6; reverse nucleobase sequence GGACGTGTGCCTCTAGGT, designated herein as SEQ ID NO: 7; probe nucleobase sequence TGACTGGCGAAGTTCTGGTTGTCC, designated herein as SEQ ID NO: 8) was used to measure DMPK RNA levels. DMPK RNA levels were normalized to total RNA content, as measured by RIBOGREEN®. Results are presented as percent of DMPK RNA, relative to the amount in untreated control cells (% UTC). The values marked by the symbol “†” indicate that the modified oligonucleotide is complementary to the amplicon region of the primer probe set. Additional assays may be used to measure the activity of the modified oligonucleotides complementary to the amplicon region. ‘N.D.’ in the tables below refers to instances where the value was Not Defined.

Each separate experiment described in this example is identified by an Assay Identification letter in the table column labeled “AID”.

TABLE 3
Reduction of DMPK RNA by 3-10-3 cEt modified oligonucleotides with uniform phosphorothioate
internucleoside linkages at a dose of 2,000 nM

| | SEQ ID | SEQ ID | SEQ ID | SEQ ID | | | | |
|---|---|---|---|---|---|---|---|---|
| | NO: 1 | NO: 1 | NO: 2 | NO: 2 | | | | |
| Compound | Start | Stop | Start | Stop | Nucleobase Sequence | DMPK | | SEQ ID |
| No. | Site | Site | Site | Site | (5′ to 3′) | (%UTC) | AID | NO |
| 569664 | 19498 | 19513 | 1359 | 1374 | TCCCGAATGTCCGACA | 16 | Q | 1337 |
| 570781 | 20812 | 20827 | N/A | N/A | TGGAATCTATCATGGC | 9 | Q | 1338 |
| 570785 | 20884 | 20899 | N/A | N/A | TTGTGATAAGCAATGC | 0.3 | Q | 1339 |
| 570786 | 20886 | 20901 | N/A | N/A | ACTTGTGATAAGCAAT | 5 | Q | 1340 |
| 570787 | 20888 | 20903 | N/A | N/A | TTACTTGTGATAAGCA | 1 | Q | 1341 |
| 570880 | 21991 | 22006 | N/A | N/A | GGTTTCTGTCTGCTTC | 5 | Q | 1343 |
| 570883 | 21994 | 22009 | N/A | N/A | CGTGGTTTCTGTCTGC | 8 | Q | 1344 |
| 1002463 | 16983 | 16998 | 1136 | 1151 | GCTGAATGAAGTCTCG | 22 | Q | 1345 |
| 1002510 | 24091 | 24106 | 2149 | 2164 | GGCTTCTGTGCCGTGC | 95 | Q | 1346 |
| 1002598 | 14185 | 14200 | N/A | N/A | CTAGATTCAGATGCAG | 14 | Q | 1347 |
| 1002623 | 14486 | 14501 | N/A | N/A | CGTGTAAGGTTCTGGG | 11 | Q | 1348 |
| 1002656 | 14982 | 14997 | N/A | N/A | GTCGAGATAGTGAGAC | 102 | Q | 1349 |
| 1002707 | 13157 | 13172 | N/A | N/A | GCGGAAGCATCCTCCT | 83 | Q | 1350 |
| 1002727 | 13331 | 13346 | N/A | N/A | AACAAGTGTCACACAC | 18 | Q | 1351 |
| 1002849 | 15743 | 15758 | N/A | N/A | ATAACCATAGAGATCT | 30 | Q | 1352 |
| 1002919 | 17243 | 17258 | N/A | N/A | AACTTTATGGAGGGAG | 27 | Q | 1353 |
| 1002931 | 17320 | 17335 | N/A | N/A | TAGTAGATGGGCACAG | 13 | Q | 1354 |
| 1002937 | 17392 | 17407 | N/A | N/A | GGCCTTACTGTCTGAA | 105 | Q | 1355 |
| 1002989 | 19409 | 19424 | N/A | N/A | GCTTACATGTTCCCCC | 7 | Q | 1356 |
| 1003045 | 20712 | 20727 | N/A | N/A | GCCTGGCACGATTTTT | 93 | Q | 1357 |
| 1003066 | 21096 | 21111 | N/A | N/A | GTGTAATGTTGTCCAG | 1 | Q | 1358 |
| 1003078 | 21118 | 21133 | N/A | N/A | CATTAATGATAAGGTA | 44 | Q | 1359 |
| 1003105 | 21234 | 21249 | N/A | N/A | GGGAACACGGCTCAGG | 32 | Q | 1360 |
| 1003124 | 21490 | 21505 | N/A | N/A | TTCTAGGGTCAGCTCA | 12 | Q | 1361 |
| 1003161 | 22094 | 22109 | N/A | N/A | AAAGATCGACTTCTCA | 14 | Q | 1362 |
| 1003178 | 22234 | 22249 | N/A | N/A | CAGGAATGATTCAGCC | 2 | Q | 1363 |
| 1003199 | 22382 | 22397 | N/A | N/A | CTAAATCTACACAGGG | 47 | Q | 1364 |
| 1003250 | 23250 | 23265 | N/A | N/A | CCCTATATCTGGACGG | 71 | Q | 1365 |
| 1003291 | 23717 | 23732 | N/A | N/A | TCGAATCCCGTCCGAA | 90 | Q | 1366 |
| 1016696 | 12012 | 12027 | 45 | 60 | TCTCTCTGCGGCCGGC | 42 | Q | 1367 |
| 1016700 | 15989 | 16004 | 743 | 7 58 | AGTCTATGGCCATGAC | 64 | Q | 1368 |
| 1016717 | 24450 | 24465 | 2508 | 2523 | TGCACTTTGCGAACCA | 30 | Q | 1369 |
| 1016723 | 13959 | 13974 | N/A | N/A | ATGTTAAACTGGGCAG | 9 | Q | 1370 |
| 1016729 | 14033 | 14048 | N/A | N/A | GGAGTGCTTTAGTCCT | 27 | Q | 1371 |
| 1016754 | 8817 | 8832 | N/A | N/A | GATCTTCTTGCACACA | 37 | Q | 1372 |
| 1016766 | 8966 | 8981 | N/A | N/A | CCACGAAAGGTCCTGC | 28 | Q | 1373 |
| 1016778 | 9088 | 9103 | N/A | N/A | CACTCTAAGGATCTGA | 15 | Q | 1374 |
| 1016790 | 9360 | 9375 | N/A | N/A | TCAGATCCTGAGTCCC | 39 | Q | 1375 |
| 1016802 | 9574 | 9589 | N/A | N/A | ACAAGATGCCAGGCCT | 16 | Q | 1376 |
| 1016814 | 9689 | 9704 | N/A | N/A | GGACTCTACGATTCCA | 55 | Q | 1377 |
| 1016826 | 9872 | 9887 | N/A | N/A | TACCTCTACCACTGAC | 30 | Q | 1378 |
| 1016838 | 10051 | 10066 | N/A | N/A | CATCTTAGCTAGCTTC | 15 | Q | 1379 |
| 1016850 | 10281 | 10296 | N/A | N/A | CGATATCCATGGCTTC | 11 | Q | 1380 |
| 1016862 | 10372 | 10387 | N/A | N/A | CCATCATGGTTAGTCT | 12 | Q | 1381 |
| 1016874 | 10446 | 10461 | N/A | N/A | TTACATCGCCCGTGTC | 43 | Q | 1382 |
| 1016886 | 10553 | 10568 | N/A | N/A | ACCAGTCACATGCTGG | 47 | Q | 1383 |
| 1016898 | 10812 | 10827 | N/A | N/A | TACATCATCTCCTCCG | 21 | Q | 1384 |
| 1016910 | 11019 | 11034 | N/A | N/A | GGCAGGATGCTCTTCT | 19 | Q | 1385 |
| 1016922 | 11539 | 11554 | N/A | N/A | GCTTTATACCAGCTTT | 10 | Q | 1386 |
| 1016934 | 11864 | 11879 | N/A | N/A | GGGAATGCATGGAGAA | 25 | Q | 1387 |
| 1016942 | 12594 | 12609 | N/A | N/A | GCTGTTGGCAGCCTAG | 74 | Q | 1388 |
| 1016951 | 13619 | 13634 | N/A | N/A | TGCAGCTCGGGCCACA | 69 | Q | 1389 |
| 1016957 | 14811 | 14826 | N/A | N/A | TCAATTTCTAAGGCCC | 71 | Q | 1390 |
| 1016965 | 15368 | 15383 | N/A | N/A | GACTTTCCCACAGACG | 19 | Q | 1391 |
| 1016969 | 15481 | 15496 | N/A | N/A | CATCTTTATAAGAGTC | 28 | Q | 1392 |
| 1016980 | 16123 | 16138 | N/A | N/A | CCAACCAAGAAGGTCC | 97 | Q | 1393 |
| 1016985 | 16300 | 16315 | N/A | N/A | GTTTGATGTCCCTGCA | 34 | Q | 1394 |
| 1016991 | 16530 | 16545 | N/A | N/A | GGTCTAATACTCCGCC | 46 | Q | 1395 |
| 1017017 | 17621 | 17636 | N/A | N/A | CCAGTATTGTTCAGCA | 2 | Q | 1396 |
| 1017025 | 17739 | 17754 | N/A | N/A | TCTTGTATCCTGTTGC | 1 | Q | 1397 |
| 1017038 | 19712 | 19727 | N/A | N/A | GGTCCTCCAACTCTGG | 39 | Q | 1398 |
| 1017041 | 19802 | 19817 | N/A | N/A | TGGTTACAAGATTCTG | 1 | Q | 1399 |
| 1017049 | 19918 | 19933 | N/A | N/A | TATATGGCTGATTCAA | 6 | Q | 1400 |
| 1017058 | 20942 | 20957 | N/A | N/A | ATCTGGTGAGCTCACG | 36 | Q | 1401 |
| 1017085 | 21872 | 21887 | N/A | N/A | TACTGTTTCATCCTGT | 56 | Q | 1402 |
| 1017093 | 22002 | 22017 | N/A | N/A | CGTCTCTCCGTGGTTT | 6 | Q | 1403 |
| 1017115 | 22498 | 22513 | N/A | N/A | TGTTTATCCCCTACTC | 87 | Q | 1404 |
| 1017124 | 22951 | 22966 | N/A | N/A | GGGACTCACCTGTGGC | 117 | Q | 1405 |
| 1017144 | 24907 | 24922 | N/A | N/A | TCGCATCCCGCTAGCT | 91 | Q | 1406 |
| 1017156 | 25216 | 25231 | N/A | N/A | GGAATTCCCGGCTCCG | 92 | Q | 1407 |
| 1017168 | 25479 | 25494 | N/A | N/A | TGTGTGTCCGTCCCCC | 163 | Q | 1408 |
| 1017180 | 25660 | 25675 | N/A | N/A | GCAACTTTGGGAAGTT | 90 | Q | 1409 |
| 1017192 | 26179 | 26194 | N/A | N/A | CCTCATGGTAGCGCGC | 128 | Q | 1410 |
| 1017204 | 26277 | 26292 | N/A | N/A | TGTCTCCTCGCCGTCC | 93 | Q | 1411 |
| 1017216 | 26601 | 26616 | N/A | N/A | GAGATTGTGAGCTGGT | 106 | Q | 1412 |
| 1017228 | 26849 | 26864 | N/A | N/A | GTGGGTAAGAGTAACG | 84 | Q | 1413 |
| 569664 | 19498 | 19513 | 1359 | 1374 | TCCCGAATGTCCGACA | 21 | R | 1337 |
| 570767 | 19665 | 19680 | N/A | N/A | AGGTTCCAAGACTGAT | 11 | R | 1415 |
| 570776 | 19713 | 19728 | N/A | N/A | AGGTCCTCCAACTCTG | 30 | R | 1416 |
| 570796 | 21049 | 21064 | N/A | N/A | TCTTAGACAAAGTAGC | 6 | R | 1417 |
| 570884 | 21995 | 22010 | N/A | N/A | CCGTGGTTTCTGTCTG | 10 | R | 1418 |
| 570901 | 22206 | 22221 | V/A | N/A | AGGAACAAATCAGGAT | 4.4† | R | 1419 |
| 1002436 | 15990 | 16005 | 744 | 759 | GAGTCTATGGCCATGA | 37 | R | 1420 |
| 1002464 | 16984 | 16999 | 1137 | 1152 | CGCTGAATGAAGTCTC | 22 | R | 1421 |
| 1002599 | 14186 | 14201 | N/A | N/A | TCTAGATTCAGATGCA | 36 | R | 1422 |
| 1002628 | 14496 | 14511 | N/A | N/A | ACATAAACACCGTGTA | 67 | R | 1423 |
| 1002716 | 13266 | 13281 | N/A | N/A | ACATATGAGGGCCAGA | 17 | R | 1424 |
| 1002728 | 13333 | 13348 | N/A | N/A | GAAACAAGTGTCACAC | 20 | R | 1425 |
| 1002751 | 13624 | 13639 | N/A | N/A | CGAGATGCAGCTCGGG | 46 | R | 1426 |
| 1002777 | 14813 | 14828 | N/A | N/A | CATCAATTTCTAAGGC | 65 | R | 1427 |
| 1002799 | 15382 | 15397 | N/A | N/A | TCTTACCGCACACAGA | 73 | R | 1428 |
| 1002826 | 15521 | 15536 | N/A | N/A | ACCTATCCCATTCCAG | 14 | R | 1429 |
| 1002852 | 15746 | 15761 | N/A | N/A | GAGATAACCATAGAGA | 12 | R | 1430 |
| 1002920 | 17244 | 17259 | N/A | N/A | CAACTTTATGGAGGGA | 17 | R | 1431 |
| 1003014 | 19719 | 19734 | N/A | N/A | AATTTAAGGTCCTCCA | 11 | R | 1432 |
| 1003033 | 19920 | 19935 | N/A | N/A | GTTATATGGCTGATTC | 6 | R | 1332 |
| 1003079 | 21119 | 21134 | N/A | N/A | TCATTAATGATAAGGT | 3 | R | 1433 |
| 1003109 | 21276 | 21291 | N/A | N/A | GTATGAAGTGGCTGTC | 36 | R | 1434 |
| 1003162 | 22095 | 22110 | N/A | N/A | CAAAGATCGACTTCTC | 8 | R | 1435 |
| 1003179 | 22235 | 22250 | N/A | N/A | TCAGGAATGATTCAGC | 3 | R | 1436 |
| 1003200 | 22383 | 22398 | N/A | N/A | TCTAAATCTACACAGG | 72 | R | 1437 |
| 1016697 | 14688 | 14703 | 398 | 413 | CGTCCCTCTGCAGTCG | 34 | R | 1438 |
| 1016711 | 24117 | 24132 | 2175 | 2190 | GTTGTGAACTGGCAGG | 12 | R | 1439 |
| 1016718 | 24455 | 24470 | 2513 | 2528 | AGCTTTGCACTTTGCG | 64 | R | 1440 |
| 1016724 | 13961 | 13976 | N/A | N/A | GAATGTTAAACTGGGC | 11 | R | 1441 |
| 1016730 | 14034 | 14049 | N/A | N/A | AGGAGTGCTTTAGTCC | 39 | R | 1442 |
| 1016743 | 8568 | 8583 | N/A | N/A | GAATGGTGTGCCAGGC | 39 | R | 1443 |
| 1016755 | 8819 | 8834 | N/A | N/A | GCGATCTTCTTGCACA | 55 | R | 1444 |
| 1016767 | 8971 | 8986 | N/A | N/A | TCTTCCCACGAAAGGT | 58 | R | 1445 |
| 1016779 | 9150 | 9165 | N/A | N/A | TCCTAGCTGGCTCTCA | 23 | R | 1446 |
| 1016791 | 9392 | 9407 | N/A | N/A | GGGATTCCCAGCCTGT | 90 | R | 1447 |
| 1016803 | 9579 | 9594 | N/A | N/A | AACTTACAAGATGCCA | 12 | R | 1448 |
| 1016815 | 9697 | 9712 | N/A | N/A | GGGACTCAGGACTCTA | 82 | R | 1449 |
| 1016827 | 9929 | 9944 | N/A | N/A | TCCCTTCTAACTTGGG | 94 | R | 1450 |
| 1016839 | 10053 | 10068 | N/A | N/A | TGCATCTTAGCTAGCT | 72 | R | 1451 |
| 1016851 | 10282 | 10297 | N/A | N/A | CCGATATCCATGGCTT | 18 | R | 1452 |
| 1016863 | 10373 | 10388 | N/A | N/A | TCCATCATGGTTAGTC | 13 | R | 1453 |
| 1016875 | 10447 | 10462 | N/A | N/A | ATTACATCGCCCGTGT | 29 | R | 1454 |
| 1016887 | 10556 | 10571 | N/A | N/A | GTGACCAGTCACATGC | 41 | R | 1455 |
| 1016899 | 10818 | 10833 | N/A | N/A | GTCCTCTACATCATCT | 20 | R | 1456 |
| 1016911 | 11120 | 11135 | N/A | N/A | GACCGAGGAGTCCCAG | 75 | R | 1457 |
| 1016923 | 11540 | 11555 | N/A | N/A | TGCTTTATACCAGCTT | 18 | R | 1458 |
| 1016935 | 12329 | 12344 | N/A | N/A | GTAGGCACTCACCCCA | 76 | R | 1459 |
| 1016943 | 12595 | 12610 | N/A | N/A | GGCTGTTGGCAGCCTA | 126 | R | 1460 |
| 1016981 | 16169 | 16184 | N/A | N/A | GCTTCTACAGTTCTGA | 71 | R | 1461 |
| 1016986 | 16394 | 16409 | N/A | N/A | CACTGGCTCACCGTTC | 89 | R | 1462 |
| 1016992 | 16531 | 16546 | N/A | N/A | TGGTCTAATACTCCGC | 60 | R | 1463 |
| 1017004 | 17321 | 17336 | N/A | N/A | GTAGTAGATGGGCACA | 10 | R | 1464 |
| 1017013 | 17408 | 17423 | N/A | N/A | GCCCTGATCACTCTGG | 124 | R | 1465 |
| 1017018 | 17622 | 17637 | N/A | N/A | CCCAGTATTGTTCAGC | 5 | R | 1466 |
| 1017026 | 17740 | 17755 | N/A | N/A | TTCTTGTATCCTGTTG | 4 | R | 1467 |
| 1017032 | 19411 | 19426 | N/A | N/A | TGGCTTACATGTTCCC | 40 | R | 1468 |
| 1017042 | 19803 | 19818 | N/A | N/A | ATGGTTACAAGATTCT | 0.3 | R | 1469 |
| 1017054 | 20809 | 20824 | N/A | N/A | AATCTATCATGGCTCA | 7 | R | 1470 |
| 1017059 | 20944 | 20959 | N/A | N/A | CCATCTGGTGAGCTCA | 76 | R | 1471 |
| 1017066 | 21097 | 21112 | N/A | N/A | TGTGTAATGTTGTCCA | 3 | R | 1472 |
| 1017077 | 21491 | 21506 | N/A | N/A | GTTCTAGGGTCAGCTC | 7 | R | 1473 |
| 1017086 | 21874 | 21889 | N/A | N/A | CTTACTGTTTCATCCT | 75 | R | 1474 |
| 1017094 | 22016 | 22031 | N/A | N/A | GTCTGAAGTAACCTCG | 9 | R | 1475 |
| 1017116 | 22504 | 22519 | N/A | N/A | AATCCTTGTTTATCCC | 20 | R | 1476 |
| 1017125 | 22958 | 22973 | N/A | N/A | CACATGAGGGACTCAC | 71 | R | 1477 |
| 1017128 | 23288 | 23303 | N/A | N/A | GGCTTCTGCCCTCTAA | 98 | R | 1478 |
| 1017135 | 23736 | 23751 | N/A | N/A | GTTAGTCCACTCGCAC | 21 | R | 1479 |
| 1017145 | 24920 | 24935 | N/A | N/A | TGATTCGGCCGCTTCG | 97 | R | 1480 |
| 1017157 | 25217 | 25232 | N/A | N/A | CGGAATTCCCGGCTCC | 73 | R | 1481 |
| 1017169 | 25482 | 25497 | N/A | N/A | TTGTGTGTGTCCGTCC | 240 | R | 1482 |
| 1017181 | 25671 | 25686 | N/A | N/A | AGCCATGTTTTGCAAC | 82 | R | 1483 |
| 1017193 | 26223 | 26238 | N/A | N/A | ATACTTGTCCACTGCG | 76 | R | 1484 |
| 1017205 | 26280 | 26295 | N/A | N/A | GACTGTCTCCTCGCCG | 114 | R | 1485 |
| 1017217 | 26602 | 26617 | N/A | N/A | TGAGATTGTGAGCTGG | 154 | R | 1486 |
| 1017229 | 26912 | 26927 | N/A | N/A | CGTTTCACAACAAAGG | 88 | R | 1487 |
| 569664 | 19498 | 19513 | 1359 | 1374 | TCCCGAATGTCCGACA | 17 | S | 1337 |
| 570745 | 19410 | 19425 | N/A | N/A | GGCTTACATGTTCCCC | 29 | S | 1491 |
| 570784 | 20882 | 20897 | N/A | N/A | GTGATAAGCAATGCAT | 2 | S | 1492 |
| 570794 | 21045 | 21060 | N/A | N/A | AGACAAAGTAGCATGA | 11 | S | 1493 |
| 570795 | 21047 | 21062 | N/A | N/A | TTAGACAAAGTAGCAT | 3 | S | 1494 |
| 1002421 | 15001 | 15016 | 456 | 471 | TTCACTACCGCTACCT | 17 | S | 1495 |
| 1002590 | 14086 | 14101 | N/A | N/A | ACATATCCCAGACTCA | 16 | S | 1496 |
| 1002602 | 14223 | 14238 | N/A | N/A | TGGGTAACGGCCCAGA | 70 | S | 1497 |
| 1002629 | 14497 | 14512 | N/A | N/A | CACATAAACACCGTGT | 97 | S | 1498 |
| 1002679 | 12694 | 12709 | N/A | N/A | GGCCATAGAGCCCACT | 90 | S | 1499 |
| 1002717 | 13267 | 13282 | N/A | N/A | GACATATGAGGGCCAG | 17 | S | 1500 |
| 1002729 | 13335 | 13350 | N/A | N/A | AGGAAACAAGTGTCAC | 16 | S | 1501 |
| 1002752 | 13625 | 13640 | N/A | N/A | GCGAGATGCAGCTCGG | 51 | S | 1502 |
| 1002778 | 14825 | 14840 | N/A | N/A | GGCTCGGTCATTCATC | 55 | S | 1503 |
| 1002800 | 15384 | 15399 | N/A | N/A | CCTCTTACCGCACACA | 17 | S | 1504 |
| 1002831 | 15549 | 15564 | N/A | N/A | GGCAGTGGCCCCGTTA | 41 | S | 1505 |
| 1002853 | 15747 | 15762 | N/A | N/A | AGAGATAACCATAGAG | 10 | S | 1506 |
| 1002875 | 16170 | 16185 | N/A | N/A | GGCTTCTACAGTTCTG | 103 | S | 1507 |
| 1002975 | 17919 | 17934 | N/A | N/A | AGTATACAGGCATGCG | 30 | S | 1508 |
| 1003015 | 19723 | 19738 | N/A | N/A | TGAAAATTTAAGGTCC | 11 | S | 1509 |
| 1003080 | 21120 | 21135 | N/A | N/A | TTCATTAATGATAAGG | 2 | S | 1510 |
| 1003140 | 21879 | 21894 | N/A | N/A | ACCAACTTACTGTTTC | 83 | S | 1511 |
| 1003156 | 22038 | 22053 | N/A | N/A | GTAACTACAGAGACCG | 16 | S | 1512 |
| 1003163 | 22096 | 22111 | N/A | N/A | TCAAAGATCGACTTCT | 20 | S | 1513 |
| 1003183 | 22299 | 22314 | N/A | N/A | CCCTAATGCCCTCACG | 31 | S | 1514 |
| 1003231 | 22959 | 22974 | N/A | N/A | ACACATGAGGGACTCA | 96 | S | 1515 |
| 1003256 | 23299 | 23314 | N/A | N/A | GCTGAATAAAGGGCTT | 109 | S | 1516 |
| 1016701 | 15999 | 16014 | 753 | 768 | CGGTGCACCGAGTCTA | 156 | S | 1517 |
| 1016712 | 24121 | 24136 | 2179 | 2194 | AGCGGTTGTGAACTGG | 16 | S | 1518 |
| 1016719 | 24456 | 24471 | 2514 | 2529 | AAGCTTTGCACTTTGC | 48 | S | 1519 |
| 1016725 | 13962 | 13977 | N/A | N/A | GGAATGTTAAACTGGG | 9 | S | 1520 |
| 1016744 | 8569 | 8584 | N/A | N/A | TGAATGGTGTGCCAGG | 26 | S | 1521 |
| 1016756 | 8820 | 8835 | N/A | N/A | GGCGATCTTCTTGCAC | 44 | S | 1522 |
| 1016768 | 9007 | 9022 | N/A | N/A | GACAGCAACAAAGCCC | 12 | S | 1523 |
| 1016780 | 9155 | 9170 | N/A | N/A | CCAATTCCTAGCTGGC | 73 | S | 1524 |
| 1016792 | 9464 | 9479 | N/A | N/A | GATGAGTTGGAGGTCA | 20 | S | 1525 |
| 1016804 | 9580 | 9595 | N/A | N/A | GAACTTACAAGATGCC | 15 | S | 1526 |
| 1016816 | 9703 | 9718 | N/A | N/A | GTTCTAGGGACTCAGG | 11 | S | 1527 |
| 1016828 | 9940 | 9955 | N/A | N/A | GAAACTGGAGCTCCCT | 25 | S | 1528 |
| 1016840 | 10054 | 10069 | N/A | N/A | TTGCATCTTAGCTAGC | 57 | S | 1529 |
| 1016852 | 10283 | 10298 | N/A | N/A | CCCGATATCCATGGCT | 19 | S | 1530 |
| 1016864 | 10374 | 10389 | N/A | N/A | ATCCATCATGGTTAGT | 13 | S | 1531 |
| 1016876 | 10449 | 10464 | N/A | N/A | CTATTACATCGCCCGT | 35 | S | 1532 |
| 1016888 | 10559 | 10574 | N/A | N/A | GGAGTGACCAGTCACA | 15 | S | 1533 |
| 1016900 | 10830 | 10845 | N/A | N/A | GTACACACACAGGTCC | 16 | S | 1534 |
| 1016912 | 11139 | 11154 | N/A | N/A | TGAGAAACTAGGAGGC | 12 | S | 1535 |
| 1016924 | 11712 | 11727 | N/A | N/A | CTTGCTCCCGACAAGC | 71 | S | 1536 |
| 1016936 | 12330 | 12345 | N/A | N/A | GGTAGGCACTCACCCC | 83 | S | 1537 |
| 1016987 | 16396 | 16411 | N/A | N/A | GGCACTGGCTCACCGT | 82 | S | 1538 |
| 1016993 | 16554 | 16569 | N/A | N/A | AATGCTTAGCCCCTCC | 36 | S | 1539 |
| 1016998 | 17245 | 17260 | N/A | N/A | CCAACTTTATGGAGGG | 70 | S | 1540 |
| 1017005 | 17322 | 17337 | N/A | N/A | AGTAGTAGATGGGCAC | 1 | S | 1541 |
| 1017014 | 17425 | 17440 | N/A | N/A | TGCACTCCATTGTCTC | 28 | S | 1542 |
| 1017019 | 17641 | 17656 | N/A | N/A | ATCTGGTCCGTGCTGG | 52 | S | 1543 |
| 1017033 | 19423 | 19438 | N/A | N/A | GCTTTTTGATCTTGGC | 26 | S | 1544 |
| 1017043 | 19804 | 19819 | N/A | N/A | AATGGTTACAAGATTC | 6 | S | 1545 |
| 1017055 | 20811 | 20826 | N/A | N/A | GGAATCTATCATGGCT | 4 | S | 1546 |
| 1017060 | 20945 | 20960 | N/A | N/A | GCCATCTGGTGAGCTC | 60 | S | 1547 |
| 1017067 | 21098 | 21113 | N/A | N/A | ATGTGTAATGTTGTCC | 1 | S | 1333 |
| 1017071 | 21277 | 21292 | N/A | N/A | GGTATGAAGTGGCTGT | 31 | S | 1548 |
| 1017078 | 21493 | 21508 | N/A | N/A | ATGTTCTAGGGTCAGC | 7 | S | 1549 |
| 1017107 | 22386 | 22401 | N/A | N/A | TCCTCTAAATCTACAC | 77 | S | 1550 |
| 1017117 | 22507 | 22522 | N/A | N/A | AGGAATCCTTGTTTAT | 88 | S | 1551 |
| 1017136 | 23743 | 23758 | N/A | N/A | AGCTGTTGTTAGTCCA | 15 | S | 1552 |
| 1017146 | 24999 | 25014 | N/A | N/A | GGTGCCTCCGGGTGGC | 90 | S | 1553 |
| 1017158 | 25250 | 25265 | N/A | N/A | CACATTCCCCATCTCG | 106 | S | 1554 |
| 1017170 | 25503 | 25518 | N/A | N/A | TCCACTCGGGTCTCTG | 98 | S | 1555 |
| 1017182 | 25673 | 25688 | N/A | N/A | GTAGCCATGTTTTGCA | 123 | S | 1556 |
| 1017194 | 26224 | 26239 | N/A | N/A | GATACTTGTCCACTGC | 84 | S | 1557 |
| 1017206 | 26283 | 26298 | N/A | N/A | GTAGACTGTCTCCTCG | 107 | S | 1558 |
| 1017218 | 26633 | 26648 | N/A | N/A | CCACACTTAGTCCCCG | 128 | S | 1559 |
| 1017230 | 26938 | 26953 | N/A | N/A | GGGAAACCGGAGCTGG | 96 | S | 1560 |
| 569401 | 15164 | 15179 | 540 | 555 | CGGAAGCACGACACCT | 60 | T | 1561 |
| 569664 | 19498 | 19513 | 1359 | 1374 | TCCCGAATGTCCGACA | 18 | T | 1337 |
| 570586 | 15647 | 15662 | N/A | N/A | TGTCACTGGGCAGATT | 33 | T | 1562 |
| 570780 | 20810 | 20825 | N/A | N/A | GAATCTATCATGGCTC | 14 | T | 1564 |
| 570958 | 23316 | 23331 | N/A | N/A | ACGGAGGGAGATCTGG | 46 | T | 1565 |
| 570968 | 23354 | 23369 | N/A | N/A | ATCTAGGGAGATCCCG | 49 | T | 1566 |
| 1002423 | 15004 | 15019 | 459 | 474 | ATCTTCACTACCGCTA | 15 | T | 1567 |
| 1002549 | 24468 | 24483 | 2526 | 2541 | TCATGCACAAGAAAGC | 43 | T | 1568 |
| 1002579 | 13963 | 13978 | N/A | N/A | AGGAATGTTAAACTGG | 11 | T | 1569 |
| 1002630 | 14498 | 14513 | N/A | N/A | CCACATAAACACCGTG | 52 | T | 1570 |
| 1002683 | 12907 | 12922 | N/A | N/A | CTGGATCGCAGAGGAG | 18 | T | 1571 |
| 1002718 | 13268 | 13283 | N/A | N/A | AGACATATGAGGGCCA | 26 | T | 1572 |
| 1002730 | 13337 | 13352 | N/A | N/A | ACAGGAAACAAGTGTC | 53 | T | 1573 |
| 1002753 | 13626 | 13641 | N/A | N/A | CGCGAGATGCAGCTCG | 78 | T | 1574 |
| 1002781 | 14832 | 14847 | N/A | N/A | GTTCTAAGGCTCGGTC | 34 | T | 1575 |
| 1002833 | 15577 | 15592 | N/A | N/A | AGAATAGGTCCCAGAC | 23 | T | 1576 |
| 1002900 | 16557 | 16572 | N/A | N/A | CCAAATGCTTAGCCCC | 64 | T | 1577 |
| 1002923 | 17252 | 17267 | N/A | N/A | CCTTACTCCAACTTTA | 34 | T | 1578 |
| 1002966 | 17644 | 17659 | N/A | N/A | CTCATCTGGTCCGTGC | 49 | T | 1579 |
| 1003016 | 19724 | 19739 | N/A | N/A | CTGAAAATTTAAGGTC | 17 | T | 1580 |
| 1003028 | 19807 | 19822 | N/A | N/A | GTTAATGGTTACAAGA | 3 | T | 1581 |
| 1003059 | 20946 | 20961 | N/A | N/A | TGCCATCTGGTGAGCT | 112 | T | 1582 |
| 1003081 | 21121 | 21136 | N/A | N/A | GTTCATTAATGATAAG | 6 | T | 1583 |
| 1003110 | 21278 | 21293 | N/A | N/A | GGGTATGAAGTGGCTG | 84 | T | 1584 |
| 1003164 | 22097 | 22112 | N/A | N/A | CTCAAAGATCGACTTC | 10 | T | 1585 |
| 1003209 | 22509 | 22524 | N/A | N/A | CTAGGAATCCTTGTTT | 73 | T | 1586 |
| 1003241 | 23017 | 23032 | N/A | N/A | TCTATACACGCCCCGC | 36 | T | 1587 |
| 1003257 | 23300 | 23315 | N/A | N/A | GGCTGAATAAAGGGCT | 97 | T | 1588 |
| 1016702 | 16763 | 16778 | 992 | 1007 | CGAATACACCCAGCGC | 62 | T | 1589 |
| 1016704 | 19494 | 19509 | 1355 | 1370 | GAATGTCCGACAGTGT | 7 | T | 1590 |
| 1016713 | 24161 | 24176 | 2219 | 2234 | CGGATCACAGGACTGG | 29 | T | 1591 |
| 1016731 | 14087 | 14102 | N/A | N/A | CACATATCCCAGACTC | 8 | T | 1592 |
| 1016737 | 14274 | 14289 | N/A | N/A | CAGTTCAGGTGCAGCC | 15 | T | 1593 |
| 1016745 | 8570 | 8585 | N/A | N/A | CTGAATGGTGTGCCAG | 73 | T | 1594 |
| 1016757 | 8848 | 8863 | N/A | N/A | GGAACAGGAGGACTGT | 60 | T | 1595 |
| 1016769 | 9052 | 9067 | N/A | N/A | TAAGATTTCCCTGGCT | 35 | T | 1596 |
| 1016781 | 9168 | 9183 | N/A | N/A | AGCCTTGGATGCCCCA | 53 | T | 1597 |
| 1016793 | 9466 | 9481 | N/A | N/A | GTGATGAGTTGGAGGT | 19 | T | 1598 |
| 1016805 | 9581 | 9596 | N/A | N/A | CGAACTTACAAGATGC | 11 | T | 1599 |
| 1016817 | 9704 | 9719 | N/A | N/A | AGTTCTAGGGACTCAG | 20 | T | 1600 |
| 1016829 | 9941 | 9956 | N/A | N/A | GGAAACTGGAGCTCCC | 81 | T | 1601 |
| 1016841 | 10059 | 10074 | N/A | N/A | GGAACTTGCATCTTAG | 13 | T | 1602 |
| 1016853 | 10321 | 10336 | N/A | N/A | GTTATGGCTAGGAGGC | 11 | T | 1603 |
| 1016865 | 10376 | 10391 | N/A | N/A | CCATCCATCATGGTTA | 27 | T | 1604 |
| 1016877 | 10450 | 10465 | N/A | N/A | GCTATTACATCGCCCG | 54 | T | 1605 |
| 1016889 | 10593 | 10608 | N/A | N/A | GGCTCTTGTGGCAGGG | 43 | T | 1606 |
| 1016901 | 10831 | 10846 | N/A | N/A | AGTACACACACAGGTC | 23 | T | 1607 |
| 1016913 | 11214 | 11229 | N/A | N/A | GGTGGACGGTTCTCCA | 40 | T | 1608 |
| 1016925 | 11748 | 11763 | N/A | N/A | CCAATTGAGGCTGAGT | 17 | T | 1609 |
| 1016937 | 12332 | 12347 | N/A | N/A | AGGGTAGGCACTCACC | 93 | T | 1610 |
| 1016966 | 15385 | 15400 | N/A | N/A | CCCTCTTACCGCACAC | 23 | T | 1611 |
| 1016973 | 15748 | 15763 | N/A | N/A | CAGAGATAACCATAGA | 19 | T | 1612 |
| 1016982 | 16196 | 16211 | N/A | N/A | CACTTCCTCGGGTTCC | 64 | T | 1613 |
| 1016988 | 16455 | 16470 | N/A | N/A | CGCTCCCACACTCTGT | 81 | T | 1614 |
| 1017006 | 17324 | 17339 | N/A | N/A | TCAGTAGTAGATGGGC | 1 | T | 1615 |
| 1017015 | 17489 | 17504 | N/A | N/A | ATAGCTCCCCACTCCA | 8 | T | 1616 |
| 1017027 | 18456 | 18471 | N/A | N/A | TCTCATGACCCACCGG | 54 | T | 1617 |
| 1017034 | 19480 | 19495 | N/A | N/A | GTCTCCTGCGCAAGAC | 95 | T | 1618 |
| 1017051 | 19926 | 19941 | N/A | N/A | CTTATTGTTATATGGC | 2 | T | 1619 |
| 1017056 | 20850 | 20865 | N/A | N/A | ACTTTTGTTGAGACCA | 3 | T | 1620 |
| 1017068 | 21100 | 21115 | N/A | N/A | GTATGTGTAATGTTGT | 0.4 | T | 1621 |
| 1017079 | 21494 | 21509 | N/A | N/A | CATGTTCTAGGGTCAG | 16 | T | 1622 |
| 1017087 | 21882 | 21897 | N/A | N/A | TCCACCAACTTACTGT | 109 | T | 1623 |
| 1017095 | 22042 | 22057 | N/A | N/A | CACTGTAACTACAGAG | 92 | T | 1624 |
| 1017104 | 22301 | 22316 | N/A | N/A | CTCCCTAATGCCCTCA | 56 | T | 1625 |
| 1017108 | 22387 | 22402 | N/A | N/A | GTCCTCTAAATCTACA | 70 | T | 1626 |
| 1017137 | 23744 | 23759 | N/A | N/A | CAGCTGTTGTTAGTCC | 76 | T | 1627 |
| 1017147 | 25139 | 25154 | N/A | N/A | TTTTCTCGAGCTTGCG | 94 | T | 1628 |
| 1017159 | 25284 | 25299 | N/A | N/A | CACGCCTCCGTCTCCA | 74 | T | 1629 |
| 1017171 | 25504 | 25519 | N/A | N/A | CTCCACTCGGGTCTCT | 111 | T | 1630 |
| 1017183 | 25674 | 25689 | N/A | N/A | GGTAGCCATGTTTTGC | 93 | T | 1631 |
| 1017195 | 26226 | 26241 | N/A | N/A | TCGATACTTGTCCACT | 86 | T | 1632 |
| 1017207 | 26285 | 26300 | N/A | N/A | CAGTAGACTGTCTCCT | 90 | T | 1633 |
| 1017219 | 26644 | 26659 | N/A | N/A | GCCTGTCCCGTCCACA | 82 | T | 1634 |
| 1017231 | 26945 | 26960 | N/A | N/A | GGTTACAGGGAAACCG | 102 | T | 1635 |
| 569393 | N/A | N/A | 532 | 547 | CGACACCTCGCCCCTC | 23 | U | 1636 |
| 569664 | 19498 | 19513 | 1359 | 1374 | TCCCGAATGTCCGACA | 17 | U | 1337 |
| 570888 | 22039 | 22054 | N/A | N/A | TGTAACTACAGAGACC | 25 | U | 1640 |
| 570970 | 23358 | 23373 | N/A | N/A | GGTTATCTAGGGAGAT | 20 | U | 1641 |
| 1002448 | 16764 | 16779 | 993 | 1008 | GCGAATACACCCAGCG | 65 | U | 1642 |
| 1002477 | 19496 | 19511 | 1357 | 1372 | CCGAATGTCCGACAGT | 3 | U | 1643 |
| 1002551 | 24502 | 24517 | 2560 | 2575 | AGAGATCGCGCCAGAC | 25 | U | 1644 |
| 1002582 | 13992 | 14007 | N/A | N/A | GGCCTCTCAGAAGTCA | 65 | U | 1645 |
| 1002664 | 12334 | 12349 | N/A | N/A | CGAGGGTAGGCACTCA | 99 | U | 1646 |
| 1002684 | 12909 | 12924 | N/A | N/A | TCCTGGATCGCAGAGG | 70 | U | 1647 |
| 1002719 | 13270 | 13285 | N/A | N/A | CCAGACATATGAGGGC | 36 | U | 1648 |
| 1002735 | 13461 | 13476 | N/A | N/A | GGCTGAATGGCCTGGC | 88 | U | 1649 |
| 1002756 | 13688 | 13703 | N/A | N/A | CCCTTTTAAGGCAGCA | 15 | U | 1650 |
| 1002834 | 15579 | 15594 | N/A | N/A | AGAGAATAGGTCCCAG | 8 | U | 1651 |
| 1002939 | 17493 | 17508 | N/A | N/A | TCAGATAGCTCCCCAC | 22 | U | 1652 |
| 1002993 | 19489 | 19504 | N/A | N/A | TCCGACAGTGTCTCCT | 14 | U | 1653 |
| 1003017 | 19734 | 19749 | N/A | N/A | GTACATAGATCTGAAA | 30 | U | 1654 |
| 1003029 | 19808 | 19823 | N/A | N/A | AGTTAATGGTTACAAG | 4 | U | 1655 |
| 1003034 | 19927 | 19942 | N/A | N/A | CCTTATTGTTATATGG | 39 | U | 1656 |
| 1003067 | 21101 | 21116 | N/A | N/A | AGTATGTGTAATGTTG | 1 | U | 1657 |
| 1003113 | 21332 | 21347 | N/A | N/A | AACTTGAGGTCAGCAC | 35 | U | 1658 |
| 1003242 | 23018 | 23033 | N/A | N/A | GTCTATACACGCCCCG | 26 | U | 1659 |
| 1016698 | 15007 | 15022 | 462 | 477 | TTCATCTTCACTACCG | 22 | U | 1660 |
| 1016714 | 24169 | 24184 | 2227 | 2242 | GGCGGGCCCGGATCAC | 103 | U | 1661 |
| 1016732 | 14089 | 14104 | N/A | N/A | GTCACATATCCCAGAC | 56 | U | 1662 |
| 1016738 | 14366 | 14381 | N/A | N/A | GTAACGGAGTCTGCAG | 44 | U | 1663 |
| 1016741 | N/A | N/A | 1342 | 1357 | TGTCTCCCCGCCCCCG | 46 | U | 1664 |
| 1016746 | 8571 | 8586 | N/A | N/A | GCTGAATGGTGTGCCA | 26 | U | 1665 |
| 1016758 | 8865 | 8880 | N/A | N/A | GATGATGCAGTCCTCC | 56 | U | 1666 |
| 1016770 | 9053 | 9068 | N/A | N/A | CTAAGATTTCCCTGGC | 26 | U | 1667 |
| 1016782 | 9228 | 9243 | N/A | N/A | TCGAGTGACAGGCAGT | 21 | U | 1668 |
| 1016794 | 9467 | 9482 | N/A | N/A | GGTGATGAGTTGGAGG | 17 | U | 1669 |
| 1016806 | 9595 | 9610 | N/A | N/A | CCCAAGACGAGCTCCG | 17 | U | 1670 |
| 1016818 | 9705 | 9720 | N/A | N/A | AAGTTCTAGGGACTCA | 51 | U | 1671 |
| 1016830 | 9991 | 10006 | N/A | N/A | GCGGATCCTGAGTGAG | 34 | U | 1672 |
| 1016842 | 10060 | 10075 | N/A | N/A | GGGAACTTGCATCTTA | 12 | U | 1673 |
| 1016854 | 10322 | 10337 | N/A | N/A | GGTTATGGCTAGGAGG | 11 | U | 1674 |
| 1016866 | 10378 | 10393 | N/A | N/A | GTCCATCCATCATGGT | 77 | U | 1675 |
| 1016878 | 10451 | 10466 | N/A | N/A | GGCTATTACATCGCCC | 110 | U | 1676 |
| 1016890 | 10683 | 10698 | N/A | N/A | AATAGCAAGGGCAGCT | 18 | U | 1677 |
| 1016902 | 10832 | 10847 | N/A | N/A | GAGTACACACACAGGT | 13 | U | 1678 |
| 1016914 | 11215 | 11230 | N/A | N/A | GGGTGGACGGTTCTCC | 12 | U | 1679 |
| 1016926 | 11749 | 11764 | N/A | N/A | GCCAATTGAGGCTGAG | 33 | U | 1680 |
| 1016958 | 14833 | 14848 | N/A | N/A | GGTTCTAAGGCTCGGT | 20 | U | 1681 |
| 1016967 | 15401 | 15416 | N/A | N/A | TCATCCACCTGACACA | 46 | U | 1682 |
| 1016974 | 15820 | 15835 | N/A | N/A | TCCATCACGGATGGCT | 82 | U | 1683 |
| 1016989 | 16463 | 16478 | N/A | N/A | TCCAGTCCCGCTCCCA | 56 | U | 1684 |
| 1016994 | 16858 | 16873 | N/A | N/A | TGCGGCCGTGCTCACC | 111 | U | 1685 |
| 1016999 | 17255 | 17270 | N/A | N/A | TGTCCTTACTCCAACT | 53 | U | 1686 |
| 1017007 | 17329 | 17344 | N/A | N/A | GGTCCTCAGTAGTAGA | 54 | U | 1687 |
| 1017020 | 17654 | 17669 | N/A | N/A | GGGACCAGAGCTCATC | 88 | U | 1688 |
| 1017028 | 18899 | 18914 | N/A | N/A | TCAATCAAGCGATTCT | 60 | U | 1689 |
| 1017057 | 20851 | 20866 | N/A | N/A | TACTTTTGTTGAGACC | 4 | U | 1690 |
| 1017061 | 20951 | 20966 | N/A | N/A | ACCACTGCCATCTGGT | 89 | U | 1691 |
| 1017069 | 21138 | 21153 | N/A | N/A | TTCAGAATCAAGCTGG | 27 | U | 1692 |
| 1017080 | 21495 | 21510 | N/A | N/A | CCATGTTCTAGGGTCA | 10 | U | 1693 |
| 1017088 | 21904 | 21919 | N/A | N/A | GTCCCTGACGGACCCC | 108 | U | 1694 |
| 1017096 | 22052 | 22067 | N/A | N/A | CTTAATGCCCCACTGT | 31 | U | 1695 |
| 1017102 | 22148 | 22163 | N/A | N/A | CCACTTGGCACCTTTC | 6 | U | 1696 |
| 1017105 | 22335 | 22350 | N/A | N/A | ACCTATGTCCCTCTGC | 16 | U | 1697 |
| 1017109 | 22395 | 22410 | N/A | N/A | TGAGTCTGGTCCTCTA | 84 | U | 1698 |
| 1017118 | 22519 | 22534 | N/A | N/A | TGGTTCCAGGCTAGGA | 59 | U | 1699 |
| 1017129 | 23335 | 23350 | N/A | N/A | GGAATCTGGTGAGGCC | 60 | U | 1700 |
| 1017138 | 23745 | 23760 | N/A | N/A | ACAGCTGTTGTTAGTC | 77 | U | 1701 |
| 1017148 | 25141 | 25156 | N/A | N/A | ACTTTTCTCGAGCTTG | 87 | U | 1702 |
| 1017160 | 25290 | 25305 | N/A | N/A | CTTCTGCACGCCTCCG | 90 | U | 1703 |
| 1017172 | 25516 | 25531 | N/A | N/A | CGAGATCCAGCTCTCC | 115 | U | 1704 |
| 1017184 | 25677 | 25692 | N/A | N/A | CAAGGTAGCCATGTTT | 94 | U | 1705 |
| 1017196 | 26227 | 26242 | N/A | N/A | GTCGATACTTGTCCAC | 77 | U | 1706 |
| 1017208 | 26287 | 26302 | N/A | N/A | AGCAGTAGACTGTCTC | 88 | U | 1707 |
| 1017220 | 26647 | 26662 | N/A | N/A | GGTGCCTGTCCCGTCC | 99 | U | 1708 |
| 569664 | 19498 | 19513 | 1359 | 1374 | TCCCGAATGTCCGACA | 22 | V | 1337 |
| 569984 | 23939 | 23954 | 1997 | 2012 | CGGCGGCACGAGACAG | 13 | V | 1710 |
| 570050 | 24773 | 24788 | 2831 | 2846 | TTTATTCGCGAGGGTC | 13 | V | 1711 |
| 570771 | 19698 | 19713 | N/A | N/A | GGCCTCTTAGGAGTCT | 102 | V | 1713 |
| 570903 | 22284 | 22299 | N/A | N/A | GACAAAAGGCCTTGCT | 33 | V | 1714 |
| 1002425 | 15029 | 15044 | 484 | 499 | GGCATACACCTGGCCC | 81 | V | 1715 |
| 1002449 | 16765 | 16780 | 994 | 1009 | GGCGAATACACCCAGC | 46 | V | 1716 |
| 1002525 | 24235 | 24250 | 2293 | 2308 | TACAAGGACCCTTCGA | 46 | V | 1717 |
| 1002591 | 14090 | 14105 | N/A | N/A | GGTCACATATCCCAGA | 13 | V | 1718 |
| 1002606 | 14367 | 14382 | N/A | N/A | GGTAACGGAGTCTGCA | 23 | V | 1719 |
| 1002636 | N/A | N/A | 1350 | 1365 | TCCGACAGTGTCTCCC | 12 | V | 1720 |
| 1002721 | 13292 | 13307 | N/A | N/A | ACCAAACACCAGTCAC | 28 | V | 1721 |
| 1002738 | 13524 | 13539 | N/A | N/A | TACTAAAGGGAGGCCA | 25 | V | 1722 |
| 1002835 | 15581 | 15596 | N/A | N/A | CCAGAGAATAGGTCCC | 11 | V | 1723 |
| 1002890 | 16468 | 16483 | N/A | N/A | CCAAATCCAGTCCCGC | 43 | V | 1724 |
| 1002907 | 16901 | 16916 | N/A | N/A | GGCGATAGCCTGGGAG | 104 | V | 1725 |
| 1002940 | 17495 | 17510 | N/A | N/A | GCTCAGATAGCTCCCC | 37 | V | 1726 |
| 1002979 | 18900 | 18915 | N/A | N/A | TTCAATCAAGCGATTC | 44 | V | 1727 |
| 1002995 | 19568 | 19583 | N/A | N/A | TGCTTACCTGAGGGCC | 97 | V | 1728 |
| 1003019 | 19736 | 19751 | N/A | N/A | AAGTACATAGATCTGA | 8 | V | 1729 |
| 1003030 | 19814 | 19829 | N/A | N/A | CGTAAAAGTTAATGGT | 7 | V | 1730 |
| 1003036 | 19929 | 19944 | N/A | N/A | GGCCTTATTGTTATAT | 81 | V | 1731 |
| 1003047 | 20883 | 20898 | N/A | N/A | TGTGATAAGCAATGCA | 2 | V | 1732 |
| 1003068 | 21105 | 21120 | N/A | N/A | GTATAGTATGTGTAAT | 9 | V | 1733 |
| 1003089 | 21174 | 21189 | N/A | N/A | GTTCAATCCTGACCCA | 30 | V | 1734 |
| 1003114 | 21334 | 21349 | N/A | N/A | GCAACTTGAGGTCAGC | 55 | V | 1735 |
| 1003142 | 21911 | 21926 | N/A | N/A | CCCAATTGTCCCTGAC | 39 | V | 1736 |
| 1003158 | 22054 | 22069 | N/A | N/A | TACTTAATGCCCCACT | 42 | V | 1737 |
| 1003170 | 22201 | 22216 | N/A | N/A | CAAATCAGGATTCCCA | 9 | V | 1738 |
| 1003202 | 22441 | 22456 | N/A | N/A | AATGATCCAAGCCCCC | 23 | V | 1739 |
| 1003243 | 23019 | 23034 | N/A | N/A | TGTCTATACACGCCCC | 34 | V | 1740 |
| 1003266 | 23355 | 23370 | N/A | N/A | TATCTAGGGAGATCCC | 66 | V | 1741 |
| 1016705 | N/A | N/A | 1537 | 1552 | AGCTGTTTCATCCTGT | 41 | V | 1742 |
| 1016720 | 24515 | 24530 | 2573 | 2588 | CGAGTAAGCAGGCAGA | 47 | V | 1743 |
| 1016726 | 13999 | 14014 | N/A | N/A | ACAATCAGGCCTCTCA | 10 | V | 1744 |
| 1016747 | 8572 | 8587 | N/A | N/A | TGCTGAATGGTGTGCC | 30 | V | 1745 |
| 1016759 | 8866 | 8881 | N/A | N/A | TGATGATGCAGTCCTC | 35 | V | 1746 |
| 1016771 | 9056 | 9071 | N/A | N/A | ACACTAAGATTTCCCT | 22 | V | 1747 |
| 1016783 | 9238 | 9253 | N/A | N/A | AGATTCTGGTTCGAGT | 16 | V | 1748 |
| 1016795 | 9478 | 9493 | N/A | N/A | CTGTTCTAAGAGGTGA | 10 | V | 1749 |
| 1016807 | 9628 | 9643 | N/A | N/A | GCAACTGAGTCCAAGC | 35 | V | 1750 |
| 1016819 | 9720 | 9735 | N/A | N/A | TCAGACTGTGCTCTCA | 16 | V | 1751 |
| 1016831 | 9998 | 10013 | N/A | N/A | GTCCTTGGCGGATCCT | 47 | V | 1752 |
| 1016843 | 10061 | 10076 | N/A | N/A | TGGGAACTTGCATCTT | 17 | V | 1753 |
| 1016855 | 10324 | 10339 | N/A | N/A | AGGGTTATGGCTAGGA | 15 | V | 1754 |
| 1016867 | 10382 | 10397 | N/A | N/A | AGCAGTCCATCCATCA | 17 | V | 1755 |
| 1016879 | 10452 | 10467 | N/A | N/A | GGGCTATTACATCGCC | 102 | V | 1756 |
| 1016891 | 10685 | 10700 | N/A | N/A | ACAATAGCAAGGGCAG | 11 | V | 1757 |
| 1016903 | 10842 | 10857 | N/A | N/A | TAGAACCACAGAGTAC | 23 | V | 1758 |
| 1016915 | 11216 | 11231 | N/A | N/A | AGGGTGGACGGTTCTC | 14 | V | 1759 |
| 1016927 | 11761 | 11776 | N/A | N/A | TGCTGAAACAGGGCCA | 35 | V | 1760 |
| 1016938 | 12465 | 12480 | N/A | N/A | CAGGTCCACACTCTGA | 72 | V | 1761 |
| 1016944 | 12921 | 12936 | N/A | N/A | CAAAGTTGTCCCTCCT | 13 | V | 1762 |
| 1016952 | 13696 | 13711 | N/A | N/A | ACATTGAGCCCTTTTA | 13 | V | 1763 |
| 1016959 | 14834 | 14849 | N/A | N/A | AGGTTCTAAGGCTCGG | 34 | V | 1764 |
| 1016968 | 15431 | 15446 | N/A | N/A | ACCATCCCCGTCTCAG | 46 | V | 1765 |
| 1016975 | 15841 | 15856 | N/A | N/A | GGTAGTCCCCTGAGGC | 88 | V | 1766 |
| 1016984 | 16257 | 16272 | N/A | N/A | TGAACCTCCCTTCTGT | 94 | V | 1767 |
| 1017000 | 17263 | 17278 | N/A | N/A | GTAGGCACTGTCCTTA | 62 | V | 1768 |
| 1017008 | 17346 | 17361 | N/A | N/A | AGTCTAGGTCACTGCT | 15 | V | 1769 |
| 1017021 | 17671 | 17686 | N/A | N/A | GCAACTGGATGAGGGC | 19 | V | 1770 |
| 1017062 | 20980 | 20995 | N/A | N/A | ACATGTGTCAGTACAC | 6 | V | 1771 |
| 1017081 | 21542 | 21557 | N/A | N/A | GGAGCTGCTCTTTCTA | 88 | V | 1772 |
| 1017106 | 22337 | 22352 | N/A | N/A | CAACCTATGTCCCTCT | 16 | V | 1773 |
| 1017119 | 22566 | 22581 | N/A | N/A | AGGCTTAAGGCTGCCT | 79 | V | 1774 |
| 1017139 | 23752 | 23767 | N/A | N/A | ACAGCCTACAGCTGTT | 40 | V | 1775 |
| 1017149 | 25142 | 25157 | N/A | N/A | AACTTTTCTCGAGCTT | 91 | V | 1776 |
| 1017161 | 25292 | 25307 | N/A | N/A | AGCTTCTGCACGCCTC | 109 | V | 1777 |
| 1017173 | 25517 | 25532 | N/A | N/A | GCGAGATCCAGCTCTC | 83 | V | 1778 |
| 1017185 | 25748 | 25763 | N/A | N/A | TCCTCTTCGGTCGCCG | 80 | V | 1779 |
| 1017197 | 26228 | 26243 | N/A | N/A | AGTCGATACTTGTCCA | 99 | V | 1780 |
| 1017209 | 26290 | 26305 | N/A | N/A | TGAAGCAGTAGACTGT | 87 | V | 1781 |
| 1017221 | 26651 | 26666 | N/A | N/A | GGCGGGTGCCTGTCCC | 83 | V | 1782 |
| 569664 | 19498 | 19513 | 1359 | 1374 | TCCCGAATGTCCGACA | 16 | W | 1337 |
| 569888 | 22917 | 22932 | 1765 | 1780 | CTCCATCCGCTCCTGC | 27 | W | 1785 |
| 570732 | 19368 | 19383 | N/A | N/A | TCTAAAGTGGCCCCTC | 8 | W | 1787 |
| 570779 | 20808 | 20823 | N/A | N/A | ATCTATCATGGCTCAC | 9 | W | 1788 |
| 1002426 | 15030 | 15045 | 485 | 500 | TGGCATACACCTGGCC | 76 | W | 1789 |
| 1002451 | 16770 | 16785 | 999 | 1014 | TCATAGGCGAATACAC | 35 | W | 1790 |
| 1002583 | 14000 | 14015 | N/A | N/A | GACAATCAGGCCTCTC | 10 | W | 1791 |
| 1002667 | 12486 | 12501 | N/A | N/A | GAGAAATGTTGCCCCA | 16 | W | 1792 |
| 1002722 | 13310 | 13325 | N/A | N/A | GTTACACACAGGCCAA | 13 | W | 1793 |
| 1002739 | 13525 | 13540 | N/A | N/A | CTACTAAAGGGAGGCC | 24 | W | 1794 |
| 1002761 | 13761 | 13776 | N/A | N/A | GGAGAGTGGGCCAGCA | 19 | W | 1795 |
| 1002803 | 15432 | 15447 | N/A | N/A | CACCATCCCCGTCTCA | 41 | W | 1796 |
| 1002878 | 16261 | 16276 | N/A | N/A | TAAATGAACCTCCCTT | 96 | W | 1797 |
| 1002891 | 16469 | 16484 | N/A | N/A | TCCAAATCCAGTCCCG | 47 | W | 1798 |
| 1002944 | 17557 | 17572 | N/A | N/A | GTGAAGAGAGACGGCC | 40 | W | 1799 |
| 1002969 | 17672 | 17687 | N/A | N/A | TGCAACTGGATGAGGG | 86 | W | 1800 |
| 1003020 | 19738 | 19753 | N/A | N/A | TGAAGTACATAGATCT | 6 | W | 1801 |
| 1003037 | 19935 | 19950 | N/A | N/A | TTAAATGGCCTTATTG | 20 | W | 1802 |
| 1003048 | 20889 | 20904 | N/A | N/A | TTTACTTGTGATAAGC | 1 | W | 1803 |
| 1003069 | 21106 | 21121 | N/A | N/A | GGTATAGTATGTGTAA | 2 | W | 1804 |
| 1003090 | 21182 | 21197 | N/A | N/A | GAGGATGGGTTCAATC | 36 | W | 1805 |
| 1003127 | 21582 | 21597 | N/A | N/A | TTAGGAAAAGCCCTGC | 73 | W | 1806 |
| 1003171 | 22203 | 22218 | N/A | N/A | AACAAATCAGGATTCC | 27 | W | 1807 |
| 1003189 | 22346 | 22361 | N/A | N/A | ACACTAACACAACCTA | 40 | W | 1808 |
| 1003244 | 23020 | 23035 | N/A | N/A | GTGTCTATACACGCCC | 87 | W | 1809 |
| 1003267 | 23357 | 23372 | N/A | N/A | GTTATCTAGGGAGATC | 36 | W | 1810 |
| 1016706 | 22726 | 22741 | 1679 | 1694 | GGTTGTCCGTGCGGAT | 17† | W | 1811 |
| 1016715 | 24316 | 24331 | 2374 | 2389 | TGTGATCCCCCCAGCA | 68 | W | 1812 |
| 1016721 | 24666 | 24681 | 2724 | 2739 | GTCCTGTAGCCTGTCA | 21 | W | 1813 |
| 1016733 | 14093 | 14108 | N/A | N/A | CATGGTCACATATCCC | 12 | W | 1814 |
| 1016739 | 14383 | 14398 | N/A | N/A | AGGATGGTTAGGGTGG | 13 | W | 1815 |
| 1016748 | 8575 | 8590 | N/A | N/A | CAATGCTGAATGGTGT | 14 | W | 1816 |
| 1016760 | 8867 | 8882 | N/A | N/A | GTGATGATGCAGTCCT | 21 | W | 1817 |
| 1016772 | 9057 | 9072 | N/A | N/A | GACACTAAGATTTCCC | 12 | W | 1818 |
| 1016784 | 9240 | 9255 | N/A | N/A | ATAGATTCTGGTTCGA | 14 | W | 1819 |
| 1016796 | 9490 | 9505 | N/A | N/A | ATCCTACAGGCTCTGT | 27 | W | 1820 |
| 1016808 | 9629 | 9644 | N/A | N/A | GGCAACTGAGTCCAAG | 62 | W | 1821 |
| 1016820 | 9725 | 9740 | N/A | N/A | GTCACTCAGACTGTGC | 44 | W | 1822 |
| 1016832 | 10010 | 10025 | N/A | N/A | GCAATAATCAAAGTCC | 12 | W | 1823 |
| 1016844 | 10173 | 10188 | N/A | N/A | GGCTTCAGCCAGTGTC | 74 | W | 1824 |
| 1016856 | 10346 | 10361 | N/A | N/A | TTGATCTGTGAGGTCA | 26 | W | 1825 |
| 1016868 | 10390 | 10405 | N/A | N/A | GGGACTGGAGCAGTCC | 81 | W | 1826 |
| 1016880 | 10492 | 10507 | N/A | N/A | TGTACTTGGCAGTGGG | 13 | W | 1827 |
| 1016892 | 10686 | 10701 | N/A | N/A | GACAATAGCAAGGGCA | 10 | W | 1828 |
| 1016904 | 10957 | 10972 | N/A | N/A | GTGACTCGCAGGTCCG | 20 | W | 1829 |
| 1016916 | 11257 | 11272 | N/A | N/A | TGCGGCTCCGAGAGCC | 81 | W | 1830 |
| 1016928 | 11774 | 11789 | N/A | N/A | AGAACCTGCCCATTGC | 12 | W | 1831 |
| 1016945 | 12967 | 12982 | N/A | N/A | GGTGGGTGCAGAACCT | 69 | W | 1832 |
| 1016960 | 15280 | 15295 | N/A | N/A | TCCCTCTTCCTAGTCA | 57 | W | 1833 |
| 1016970 | 15638 | 15653 | N/A | N/A | GCAGATTCACTCCCCC | 7 | W | 1834 |
| 1016976 | 15844 | 15859 | N/A | N/A | GTTGGTAGTCCCCTGA | 79 | W | 1835 |
| 1016995 | 16926 | 16941 | N/A | N/A | GGTGCTCCTGCTCAGA | 109 | W | 1836 |
| 1017001 | 17266 | 17281 | N/A | N/A | AAGGTAGGCACTGTCC | 12 | W | 1837 |
| 1017009 | 17349 | 17364 | N/A | N/A | GTAAGTCTAGGTCACT | 5 | W | 1838 |
| 1017029 | 19089 | 19104 | N/A | N/A | AGCTGTTGTAGTCCCA | 37 | W | 1839 |
| 1017035 | 19570 | 19585 | N/A | N/A | AGTGCTTACCTGAGGG | 105 | W | 1840 |
| 1017044 | 19829 | 19844 | N/A | N/A | TCTGTGTACTACAGAC | 41 | W | 1841 |
| 1017063 | 20981 | 20996 | N/A | N/A | AACATGTGTCAGTACA | 1 | W | 1842 |
| 1017072 | 21380 | 21395 | N/A | N/A | ACCTCCACAGATGTGG | 77 | W | 1843 |
| 1017089 | 21916 | 21931 | N/A | N/A | TCTCTCCCAATTGTCC | 55 | W | 1844 |
| 1017097 | 22056 | 22071 | N/A | N/A | CTTACTTAATGCCCCA | 13 | W | 1845 |
| 1017110 | 22443 | 22458 | N/A | N/A | GCAATGATCCAAGCCC | 60 | W | 1846 |
| 1017120 | 22743 | 22758 | N/A | N/A | CGACCTGGCGAAGTTC | 68† | W | 1847 |
| 1017140 | 23792 | 23807 | N/A | N/A | GGTGGGCCCGCACTCT | 91 | W | 1848 |
| 1017150 | 25144 | 25159 | N/A | N/A | GCAACTTTTCTCGAGC | 78 | W | 1849 |
| 1017162 | 25302 | 25317 | N/A | N/A | CCCGAGACTGAGCTTC | 94 | W | 1850 |
| 1017174 | 25518 | 25533 | N/A | N/A | GGCGAGATCCAGCTCT | 87 | W | 1851 |
| 1017186 | 25771 | 25786 | N/A | N/A | AGAGCTGGCGCGCTTC | 94 | W | 1852 |
| 1017198 | 26229 | 26244 | N/A | N/A | CAGTCGATACTTGTCC | 103 | W | 1853 |
| 1017210 | 26329 | 26344 | N/A | N/A | CGCGGTAGCAGGCCTT | 76 | W | 1854 |
| 1017222 | 26735 | 26750 | N/A | N/A | GTGATTCCCCAACACC | 89 | W | 1855 |
| 569495 | 16314 | 16329 | 796 | 811 | CAGGATGTTGTCGGGT | 10 | X | 1858 |
| 569664 | 19498 | 19513 | 1359 | 1374 | TCCCGAATGTCCGACA | 15 | X | 1337 |
| 570049 | 24772 | 24787 | 2830 | 2845 | TTATTCGCGAGGGTCG | 20 | X | 1859 |
| 570051 | 24774 | 24789 | 2832 | 2847 | TTTTATTCGCGAGGGT | 13 | X | 1860 |
| 570769 | 19694 | 19709 | N/A | N/A | TCTTAGGAGTCTTTGG | 5 | X | 1861 |
| 1002453 | 16772 | 16787 | 1001 | 1016 | TTTCATAGGCGAATAC | 51 | × | 1862 |
| 1002584 | 14001 | 14016 | N/A | N/A | TGACAATCAGGCCTCT | 17 | X | 1863 |
| 1002609 | 14386 | 14401 | N/A | N/A | TGCAGGATGGTTAGGG | 34 | X | 1864 |
| 1002668 | 12487 | 12502 | N/A | N/A | AGAGAAATGTTGCCCC | 10 | X | 1865 |
| 1002690 | 13000 | 13015 | N/A | N/A | CATAGGAACCGAGACT | 33 | X | 1866 |
| 1002723 | 13311 | 13326 | N/A | N/A | TGTTACACACAGGCCA | 16 | X | 1867 |
| 1002740 | 13526 | 13541 | N/A | N/A | TCTACTAAAGGGAGGC | 21 | X | 1868 |
| 1002804 | 15434 | 15449 | N/A | N/A | GACACCATCCCCGTCT | 91 | X | 1869 |
| 1002879 | 16262 | 16277 | N/A | N/A | ATAAATGAACCTCCCT | 11 | X | 1870 |
| 1002893 | 16506 | 16521 | N/A | N/A | GCACACTTAAGCCTGG | 18 | X | 1871 |
| 1002912 | 17192 | 17207 | N/A | N/A | GGTACCTACCCCGCCC | 86 | X | 1872 |
| 1002934 | 17350 | 17365 | N/A | N/A | TGTAAGTCTAGGTCAC | 3 | X | 1873 |
| 1002947 | 17565 | 17580 | N/A | N/A | GTTACACGGTGAAGAG | 4 | X | 1874 |
| 1002971 | 17678 | 17693 | N/A | N/A | CTAGACTGCAACTGGA | 25 | X | 1875 |
| 1003002 | 19647 | 19662 | N/A | N/A | TGCAACTCCATTGGCT | 42 | X | 1876 |
| 1003038 | 19951 | 19966 | N/A | N/A | GCCTAAATTAATACTT | 77 | X | 1877 |
| 1003049 | 20925 | 20940 | N/A | N/A | GATATCAACTTCCTTT | 11 | X | 1878 |
| 1003061 | 20982 | 20997 | N/A | N/A | AAACATGTGTCAGTAC | 3 | X | 1879 |
| 1003070 | 21107 | 21122 | N/A | N/A | AGGTATAGTATGTGTA | 1 | X | 1880 |
| 1003094 | 21189 | 21204 | N/A | N/A | TCTAATAGAGGATGGG | 35 | X | 1881 |
| 1003129 | 21602 | 21617 | N/A | N/A | GCCAATAGCCCCTCCT | 16 | × | 1882 |
| 1003159 | 22058 | 22073 | N/A | N/A | CCCTTACTTAATGCCC | 29 | X | 1883 |
| 1003172 | 22204 | 22219 | N/A | N/A | GAACAAATCAGGATTC | 76 | X | 1884 |
| 1003190 | 22347 | 22362 | N/A | N/A | AACACTAACACAACCT | 46 | X | 1885 |
| 1003217 | 22751 | 22766 | N/A | N/A | CCCGATCCCGACCTGG | 93† | X | 1886 |
| 1016699 | 15032 | 15047 | 487 | 502 | CATGGCATACACCTGG | 33 | X | 1887 |
| 1016707 | 22733 | 22748 | 1686 | 1701 | AAGTTCTGGTTGTCCG | 17† | X | 1888 |
| 1016716 | 24323 | 24338 | 2381 | 2396 | AATGGTCTGTGATCCC | 34 | X | 1889 |
| 1016722 | 24667 | 24682 | 2725 | 2740 | GGTCCTGTAGCCTGTC | 33 | X | 1890 |
| 1016734 | 14101 | 14116 | N/A | N/A | AAAGGTAGCATGGTCA | 11 | X | 1891 |
| 1016742 | 14915 | 14930 | N/A | N/A | CGCTTCTGCACCCAGC | 95 | X | 1892 |
| 1016749 | 8577 | 8592 | N/A | N/A | GCCAATGCTGAATGGT | 65 | X | 1893 |
| 1016761 | 8868 | 8883 | N/A | N/A | AGTGATGATGCAGTCC | 15 | X | 1894 |
| 1016773 | 9058 | 9073 | N/A | N/A | AGACACTAAGATTTCC | 6 | X | 1895 |
| 1016785 | 9241 | 9256 | N/A | N/A | AATAGATTCTGGTTCG | 8 | X | 1896 |
| 1016797 | 9514 | 9529 | N/A | N/A | GGCAAGCACATCCACC | 22 | X | 1897 |
| 1016809 | 9653 | 9668 | N/A | N/A | CAAGCCACGGCCTCCA | 34 | X | 1898 |
| 1016821 | 9767 | 9782 | N/A | N/A | GACTCAACCAGACTCC | 53 | X | 1899 |
| 1016833 | 10011 | 10026 | N/A | N/A | CGCAATAATCAAAGTC | 10 | X | 1900 |
| 1016845 | 10271 | 10286 | N/A | N/A | GGCTTCACACCACTGT | 15 | X | 1901 |
| 1016857 | 10347 | 10362 | N/A | N/A | GTTGATCTGTGAGGTC | 11 | X | 1902 |
| 1016869 | 10407 | 10422 | N/A | N/A | AATTTTGTGCAGGTGG | 11 | X | 1903 |
| 1016881 | 10493 | 10508 | N/A | N/A | TTGTACTTGGCAGTGG | 12 | X | 1904 |
| 1016893 | 10714 | 10729 | N/A | N/A | CCATAATTTAACACTC | 7 | X | 1905 |
| 1016905 | 10959 | 10974 | N/A | N/A | GTGTGACTCGCAGGTC | 11 | X | 1906 |
| 1016917 | 11276 | 11291 | N/A | N/A | CGCGGGAAGACACACT | 40 | X | 1907 |
| 1016929 | 11793 | 11808 | N/A | N/A | GGTGTGATGAATTTCA | 11 | X | 1908 |
| 1016953 | 13916 | 13931 | N/A | N/A | GGCTTGTGGCTCAGGG | 23 | X | 1909 |
| 1016961 | 15288 | 15303 | N/A | N/A | GGCTCTTGTCCCTCTT | 76 | X | 1910 |
| 1016971 | 15646 | 15661 | N/A | N/A | GTCACTGGGCAGATTC | 10 | X | 1911 |
| 1016977 | 15848 | 15863 | N/A | N/A | GGTGGTTGGTAGTCCC | 60 | X | 1912 |
| 1017002 | 17283 | 17298 | N/A | N/A | GTGATTCAGGACCCCA | 2 | X | 1913 |
| 1017030 | 19091 | 19106 | N/A | N/A | TCAGCTGTTGTAGTCC | 49 | X | 1914 |
| 1017039 | 19794 | 19809 | N/A | N/A | AGATTCTGGGAAGCCC | 8 | X | 1915 |
| 1017045 | 19832 | 19847 | N/A | N/A | GGCTCTGTGTACTACA | 6 | X | 1916 |
| 1017073 | 21398 | 21413 | N/A | N/A | GACTGCTTGGCTCTGG | 6 | X | 1917 |
| 1017090 | 21949 | 21964 | N/A | N/A | ACAGTGCACGCCACCC | 40 | X | 1918 |
| 1017111 | 22452 | 22467 | N/A | N/A | ATAGCTCCTGCAATGA | 52 | X | 1919 |
| 1017126 | 23021 | 23036 | N/A | N/A | GGTGTCTATACACGCC | 77 | X | 1920 |
| 1017130 | 23656 | 23671 | N/A | N/A | GAAGATCCGCCCTCCT | 74 | X | 1921 |
| 1017141 | 23795 | 23810 | N/A | N/A | ATAGGTGGGCCCGCAC | 63 | X | 1922 |
| 1017151 | 25145 | 25160 | N/A | N/A | AGCAACTTTTCTCGAG | 85 | X | 1923 |
| 1017163 | 25327 | 25342 | N/A | N/A | AGGACTAAGGGCGCGA | 84 | X | 1924 |
| 1017175 | 25523 | 25538 | N/A | N/A | GGGAAGGCGAGATCCA | 70 | X | 1925 |
| 1017187 | 25790 | 25805 | N/A | N/A | GCCGCCTGCAAAGTCT | 125 | X | 1926 |
| 1017199 | 26239 | 26254 | N/A | N/A | ACTTCTTGCGCAGTCG | 76 | X | 1927 |
| 1017211 | 26335 | 26350 | N/A | N/A | GGTTGCCGCGGTAGCA | 82 | X | 1928 |
| 1017223 | 26742 | 26757 | N/A | N/A | CCGCAGTGTGATTCCC | 109 | X | 1929 |
| 569664 | 19498 | 19513 | 1359 | 1374 | TCCCGAATGTCCGACA | 20 | Y | 1337 |
| 569983 | 23938 | 23953 | 1996 | 2011 | GGCGGCACGAGACAGA | 12 | Y | 1931 |
| 570762 | 19655 | 19670 | N/A | N/A | ACTGATCCTGCAACTC | 7 | Y | 1933 |
| 570921 | 22336 | 22351 | N/A | N/A | AACCTATGTCCCTCTG | 20 | Y | 1936 |
| 1002427 | 15035 | 15050 | 490 | 505 | CTTCATGGCATACACC | 28 | Y | 1937 |
| 1002456 | 16784 | 16799 | 1013 | 1028 | GCCCATAGAACATTTC | 56 | Y | 1938 |
| 1002536 | 24409 | 24424 | 2467 | 2482 | TGGAACACGGACGGCC | 82 | Y | 1939 |
| 1002574 | 24796 | 24811 | 2854 | 2869 | AGCTTTGGGCAGATGG | 61 | Y | 1940 |
| 1002593 | 14103 | 14118 | N/A | N/A | ACAAAGGTAGCATGGT | 16 | Y | 1941 |
| 1002643 | 14948 | 14963 | N/A | N/A | AAACAAAAGGGCTCGC | 75 | Y | 1942 |
| 1002693 | 13003 | 13018 | N/A | N/A | GCTCATAGGAACCGAG | 61 | Y | 1943 |
| 1002745 | 13553 | 13568 | N/A | N/A | GTTTACCCTGCCAACC | 52 | Y | 1944 |
| 1002792 | 15298 | 15313 | N/A | N/A | TGACAGCACGGGCTCT | 52 | Y | 1945 |
| 1002818 | 15477 | 15492 | N/A | N/A | TTTATAAGAGTCCCCC | 20 | Y | 1946 |
| 1002880 | 16263 | 16278 | N/A | N/A | GATAAATGAACCTCCC | 72 | Y | 1947 |
| 1002927 | 17284 | 17299 | N/A | N/A | AGTGATTCAGGACCCC | 6 | Y | 1948 |
| 1002948 | 17566 | 17581 | N/A | N/A | TGTTACACGGTGAAGA | 16 | Y | 1949 |
| 1002985 | 19369 | 19384 | N/A | N/A | ATCTAAAGTGGCCCCT | 16 | Y | 1950 |
| 1003050 | 20926 | 20941 | N/A | N/A | AGATATCAACTTCCTT | 4 | Y | 1951 |
| 1003071 | 21108 | 21123 | N/A | N/A | AAGGTATAGTATGTGT | 3 | Y | 1952 |
| 1003148 | 21955 | 21970 | N/A | N/A | GGCTCTACAGTGCACG | 70 | Y | 1953 |
| 1003174 | 22219 | 22234 | N/A | N/A | CAAACTACCTTTCAGG | 100 | Y | 1954 |
| 1003192 | 22353 | 22368 | N/A | N/A | TGCTCAAACACTAACA | 78 | Y | 1955 |
| 1003205 | 22462 | 22477 | N/A | N/A | TGGAATCCCCATAGCT | 144 | Y | 1956 |
| 1003247 | 23164 | 23179 | N/A | N/A | TTACATGGGAAGGTGG | 57 | Y | 1957 |
| 1003302 | 23798 | 23813 | N/A | N/A | CCCATAGGTGGGCCCG | 89 | Y | 1958 |
| 1016708 | 22734 | 22749 | 1687 | 1702 | GAAGTTCTGGTTGTCC | 5† | Y | 1959 |
| 1016727 | 14002 | 14017 | N/A | N/A | ATGACAATCAGGCCTC | 17 | Y | 1960 |
| 1016740 | 14389 | 14404 | N/A | N/A | GTGTGCAGGATGGTTA | 17 | Y | 1961 |
| 1016750 | 8606 | 8621 | N/A | N/A | CGCTCCTGCAGTGTGA | 40 | Y | 1962 |
| 1016762 | 8892 | 8907 | N/A | N/A | GCAGATGAGGCCCTCC | 32 | Y | 1963 |
| 1016774 | 9065 | 9080 | N/A | N/A | TGTACTGAGACACTAA | 35 | Y | 1964 |
| 1016786 | 9276 | 9291 | N/A | N/A | AAGTTTCACGATTTAA | 13 | Y | 1965 |
| 1016798 | 9550 | 9565 | N/A | N/A | TACTATGACCTTCCCA | 15 | Y | 1966 |
| 1016810 | 9667 | 9682 | N/A | N/A | AGGATCTGAGGAACCA | 33 | Y | 1967 |
| 1016822 | 9798 | 9813 | N/A | N/A | TGAACTACGGAGACAG | 65 | Y | 1968 |
| 1016834 | 10019 | 10034 | N/A | N/A | CACTTTCACGCAATAA | 25 | Y | 1969 |
| 1016846 | 10274 | 10289 | N/A | N/A | CATGGCTTCACACCAC | 39 | Y | 1970 |
| 1016858 | 10364 | 10379 | N/A | N/A | GTTAGTCTTGTTAATA | 13 | Y | 1971 |
| 1016870 | 10408 | 10423 | N/A | N/A | AAATTTTGTGCAGGTG | 10 | Y | 1972 |
| 1016882 | 10497 | 10512 | N/A | N/A | GTCATTGTACTTGGCA | 8 | Y | 1973 |
| 1016894 | 10748 | 10763 | N/A | N/A | GGTCCTGCGGGACAGG | 73 | Y | 1974 |
| 1016906 | 10962 | 10977 | N/A | N/A | GTTGTGTGACTCGCAG | 14 | Y | 1975 |
| 1016918 | 11305 | 11320 | N/A | N/A | ACAGAAACATTTCGGG | 14 | Y | 1976 |
| 1016930 | 11853 | 11868 | N/A | N/A | GAGAATCTCAGCTGTC | 16 | Y | 1977 |
| 1016939 | 12488 | 12503 | N/A | N/A | CAGAGAAATGTTGCCC | 12 | Y | 1978 |
| 1016947 | 13312 | 13327 | N/A | N/A | TTGTTACACACAGGCC | 30 | Y | 1979 |
| 1016954 | 13919 | 13934 | N/A | N/A | GGAGGCTTGTGGCTCA | 40 | Y | 1980 |
| 1016972 | 15660 | 15675 | N/A | N/A | AACTTTCCTGGGATGT | 23 | Y | 1981 |
| 1016978 | 15849 | 15864 | N/A | N/A | GGGTGGTTGGTAGTCC | 70 | Y | 1982 |
| 1016990 | 16525 | 16540 | N/A | N/A | AATACTCCGCCACACA | 25 | Y | 1983 |
| 1016996 | 17196 | 17211 | N/A | N/A | CACAGGTACCTACCCC | 69 | Y | 1984 |
| 1017010 | 17353 | 17368 | N/A | N/A | GACTGTAAGTCTAGGT | 48 | Y | 1985 |
| 1017022 | 17684 | 17699 | N/A | N/A | AATTCTCTAGACTGCA | 26 | Y | 1986 |
| 1017036 | 19671 | 19686 | N/A | N/A | ACAGTAAGGTTCCAAG | 2 | Y | 1987 |
| 1017040 | 19795 | 19810 | N/A | N/A | AAGATTCTGGGAAGCC | 4 | Y | 1988 |
| 1017046 | 19851 | 19866 | N/A | N/A | CCAAGTTCTGAAGTCC | 1 | Y | 1989 |
| 1017052 | 20460 | 20475 | N/A | N/A | GGGTTCTGATTCTCCT | 36 | Y | 1990 |
| 1017064 | 21003 | 21018 | N/A | N/A | AACACCTGTTCTCCAC | 30 | Y | 1991 |
| 1017070 | 21193 | 21208 | N/A | N/A | GGGTTCTAATAGAGGA | 12 | Y | 1992 |
| 1017074 | 21461 | 21476 | N/A | N/A | CATGTGACCGCTGCAG | 84 | Y | 1993 |
| 1017082 | 21654 | 21669 | N/A | N/A | CCTGATTTGAGGAAGG | 65 | Y | 1994 |
| 1017098 | 22059 | 22074 | N/A | N/A | ACCCTTACTTAATGCC | 84 | Y | 1995 |
| 1017121 | 22787 | 22802 | N/A | N/A | GTTGCCACCGGCCCGC | 137† | Y | 1996 |
| 1017131 | 23671 | 23686 | N/A | N/A | GTTGATTGGCTGCCCG | 23 | Y | 1997 |
| 1017152 | 25148 | 25163 | N/A | N/A | TGCAGCAACTTTTCTC | 111 | Y | 1998 |
| 1017164 | 25344 | 25359 | N/A | N/A | GTAACGGGCCGTCCAG | 139 | Y | 1999 |
| 1017176 | 25559 | 25574 | N/A | N/A | TCGATCTTCTTTCTGG | 128 | Y | 2000 |
| 1017188 | 26009 | 26024 | N/A | N/A | GGCAGTGCGCCCAGGA | 90 | Y | 2001 |
| 1017200 | 26242 | 26257 | N/A | N/A | GGAACTTCTTGCGCAG | 105 | Y | 2002 |
| 1017212 | 26414 | 26429 | N/A | N/A | TTGAACCAGTTGCTGA | 84 | Y | 2003 |
| 1017224 | 26761 | 26776 | N/A | N/A | TCAGTCCCTCTAGTGC | 122 | Y | 2004 |
| 569396 | N/A | N/A | 535 | 550 | GCACGACACCTCGCCC | 27 | Z | 2007 |
| 569398 | N/A | N/A | 537 | 552 | AAGCACGACACCTCGC | 40 | Z | 2008 |
| 569539 | 16778 | 16793 | 1007 | 1022 | AGAACATTTCATAGGC | 10 | Z | 2009 |
| 569664 | 19498 | 19513 | 1359 | 1374 | TCCCGAATGTCCGACA | 11 | Z | 1337 |
| 570218 | 13986 | 14001 | N/A | N/A | TCAGAAGTCAAGGTCC | 8 | Z | 2010 |
| 1002428 | 15037 | 15052 | 492 | 507 | ATCTTCATGGCATACA | 63 | Z | 2011 |
| 1002458 | 16834 | 16849 | 1063 | 1078 | GACGATCTTGCCATAG | 32 | Z | 2012 |
| 1002488 | N/A | N/A | 1696 | 1711 | TTGACTGGCGAAGTTC | 13† | Z | 2013 |
| 1002539 | 24414 | 24429 | 2472 | 2487 | GAGGATGGAACACGGA | 15 | Z | 2014 |
| 1002576 | 13956 | 13971 | N/A | N/A | TTAAACTGGGCAGCCT | 9 | Z | 2015 |
| 1002585 | 14003 | 14018 | N/A | N/A | GATGACAATCAGGCCT | 34 | Z | 2016 |
| 1002619 | 14456 | 14471 | N/A | N/A | TCCCATAGAGGTGAGA | 32 | Z | 2017 |
| 1002644 | 14949 | 14964 | N/A | N/A | AAAACAAAAGGGCTCG | 76 | Z | 2018 |
| 1002669 | 12500 | 12515 | N/A | N/A | GGTGGCATAGGACAGA | 11 | Z | 2019 |
| 1002701 | 13057 | 13072 | N/A | N/A | AGAACTAAAGGACGCA | 19 | Z | 2020 |
| 1002819 | 15478 | 15493 | N/A | N/A | CTTTATAAGAGTCCCC | 9 | Z | 2021 |
| 1002842 | 15722 | 15737 | N/A | N/A | GCCAAATCAGAGACCA | 18 | Z | 2022 |
| 1002881 | 16264 | 16279 | N/A | N/A | GGATAAATGAACCTCC | 45 | Z | 2023 |
| 1002897 | 16527 | 16542 | N/A | N/A | CTAATACTCCGCCACA | 12 | Z | 2024 |
| 1002973 | 17688 | 17703 | N/A | N/A | CTCTAATTCTCTAGAC | 84 | Z | 2025 |
| 1003007 | 19673 | 19688 | N/A | N/A | AAACAGTAAGGTTCCA | 2 | Z | 2026 |
| 1003025 | 19796 | 19811 | N/A | N/A | CAAGATTCTGGGAAGC | 5 | Z | 2027 |
| 1003051 | 20927 | 20942 | N/A | N/A | GAGATATCAACTTCCT | 2 | Z | 2028 |
| 1003064 | 21060 | 21075 | N/A | N/A | ACCAACTGTTCTCTTA | 3 | Z | 2029 |
| 1003072 | 21110 | 21125 | N/A | N/A | ATAAGGTATAGTATGT | 6 | Z | 2030 |
| 1003097 | 21205 | 21220 | N/A | N/A | GGACATGCGCCTGGGT | 8 | Z | 2031 |
| 1003175 | 22220 | 22235 | N/A | N/A | CCAAACTACCTTTCAG | 21 | Z | 2032 |
| 1003196 | 22378 | 22393 | N/A | N/A | ATCTACACAGGGACCA | 55 | Z | 2033 |
| 1003248 | 23166 | 23181 | N/A | N/A | TCTTACATGGGAAGGT | 61 | Z | 2034 |
| 1016735 | 14174 | 14189 | N/A | N/A | TGCAGGTGGTTCTTGA | 35 | Z | 2035 |
| 1016751 | 8777 | 8792 | N/A | N/A | TGGATGCGCGGGCACA | 14 | Z | 2036 |
| 1016763 | 8893 | 8908 | N/A | N/A | TGCAGATGAGGCCCTC | 100 | Z | 2037 |
| 1016775 | 9068 | 9083 | N/A | N/A | TCTTGTACTGAGACAC | 12 | Z | 2038 |
| 1016787 | 9281 | 9296 | N/A | N/A | CAAAAAAGTTTCACGA | 54 | Z | 2039 |
| 1016799 | 9551 | 9566 | N/A | N/A | CTACTATGACCTTCCC | 17 | Z | 2040 |
| 1016811 | 9682 | 9697 | N/A | N/A | ACGATTCCAAAACTGA | 10 | Z | 2041 |
| 1016823 | 9799 | 9814 | N/A | N/A | GTGAACTACGGAGACA | 81 | Z | 2042 |
| 1016835 | 10028 | 10043 | N/A | N/A | GGCAGTCAGCACTTTC | 17 | Z | 2043 |
| 1016847 | 10276 | 10291 | N/A | N/A | TCCATGGCTTCACACC | 13 | Z | 2044 |
| 1016859 | 10367 | 10382 | N/A | N/A | ATGGTTAGTCTTGTTA | 9 | Z | 2045 |
| 1016871 | 10409 | 10424 | N/A | N/A | CAAATTTTGTGCAGGT | 8 | Z | 2046 |
| 1016883 | 10498 | 10513 | N/A | N/A | GGTCATTGTACTTGGC | 9 | Z | 2047 |
| 1016895 | 10759 | 10774 | N/A | N/A | TATAAATAAGAGGTCC | 9 | Z | 2048 |
| 1016907 | 10969 | 10984 | N/A | N/A | GTCACCAGTTGTGTGA | 206 | Z | 2049 |
| 1016919 | 11306 | 11321 | N/A | N/A | AACAGAAACATTTCGG | 10 | Z | 2050 |
| 1016931 | 11854 | 11869 | N/A | N/A | GGAGAATCTCAGCTGT | 14 | Z | 2051 |
| 1016948 | 13317 | 13332 | N/A | N/A | ACAGTTTGTTACACAC | 6 | Z | 2052 |
| 1016950 | 13598 | 13613 | N/A | N/A | AGTGCTCCTCCCGGGA | 67 | Z | 2053 |
| 1016955 | 14763 | 14778 | N/A | N/A | TCAAGTCAGGCTCCCG | 47 | Z | 2054 |
| 1016962 | 15303 | 15318 | N/A | N/A | TCCAGTGACAGCACGG | 83 | Z | 2055 |
| 1016979 | 15854 | 15869 | N/A | N/A | GTCATGGGTGGTTGGT | 125 | Z | 2056 |
| 1016997 | 17221 | 17236 | N/A | N/A | GAGGTTCCCGCAGCCG | 88 | Z | 2057 |
| 1017003 | 17290 | 17305 | N/A | N/A | GGAATGAGTGATTCAG | 3 | Z | 2058 |
| 1017011 | 17355 | 17370 | N/A | N/A | TGGACTGTAAGTCTAG | 64 | Z | 2059 |
| 1017016 | 17569 | 17584 | N/A | N/A | GCTTGTTACACGGTGA | 2 | Z | 2060 |
| 1017031 | 19371 | 19386 | N/A | N/A | TTATCTAAAGTGGCCC | 55 | Z | 2061 |
| 1017047 | 19880 | 19895 | N/A | N/A | TGATTGTAAAAGTCTA | 77 | Z | 2062 |
| 1017053 | 20570 | 20585 | N/A | N/A | GCCTGTAAATTAGTAC | 56 | Z | 2063 |
| 1017075 | 21462 | 21477 | N/A | N/A | ACATGTGACCGCTGCA | 53 | Z | 2064 |
| 1017083 | 21695 | 21710 | N/A | N/A | AACTTCTGGCCTGTGG | 46 | Z | 2065 |
| 1017091 | 21956 | 21971 | N/A | N/A | GGGCTCTACAGTGCAC | 145 | Z | 2066 |
| 1017099 | 22081 | 22096 | N/A | N/A | TCAGATCCCCAGCAAC | 15 | Z | 2067 |
| 1017112 | 22468 | 22483 | N/A | N/A | CATTTCTGGAATCCCC | 36 | Z | 2068 |
| 1017122 | 22825 | 22840 | N/A | N/A | GTGAGTCCGTCCGGGC | 122† | Z | 2069 |
| 1017132 | 23672 | 23687 | N/A | N/A | TGTTGATTGGCTGCCC | 41 | Z | 2070 |
| 1017142 | 23824 | 23839 | N/A | N/A | GGCTCCTGGGACTCGC | 59 | Z | 2071 |
| 1017153 | 25159 | 25174 | N/A | N/A | GCTAGAAAGTTTGCAG | 80 | Z | 2072 |
| 1017165 | 25348 | 25363 | N/A | N/A | GAAGGTAACGGGCCGT | 76 | Z | 2073 |
| 1017177 | 25566 | 25581 | N/A | N/A | GCTCTTCTCGATCTTC | 58 | Z | 2074 |
| 1017189 | 26048 | 26063 | N/A | N/A | CGCAACACCGGGTCGC | 154 | Z | 2075 |
| 1017201 | 26246 | 26261 | N/A | N/A | AGCGGGAACTTCTTGC | 183 | Z | 2076 |
| 1017213 | 26437 | 26452 | N/A | N/A | TCCGGTCGCGCTGTCG | 94 | Z | 2077 |
| 1017225 | 26842 | 26857 | N/A | N/A | AGAGTAACGGTCAGTG | 78 | Z | 2078 |
| 569664 | 19498 | 19513 | 1359 | 1374 | TCCCGAATGTCCGACA | 15 | AA | 1337 |
| 570217 | 13985 | 14000 | N/A | N/A | CAGAAGTCAAGGTCCT | 13 | AA | 2080 |
| 570266 | 14240 | 14255 | N/A | N/A | TCTCATGTAGAATGTC | 12 | AA | 2081 |
| 570428 | 13334 | 13349 | N/A | N/A | GGAAACAAGTGTCACA | 16 | AA | 2083 |
| 570445 | 13558 | 13573 | N/A | N/A | TCTCAGTTTACCCTGC | 15 | AA | 2084 |
| 1002429 | 15224 | 15239 | 600 | 615 | GCGAAGTGCAGCTGCG | 96 | AA | 2086 |
| 1002543 | 24441 | 24456 | 2499 | 2514 | CGAACCAACGATAGGT | 31 | AA | 2087 |
| 1002577 | 13957 | 13972 | N/A | N/A | GTTAAACTGGGCAGCC | 16 | AA | 2088 |
| 1002597 | 14179 | 14194 | N/A | N/A | TCAGATGCAGGTGGTT | 18 | AA | 2089 |
| 1002620 | 14468 | 14483 | N/A | N/A | CCAAAAATGCCCTCCC | 32 | AA | 2090 |
| 1002654 | 14980 | 14995 | N/A | N/A | CGAGATAGTGAGACAG | 137 | AA | 2091 |
| 1002725 | 13321 | 13336 | N/A | N/A | ACACACAGTTTGTTAC | 15 | AA | 2092 |
| 1002749 | 13607 | 13622 | N/A | N/A | CACAAAAGGAGTGCTC | 50 | AA | 2093 |
| 1002768 | 14764 | 14779 | N/A | N/A | GTCAAGTCAGGCTCCC | 41 | AA | 2094 |
| 1002820 | 15479 | 15494 | N/A | N/A | TCTTTATAAGAGTCCC | 16 | AA | 2095 |
| 1002847 | 15740 | 15755 | N/A | N/A | ACCATAGAGATCTGCC | 11 | AA | 2096 |
| 1002860 | 15855 | 15870 | N/A | N/A | CGTCATGGGTGGTTGG | 73 | AA | 2097 |
| 1002882 | 16265 | 16280 | N/A | N/A | GGGATAAATGAACCTC | 109 | AA | 2098 |
| 1002898 | 16528 | 16543 | N/A | N/A | TCTAATACTCCGCCAC | 20 | AA | 2099 |
| 1002917 | 17241 | 17256 | N/A | N/A | CTTTATGGAGGGAGCA | 23 | AA | 2100 |
| 1002929 | 17291 | 17306 | N/A | N/A | GGGAATGAGTGATTCA | 4 | AA | 2101 |
| 1002935 | 17356 | 17371 | N/A | N/A | CTGGACTGTAAGTCTA | 102 | AA | 2102 |
| 1002953 | 17579 | 17594 | N/A | N/A | TGCCATGACAGCTTGT | 15 | AA | 2103 |
| 1002986 | 19373 | 19388 | N/A | N/A | GCTTATCTAAAGTGGC | 84 | AA | 2104 |
| 1003009 | 19675 | 19690 | N/A | N/A | CAAAACAGTAAGGTTC | 16 | AA | 2105 |
| 1003026 | 19800 | 19815 | N/A | N/A | GTTACAAGATTCTGGG | 3 | AA | 2106 |
| 1003041 | 20706 | 20721 | N/A | N/A | CACGATTTTTTCAATT | 16 | AA | 2107 |
| 1003052 | 20928 | 20943 | N/A | N/A | CGAGATATCAACTTCC | 15 | AA | 2108 |
| 1003065 | 21072 | 21087 | N/A | N/A | GCTGACTAGAAAACCA | 73 | AA | 2109 |
| 1003075 | 21113 | 21128 | N/A | N/A | ATGATAAGGTATAGTA | 3 | AA | 2110 |
| 1003098 | 21206 | 21221 | N/A | N/A | TGGACATGCGCCTGGG | 26 | AA | 2111 |
| 1003123 | 21466 | 21481 | N/A | N/A | CTTGACATGTGACCGC | 3 | AA | 2112 |
| 1003150 | 21963 | 21978 | N/A | N/A | CCCTAGAGGGCTCTAC | 100 | AA | 2113 |
| 1003177 | 22230 | 22245 | N/A | N/A | AATGATTCAGCCAAAC | 16 | AA | 2114 |
| 1003197 | 22380 | 22395 | N/A | N/A | AAATCTACACAGGGAC | 52 | AA | 2115 |
| 1003305 | 23842 | 23857 | N/A | N/A | CGGCATGGGCCTCTGA | 117 | AA | 2116 |
| 1016703 | 16977 | 16992 | 1130 | 1145 | TGAAGTCTCGAGCCTC | 43 | AA | 2117 |
| 1016709 | N/A | N/A | 1697 | 1712 | GTTGACTGGCGAAGTT | 18† | AA | 2118 |
| 1016728 | 14005 | 14020 | N/A | N/A | CAGATGACAATCAGGC | 19 | AA | 2119 |
| 1016752 | 8778 | 8793 | N/A | N/A | GTGGATGCGCGGGCAC | 67 | AA | 2120 |
| 1016776 | 9069 | 9084 | N/A | N/A | GTCTTGTACTGAGACA | 45 | AA | 2121 |
| 1016788 | 9282 | 9297 | N/A | N/A | TCAAAAAAGTTTCACG | 39 | AA | 2122 |
| 1016800 | 9552 | 9567 | N/A | N/A | CCTACTATGACCTTCC | 8 | AA | 2123 |
| 1016812 | 9683 | 9698 | N/A | N/A | TACGATTCCAAAACTG | 17 | AA | 2124 |
| 1016824 | 9818 | 9833 | N/A | N/A | GGCCTCGGTCTCCTCG | 72 | AA | 2125 |
| 1016836 | 10049 | 10064 | N/A | N/A | TCTTAGCTAGCTTCCT | 18 | AA | 2126 |
| 1016848 | 10277 | 10292 | N/A | N/A | ATCCATGGCTTCACAC | 16 | AA | 2127 |
| 1016860 | 10369 | 10384 | N/A | N/A | TCATGGTTAGTCTTGT | 15 | AA | 2128 |
| 1016872 | 10410 | 10425 | N/A | N/A | CCAAATTTTGTGCAGG | 13 | AA | 2129 |
| 1016884 | 10523 | 10538 | N/A | N/A | GTAACACTGATGTTTC | 12 | AA | 2130 |
| 1016896 | 10760 | 10775 | N/A | N/A | GTATAAATAAGAGGTC | 11 | AA | 2131 |
| 1016908 | 10977 | 10992 | N/A | N/A | TGGTGTGGGTCACCAG | 114 | AA | 2132 |
| 1016920 | 11365 | 11380 | N/A | N/A | CAAAATAGCTCCTTGG | 46 | AA | 2133 |
| 1016932 | 11859 | 11874 | N/A | N/A | TGCATGGAGAATCTCA | 17 | AA | 2134 |
| 1016940 | 12524 | 12539 | N/A | N/A | GACCTTACTCTGCCCC | 11 | AA | 2135 |
| 1016946 | 13098 | 13113 | N/A | N/A | GTTTTGTGGGACAGCT | 12 | AA | 2136 |
| 1016963 | 15336 | 15351 | N/A | N/A | CCAATCCTAGAGCTTC | 28 | AA | 2137 |
| 1017023 | 17735 | 17750 | N/A | N/A | GTATCCTGTTGCTTCC | 3 | AA | 2138 |
| 1017048 | 19881 | 19896 | N/A | N/A | CTGATTGTAAAAGTCT | 48 | AA | 2139 |
| 1017084 | 21697 | 21712 | N/A | N/A | AGAACTTCTGGCCTGT | 82 | AA | 2140 |
| 1017100 | 22087 | 22102 | N/A | N/A | GACTTCTCAGATCCCC | 4 | AA | 2141 |
| 1017113 | 22487 | 22502 | N/A | N/A | TACTCCTCCGTCCCCT | 79 | AA | 2142 |
| 1017123 | 22829 | 22844 | N/A | N/A | GACGGTGAGTCCGTCC | 76† | AA | 2143 |
| 1017127 | 23168 | 23183 | N/A | N/A | GGTCTTACATGGGAAG | 67 | AA | 2144 |
| 1017133 | 23673 | 23688 | N/A | N/A | GTGTTGATTGGCTGCC | 27 | AA | 2145 |
| 1017154 | 25160 | 25175 | N/A | N/A | GGCTAGAAAGTTTGCA | 130 | AA | 2146 |
| 1017166 | 25415 | 25430 | N/A | N/A | CGCTTTCTGCCTCCCC | 108 | AA | 2147 |
| 1017178 | 25658 | 25673 | N/A | N/A | AACTTTGGGAAGTTCC | 178 | AA | 2148 |
| 1017190 | 26162 | 26177 | N/A | N/A | CGCAGGTAGAGGTCCT | 137 | AA | 2149 |
| 1017202 | 26257 | 26272 | N/A | N/A | TGGTCTTGGGCAGCGG | 63 | AA | 2150 |
| 1017214 | 26496 | 26511 | N/A | N/A | TGGACTTGCGCCGCCC | 109 | AA | 2151 |
| 1017226 | 26843 | 26858 | N/A | N/A | AAGAGTAACGGTCAGT | 115 | AA | 2152 |
| 569664 | 19498 | 19513 | 1359 | 1374 | TCCCGAATGTCCGACA | 33 | BB | 1337 |
| 570263 | 14236 | 14251 | N/A | N/A | ATGTAGAATGTCCTGG | 22 | BB | 2154 |
| 570280 | 14320 | 14335 | N/A | N/A | GGAAATAAGACCCAGT | 9 | BB | 2155 |
| 570281 | 14323 | 14338 | N/A | N/A | GAAGGAAATAAGACCC | 17 | BB | 2156 |
| 1002435 | 15988 | 16003 | 742 | 757 | GTCTATGGCCATGACA | 64 | BB | 2159 |
| 1002462 | 16982 | 16997 | 1135 | 1150 | CTGAATGAAGTCTCGA | 19 | BB | 2160 |
| 1002546 | 24448 | 24463 | 2506 | 2521 | CACTTTGCGAACCAAC | 19 | BB | 2161 |
| 1002578 | 13958 | 13973 | N/A | N/A | TGTTAAACTGGGCAGC | 17 | BB | 2162 |
| 1002586 | 14029 | 14044 | N/A | N/A | TGCTTTAGTCCTACCC | 13 | BB | 2163 |
| 1002621 | 14469 | 14484 | N/A | N/A | GCCAAAAATGCCCTCC | 26 | BB | 2164 |
| 1002655 | 14981 | 14996 | N/A | N/A | TCGAGATAGTGAGACA | 92 | BB | 2165 |
| 1002704 | 13099 | 13114 | N/A | N/A | GGTTTTGTGGGACAGC | 18 | BB | 2166 |
| 1002750 | 13609 | 13624 | N/A | N/A | GCCACAAAAGGAGTGC | 99 | BB | 2167 |
| 1002821 | 15480 | 15495 | N/A | N/A | ATCTTTATAAGAGTCC | 26 | BB | 2168 |
| 1002848 | 15741 | 15756 | N/A | N/A | AACCATAGAGATCTGC | 12 | BB | 2169 |
| 1002862 | 16046 | 16061 | N/A | N/A | TGCTATCCCCTCGGCC | 84 | BB | 2170 |
| 1002883 | 16266 | 16281 | N/A | N/A | TGGGATAAATGAACCT | 75 | BB | 2171 |
| 1002899 | 16529 | 16544 | N/A | N/A | GTCTAATACTCCGCCA | 35 | BB | 2172 |
| 1002918 | 17242 | 17257 | N/A | N/A | ACTTTATGGAGGGAGC | 19 | BB | 2173 |
| 1002930 | 17319 | 17334 | N/A | N/A | AGTAGATGGGCACAGA | 3 | BB | 2174 |
| 1002965 | 17620 | 17635 | N/A | N/A | CAGTATTGTTCAGCAA | 6 | BB | 2175 |
| 1002988 | 19407 | 19422 | N/A | N/A | TTACATGTTCCCCCCA | 19 | BB | 2176 |
| 1003027 | 19801 | 19816 | N/A | N/A | GGTTACAAGATTCTGG | 1 | BB | 2177 |
| 1003031 | 19882 | 19897 | N/A | N/A | ACTGATTGTAAAAGTC | 97 | BB | 2178 |
| 1003043 | 20708 | 20723 | N/A | N/A | GGCACGATTTTTTCAA | 44 | BB | 2179 |
| 1003053 | 20929 | 20944 | N/A | N/A | ACGAGATATCAACTTC | 4 | BB | 2180 |
| 1003076 | 21114 | 21129 | N/A | N/A | AATGATAAGGTATAGT | 14 | BB | 2181 |
| 1003100 | 21214 | 21229 | N/A | N/A | AGCTATCCTGGACATG | 117 | BB | 2182 |
| 1003139 | 21725 | 21740 | N/A | N/A | CAGAATGCTGATTCTC | 67 | BB | 2183 |
| 1003198 | 22381 | 22396 | N/A | N/A | TAAATCTACACAGGGA | 86 | BB | 2184 |
| 1003226 | 22834 | 22849 | N/A | N/A | GTAAGGACGGTGAGTC | 99† | BB | 2185 |
| 1003249 | 23249 | 23264 | N/A | N/A | CCTATATCTGGACGGG | 65 | BB | 2186 |
| 1016710 | 24071 | 24086 | 2129 | 2144 | GGCACTCAGTCTTCCA | 83 | BB | 2187 |
| 1016736 | 14184 | 14199 | N/A | N/A | TAGATTCAGATGCAGG | 14 | BB | 2188 |
| 1016753 | 8813 | 8828 | N/A | N/A | TTCTTGCACACAAGGG | 48 | BB | 2189 |
| 1016765 | 8964 | 8979 | N/A | N/A | ACGAAAGGTCCTGCCA | 64 | BB | 2190 |
| 1016777 | 9081 | 9096 | N/A | N/A | AGGATCTGAGAGGTCT | 36 | BB | 2191 |
| 1016789 | 9303 | 9318 | N/A | N/A | CCTAGTTTTGGCCTGA | 16 | BB | 2192 |
| 1016801 | 9553 | 9568 | N/A | N/A | GCCTACTATGACCTTC | 13 | BB | 2193 |
| 1016813 | 9688 | 9703 | N/A | N/A | GACTCTACGATTCCAA | 10 | BB | 2194 |
| 1016825 | 9849 | 9864 | N/A | N/A | GTGACCTGGGCCAACT | 69 | BB | 2195 |
| 1016837 | 10050 | 10065 | N/A | N/A | ATCTTAGCTAGCTTCC | 19 | BB | 2196 |
| 1016849 | 10280 | 10295 | N/A | N/A | GATATCCATGGCTTCA | 46 | BB | 2197 |
| 1016861 | 10371 | 10386 | N/A | N/A | CATCATGGTTAGTCTT | 24 | BB | 2198 |
| 1016873 | 10411 | 10426 | N/A | N/A | CCCAAATTTTGTGCAG | 22 | BB | 2199 |
| 1016885 | 10529 | 10544 | N/A | N/A | ATGAGGGTAACACTGA | 15 | BB | 2200 |
| 1016897 | 10773 | 10788 | N/A | N/A | ACAGGGAACTTTAGTA | 33 | BB | 2201 |
| 1016909 | 10980 | 10995 | N/A | N/A | CGCTGGTGTGGGTCAC | 55 | BB | 2202 |
| 1016921 | 11403 | 11418 | N/A | N/A | AGTGGTGGAGCCAAGC | 19 | BB | 2203 |
| 1016933 | 11860 | 11875 | N/A | N/A | ATGCATGGAGAATCTC | 16 | BB | 2204 |
| 1016941 | 12580 | 12595 | N/A | N/A | AGCCTGCTGTGACTCC | 63 | BB | 2205 |
| 1016949 | 13324 | 13339 | N/A | N/A | GTCACACACAGTTTGT | 22 | BB | 2206 |
| 1016956 | 14809 | 14824 | N/A | N/A | AATTTCTAAGGCCCCG | 83 | BB | 2207 |
| 1016964 | 15354 | 15369 | N/A | N/A | CGTTTCCGGGCAGCAC | 18 | BB | 2208 |
| 1017012 | 17386 | 17401 | N/A | N/A | ACTGTCTGAAGACTGC | 70 | BB | 2209 |
| 1017024 | 17737 | 17752 | N/A | N/A | TTGTATCCTGTTGCTT | 6 | BB | 2210 |
| 1017037 | 19705 | 19720 | N/A | N/A | CAACTCTGGCCTCTTA | 27 | BB | 2211 |
| 1017065 | 21094 | 21109 | N/A | N/A | GTAATGTTGTCCAGTA | 1 | BB | 2212 |
| 1017076 | 21469 | 21484 | N/A | N/A | TCCCTTGACATGTGAC | 48 | BB | 2213 |
| 1017092 | 22001 | 22016 | N/A | N/A | GTCTCTCCGTGGTTTC | 9 | BB | 2214 |
| 1017101 | 22093 | 22108 | N/A | N/A | AAGATCGACTTCTCAG | 15 | BB | 2215 |
| 1017103 | 22232 | 22247 | N/A | N/A | GGAATGATTCAGCCAA | 16 | BB | 2216 |
| 1017114 | 22497 | 22512 | N/A | N/A | GTTTATCCCCTACTCC | 41 | BB | 2217 |
| 1017134 | 23698 | 23713 | N/A | N/A | GTCATTGGCTGCTTCC | 29 | BB | 2218 |
| 1017143 | 24814 | 24829 | N/A | N/A | GGACACTGTGGAGTCC | 93 | BB | 2219 |
| 1017155 | 25189 | 25204 | N/A | N/A | GGTCTGGCCGGGAGGA | 121 | BB | 2220 |
| 1017167 | 25438 | 25453 | N/A | N/A | TGTTTTCCCTCCGCCT | 134 | BB | 2221 |
| 1017179 | 25659 | 25674 | N/A | N/A | CAACTTTGGGAAGTTC | 116 | BB | 2222 |
| 1017191 | 26177 | 26192 | N/A | N/A | TCATGGTAGCGCGCGC | 78 | BB | 2223 |
| 1017203 | 26262 | 26277 | N/A | N/A | CCAGATGGTCTTGGGC | 77 | BB | 2224 |
| 1017215 | 26521 | 26536 | N/A | N/A | GTGGACGGGATGTCCC | 122 | BB | 2225 |
| 1017227 | 26844 | 26859 | N/A | N/A | TAAGAGTAACGGTCAG | 105 | BB | 2226 |

TABLE 4
Reduction of DMPK RNA by 3-10-3 cEt modified oligonucleotides with uniform
phosphorothioate internucleoside linkages at a dose of 2,000 nM

| | SEQ ID | SEQ ID | | | | |
|---|---|---|---|---|---|---|
| | NO: 3 | NO: 3 | | | | |
| Compound | Start | Stop | Nucleobase Sequence | DMPK | | SEQ ID |
| No. | Site | Site | (5′ to 3′) | (%UTC) | AID | NO |
| 1017240 | 18779 | 18794 | CGCATAGCCAGCCAGC | 93 | Q | 2227 |
| 1017241 | 18821 | 18836 | GAAATGGCTGTCAGCT | 105 | R | 2228 |
| 1017242 | 18825 | 18840 | CGGAGAAATGGCTGTC | 128 | T | 2229 |
| 1017232 | 18561 | 18576 | AAAGTTCCTTGGTTGG | 78 | U | 2230 |
| 1017243 | 18844 | 18859 | TCCCATCAGACTCGCT | 91 | U | 2231 |
| 1017244 | 18846 | 18861 | ATTCCCATCAGACTCG | 105 | U | 2232 |
| 1017233 | 18568 | 18583 | TGCCCTAAAAGTTCCT | 99 | V | 2233 |
| 1017245 | 18852 | 18867 | CGTGGGATTCCCATCA | 102 | V | 2234 |
| 1017234 | 18678 | 18693 | GCCTTTTGCAAGCCCT | 85 | W | 2235 |
| 1017246 | 18879 | 18894 | AGGACTTCGGCTGGAC | 106 | W | 2236 |
| 1017235 | 18679 | 18694 | GGCCTTTTGCAAGCCC | 69 | X | 2237 |
| 1017247 | 18886 | 18901 | GGTCCTCAGGACTTCG | 106 | X | 2238 |
| 1017236 | 18697 | 18712 | GACCACTCCAAACTCC | 101 | Y | 2239 |
| 1017237 | 18731 | 18746 | GTGGATCATTCCAGGG | 94 | Z | 2240 |
| 1017248 | 18942 | 18957 | GAATATGGAGCCCTGG | 122 | Z | 2241 |
| 1017238 | 18738 | 18753 | AGTTTCGGTGGATCAT | 87 | AA | 2242 |
| 1017249 | 18943 | 18958 | GGAATATGGAGCCCTG | 152 | AA | 2243 |
| 1017239 | 18739 | 18754 | AAGTTTCGGTGGATCA | 145 | BB | 2244 |
| 1017250 | 18945 | 18960 | CAGGAATATGGAGCCC | 58 | BB | 2245 |

TABLE 5
Reduction of DMPK RNA by 3-10-3 cEt modified oligonucleotides with uniform
phosphorothioate internucleoside linkages at a dose of 2,000 nM

| | SEQ ID | SEQ ID | | | | |
|---|---|---|---|---|---|---|
| | NO: 4 | NO: 4 | | | | |
| Compound | Start | Stop | Nucleobase Sequence | DMPK | | SEQ ID |
| No. | Site | Site | (5′ to 3′) | (%UTC) | AID | NO |
| 1002637 | 411 | 426 | CGCTCTAGATTCAGAT | 78 | W | 2246 |

TABLE 6
Reduction of DMPK RNA by 3-10-3 cEt modified oligonucleotides with uniform
phosphorothioate internucleoside linkages at a dose of 2,000 nM

| | SEQ ID | SEQ ID | | | | |
|---|---|---|---|---|---|---|
| | NO: 5 | NO: 5 | | | | |
| Compound | Start | Stop | Nucleobase Sequence | DMPK | | SEQ ID |
| No. | Site | Site | (5′ to 3′) | (%UTC) | AID | NO |
| 1002476 | 1335 | 1350 | TCCGACAGTGTCTCCA | 14 | W | 2247 |

### Example 3: Effect of 3-10-3 cET Modified Oligonucleotides with Uniform Phosphorothioate Internucleoside Linkages on Human DMPK In Vitro, Single Dose

Modified oligonucleotides complementary to human DMPK nucleic acid were synthesized and tested for their effect on DMPK RNA levels in vitro. The results are presented in the table below.

The modified oligonucleotides in the table below are 3-10-3 cEt modified oligonucleotides with uniform phosphorothioate internucleoside linkages. The modified oligonucleotides are 16 nucleosides in length. The sugar motif for the modified oligonucleotides is (from 5′ to 3′): kkkddddddddddkkk; wherein each ‘d’ represents a 2′-β-D-deoxyribosyl sugar moiety, and each ‘k’ represents a cEt sugar moiety. The internucleoside linkage motif for the modified oligonucleotides is (from 5′ to 3′): sssssssssssssss wherein each ‘s’ represents a phosphorothioate internucleoside linkage. All cytosine nucleobases are 5-methylcytosines.

“Start site” indicates the 5′-most nucleoside of the target sequence to which the modified oligonucleotide is complementary. “Stop site” indicates the 3′-most nucleoside of the target sequence to which the modified oligonucleotide is complementary. As shown in the tables below, the modified oligonucleotides are complementary to SEQ TD NO: 1 (described herein above) and/or SEQ ID NO: 2 (described herein above). ‘N/A’ indicates that the modified oligonucleotide is not 100% complementary to that particular target sequence.

Cultured A431 cells at a density of 10,000 cells per well were treated with 500 nM of modified oligonucleotide by free uptake as indicated in the tables below. After a treatment period of approximately 48 hours, RNA was isolated from the cells and DMPK RNA levels were measured by quantitative real-time RTPCR. Human DMPK primer probe set RTS38095 (described herein above) was used to measure DMPK RNA levels. DMPK RNA levels were normalized to total RNA content, as measured by RIBOGREEN®. Results are presented as percent of DMPK RNA, relative to the amount in untreated control cells (% UTC). ‘N.D.’ in the tables below refers to instances where the value was Not Defined.

Each separate experiment described in this example is identified by an Assay Identification letter in the table column labeled “AID”.

TABLE 7
Reduction of DMPK RNA by 3-10-3 cEt modified oligonucleotides with uniform phosphorothioate
internucleoside linkages at a dose of 500 nM

| | SEQ ID | SEQ ID | SEQ ID | SEQ ID | | | | |
|---|---|---|---|---|---|---|---|---|
| | NO: 1 | NO: 1 | NO: 2 | NO: 2 | | | | |
| Compound | Start | Stop | Start | Stop | Nucleobase Sequence | DMPK | | SEQ |
| No. | Site | Site | Site | Site | (5′ to 3′) | (%UTC) | AID | ID NO |
| 486107 | 24395 | 24410 | 2453 | 2468 | CCCGGCTTGCTGCCTT | 83 | CC | 2248 |
| 486108 | 24443 | 24458 | 2501 | 2516 | TGCGAACCAACGATAG | 26 | CC | 2249 |
| 569381 | 15023 | 15038 | 478 | 493 | CACCTGGCCCGTCTGC | 12 | CC | 2250 |
| 569394 | N/A | N/A | 533 | 548 | ACGACACCTCGCCCCT | 11 | CC | 2251 |
| 569400 | N/A | N/A | 539 | 554 | GGAAGCACGACACCTC | 32 | CC | 2252 |
| 569579 | 17021 | 17036 | 1174 | 1189 | ACCCCGGCCCAGCCGT | 90 | CC | 2253 |
| 569634 | 17136 | 17151 | 1289 | 1304 | AGTTGCATGTGTCGGT | N.D. | CC | 2254 |
| 569643 | 17154 | 17169 | 1307 | 1322 | CGTCCTCCACCAAGTC | 13 | CC | 2255 |
| 569644 | 17155 | 17170 | 1308 | 1323 | CCGTCCTCCACCAAGT | 27 | CC | 2256 |
| 569681 | 19542 | 19557 | 1403 | 1418 | AGGAGTAGCCCACAAA | 11 | CC | 2257 |
| 569684 | 19545 | 19560 | 1406 | 1421 | AGTAGGAGTAGCCCAC | 37 | CC | 2258 |
| 569800 | 21793 | 21808 | 1458 | 1473 | AGTTCCATGGGTGTGG | 27 | CC | 2259 |
| 569801 | 21794 | 21809 | 1459 | 1474 | CAGTTCCATGGGTGTG | N.D. | CC | 2260 |
| 569986 | 23941 | 23956 | 1999 | 2014 | GGCGGCGGCACGAGAC | 14 | CC | 2261 |
| 570433 | 13473 | 13488 | N/A | N/A | GGTTTTTCCAGAGGCT | N.D. | CC | 2262 |
| 570434 | 13474 | 13489 | N/A | N/A | AGGTTTTTCCAGAGGC | N.D. | CC | 2263 |
| 570435 | 13475 | 13490 | N/A | N/A | AAGGTTTTTCCAGAGG | N.D. | CC | 2264 |
| 570573 | 15621 | 15636 | N/A | N/A | GAGATGTTCTGGGAAA | N.D. | CC | 2265 |
| 570581 | 15637 | 15652 | N/A | N/A | CAGATTCACTCCCCCT | 3 | CC | 2266 |
| 570587 | 15707 | 15722 | N/A | N/A | ACCTGCGGCCCCCGCC | 47 | CC | 2267 |
| 570623 | 16301 | 16316 | N/A | N/A | GGTTTGATGTCCCTGC | 1 | CC | 2268 |
| 570724 | 17710 | 17725 | N/A | N/A | ACCTGCCACACTCTCC | 2 | CC | 2269 |
| 570725 | 17734 | 17749 | N/A | N/A | TATCCTGTTGCTTCCC | N.D. | CC | 2270 |
| 570730 | 19364 | 19379 | N/A | N/A | AAGTGGCCCCTCCAGC | N.D. | CC | 2271 |
| 570766 | 19663 | 19678 | N/A | N/A | GTTCCAAGACTGATCC | 8 | CC | 2272 |
| 570777 | 19715 | 19730 | N/A | N/A | TAAGGTCCTCCAACTC | 6 | CC | 2273 |
| 570793 | 20965 | 20980 | N/A | N/A | CGTGTGAGCCAGGGAC | N.D. | CC | 2274 |
| 570798 | 21053 | 21068 | N/A | N/A | GTTCTCTTAGACAAAG | 2 | CC | 2275 |
| 570800 | 21057 | 21072 | N/A | N/A | AACTGTTCTCTTAGAC | 8 | CC | 2276 |
| 570879 | 21984 | 21999 | N/A | N/A | GTCTGCTTCTGTTCAG | 30 | CC | 2277 |
| 570885 | 21996 | 22011 | N/A | N/A | TCCGTGGTTTCTGTCT | N.D. | CC | 2278 |
| 570886 | 22018 | 22033 | N/A | N/A | ATGTCTGAAGTAACCT | 25 | CC | 2279 |
| 570898 | 22160 | 22175 | N/A | N/A | GCTCTCGCCTGACCAC | 51 | CC | 2280 |
| 570900 | 22198 | 22213 | N/A | N/A | ATCAGGATTCCCACCT | 9 | CC | 2281 |
| 570917 | 22321 | 22336 | N/A | N/A | GCTGCTCAAAATCCCT | 49 | CC | 2282 |
| 570919 | 22332 | 22347 | N/A | N/A | TATGTCCCTCTGCTGC | 17 | CC | 2283 |
| 570925 | 22440 | 22455 | N/A | N/A | ATGATCCAAGCCCCCT | 10 | CC | 2284 |
| 570931 | 22467 | 22482 | N/A | N/A | ATTTCTGGAATCCCCA | 37 | CC | 2285 |
| 610291 | 24664 | 24679 | 2722 | 2737 | CCTGTAGCCTGTCAGC | 42 | CC | 2286 |
| 1016712 | 24121 | 24136 | 2179 | 2194 | AGCGGTTGTGAACTGG | 3 | CC | 1518 |
| 1277242 | 16713 | 16728 | 942 | 957 | CCAGGCCCACCGCCCA | 64 | CC | 2287 |
| 1277243 | 14525 | 14540 | N/A | N/A | AGGACTGTCTGCTTCC | 80 | CC | 2288 |
| 1277244 | 15508 | 15523 | N/A | N/A | CAGGTAAGAGACCCCC | 47 | CC | 2289 |
| 1277245 | 17274 | 17289 | N/A | N/A | GACCCCAGAAGGTAGG | 62 | CC | 2290 |
| 1277246 | 17469 | 17484 | N/A | N/A | GCCCTCCAGGACCTTC | 59 | CC | 2291 |
| 1277247 | 21063 | 21078 | N/A | N/A | AAAACCAACTGTTCTC | 2 | CC | 2292 |
| 1277248 | 21064 | 21079 | N/A | N/A | GAAAACCAACTGTTCT | 60 | CC | 2293 |
| 1277249 | 21162 | 21177 | N/A | N/A | CCCACCGTTCAGGCCC | 86 | CC | 2294 |
| 1277250 | 22333 | 22348 | N/A | N/A | CTATGTCCCTCTGCTG | N.D. | CC | 2295 |
| 1277255 | 24123 | 24138 | 2181 | 2196 | GGAGCGGTTGTGAACT | 10 | CC | 2296 |

### Example 4: Dose-Dependent Inhibition of Human DMPK in A431 Cells by Modified Oligonucleotides

Modified oligonucleotides selected from the examples above were tested at various doses in A431 cells. The modified oligonucleotides were tested in a series of experiments using the same culture conditions. The results for each experiment are presented in separate tables shown below. Cells were plated at a density of 10,000 cells per well and were transfected using free uptake with modified oligonucleotides at various doses, as specified in the tables below. After a treatment period of approximately 48 hours, DMPK RNA levels were measured as previously described using the human DMPK primer-probe set RTS38095 (described herein above). DMPK RNA levels were normalized to total RNA, as measured by RIBOGREEN®. Results are presented as percent DMPK RNA, relative to untreated control cells (% UTC).

The half maximal inhibitory concentration (IC₅₀) of each modified oligonucleotide was calculated using a linear regression on a log/linear plot of the data in Excel and is also presented in the tables below.

TABLE 8
Dose-dependent reduction of human DMPK RNA
in A431 cells by modified oligonucleotides

| Compound | DMPK RNA (% UTC) | | | | IC₅₀ |
|---|---|---|---|---|---|
| No. | 94 nM | 375 nM | 1500 nM | 6000 nM | (μM) |
| 1052867 | 92 | 54 | 42 | 27 | 0.96 |
| 1052881 | 118 | 88 | 50 | 21 | 1.69 |
| 1380447 | 83 | 61 | 52 | 32 | 1.39 |
| 1380564 | 70 | 43 | 26 | 36 | 0.37 |
| 1380586 | 69 | 59 | 38 | 24 | 0.6 |
| 1380634 | 72 | 64 | 59 | 49 | 5.65 |
| 1380656 | 65 | 52 | 32 | 20 | 0.37 |
| 1380659 | 37 | 15 | 4 | 2 | <0.09 |
| 1380700 | 82 | 61 | 54 | 46 | 2.77 |
| 1380725 | 94 | 66 | 36 | 16 | 0.89 |
| 1380740 | 43 | 30 | 16 | 15 | <0.09 |
| 1380782 | 81 | 48 | 33 | 23 | 0.57 |
| 1381015 | 105 | 75 | 54 | 45 | 2.89 |
| 1381203 | 70 | 55 | 25 | 20 | 0.42 |
| 1381332 | 61 | 36 | 22 | 14 | 0.17 |
| 1381407 | 87 | 81 | 49 | 42 | 2.5 |
| 1381435 | 76 | 51 | 32 | 25 | 0.54 |
| 1381513 | 56 | 23 | 10 | 4 | 0.08 |
| 1381668 | 44 | 22 | 18 | 12 | <0.09 |

TABLE 9
Dose-dependent reduction of human DMPK RNA
in A431 cells by modified oligonucleotides

| Compound | DMPK RNA (% UTC) | | | | IC₅₀ |
|---|---|---|---|---|---|
| No. | 94 nM | 375 nM | 1500 nM | 6000 nM | (μM) |
| 1380304 | 84 | 73 | 51 | 47 | 3.11 |
| 1380432 | 99 | 62 | 43 | 31 | 1.30 |
| 1380454 | 120 | 87 | 67 | 46 | 4.10 |
| 1380469 | 70 | 49 | 34 | 19 | 0.42 |
| 1380652 | 83 | 60 | 41 | 23 | 0.85 |
| 1380659 | 45 | 14 | 6 | 3 | <0.09 |
| 1380686 | 85 | 45 | 22 | 15 | 0.45 |
| 1380798 | 86 | 75 | 70 | 58 | >6 |
| 1380828 | 54 | 37 | 30 | 26 | 0.10 |
| 1380847 | 82 | 61 | 46 | 30 | 1.10 |
| 1381061 | 97 | 62 | 42 | 36 | 1.43 |
| 1381178 | 80 | 55 | 34 | 26 | 0.67 |
| 1381200 | 126 | 98 | 69 | 48 | 4.74 |
| 1381215 | 81 | 61 | 48 | 32 | 1.19 |
| 1381217 | 61 | 49 | 27 | 19 | 0.27 |
| 1381285 | 70 | 49 | 31 | 19 | 0.39 |
| 1381507 | 42 | 29 | 20 | 19 | <0.09 |
| 1381524 | 82 | 59 | 32 | 28 | 0.76 |
| 1381677 | 69 | 42 | 26 | 27 | 0.31 |

TABLE 10
Dose-dependent reduction of human DMPK RNA
in A431 cells by modified oligonucleotides

| Compound | DMPK RNA (% UTC) | | | | IC₅₀ |
|---|---|---|---|---|---|
| No. | 94 nM | 375 nM | 1500 nM | 6000 nM | (μM) |
| 1380294 | 81 | 74 | 57 | 40 | 2.69 |
| 1380322 | 62 | 57 | 36 | 28 | 0.46 |
| 1380405 | 123 | 113 | 106 | 123 | >6 |
| 1380647 | 97 | 53 | 60 | 51 | 3.73 |
| 1380693 | 57 | 38 | 22 | 12 | 0.14 |
| 1380765 | 100 | 90 | 106 | 86 | >6 |
| 1380777 | 119 | 93 | 56 | 15 | 1.72 |
| 1380785 | 96 | 85 | 61 | 62 | >6 |
| 1380857 | 92 | 75 | 57 | 40 | 2.63 |
| 1380862 | 83 | 83 | 69 | 56 | >6 |
| 1380868 | 76 | 58 | 52 | 41 | 1.72 |
| 1380870 | 78 | 56 | 49 | 25 | 0.89 |
| 1380892 | 135 | 96 | 64 | 38 | 3.17 |
| 1381156 | 91 | 65 | 56 | 45 | 2.83 |
| 1381159 | 87 | 67 | 50 | 35 | 1.63 |
| 1381170 | 70 | 54 | 45 | 29 | 0.7 |
| 1381214 | 85 | 93 | 60 | 52 | 8.16 |
| 1381657 | 73 | 61 | 42 | 34 | 0.98 |

TABLE 11
Dose-dependent reduction of human DMPK RNA
in A431 cells by modified oligonucleotides

| Compound | DMPK RNA (% UTC) | | | | IC₅₀ |
|---|---|---|---|---|---|
| No. | 94 nM | 375 nM | 1500 nM | 6000 nM | (μM) |
| 1380286 | 75 | 58 | 45 | 32 | 0.96 |
| 1380289 | 59 | 35 | 18 | 8 | 0.15 |
| 1380320 | 78 | 71 | 50 | 41 | 2.22 |
| 1380345 | 93 | 100 | 127 | 132 | >6 |
| 1380380 | 89 | 74 | 44 | 32 | 1.45 |
| 1380460 | 140 | 141 | 150 | 146 | >6 |
| 1380489 | 51 | 23 | 9 | 6 | 0.06 |
| 1380502 | 99 | 109 | 116 | 169 | >6 |
| 1380522 | 104 | 113 | 135 | 184 | >6 |
| 1380605 | 120 | 95 | 93 | 81 | >6 |
| 1380791 | 40 | 24 | 16 | 19 | <0.09 |
| 1380864 | 102 | 64 | 50 | 44 | 2.3 |
| 1380995 | 60 | 29 | 18 | 16 | 0.11 |
| 1381153 | 95 | 64 | 49 | 37 | 1.71 |
| 1381320 | 87 | 93 | 81 | 109 | >6 |
| 1381456 | 120 | 152 | 162 | 182 | >6 |
| 1381490 | 102 | 78 | 51 | 17 | 1.34 |
| 1381619 | 89 | 81 | 45 | 13 | 1.1 |

TABLE 12
Dose-dependent reduction of human DMPK RNA
in A431 cells by modified oligonucleotides

| Compound | DMPK RNA (% UTC) | | | | IC₅₀ |
|---|---|---|---|---|---|
| No. | 94 nM | 375 nM | 1500 nM | 6000 nM | (μM) |
| 1052879 | 36 | 23 | 9 | 4 | <0.09 |
| 1380330 | 100 | 113 | 99 | 134 | >10 |
| 1380358 | 108 | 81 | 52 | 39 | 2.4 |
| 1380431 | 69 | 48 | 31 | 25 | 0.39 |
| 1380598 | 72 | 54 | 44 | 27 | 0.7 |
| 1380630 | 55 | 44 | 37 | 26 | 0.19 |
| 1380679 | 61 | 34 | 22 | 12 | 0.16 |
| 1380701 | 90 | 62 | 56 | 61 | 9.48 |
| 1380721 | 69 | 41 | 28 | 28 | 0.31 |
| 1380821 | 67 | 43 | 24 | 23 | 0.28 |
| 1381021 | 65 | 49 | 33 | 37 | 0.43 |
| 1381059 | 79 | 45 | 31 | 19 | 0.48 |
| 1381083 | 60 | 70 | 46 | 37 | 1.2 |
| 1381132 | 119 | 73 | 65 | 39 | 2.86 |
| 1381176 | 78 | 57 | 41 | 26 | 0.79 |
| 1381183 | 87 | 58 | 44 | 39 | 1.4 |
| 1381554 | 85 | 64 | 46 | 47 | 2.32 |
| 1381603 | 97 | 79 | 65 | 39 | 3.27 |

TABLE 13
Dose-dependent reduction of human DMPK RNA
in A431 cells by modified oligonucleotides

| Compound | DMPK RNA (% UTC) | | | | IC₅₀ |
|---|---|---|---|---|---|
| No. | 94 nM | 375 nM | 1500 nM | 6000 nM | (μM) |
| 1380496 | 92 | 102 | 90 | 69 | >10 |
| 1380516 | 75 | 45 | 28 | 24 | 0.43 |
| 1380665 | 72 | 41 | 31 | 34 | 0.4 |
| 1380835 | 123 | 90 | 75 | 53 | 6.57 |
| 1382666 | 49 | 36 | 26 | 24 | <0.09 |
| 1382683 | 105 | 83 | 57 | 50 | 4.19 |
| 1382684 | 44 | 28 | 26 | 25 | <0.09 |
| 1382695 | 96 | 52 | 32 | 28 | 0.85 |
| 1382703 | 89 | 80 | 79 | 92 | >10 |
| 1382715 | 109 | 78 | 56 | 43 | 2.9 |
| 1382732 | 42 | 27 | 42 | 32 | N.D. |
| 1382734 | 55 | 44 | 38 | 38 | 0.15 |
| 1382741 | 60 | 34 | 23 | 26 | 0.13 |
| 1382751 | 60 | 46 | 41 | 33 | 0.32 |
| 1382753 | 50 | 34 | 28 | 20 | 0.06 |
| 1382755 | 60 | 38 | 25 | 32 | 0.15 |
| 1382760 | 68 | 45 | 37 | 34 | 0.45 |
| 1382761 | 133 | 65 | 59 | 55 | 3.74 |

TABLE 14
Dose-dependent reduction of human DMPK RNA
in A431 cells by modified oligonucleotides

| Compound | DMPK RNA (% UTC) | | | | IC₅₀ |
|---|---|---|---|---|---|
| No. | 94 nM | 375 nM | 1500 nM | 6000 nM | (μM) |
| 1380345 | 96 | 83 | 69 | 59 | >10 |
| 1380405 | 97 | 80 | 71 | 52 | >10 |
| 1380460 | 77 | 50 | 25 | 12 | 0.43 |
| 1380502 | 88 | 61 | 36 | 19 | 0.79 |
| 1380522 | 85 | 82 | 62 | 40 | 3.61 |
| 1380659 | 33 | 13 | 5 | 2 | 0.01 |
| 1380765 | 47 | 24 | 17 | 15 | <0.09 |
| 1381320 | 61 | 38 | 24 | 18 | 0.18 |
| 1381456 | 69 | 50 | 32 | 19 | 0.4 |

### Example 5: Dose-Dependent Inhibition of Human DMPK in A431 Cells by Modified Oligonucleotides

Modified oligonucleotides selected from the examples above were tested at various doses in A431 cells. The modified oligonucleotides were tested in a series of experiments using the same culture conditions. The results for each experiment are presented in separate tables shown below. Cells were plated at a density of 10,000 cells per well and were treated using free uptake with modified oligonucleotides at various doses, as specified in the tables below. After a treatment period of approximately 48 hours, DMPK RNA levels were measured as previously described using the human DMPK primer-probe set RTS38095 (described herein above). DMPK RNA levels were normalized to total RNA, as measured by RIBOGREEN®. Results are presented as percent DMPK RNA, relative to the amount in untreated control cells (% UTC). Modified oligonucleotides marked with a “†” indicate that the modified oligonucleotide is complementary to the amplicon region of the primer probe set. Additional assays may be used to measure the potency and efficacy of the modified oligonucleotides complementary to the amplicon region.

The half maximal inhibitory concentration (IC₅₀) of each modified oligonucleotide was calculated using a linear regression on a log/linear plot of the data in Excel and is also presented in the tables below.

Compound No. 486178 was previously described in WO 2015/021457 A2, WO 2017/053995 A1, and WO 2019/118916 A1 and consists of the nucleobase sequence (from 5′ to 3′): ACAATAAATACCGAGG, designated herein as SEQ ID NO: 1336. The sugar motif for Compound No. 486178 is (from 5′ to 3′): kkkddddddddddkkk; wherein each “d” represents a 2′-β-D-deoxyribosyl sugar moiety, and each “k” represents a cEt sugar moiety. The internucleoside linkage motif for Compound No. 486178 is (from 5′ to 3′): sssssssssssssss; wherein each “s” represents a phosphorothioate internucleoside linkage. Each cytosine nucleobase in Compound No. 486178 is a 5-methylcytosine.

Compound No. 598769 was previously described in WO 2015/021457 A2 and consists of the nucleobase sequence (from 5′ to 3′): TCCCGAATGTCCGACA, designated herein as SEQ ID NO: 1337. The sugar motif for Compound No. 598769 is (from 5′ to 3′): eekkddddddddkkee; wherein each “d” represents a 2′-β-D-deoxyribosyl sugar moiety, and each “k” represents a cEt sugar moiety. The internucleoside linkage motif for Compound No. 598769 is (from 5′ to 3′): sssssssssssssss; wherein each “s” represents a phosphorothioate internucleoside linkage. Each cytosine nucleobase in Compound No. 598769 is a 5-methylcytosine.

TABLE 15
Dose-dependent reduction of human DMPK RNA
in A431 cells by modified oligonucleotides

| Compound | DMPK RNA (% UTC) | | | | IC₅₀ |
|---|---|---|---|---|---|
| No. | 31 nM | 125 nM | 500 nM | 2000 nM | (μM) |
| 570785 | 24 | 6 | 2 | 3 | <0.03 |
| 570786 | 77 | 46 | 19 | 10 | 0.12 |
| 570787 | 34 | 8 | 3 | 3 | <0.03 |
| 570880 | 44 | 17 | 9 | 7 | <0.03 |
| 570901† | 38 | 15 | 6 | 5 | <0.03 |
| 1002989 | 52 | 22 | 9 | 5 | <0.03 |
| 1003066 | 12 | 5 | 4 | 3 | <0.03 |
| 1003079 | 50 | 20 | 6 | 4 | <0.03 |
| 1003178 | 40 | 18 | 7 | 5 | <0.03 |
| 1003179 | 63 | 18 | 8 | 7 | 0.03 |
| 1017017 | 46 | 13 | 6 | 4 | <0.03 |
| 1017025 | 26 | 10 | 3 | 4 | <0.03 |
| 1017026 | 44 | 14 | 7 | 3 | <0.03 |
| 1017041 | 19 | 4 | 1 | 2 | <0.03 |
| 1017042 | 16 | 4 | 2 | 2 | <0.03 |
| 1017049 | 46 | 20 | 11 | 8 | <0.03 |
| 1017066 | 11 | 4 | 3 | 4 | <0.03 |
| 1017093 | 34 | 15 | 7 | 8 | <0.03 |

TABLE 16
Dose-dependent reduction of human DMPK RNA
in A431 cells by modified oligonucleotides

| Compound | DMPK RNA (% UTC) | | | | IC₅₀ |
|---|---|---|---|---|---|
| No. | 31 nM | 125 nM | 500 nM | 2000 nM | (μM) |
| 570784 | 33 | 8 | 2 | 1 | <0.03 |
| 570795 | 49 | 19 | 7 | 2 | <0.03 |
| 570796 | 62 | 27 | 8 | 4 | 0.04 |
| 1002853 | 61 | 28 | 14 | 7 | 0.04 |
| 1003033 | 8 | 4 | 3 | 4 | <0.03 |
| 1003080 | 26 | 6 | 2 | 1 | <0.03 |
| 1003162 | 66 | 38 | 14 | 6 | 0.07 |
| 1016725 | 25 | 10 | 5 | 4 | <0.03 |
| 1017005 | 16 | 3 | 2 | 1 | <0.03 |
| 1017018 | 40 | 14 | 5 | 4 | <0.03 |
| 1017043 | 50 | 25 | 7 | 3 | <0.03 |
| 1017054 | 46 | 24 | 8 | 3 | <0.03 |
| 1017055 | 29 | 11 | 4 | 4 | <0.03 |
| 1017067 | 4 | 1 | 1 | 1 | <0.03 |
| 1017068 | 8 | 1 | 1 | 0 | <0.03 |
| 1017077 | 36 | 15 | 6 | 5 | <0.03 |
| 1017078 | 66 | 30 | 11 | 5 | 0.06 |
| 1017094 | 50 | 23 | 12 | 4 | <0.03 |

TABLE 17
Dose-dependent reduction of human DMPK RNA
in A431 cells by modified oligonucleotides

| Compound | DMPK RNA (% UTC) | | | | IC₅₀ |
|---|---|---|---|---|---|
| No. | 31 nM | 125 nM | 500 nM | 2000 nM | (μM) |
| 1002477 | 29 | 14 | 5 | 3 | <0.03 |
| 1002834 | 53 | 24 | 10 | 7 | <0.03 |
| 1003028 | 39 | 15 | 5 | 3 | <0.03 |
| 1003029 | 43 | 16 | 6 | 2 | <0.03 |
| 1003067 | 11 | 3 | 1 | 0 | <0.03 |
| 1003081 | 83 | 42 | 16 | 8 | 0.12 |
| 1016704 | 55 | 27 | 9 | 5 | <0.03 |
| 1016731 | 39 | 17 | 11 | 7 | <0.03 |
| 1016842 | 66 | 33 | 15 | 11 | 0.06 |
| 1016854 | 20 | 9 | 6 | 8 | <0.03 |
| 1017006 | 29 | 9 | 4 | 4 | <0.03 |
| 1017015 | 68 | 38 | 16 | 10 | 0.07 |
| 1017051 | 25 | 5 | 2 | 1 | <0.03 |
| 1017056 | 51 | 22 | 8 | 3 | <0.03 |
| 1017057 | 55 | 34 | 13 | 6 | 0.04 |
| 1017080 | 50 | 22 | 15 | 10 | <0.03 |
| 1017102 | 35 | 13 | 9 | 6 | <0.03 |

TABLE 18
Dose-dependent reduction of human DMPK RNA
in A431 cells by modified oligonucleotides

| Compound | DMPK RNA (% UTC) | | | | IC₅₀ |
|---|---|---|---|---|---|
| No. | 31 nM | 125 nM | 500 nM | 2000 nM | (μM) |
| 1002636 | 63 | 32 | 16 | 15 | 0.05 |
| 1002835 | 57 | 27 | 15 | 12 | 0.03 |
| 1002993 | 58 | 35 | 20 | 12 | 0.05 |
| 1003019 | 68 | 42 | 20 | 9 | 0.09 |
| 1003030 | 61 | 31 | 13 | 7 | 0.04 |
| 1003047 | 18 | 4 | 2 | 1 | <0.03 |
| 1003048 | 32 | 9 | 3 | 1 | <0.03 |
| 1003068 | 68 | 29 | 23 | 9 | 0.06 |
| 1003069 | 15 | 3 | 2 | 2 | <0.03 |
| 1003170 | 68 | 33 | 17 | 5 | 0.07 |
| 1016726 | 37 | 14 | 10 | 6 | <0.03 |
| 1016795 | 42 | 19 | 9 | 8 | <0.03 |
| 1016891 | 37 | 18 | 9 | 7 | <0.03 |
| 1016902 | 48 | 18 | 12 | 12 | <0.03 |
| 1016914 | 61 | 37 | 20 | 15 | 0.06 |
| 1017062 | 54 | 22 | 10 | 5 | <0.03 |
| 1017063 | 37 | 9 | 4 | 3 | <0.03 |

TABLE 19
Dose-dependent reduction of human DMPK RNA
in A431 cells by modified oligonucleotides

| Compound | DMPK RNA (% UTC) | | | | IC₅₀ |
|---|---|---|---|---|---|
| No. | 31 nM | 125 nM | 500 nM | 2000 nM | (μM) |
| 570732 | 91 | 50 | 20 | 13 | 0.18 |
| 570769 | 60 | 31 | 14 | 7 | 0.04 |
| 570779 | 60 | 28 | 12 | 5 | 0.04 |
| 1002583 | 26 | 12 | 8 | 7 | <0.03 |
| 1002934 | 46 | 16 | 6 | 9 | <0.03 |
| 1002947 | 52 | 20 | 7 | 3 | <0.03 |
| 1003020 | 71 | 42 | 14 | 6 | 0.09 |
| 1003061 | 65 | 23 | 7 | 2 | 0.04 |
| 1003070 | 18 | 5 | 3 | 16 | <0.03 |
| 1016773 | 39 | 15 | 9 | 13 | <0.03 |
| 1016892 | 67 | 26 | 13 | 12 | 0.05 |
| 1016970 | 55 | 26 | 12 | 8 | <0.03 |
| 1017001 | 85 | 44 | 20 | 17 | 0.15 |
| 1017002 | 59 | 30 | 8 | 2 | 0.04 |
| 1017009 | 52 | 24 | 9 | 10 | <0.03 |
| 1017045 | 71 | 36 | 20 | 6 | 0.08 |
| 1017073 | 49 | 27 | 9 | 5 | <0.03 |

TABLE 20
Dose-dependent reduction of human DMPK RNA
in A431 cells by modified oligonucleotides

| Compound | DMPK RNA (% UTC) | | | | IC₅₀ |
|---|---|---|---|---|---|
| No. | 31 nM | 125 nM | 500 nM | 2000 nM | (μM) |
| 570762 | 72 | 53 | 23 | 13 | 0.13 |
| 1002668 | 41 | 23 | 13 | 9 | <0.03 |
| 1002927 | 52 | 24 | 13 | 4 | <0.03 |
| 1003050 | 61 | 28 | 12 | 5 | 0.04 |
| 1003071 | 29 | 10 | 4 | 4 | <0.03 |
| 1016708† | 40 | 28 | 16 | 8 | <0.03 |
| 1016785 | 27 | 11 | 8 | 8 | <0.03 |
| 1016870 | 28 | 10 | 6 | 6 | <0.03 |
| 1016882 | 64 | 28 | 11 | 5 | 0.05 |
| 1016893 | 40 | 16 | 5 | 8 | <0.03 |
| 1016939 | 48 | 21 | 13 | 10 | <0.03 |
| 1016971 | 50 | 20 | 18 | 18 | <0.03 |
| 1017036 | 47 | 19 | 6 | 3 | <0.03 |
| 1017039 | 79 | 37 | 16 | 9 | 0.10 |
| 1017040 | 43 | 19 | 7 | 4 | <0.03 |
| 1017046 | 15 | 4 | 1 | 1 | <0.03 |
| 1017070 | 76 | 42 | 20 | 16 | 0.11 |

TABLE 21
Dose-dependent reduction of human DMPK RNA
in A431 cells by modified oligonucleotides

| Compound | DMPK RNA (% UTC) | | | | IC₅₀ |
|---|---|---|---|---|---|
| No. | 31 nM | 125 nM | 500 nM | 2000 nM | (μM) |
| 570218 | 29 | 13 | 7 | 7 | <0.03 |
| 1002576 | 65 | 44 | 34 | 18 | 0.10 |
| 1002929 | 64 | 28 | 12 | 15 | 0.04 |
| 1003007 | 59 | 26 | 7 | 4 | 0.04 |
| 1003025 | 70 | 27 | 10 | 14 | 0.06 |
| 1003026 | 41 | 14 | 10 | 2 | <0.03 |
| 1003051 | 60 | 17 | 5 | 1 | <0.03 |
| 1003064 | 54 | 57 | 10 | 5 | 0.07 |
| 1003072 | 50 | 34 | 11 | 15 | <0.03 |
| 1003075 | 54 | 22 | 6 | 3 | <0.03 |
| 1003097 | 71 | 41 | 24 | 11 | 0.10 |
| 1003123 | 48 | 24 | 6 | 4 | <0.03 |
| 1016871 | 17 | 7 | 6 | 12 | <0.03 |
| 1016948 | 48 | 23 | 16 | 9 | <0.03 |
| 1017003 | 74 | 38 | 12 | 5 | 0.09 |
| 1017016 | 46 | 14 | 2 | 2 | <0.03 |
| 1017023 | 37 | 13 | 11 | 13 | <0.03 |
| 1017100 | 43 | 17 | 7 | 4 | <0.03 |

TABLE 22
Dose-dependent reduction of human DMPK RNA
in A431 cells by modified oligonucleotides

| Compound | DMPK RNA (% UTC) | | | | IC₅₀ |
|---|---|---|---|---|---|
| No. | 31 nM | 125 nM | 500 nM | 2000 nM | (μM) |
| 486178 | 108 | 83 | 78 | 54 | >2.0 |
| 570280 | 53 | 36 | 11 | 5 | 0.04 |
| 1002586 | 25 | 11 | 8 | 22 | <0.03 |
| 1002847 | 68 | 46 | 22 | 10 | 0.10 |
| 1002848 | 71 | 43 | 20 | 12 | 0.10 |
| 1002930 | 48 | 18 | 8 | 4 | <0.03 |
| 1002965 | 72 | 32 | 16 | 7 | 0.07 |
| 1003027 | 13 | 4 | 1 | 2 | <0.03 |
| 1003053 | 67 | 38 | 9 | 6 | 0.07 |
| 1016800 | 60 | 28 | 10 | 12 | 0.04 |
| 1016801 | 52 | 19 | 13 | 10 | <0.03 |
| 1016813 | 39 | 13 | 9 | 7 | <0.03 |
| 1016896 | 59 | 36 | 15 | 8 | 0.05 |
| 1017024 | 41 | 20 | 8 | 3 | <0.03 |
| 1017065 | 15 | 4 | 16 | 1 | <0.03 |
| 1017092 | 54 | 22 | 17 | 17 | <0.03 |

TABLE 23
Dose-dependent reduction of human DMPK RNA
in A431 cells by modified oligonucleotides

| | | |
|---|---|---|
| Compound | DMPK RNA (% UTC) | IC₅₀ |

| No. | 8 nM | 31 nM | 125 nM | 500 nM | (μM) |
|---|---|---|---|---|---|
| 569381 | 122 | 70 | 39 | 21 | 0.11 |
| 569394 | 95 | 53 | 30 | 17 | 0.06 |
| 569681 | 79 | 69 | 41 | 16 | 0.07 |
| 570581 | 63 | 54 | 26 | 15 | 0.03 |
| 570623 | 72 | 45 | 26 | 49 | 0.05 |
| 570724 | 68 | 53 | 41 | 18 | 0.04 |
| 570766 | 90 | 77 | 28 | 19 | 0.08 |
| 570777 | 78 | 71 | 40 | 21 | 0.07 |
| 570798 | 94 | 64 | 30 | 10 | 0.06 |
| 570800 | 88 | 83 | 51 | 23 | 0.12 |
| 570900 | 47 | 62 | 34 | 20 | 0.02 |
| 570925 | 72 | 60 | 32 | 19 | 0.05 |
| 1016712 | 87 | 63 | 35 | 22 | 0.07 |
| 1277247 | 86 | 77 | 51 | 22 | 0.11 |
| 1277255 | 63 | 53 | 31 | 19 | 0.03 |

TABLE 24
Dose-dependent reduction of human DMPK RNA
in A431 cells by modified oligonucleotides

| | | |
|---|---|---|
| Compound | DMPK RNA (% UTC) | IC₅₀ |

| No. | 8 nM | 31 nM | 125 nM | 500 nM | (μM) |
|---|---|---|---|---|---|
| 569634 | 88 | 56 | 18 | 5 | 0.04 |
| 569643 | 69 | 57 | 34 | 21 | 0.04 |
| 569801 | 86 | 65 | 31 | 22 | 0.07 |
| 570433 | 43 | 24 | 8 | 7 | <0.01 |
| 570434 | 35 | 14 | 6 | 4 | <0.01 |
| 570435 | 61 | 32 | 10 | 7 | 0.01 |
| 570573 | 66 | 51 | 19 | 10 | 0.03 |
| 570725 | 85 | 57 | 21 | 4 | 0.04 |
| 570730 | 94 | 51 | 19 | 10 | 0.05 |
| 570793 | 63 | 49 | 19 | 9 | 0.02 |
| 570885 | 109 | 68 | 31 | 13 | 0.08 |
| 598769 | 109 | 75 | 42 | 27 | 0.12 |
| 1277250 | 98 | 63 | 41 | 22 | 0.09 |

### Example 6: Design of Modified Oligonucleotides Complementary to a Human DMPK Nucleic Acid

Modified oligonucleotides complementary to human DMPK nucleic acid were designed and synthesized. “Start site” indicates the 5′-most nucleoside of the target sequence to which the modified oligonucleotide is complementary. “Stop site” indicates the 3′-most nucleoside of the target sequence to which the modified oligonucleotide is complementary. As shown in the tables below, the modified oligonucleotides are complementary to SEQ ID NO: 1 (described herein above) and/or SEQ ID NO: 2 (described herein above). ‘N/A’ indicates that the modified oligonucleotide is not 100% complementary to that particular target sequence.

The modified oligonucleotides in the table below are 4-10-6 MOE modified oligonucleotides with mixed PO/PS backbone internucleoside linkages. The modified oligonucleotides are 20 nucleosides in length. The sugar motif for the modified oligonucleotides is (from 5′ to 3′): eeeeddddddddddeeeeee; wherein each ‘d’ represents a 2′-β-D-deoxyribosyl sugar moiety, and each ‘e’ represents a 2′-MOE sugar moiety. The internucleoside linkage motif for the modified oligonucleotides is (from 5′ to 3′): sooossssssssssoooss wherein each ‘s’ represents a phosphorothioate internucleoside linkage, and each ‘o’ represents a phosphodiester internucleoside linkage. All cytosine nucleobases are 5-methylcytosines.

TABLE 25
4-10-6 MOE modified oligonucleotides with mixed PS/PO internucleoside linkages
complementary to human DMPK

| | | SEQ ID | SEQ ID | SEQ ID | SEQ ID | |
|---|---|---|---|---|---|---|
| | | No: 1 | No: 1 | No: 2 | No: 2 | |
| Compound | Nucleobase Sequence | Start | Stop | Start | Stop | SEQ ID |
| No. | (5′ to 3′) | Site | Site | Site | Site | No. |
| 1459324 | GGGACAGACAATAAATACCG | 24733 | 24752 | 2791 | 2810 | 437 |
| 1459325 | GGACAGACAATAAATACCGA | 24732 | 24751 | 2790 | 2809 | 366 |
| 1459326 | GACAGACAATAAATACCGAG | 24731 | 24750 | 2789 | 2808 | 250 |
| 1459327 | ACAGACAATAAATACCGAGG | 24730 | 24749 | 2788 | 2807 | 173 |
| 1459328 | CAGACAATAAATACCGAGGA | 24729 | 24748 | 2787 | 2806 | 96 |
| 1459329 | AGACAATAAATACCGAGGAA | 24728 | 24747 | 2786 | 2805 | 18 |
| 1459330 | GACAATAAATACCGAGGAAT | 24727 | 24746 | 2785 | 2804 | 1020 |
| 1459343 | GCTTTAGTCCTACCCCTTAT | 14024 | 14043 | N/A | N/A | 402 |
| 1459344 | CTTTAGTCCTACCCCTTATT | 14023 | 14042 | N/A | N/A | 293 |
| 1459345 | AAGAATTTGCATTCTTTTAC | 19899 | 19918 | N/A | N/A | 1248 |
| 1459346 | AGAATTTGCATTCTTTTACA | 19898 | 19917 | N/A | N/A | 1249 |
| 1459348 | AATTTGCATTCTTTTACAAC | 19896 | 19915 | N/A | N/A | 1250 |
| 1459349 | ATTTGCATTCTTTTACAACT | 19895 | 19914 | N/A | N/A | 1251 |
| 1459351 | TTTGCATTCTTTTACAACTG | 19894 | 19913 | N/A | N/A | 313 |
| 1459352 | TTGCATTCTTTTACAACTGA | 19893 | 19912 | N/A | N/A | 249 |
| 1459353 | TGCATTCTTTTACAACTGAT | 19892 | 19911 | N/A | N/A | 1252 |
| 1459354 | GCATTCTTTTACAACTGATT | 19891 | 19910 | N/A | N/A | 160 |
| 1459355 | CATTCTTTTACAACTGATTG | 19890 | 19909 | N/A | N/A | 1253 |
| 1459356 | ATTCTTTTACAACTGATTGT | 19889 | 19908 | N/A | N/A | 1254 |
| 1459406 | CCATGGCTTGTTTCTCCTTC | 22125 | 22144 | N/A | N/A | 777 |
| 1459407 | CATGGCTTGTTTCTCCTTCA | 22124 | 22143 | N/A | N/A | 1255 |
| 1459408 | ATGGCTTGTTTCTCCTTCAC | 22123 | 22142 | N/A | N/A | 1256 |
| 1459409 | TGGCTTGTTTCTCCTTCACC | 22122 | 22141 | N/A | N/A | 1257 |
| 1459412 | CTTGTTTCTCCTTCACCAGC | 22119 | 22138 | N/A | N/A | 1258 |
| 1459413 | TTGTTTCTCCTTCACCAGCG | 22118 | 22137 | N/A | N/A | 1259 |
| 1459417 | GAGTGCTTTAGTCCTACCCC | 14028 | 14047 | N/A | N/A | 618 |
| 1459418 | AGTGCTTTAGTCCTACCCCT | 14027 | 14046 | N/A | N/A | 556 |
| 1459419 | GTGCTTTAGTCCTACCCCTT | 14026 | 14045 | N/A | N/A | 471 |
| 1459420 | TGCTTTAGTCCTACCCCTTA | 14025 | 14044 | N/A | N/A | 1260 |
| 1459985 | GCACCTTCCCGAATGTCCGA | 19500 | 19519 | 1361 | 1380 | 257 |
| 1459986 | TTCCCGAATGTCCGACAGTG | 19495 | 19514 | 1356 | 1375 | 1261 |
| 1459987 | TCCCGAATGTCCGACAGTGT | 19494 | 19513 | 1355 | 1374 | 1262 |
| 1459988 | TATTGTTATATGGCTGATTC | 19920 | 19939 | N/A | N/A | 647 |
| 1459989 | ATTGTTATATGGCTGATTCA | 19919 | 19938 | N/A | N/A | 588 |
| 1459990 | TTGTTATATGGCTGATTCAA | 19918 | 19937 | N/A | N/A | 1263 |
| 1459991 | TGTTATATGGCTGATTCAAA | 19917 | 19936 | N/A | N/A | 1264 |

The modified oligonucleotides in the table below are 4-8-6 MOE modified oligonucleotides with mixed PO/PS backbone internucleoside linkages. The modified oligonucleotides are 18 nucleosides in length. The sugar motif for the modified oligonucleotides is (from 5′ to 3′): eeeeddddddddeeeeee; wherein each ‘d’ represents a 2′-β-D-deoxyribosyl sugar moiety, and each ‘e’ represents a 2′-MOE sugar moiety. The internucleoside linkage motif for the modified oligonucleotides is (from 5′ to 3′): soosssssssssoooss wherein each ‘s’ represents a phosphorothioate internucleoside linkage, and each ‘o’ represents a phosphodiester internucleoside linkage. All cytosine nucleobases are 5-methylcytosines.

TABLE 26
4-8-6 MOE modified oligonucleotides with mixed PS/PO
internucleoside linkages complementary to human DMPK

| | | SEQ | SEQ | |
|---|---|---|---|---|
| | | ID | ID | |
| | | No: 1 | No: 1 | |
| Compound | Nucleobase Sequence | Start | Stop | SEQ ID |
| No. | (5′ to 3′) | Site | Site | No. |
| 1459421 | ATGGCTTGTTTCTCCTTC | 22125 | 22142 | 1265 |
| 1459422 | ATTTGCATTCTTTTACAA | 19897 | 19914 | 1266 |
| 1459423 | TTTGCATTCTTTTACAAC | 19896 | 19913 | 1267 |
| 1459424 | TTGCATTCTTTTACAACT | 19895 | 19912 | 1268 |
| 1459425 | TGCATTCTTTTACAACTG | 19894 | 19911 | 1269 |
| 1459426 | GCATTCTTTTACAACTGA | 19893 | 19910 | 1270 |
| 1459427 | CATTCTTTTACAACTGAT | 19892 | 19909 | 1271 |
| 1459428 | ATTCTTTTACAACTGATT | 19891 | 19908 | 1272 |
| 1459429 | TTCTTTTACAACTGATTG | 19890 | 19907 | 1273 |
| 1459450 | CTTGTTTCTCCTTCACCA | 22121 | 22138 | 1274 |
| 1459455 | TTGTTTCTCCTTCACCAG | 22120 | 22137 | 1275 |
| 1459462 | TGTTTCTCCTTCACCAGC | 22119 | 22136 | 1276 |
| 1459465 | GTTTCTCCTTCACCAGCG | 22118 | 22135 | 1277 |

The modified oligonucleotides in the table below are 5-10-5 MOE modified oligonucleotides with mixed PO/PS backbone internucleoside linkages. The modified oligonucleotides are 20 nucleosides in length. The sugar motif for the modified oligonucleotides is (from 5′ to 3′): eeeeeddddddddddeeeee; wherein each ‘d’ represents a 2′-β-D-deoxyribosyl sugar moiety, and each ‘e’ represents a 2′-MOE sugar moiety. The internucleoside linkage motif for the modified oligonucleotides is (from 5′ to 3′): soooossssssssssooss wherein each ‘s’ represents a phosphorothioate internucleoside linkage, and each ‘o’ represents a phosphodiester internucleoside linkage. All cytosine nucleobases are 5-methylcytosines.

TABLE 27
5-10-5 MOE modified oligonucleotides with mixed PS/PO internucleoside linkages
complementary to human DMPK

| | | SEQ ID | SEQ ID | SEQ ID | SEQ ID | |
|---|---|---|---|---|---|---|
| | | No: 1 | No: 1 | No: 2 | No: 2 | |
| Compound | Nucleobase Sequence | Start | Stop | Start | Stop | SEQ ID |
| No. | (5′ to 3′) | Site | Site | Site | Site | No. |
| 1052871 | TCCCGAATGTCCGACAGTGT | 19494 | 19513 | 1355 | 1374 | 1262 |
| 1052877 | GTAATGTTGTCCAGTAATAA | 21090 | 21109 | N/A | N/A | 1278 |
| 1052883 | GTATGTGTAATGTTGTCCAG | 21096 | 21115 | N/A | N/A | 1279 |
| 1052887 | CGGAGCGGTTGTGAACTGGC | 24120 | 24139 | 2178 | 2197 | 1280 |
| 1052889 | CTCGGAGCGGTTGTGAACTG | 24122 | 24141 | 2180 | 2199 | 1281 |
| 1380457 | TTGTTATATGGCTGATTCAA | 19918 | 19937 | N/A | N/A | 1263 |
| 1380571 | TGTTATATGGCTGATTCAAA | 19917 | 19936 | N/A | N/A | 1264 |
| 1380962 | CTCAGTAGTAGATGGGCACA | 17321 | 17340 | N/A | N/A | 1282 |
| 1459383 | CATGGCTTGTTTCTCCTTCA | 22124 | 22143 | N/A | N/A | 1255 |
| 1459386 | GCTTGTTTCTCCTTCACCAG | 22120 | 22139 | N/A | N/A | 1283 |
| 1459387 | CTTGTTTCTCCTTCACCAGC | 22119 | 22138 | N/A | N/A | 1258 |
| 1459392 | TGCTTTAGTCCTACCCCTTA | 14025 | 14044 | N/A | N/A | 1260 |
| 1459393 | AAGAATTTGCATTCTTTTAC | 19899 | 19918 | N/A | N/A | 1248 |
| 1459394 | AGAATTTGCATTCTTTTACA | 19898 | 19917 | N/A | N/A | 1249 |
| 1459395 | GAATTTGCATTCTTTTACAA | 19897 | 19916 | N/A | N/A | 1284 |
| 1459396 | AATTTGCATTCTTTTACAAC | 19896 | 19915 | N/A | N/A | 1250 |
| 1459398 | ATTTGCATTCTTTTACAACT | 19895 | 19914 | N/A | N/A | 1251 |
| 1459399 | TGCATTCTTTTACAACTGAT | 19892 | 19911 | N/A | N/A | 1252 |
| 1459400 | CATTCTTTTACAACTGATTG | 19890 | 19909 | N/A | N/A | 1253 |
| 1459401 | ATTCTTTTACAACTGATTGT | 19889 | 19908 | N/A | N/A | 1254 |
| 1459402 | TTCTTTTACAACTGATTGTA | 19888 | 19907 | N/A | N/A | 1285 |
| 1460162 | CTCAACATTTCTGGAATCCC | 22469 | 22488 | N/A | N/A | 1286 |
| 1460163 | ACCCCCATGTTCTAGGGTCA | 21495 | 21514 | N/A | N/A | 1287 |
| 1460164 | ACATCTTTATAAGAGTCCCC | 15478 | 15497 | N/A | N/A | 1288 |
| 1460166 | GCCTATTTTTTAATTTCAGT | 18057 | 18076 | N/A | N/A | 1289 |
| 1460168 | GCCCCATCATTTTTTCTTGT | 17749 | 17768 | N/A | N/A | 1290 |
| 1460170 | CACACCACCTCTTTTCCCCT | 16484 | 16503 | N/A | N/A | 1291 |
| 1460171 | CCTATTTTTTAATTTCAGTT | 18056 | 18075 | N/A | N/A | 1292 |
| 1460174 | CTCAGATAGGGAAGGCCCCT | 15416 | 15435 | N/A | N/A | 1293 |
| 1460178 | CTTACATGTTCCCCCCAAAC | 19404 | 19423 | N/A | N/A | 1294 |
| 1460179 | CTGCCCAAGGCCTTTGCCCT | 22180 | 22199 | N/A | N/A | 1295 |
| 1460184 | CACACAGTTTGTTACACACA | 13316 | 13335 | N/A | N/A | 1296 |
| 1460185 | CTTGTTTATCCCCTACTCCT | 22496 | 22515 | N/A | N/A | 1297 |
| 1511076 | GCACTCTTCCCTGCGCCCCG | 23779 | 23798 | N/A | N/A | 1298 |
| 1511078 | CTCACCTGTGGCTCCCTCTG | 22943 | 22962 | N/A | N/A | 1299 |
| 1511079 | ATCCTTGTTTATCCCCTACT | 22499 | 22518 | N/A | N/A | 1300 |
| 1511080 | CCTTCCATGGCTTGTTTCTC | 22129 | 22148 | N/A | N/A | 1301 |
| 1511081 | GCCCTCACCTTTTCTCTCCC | 21924 | 21943 | N/A | N/A | 1302 |
| 1511082 | CTCACCTTTTCTCTCCCAAT | 21921 | 21940 | N/A | N/A | 1303 |
| 1511083 | ACTTCCTTTTCCTTATCTGT | 20914 | 20933 | N/A | N/A | 1304 |
| 1511084 | CTTCCTTTTCCTTATCTGTA | 20913 | 20932 | N/A | N/A | 1305 |
| 1511085 | TTCCTTATCTGTATTTTCTA | 20906 | 20925 | N/A | N/A | 1306 |
| 1511086 | TCTCCATCTTCTGACCTCAA | 19121 | 19140 | N/A | N/A | 1307 |
| 1511087 | CTCAGCTGTTGTAGTCCCAA | 19088 | 19107 | N/A | N/A | 1308 |
| 1511088 | GCCCAGTTCTGTTTTTTTTT | 18994 | 19013 | N/A | N/A | 1309 |
| 1511089 | ATCATTTTTTCTTGTATCCT | 17744 | 17763 | N/A | N/A | 1310 |
| 1511090 | ACTCCATTGTCTCAGCCCTG | 17418 | 17437 | N/A | N/A | 1311 |
| 1511091 | CTTGCCATAGGTCTCCGCCG | 16824 | 16843 | 1053 | 1072 | 1312 |
| 1511092 | ACCACCTCTTTTCCCCTCCA | 16481 | 16500 | N/A | N/A | 1313 |
| 1511093 | CCACCTCTTTTCCCCTCCAA | 16480 | 16499 | N/A | N/A | 1314 |
| 1511094 | ACCTCTTTTCCCCTCCAAAT | 16478 | 16497 | N/A | N/A | 1315 |
| 1511095 | GCTCTTGTCCCTCTTCCTAG | 15283 | 15302 | N/A | N/A | 1316 |
| 1511096 | GTCCCTCTTCCTAGTCACCC | 15277 | 15296 | N/A | N/A | 1317 |
| 1511097 | TCCCTCTTCCTAGTCACCCC | 15276 | 15295 | N/A | N/A | 1318 |
| 1511098 | TCCACCCGCTTCTGCACCCA | 14917 | 14936 | N/A | N/A | 1319 |
| 1511099 | CCCTCTGTCTGTCTCCCCTT | 14565 | 14584 | N/A | N/A | 1320 |
| 1511100 | CTCCCCTTCTCTCTGCCTCT | 14553 | 14572 | N/A | N/A | 1321 |
| 1511101 | CCTTCTCTCTGCCTCTCAGC | 14549 | 14568 | N/A | N/A | 1322 |
| 1511102 | CTGCCTCTCAGCTTCACCCT | 14541 | 14560 | N/A | N/A | 1323 |
| 1511103 | CTGGCCCTTCTTGGCCTCCA | 13446 | 13465 | N/A | N/A | 1324 |
| 1511104 | GCCCTTCTTGGCCTCCACCT | 13443 | 13462 | N/A | N/A | 1325 |
| 1511105 | CCTTCCAGCCCTGGCCCCAG | 13140 | 13159 | N/A | N/A | 1326 |
| 1511106 | CCTGCCATCCTGCCCCCCCA | 12413 | 12432 | N/A | N/A | 1327 |
| 1511107 | CTGCCATCCTGCCCCCCCAA | 12412 | 12431 | N/A | N/A | 1328 |
| 1511077 | CTGCCGTGGGCCCAGCCCCG | 23638 | 23657 | N/A | N/A | 1329 |

The modified oligonucleotides in the table below are 5-8-5 MOE modified oligonucleotides with mixed PO/PS backbone internucleoside linkages. The modified oligonucleotides are 18 nucleosides in length. The sugar motif for the modified oligonucleotides is (from 5′ to 3′): eeeeeddddddddeeeee; wherein each ‘d’ represents a 2′-β-D-deoxyribosyl sugar moiety, and each ‘e’ represents a 2′-MOE sugar moiety. The internucleoside linkage motif for the modified oligonucleotides is (from 5′ to 3′): sooosssssssssooss wherein each ‘s’ represents a phosphorothioate internucleoside linkage, and each ‘o’ represents a phosphodiester internucleoside linkage. All cytosine nucleobases are 5-methylcytosines.

TABLE 28
5-8-5 MOE modified oligonucleotides with mixed PS/PO
internucleoside linkages complementary to human DMPK

| | | SEQ | SEQ | |
|---|---|---|---|---|
| | | ID | ID | |
| | | No: 1 | No: 1 | |
| Compound | Nucleobase Sequence | Start | Stop | SEQ ID |
| No. | (5′ to 3′) | Site | Site | No. |
| 1459449 | GGCTTGTTTCTCCTTCAC | 22123 | 22140 | 1330 |
| 1459451 | CTTGTTTCTCCTTCACCA | 22121 | 22138 | 1274 |
| 1459452 | TTGTTTCTCCTTCACCAG | 22120 | 22137 | 1275 |
| 1459453 | TGTTTCTCCTTCACCAGC | 22119 | 22136 | 1276 |
| 1459456 | ATTTGCATTCTTTTACAA | 19897 | 19914 | 1266 |
| 1459457 | TTTGCATTCTTTTACAAC | 19896 | 19913 | 1267 |
| 1459458 | TTGCATTCTTTTACAACT | 19895 | 19912 | 1268 |
| 1459459 | TGCATTCTTTTACAACTG | 19894 | 19911 | 1269 |
| 1459461 | CATTCTTTTACAACTGAT | 19892 | 19909 | 1271 |
| 1459463 | ATTCTTTTACAACTGATT | 19891 | 19908 | 1272 |
| 1459464 | TTCTTTTACAACTGATTG | 19890 | 19907 | 1273 |

The modified oligonucleotides in the table below are 6-10-4 MOE modified oligonucleotides with mixed PO/PS backbone internucleoside linkages. The modified oligonucleotides are 20 nucleosides in length. The sugar motif for the modified oligonucleotides is (from 5′ to 3′): eeeeeeddddddddddeeee; wherein each ‘d’ represents a 2′-β-D-deoxyribosyl sugar moiety, and each ‘e’ represents a 2′-MOE sugar moiety. The internucleoside linkage motif for the modified oligonucleotides is (from 5′ to 3′): sooooossssssssssoss wherein each ‘s’ represents a phosphorothioate internucleoside linkage, and each ‘o’ represents a phosphodiester internucleoside linkage. All cytosine nucleobases are 5-methylcytosines.

TABLE 29
6-10-4 MOE modified oligonucleotides with mixed PS/PO internucleoside linkages
complementary to human DMPK

| | | SEQ | SEQ | SEQ | SEQ | |
|---|---|---|---|---|---|---|
| | | ID | ID | ID | ID | |
| | | No: 1 | No: 1 | No: 2 | No: | |
| Compound | Nucleobase Sequence | Start | Stop | Start | 2 Stop | SEQ |
| No. | (5′ to 3′) | Site | Site | Site | Site | ID No. |
| 1459314 | GGGACAGACAATAAATACCG | 24733 | 24752 | 2791 | 2810 | 437 |
| 1459315 | GCCTTATTGTTATATGGCTG | 19924 | 19943 | N/A | N/A | 972 |
| 1459320 | GGACAGACAATAAATACCGA | 24732 | 24751 | 2790 | 2809 | 366 |
| 1459322 | GACAGACAATAAATACCGAG | 24731 | 24750 | 2789 | 2808 | 250 |
| 1459323 | ACAGACAATAAATACCGAGG | 24730 | 24749 | 2788 | 2807 | 173 |
| 1459359 | GGAGTGCTTTAGTCCTACCC | 14029 | 14048 | N/A | N/A | 692 |
| 1459360 | GAGTGCTTTAGTCCTACCCC | 14028 | 14047 | N/A | N/A | 618 |
| 1459364 | TGCTTTAGTCCTACCCCTTA | 14025 | 14044 | N/A | N/A | 1260 |
| 1459365 | GCTTTAGTCCTACCCCTTAT | 14024 | 14043 | N/A | N/A | 402 |
| 1459366 | CTTTAGTCCTACCCCTTATT | 14023 | 14042 | N/A | N/A | 293 |
| 1459367 | AAGAATTTGCATTCTTTTAC | 19899 | 19918 | N/A | N/A | 1248 |
| 1459368 | AGAATTTGCATTCTTTTACA | 19898 | 19917 | N/A | N/A | 1249 |
| 1459369 | GAATTTGCATTCTTTTACAA | 19897 | 19916 | N/A | N/A | 1284 |
| 1459372 | ATTTGCATTCTTTTACAACT | 19895 | 19914 | N/A | N/A | 1251 |
| 1459373 | TTTGCATTCTTTTACAACTG | 19894 | 19913 | N/A | N/A | 313 |
| 1459374 | TTGCATTCTTTTACAACTGA | 19893 | 19912 | N/A | N/A | 249 |
| 1459375 | TGCATTCTTTTACAACTGAT | 19892 | 19911 | N/A | N/A | 1252 |
| 1459377 | CATTCTTTTACAACTGATTG | 19890 | 19909 | N/A | N/A | 1253 |
| 1459378 | ATTCTTTTACAACTGATTGT | 19889 | 19908 | N/A | N/A | 1254 |
| 1459379 | TTCTTTTACAACTGATTGTA | 19888 | 19907 | N/A | N/A | 1285 |
| 1459415 | CTTGTTTCTCCTTCACCAGC | 22119 | 22138 | N/A | N/A | 1258 |
| 1459973 | GCGCACCTTCCCGAATGTCC | 19502 | 19521 | 1363 | 1382 | 434 |
| 1459974 | CGCACCTTCCCGAATGTCCG | 19501 | 19520 | 1362 | 1381 | 358 |
| 1459975 | GCACCTTCCCGAATGTCCGA | 19500 | 19519 | 1361 | 1380 | 257 |
| 1459976 | CACCTTCCCGAATGTCCGAC | 19499 | 19518 | 1360 | 1379 | 174 |
| 1459977 | ACCTTCCCGAATGTCCGACA | 19498 | 19517 | 1359 | 1378 | 97 |
| 1459978 | TTCCCGAATGTCCGACAGTG | 19495 | 19514 | 1356 | 1375 | 1261 |
| 1459979 | TCCCGAATGTCCGACAGTGT | 19494 | 19513 | 1355 | 1374 | 1262 |
| 1459980 | TATTGTTATATGGCTGATTC | 19920 | 19939 | N/A | N/A | 647 |
| 1459981 | ATTGTTATATGGCTGATTCA | 19919 | 19938 | N/A | N/A | 588 |
| 1459982 | TTGTTATATGGCTGATTCAA | 19918 | 19937 | N/A | N/A | 1263 |
| 1459983 | TGTTATATGGCTGATTCAAA | 19917 | 19936 | N/A | N/A | 1264 |

The modified oligonucleotides in the table below are 6-8-4 MOE modified oligonucleotides with mixed PO/PS backbone internucleoside linkages. The modified oligonucleotides are 18 nucleosides in length. The sugar motif for the modified oligonucleotides is (from 5′ to 3′): eeeeeeddddddddeeee; wherein each ‘d’ represents a 2′-β-D-deoxyribosyl sugar moiety, and each ‘e’ represents a 2′-MOE sugar moiety. The internucleoside linkage motif for the modified oligonucleotides is (from 5′ to 3′): soooosssssssssoss wherein each ‘s’ represents a phosphorothioate internucleoside linkage, and each ‘o’ represents a phosphodiester internucleoside linkage. All cytosine nucleobases are 5-methylcytosines.

TABLE 30
6-8-4 MOE modified oligonucleotides with mixed PS/PO
internucleoside linkages complementary to human DMPK

| | | SEQ | SEQ | |
|---|---|---|---|---|
| | | ID | ID | |
| | | No: 1 | No: 1 | |
| Compound | Nucleobase Sequence | Start | Stop | SEQ ID |
| No. | (5′ to 3′) | Site | Site | No. |
| 1459433 | TGGCTTGTTTCTCCTTCA | 22124 | 22141 | 1331 |
| 1459434 | GGCTTGTTTCTCCTTCAC | 22123 | 22140 | 1330 |
| 1459435 | CTTGTTTCTCCTTCACCA | 22121 | 22138 | 1274 |
| 1459436 | TTGTTTCTCCTTCACCAG | 22120 | 22137 | 1275 |
| 1459437 | TGTTTCTCCTTCACCAGC | 22119 | 22136 | 1276 |
| 1459438 | GTTTCTCCTTCACCAGCG | 22118 | 22135 | 1277 |
| 1459439 | ATTTGCATTCTTTTACAA | 19897 | 19914 | 1266 |
| 1459440 | TTTGCATTCTTTTACAAC | 19896 | 19913 | 1267 |
| 1459441 | TTGCATTCTTTTACAACT | 19895 | 19912 | 1268 |
| 1459442 | TGCATTCTTTTACAACTG | 19894 | 19911 | 1269 |
| 1459443 | GCATTCTTTTACAACTGA | 19893 | 19910 | 1270 |
| 1459444 | CATTCTTTTACAACTGAT | 19892 | 19909 | 1271 |
| 1459445 | ATTCTTTTACAACTGATT | 19891 | 19908 | 1272 |
| 1459446 | TTCTTTTACAACTGATTG | 19890 | 19907 | 1273 |

The modified oligonucleotides in the table below are modified oligonucleotides with mixed sugars and uniform phosphorothioate internucleoside linkages. The modified oligonucleotides are 16 nucleosides in length. The sugar motif for the modified oligonucleotides is described in the column “Sugar Motif (from 5′ to 3′)” in the table below; wherein each ‘d’ represents a 2′-β-D-deoxyribosyl sugar moiety, each ‘e’ represents a 2′-MOE sugar moiety, each “y” represents a 2′-O-methylribosyl sugar moiety and each ‘k’ represents a cEt sugar moiety. The internucleoside linkage motif for the modified oligonucleotides is (from 5′ to 3′): sssssssssssssss wherein each ‘s’ represents a phosphorothioate internucleoside linkage. All cytosine nucleobases are 5-methylcytosines.

TABLE 31
Mixed cET/MOE modified oligonucleotides with uniform phosphorothioate internucleoside
linkages complementary to human DMPK

| | | SEQ | SEQ | SEQ | SEQ | | |
|---|---|---|---|---|---|---|---|
| | | ID | ID | ID | ID | | |
| | | No: 1 | No: 1 | No: 2 | No: 2 | | |
| Compound | Nucleobase Sequence | Start | Stop | Start | Stop | Sugar Motif | SEQ |
| No. | (5′ to 3′) | Site | Site | Site | Site | (from 5′ to 3′) | ID No. |
| 1338115 | GTTATATGGCTGATTC | 19920 | 19935 | N/A | N/A | kkkedddddddddkkk | 1332 |
| 1400761 | ATGTGTAATGTTGTCC | 21098 | 21113 | N/A | N/A | kkkedddddddddkkk | 1333 |
| 1400744 | ACCTUCCCGAATGTCC | 19502 | 19517 | 1363 | 1378 | kkkdyddddddddkkk | 1334 |
| 1400776 | CTTTTATTCGCGAGGG | 24775 | 24790 | 2833 | 2848 | kkeddddddddddkkk | 1335 |

The modified oligonucleotide in the table below is 16 nucleosides in length. The sugar motif for the modified oligonucleotide is (from 5′ to 3′): kekddddddddddkkk; wherein each ‘d’ represents a 2′-β-D-deoxyribosyl sugar moiety, each ‘e’ represents a 2′-MOE sugar moiety, and each ‘k’ represents a cEt sugar moiety. The internucleoside linkage motif for the modified oligonucleotide is (from 5′ to 3′): sssssssssssssss wherein each ‘s’ represents a phosphorothioate internucleoside linkage. All cytosine nucleobases are 5-methylcytosines. Further, the modified oligonucleotide in the table below is conjugated to a 6-palmitamidohexyl phosphate conjugate group attached to the 5′-OH of the oligonucleotide. The structure for the conjugate group is:

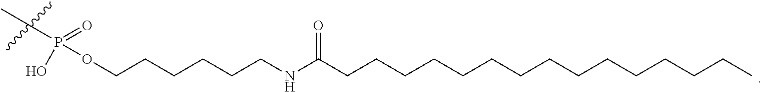 [SMILES: *P(=O)(O)OCCCCCCNC(=O)CCCCCCCCCCCCCCC]

TABLE 32
6-palmitamidohexyl phosphate conjugated mixed cET/MOE
modified oligonucleotide with uniform phosphorothioate
internucleoside linkages complementary to human DMPK

| | | SEQ ID | SEQ ID | |
|---|---|---|---|---|
| | | No: 1 | No: 1 | SEQ |
| Compound | Nucleobase Sequence | Start | Stop | ID |
| No. | (5′ to 3′) | Site | Site | No. |
| 1273291 | GTTATATGGCTGATTC | 19920 | 19935 | 1332 |

### Example 7: Design of Modified Oligonucleotides Complementary to a Human DMPK Nucleic Acid

Modified oligonucleotides complementary to human DMPK nucleic acid were synthesized. “Start site” indicates the 5′-most nucleoside of the target sequence to which the modified oligonucleotide is complementary. “Stop site” indicates the 3′-most nucleoside of the target sequence to which the modified oligonucleotide is complementary. As shown in the tables below, the modified oligonucleotides are complementary to SEQ ID NO: 1 (the complement of GENBANK Accession No. NT_011109.16 truncated from nucleotides 18539000 to 18566000) and/or SEQ ID NO: 2 (GENBANK Accession No. NM_004409.4). ‘N/A’ indicates that the modified oligonucleotide is not 100% complementary to that particular target sequence.

The modified oligonucleotides in the table below are 16 nucleosides in length. The sugar motif for the modified oligonucleotides in the table below are described in the column labeled “Sugar Motif (5′ to 3′),” wherein each “d” represents a 2′-β-D-deoxyribosyl sugar moiety, each “k” represents a cEt sugar moiety, each “e” represents a 2′-MOE sugar moiety, and each “y” represents a 2′-O-methylribosyl sugar moiety. The internucleoside linkage motif for the modified oligonucleotide is (from 5′ to 3′): sssssssssssssss; wherein each “s” represents a phosphorothioate internucleoside linkage. Each cytosine residue is a 5-methylcytosine unless otherwise indicated. Non-methylated cytosines are represented in bold underlined italicized font as “C”.

TABLE 33
Modified oligonucleotides with uniform phosphorothioate linkages complementary to human DMPK

| | | SEQ | SEQ | SEQ | SEQ | | |
|---|---|---|---|---|---|---|---|
| | | ID | ID | ID | ID | | |
| | | NO: 1 | NO: 1 | NO: 2 | NO: 2 | | SEQ |
| Compound | Nucleobase Sequence | Start | Stop | Start | Stop | Sugar Motif | ID |
| No. | (5′ to 3′) | Site | Site | Site | Site | (5′ to 3′) | NO |
| 570052 | CTTTTATTCGCGAGGG | 24775 | 24790 | 2833 | 2848 | kkkddddddddddkkk | 1335 |
| 1003033 | GTTATATGGCTGATTC | 19920 | 19935 | N/A | N/A | kkkddddddddddkkk | 1332 |
| 1338115 | GTTATATGGCTGATTC | 19920 | 19935 | N/A | N/A | kkkedddddddddkkk | 1332 |
| 1400737 | CGAAUGTCCGACAGTG | 19495 | 19510 | 1356 | 1371 | kkkdyddddddddkkk | 2297 |
| 1400739 | CGAATGTCCGACAGTG | 19495 | 19510 | 1356 | 1371 | kkeddddddddddkkk | 2298 |
| 1400742 | TTCCCGAATGTCCGAC | 19499 | 19514 | 1360 | 1375 | kkeddddddddddkkk | 2299 |
| 1400743 | TTCCCGAATGTCCGAC | 19499 | 19514 | 1360 | 1375 | kkkedddddddddkkk | 2299 |
| 1400744 | ACCTUCCCGAATGTCC | 19502 | 19517 | 1363 | 1378 | kkkdyddddddddkkk | 1334 |
| 1400748 | CACCUTCCCGAATGTC | 19503 | 19518 | 1364 | 1379 | kkkdyddddddddkkk | 2300 |
| 1400750 | CACCTTCCCGAATGTC | 19503 | 19518 | 1364 | 1379 | kkkedddddddddkkk | 2301 |
| 1400752 | GCACCTTCCCGAATGT | 19504 | 19519 | 1365 | 1380 | kkkdyddddddddkkk | 2302 |
| 1400755 | TGTAATGTTGTCCAGT | 21095 | 21110 | N/A | N/A | ekkddddddddddkke | 1342 |
| 1400760 | ATGTGTAATGTTGTCC | 21098 | 21113 | N/A | N/A | kkkdyddddddddkkk | 1333 |
| 1400761 | ATGTGTAATGTTGTCC | 21098 | 21113 | N/A | N/A | kkkedddddddddkkk | 1333 |
| 1400769 | GTTAUATGGCTGATTC | 19920 | 19935 | N/A | N/A | kkkdyddddddddkkk | 2303 |
| 1400772 | GTTATATGGCTGATTC | 19920 | 19935 | N/A | N/A | kkeddddddddddkkk | 1332 |
| 1400775 | CTTTUATTCGCGAGGG | 24775 | 24790 | 2833 | 2848 | kkkdyddddddddkkk | 2304 |
| 1400776 | CTTTTATTCGCGAGGG | 24775 | 24790 | 2833 | 2848 | kkeddddddddddkkk | 1335 |
| 1400777 | CTTTTATTCGCGAGGG | 24775 | 24790 | 2833 | 2848 | kkkedddddddddkkk | 1335 |
| 1400778 | GGAAUCTATCATGGCT | 20811 | 20826 | N/A | N/A | kkkdyddddddddkkk | 2305 |

The modified oligonucleotide in the table below is 16 nucleosides in length. The sugar motif for the modified oligonucleotide in the table below is described in the column labeled “Sugar Motif (5′ to 3′),” wherein each “d” represents a 2′-β-D-deoxyribosyl sugar moiety, and each “k” represents a cEt sugar moiety. The internucleoside linkage motif for the modified oligonucleotide is (from 5′ to 3′): ssssxssssssssss; wherein each “s” represents a phosphorothioate internucleoside linkage and each “x” represents a methoxypropyl phosphonate internucleoside linkage. Each cytosine residue is a 5-methylcytosine.

TABLE 34
3-10-3 cET modified oligonucleotides with mixed internucleoside linkages complementary
to human DMPK

| | | SEQ | SEQ | SEQ | SEQ | | |
|---|---|---|---|---|---|---|---|
| | | ID | ID | ID | ID | | |
| | | NO: 1 | NO: 1 | NO: 2 | NO: 2 | | SEQ |
| Compound | Nucleobase Sequence | Start | Stop | Start | Stop | Sugar Motif | ID |
| No. | (5′ to 3′) | Site | Site | Site | Site | (5′ to 3′) | NO |
| 1273313 | ATGTGTAATGTTGTCC | 21098 | 21113 | N/A | N/A | kkkddddddddddkkk | 1333 |

The modified oligonucleotides in the table below are 3-10-3 cEt modified oligonucleotides with uniform phosphorothioate backbone internucleoside linkages. The modified oligonucleotides are 16 nucleosides in length. The sugar motif for the modified oligonucleotides is (from 5′ to 3′): kkkddddddddddkkk; wherein each “d” represents a 2′-β-D-deoxyribosyl sugar moiety, and each “k” represents a cEt sugar moiety. The internucleoside linkage motif for the modified oligonucleotides is (from 5′ to 3′): sssssssssssssss; wherein each “s” represents a phosphorothioate internucleoside linkage. All cytosine nucleobases are 5-methylcytosines. Inosine nucleobases are represented by the letter “I” in the Nucleoside Sequence column in the table below. Further, the modified oligonucleotides in the table below are conjugated to a 6-palmitamidohexyl phosphate conjugate group attached to the 5′-OH of the oligonucleotide.

The structure for the conjugate group is:

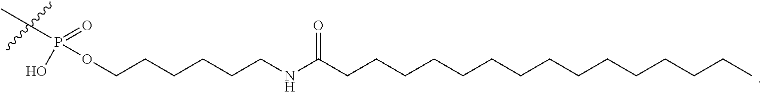 [SMILES: *P(=O)(O)OCCCCCCNC(=O)CCCCCCCCCCCCCCC]

TABLE 35
6-Palmitamidohexyl conjugated 3-10-3 cEt modified oligonucleotides with
uniform phosphorothioate internucleoside linkages complementary to human DMPK

| | | SEQ | SEQ | SEQ | SEQ | |
|---|---|---|---|---|---|---|
| | | ID | ID | ID | ID | |
| | | NO: 1 | NO: 1 | NO: 2 | NO: 2 | SEQ |
| Compound | Nucleobase Sequence | Start | Stop | Start | Stop | ID |
| No. | (5′ to 3′) | Site | Site | Site | Site | NO |
| 1046919 | CCCGAATGTCCGACAG | 19497 | 19512 | 1358 | 1373 | 2306 |
| 1046921 | GCACTTTGCGAACCAA | 24449 | 24464 | 2507 | 2522 | 2307 |
| 1046922 | TGCCCCGGGCACTCAG | 24078 | 24093 | 2136 | 2151 | 2308 |
| 1046924 | GAGTATACAGGCATGC | 17920 | 17935 | N/A | N/A | 2309 |
| 1046926 | GCAAATTTCCCGAGTA | 24525 | 24540 | 2583 | 2598 | 1414 |
| 1046933 | GAACTGGCAGGCGGTG | 24112 | 24127 | 2170 | 2185 | 2310 |
| 1046934 | ACCTGGCCCGTCTGCT | 15022 | 15037 | 477 | 492 | 2311 |
| 1046935 | AAAGCAAATTTCCCGA | 24528 | 24543 | 2586 | 2601 | 2312 |
| 1046936 | AGTCGGAGGACGAGGT | 24647 | 24662 | 2705 | 2720 | 2313 |
| 1046937 | CCTCTTAGGAGTCTTT | 19696 | 19711 | N/A | N/A | 2314 |
| 1059473 | AAATACCGAGGAATGT | 24725 | 24740 | 2783 | 2798 | 2315 |
| 1059474 | TAAATACCGAGGAATG | 24726 | 24741 | 2784 | 2799 | 2316 |
| 1059475 | ATAAATACCGAGGAAT | 24727 | 24742 | 2785 | 2800 | 2317 |
| 1059476 | AATAAATACCGAGGAA | 24728 | 24743 | 2786 | 2801 | 2318 |
| 1059477 | CAATAAATACCGAGGA | 24729 | 24744 | 2787 | 2802 | 2319 |
| 1059478 | GACAATAAATACCGAG | 24731 | 24746 | 2789 | 2804 | 2320 |
| 1059892 | ATAAGCAATGCATTAT | 20879 | 20894 | N/A | N/A | 2321 |
| 1059894 | TGATAAGCAATGCATT | 20881 | 20896 | N/A | N/A | 2322 |
| 1059903 | ATTTACTTGTGATAAG | 20890 | 20905 | N/A | N/A | 2323 |
| 1060860 | GTTATATGGCTGATTC | 19920 | 19935 | N/A | N/A | 1332 |
| 1060864 | ATGGTTACAAGATTCT | 19803 | 19818 | N/A | N/A | 1469 |
| 1060866 | CAAATTTTGTGCAGGT | 10409 | 10424 | N/A | N/A | 2046 |
| 1060867 | AGGTATAGTATGTGTA | 21107 | 21122 | N/A | N/A | 1880 |
| 1060869 | GGTTATGGCTAGGAGG | 10322 | 10337 | N/A | N/A | 1674 |
| 1060870 | CTTATTGTTATATGGC | 19926 | 19941 | N/A | N/A | 1619 |
| 1060872 | TGCTTTAGTCCTACCC | 14029 | 14044 | N/A | N/A | 2163 |
| 1060873 | GACAATCAGGCCTCTC | 14000 | 14015 | N/A | N/A | 1791 |
| 1060875 | TTCATTAATGATAAGG | 21120 | 21135 | N/A | N/A | 1510 |
| 1060876 | AATAGATTCTGGTTCG | 9241 | 9256 | V/A | N/A | 1896 |
| 1060877 | AAATTTTGTGCAGGTG | 10408 | 10423 | N/A | N/A | 1972 |
| 1060878 | AAGGTATAGTATGTGT | 21108 | 21123 | N/A | N/A | 1952 |
| 1060880 | ACAATAGCAAGGGCAG | 10685 | 10700 | N/A | N/A | 1757 |
| 1060882 | GACTCTACGATTCCAA | 9688 | 9703 | N/A | N/A | 2194 |
| 1060883 | GTTAATGGTTACAAGA | 19807 | 19822 | N/A | N/A | 1581 |
| 1060884 | AGACACTAAGATTTCC | 9058 | 9073 | N/A | N/A | 1895 |
| 1060886 | CCATAATTTAACACTC | 10714 | 10729 | V/A | N/A | 1905 |
| 1060887 | AGAGAAATGTTGCCCC | 12487 | 12502 | N/A | N/A | 1865 |
| 1060889 | GTTACAAGATTCTGGG | 19800 | 19815 | N/A | N/A | 2106 |
| 1060891 | AGTTAATGGTTACAAG | 19808 | 19823 | N/A | N/A | 1655 |
| 1060892 | TGTAAGTCTAGGTCAC | 17350 | 17365 | N/A | N/A | 1873 |
| 1060893 | ACAGTAAGGTTCCAAG | 19671 | 19686 | N/A | N/A | 1987 |
| 1060896 | AGTAGATGGGCACAGA | 17319 | 17334 | N/A | N/A | 2174 |
| 1060897 | TTAGACAAAGTAGCAT | 21047 | 21062 | N/A | N/A | 1494 |
| 1060898 | TCATTAATGATAAGGT | 21119 | 21134 | N/A | N/A | 1433 |
| 1060899 | GAGAATAGGTCCCAGA | 15578 | 15593 | N/A | N/A | 1786 |
| 1060900 | ATAAGGTATAGTATGT | 21110 | 21125 | N/A | N/A | 2030 |
| 1060901 | GTTACACGGTGAAGAG | 17565 | 17580 | N/A | N/A | 1874 |
| 1060903 | GCTTACATGTTCCCCC | 19409 | 19424 | N/A | N/A | 1356 |
| 1060904 | GCCTACTATGACCTTC | 9553 | 9568 | N/A | N/A | 2193 |
| 1060905 | AGAGAATAGGTCCCAG | 15579 | 15594 | N/A | N/A | 1651 |
| 1060906 | ATGATAAGGTATAGTA | 21113 | 21128 | N/A | N/A | 2110 |
| 1060908 | GAGATATCAACTTCCT | 20927 | 20942 | N/A | N/A | 2028 |
| 1060909 | TTCAATCAAGCGATTC | 18900 | 18915 | N/A | N/A | 1727 |
| 1060911 | CAGCGAGTCGGAGGAC | 24652 | 24667 | 2710 | 2725 | 2005 |
| 1060914 | AAGCAAATTTCCCGAG | 24527 | 24542 | 2585 | 2600 | 1856 |
| 1060915 | TGTTAGTCCACTCGCA | 23737 | 23752 | N/A | N/A | 2324 |
| 1060916 | GTCGAAGACAGTTCTA | 24042 | 24057 | 2100 | 2115 | 2325 |
| 1060917 | TAAATATCCAAACCGC | 24628 | 24643 | 2686 | 2701 | 2326 |
| 1060918 | GCAAAAGCAAATTTCC | 24531 | 24546 | 2589 | 2604 | 2327 |
| 1060921 | TATCTAAAGTGGCCCC | 19370 | 19385 | N/A | N/A | 2328 |
| 1060923 | CTTTTATTCGCGAGGG | 24775 | 24790 | 2833 | 2848 | 1335 |
| 1162627 | ATTCGCGAGGGTCGGG | 24770 | 24785 | 2828 | 2843 | 2329 |
| 1162628 | TATTCGCGAGGGTCGG | 24771 | 24786 | 2829 | 2844 | 2330 |
| 1162632 | CCTTTTATTCGCGAGG | 24776 | 24791 | 2834 | 2849 | 2331 |
| 1162633 | GCCTTTTATTCGCGAG | 24777 | 24792 | 2835 | 2850 | 2332 |
| 1162634 | GGCCTTTTATTCGCGA | 24778 | 24793 | 2836 | 2851 | 2333 |
| 1162635 | GGGCCTTTTATTCGCG | 24779 | 24794 | 2837 | 2852 | 2334 |
| 1162636 | AGGGCCTTTTATTCGC | 24780 | 24795 | 2838 | 2853 | 2158 |
| 1176162 | ATGTITAATGTTGTCC | 21098 | 21113 | N/A | N/A | 2157 |
| 1207018 | ATATGGCTGATTCAAA | 19917 | 19932 | N/A | N/A | 2153 |
| 1207019 | TATATGGCTGATTCAA | 19918 | 19933 | N/A | N/A | 1400 |
| 1207020 | TTATATGGCTGATTCA | 19919 | 19934 | N/A | N/A | 2085 |
| 1207021 | TGTTATATGGCTGATT | 19921 | 19936 | N/A | N/A | 2082 |
| 1207024 | TATTGTTATATGGCTG | 19924 | 19939 | N/A | N/A | 2079 |
| 1207025 | TTATTGTTATATGGCT | 19925 | 19940 | N/A | N/A | 2006 |

The modified oligonucleotides in the table below are 2-10-2 cEt modified oligonucleotides with uniform phosphorothioate backbone internucleoside linkages. The modified oligonucleotides are 14 nucleosides in length. The sugar motif for the modified oligonucleotides is (from 5′ to 3′): kkddddddddddkk; wherein each “d” represents a 2′-β-D-deoxyribosyl sugar moiety, and each “k” represents a cEt sugar moiety. The internucleoside linkage motif for the modified oligonucleotides is (from 5′ to 3′): sssssssssssss; wherein each “s” represents a phosphorothioate internucleoside linkage. All cytosine nucleobases are 5-methylcytosines. Further, the modified oligonucleotides in the table below are conjugated to a 6-palmitamidohexyl phosphate conjugate group (shown herein above) attached to the 5′-OH of the oligonucleotide.

TABLE 36
6-Palmitamidohexyl conjugated 2-10-2 cEt modified
oligonucleotides with uniform phosphorothioate internucleoside
linkages complementary to human DMPK

| | | SEQ | SEQ | |
|---|---|---|---|---|
| | | ID | ID | |
| | | NO: 1 | NO: 1 | SEQ |
| Compound | Nucleobase Sequence | Start | Stop | ID |
| No. | (5′ to 3′) | Site | Site | NO |
| 1059502 | GTAATGTTGTCCAG | 21096 | 21109 | 1935 |
| 1059503 | TGTAATGTTGTCCA | 21097 | 21110 | 1934 |
| 1059506 | ATGTGTAATGTTGT | 21100 | 21113 | 1932 |

The modified oligonucleotides in the table below are 3-10-3 cEt modified oligonucleotides with uniform phosphorothioate backbone internucleoside linkages. The modified oligonucleotides are 16 nucleosides in length. The sugar motif for the modified oligonucleotides is (from 5′ to 3′): kkkddddddddddkkk; wherein each “d” represents a 2′-β-D-deoxyribosyl sugar moiety, and each “k” represents a cEt sugar moiety. The internucleoside linkage motif for the modified oligonucleotides is (from 5′ to 3′): ssssxssssssssss; wherein each “s” represents a phosphorothioate internucleoside linkage, and each “x” represents a methoxypropyl phosphonate internucleoside linkage. All cytosine nucleobases are 5-methylcytosines. Further, the modified oligonucleotides in the table below are conjugated to a 6-palmitamidohexyl phosphate conjugate group (shown herein above) attached to the 5′-OH of the oligonucleotide.

TABLE 37
6-Palmitamidohexyl conjugated 3-10-3 cEt modified oligonucleotides with mixed
internucleoside linkages complementary to human DMPK

| | | SEQ | SEQ | SEQ | SEQ | |
|---|---|---|---|---|---|---|
| | | ID | ID | ID | ID | |
| | | NO: 1 | NO: 1 | NO: 2 | NO: 2 | SEQ |
| Compound | Nucleobase Sequence | Start | Stop | Start | Stop | ID |
| No. | (5′ to 3′) | Site | Site | Site | Site | NO |
| 1059887 | TGTAATGTTGTCCAGT | 21095 | 21110 | N/A | N/A | 1342 |
| 1059889 | ATGTGTAATGTTGTCC | 21098 | 21113 | N/A | N/A | 1333 |
| 1059890 | TATGTGTAATGTTGTC | 21099 | 21114 | N/A | N/A | 1930 |
| 1176128 | ATGTCCGACAGTGTCT | 19492 | 19507 | 1353 | 1368 | 1857 |
| 1176129 | AATGTCCGACAGTGTC | 19493 | 19508 | 1354 | 1369 | 1786 |
| 1176132 | CCGAATGTCCGACAGT | 19496 | 19511 | 1357 | 1372 | 1643 |
| 1176133 | CCCGAATGTCCGACAG | 19497 | 19512 | 1358 | 1373 | 2306 |
| 1176134 | TCCCGAATGTCCGACA | 19498 | 19513 | 1359 | 1374 | 1337 |
| 1176135 | TTCCCGAATGTCCGAC | 19499 | 19514 | 1360 | 1375 | 2299 |
| 1176138 | ACCTTCCCGAATGTCC | 19502 | 19517 | 1363 | 1378 | 1784 |
| 1176139 | CACCTTCCCGAATGTC | 19503 | 19518 | 1364 | 1379 | 2301 |
| 1176140 | GCACCTTCCCGAATGT | 19504 | 19519 | 1365 | 1380 | 2302 |
| 1176142 | AATGTTGTCCAGTAAT | 21092 | 21107 | N/A | N/A | 1783 |
| 1176143 | TAATGTTGTCCAGTAA | 21093 | 21108 | N/A | N/A | 1712 |
| 1176145 | GTATGTGTAATGTTGT | 21100 | 21115 | N/A | N/A | 1621 |
| 1184172 | GAATGTCCGACAGTGT | 19494 | 19509 | 1355 | 1370 | 1590 |
| 1213275 | TATGGCTGATTCAAAG | 19916 | 19931 | N/A | N/A | 1709 |
| 1213276 | ATGGCTGATTCAAAGA | 19915 | 19930 | N/A | N/A | 1639 |
| 1213282 | ATTGTTATATGGCTGA | 19923 | 19938 | N/A | N/A | 1638 |
| 1215869 | GTTATATGGCTGATTC | 19920 | 19935 | N/A | N/A | 1332 |
| 1215870 | TTGTTATATGGCTGAT | 19922 | 19937 | N/A | N/A | 1637 |
| 1241201 | CTTTTATTCGCGAGGG | 24775 | 24790 | 2833 | 2848 | 1335 |
| 1243719 | CGAATGTCCGACAGTG | 19495 | 19510 | 1356 | 1371 | 2298 |
| 1309459 | ACTTTGCGAACCAACG | 24447 | 24462 | 2505 | 2520 | 1563 |
| 1309468 | GGAATGTTAAACTGGG | 13962 | 13977 | N/A | N/A | 1520 |
| 1309469 | TCTTGTATCCTGTTGC | 17739 | 17754 | N/A | N/A | 1397 |
| 1309473 | TTGTATCCTGTTGCTT | 17737 | 17752 | N/A | N/A | 2210 |
| 1309475 | GGAATCTATCATGGCT | 20811 | 20826 | N/A | N/A | 1546 |
| 1309479 | AACATGTGTCAGTACA | 20981 | 20996 | N/A | N/A | 1842 |
| 1309482 | GGAGCGGTTGTGAACT | 24123 | 24138 | 2181 | 2196 | 2296 |
| 1309484 | GTTCTCTTAGACAAAG | 21053 | 21068 | N/A | N/A | 2275 |
| 1309485 | TCAGTAGTAGATGGGC | 17324 | 17339 | N/A | N/A | 1615 |

The modified oligonucleotides in the table below are 3-10-3 cEt modified oligonucleotides with mixed internucleoside linkages. The modified oligonucleotides in the table below are 16 nucleosides in length. The sugar motif for the modified oligonucleotides is (from 5′ to 3′): kkkddddddddddkkk; wherein each “d” represents a 2′-β-D-deoxyribosyl sugar moiety, and each “k” represents a cEt sugar moiety. The internucleoside linkage motifs for the modified oligonucleotides are presented in the column labeled “Internucleoside Linkages (5′ to 3′)” in the table below, wherein each “s” represents a phosphorothioate internucleoside linkage, each “o” represents a phosphodiester internucleoside linkage, and each “x” represents a methoxypropyl phosphonate internucleoside linkage. All cytosine nucleobases are 5-methylcytosines. Inosine nucleobases are represented by the letter “I” in the Nucleoside Sequence column in the table below. Further, the modified oligonucleotides in the table below are conjugated to a 6-palmitamidohexyl phosphate conjugate group (shown herein above) attached to the 5′-OH of the oligonucleotide.

TABLE 38
6-Palmitamidohexyl conjugated 3-10-3 cEt modified oligonucleotides with mixed internucleoside
linkages complementary to human DMPK

| | | SEQ | SEQ | SEQ | SEQ | | |
|---|---|---|---|---|---|---|---|
| | | ID | ID | ID | ID | | |
| | | NO: 1 | NO: 1 | NO: 2 | NO: 2 | Internucleoside | SEQ |
| Compound | Nucleobase Sequence | Start | Stop | Start | Stop | Linkages | ID |
| No. | (5′ to 3′) | Site | Site | Site | Site | (5′ to 3′) | NO |
| 1162310 | ACAATAAATACCGAGG | 24730 | 24745 | 2788 | 2803 | soossssssssssos | 1336 |
| 1162311 | ACAATAAATACCGAGG | 24730 | 24745 | 2788 | 2803 | sosssssssssssos | 1336 |
| 1162638 | TTCGCGAGGGTCGGGG | 24769 | 24784 | 2827 | 2842 | soossssssssssos | 1490 |
| 1162641 | TTATTCGCGAGGGTCG | 24772 | 24787 | 2830 | 2845 | soossssssssssos | 1859 |
| 1162643 | TTTTATTCGCGAGGGT | 24774 | 24789 | 2832 | 2847 | soossssssssssos | 1860 |
| 1162645 | CCTTTTATTCGCGAGG | 24776 | 24791 | 2834 | 2849 | soossssssssssos | 2331 |
| 1162647 | GGCCTTTTATTCGCGA | 24778 | 24793 | 2836 | 2851 | soossssssssssos | 2333 |
| 1162654 | TTATTCGCGAGGGTCG | 24772 | 24787 | 2830 | 2845 | sosssssssssssos | 1859 |
| 1162655 | TTTATTCGCGAGGGTC | 24773 | 24788 | 2831 | 2846 | sosssssssssssos | 1711 |
| 1162658 | CCTTTTATTCGCGAGG | 24776 | 24791 | 2834 | 2849 | sosssssssssssos | 2331 |
| 1176163 | ATGTITAATGTTGTCC | 21098 | 21113 | N/A | N/A | soossssssssssos | 2157 |
| 1176164 | ATGTGTAATGTTGTCC | 21098 | 21113 | N/A | N/A | soosxssssssssos | 1333 |
| 1213273 | TGGCTGATTCAAAGAA | 19914 | 19929 | N/A | N/A | ooooxoooooooooo | 1489 |
| 1309477 | AGGAACAAATCAGGAT | 22206 | 22221 | N/A | N/A | sssssxsssssssss | 1419 |

The modified oligonucleotides in the table below are 16 nucleosides in length. The sugar motif for the modified oligonucleotides is (from 5′ to 3′): eekkddddddddkkee; wherein each “d” represents a 2′-β-D-deoxyribosyl sugar moiety, each “e” represents a 2′-MOE sugar moiety, and each “k” represents a cEt sugar moiety. The internucleoside linkage motifs for the modified oligonucleotides are presented in the column labeled “Internucleoside Linkages (5′ to 3′)” in the table below, wherein each “s” represents a phosphorothioate internucleoside linkage, each “o” represents a phosphodiester internucleoside linkage, and each “x” represents a methoxypropyl phosphonate internucleoside linkage. All cytosine nucleobases are 5-methylcytosines. Further, the modified oligonucleotides in the table below are conjugated to a 6-palmitamidohexyl phosphate conjugate group (shown herein above) attached to the 5′-OH of the oligonucleotide.

TABLE 39
6-Palmitamidohexyl conjugated modified oligonucleotides with a mixed sugar motif and mixed
internucleoside linkages complementary to human DMPK

| | | SEQ | SEQ | SEQ | SEQ | | |
|---|---|---|---|---|---|---|---|
| | | ID | ID | ID | ID | | |
| | | NO: 1 | NO: 1 | NO: 2 | NO: 2 | Internucleoside | SEQ |
| Compound | Nucleobase Sequence | Start | Stop | Start | Stop | Linkages | ID |
| No. | (5′ to 3′) | Site | Site | Site | Site | (5′ to 3′) | NO |
| 1046939 | CCCGAATGTCCGACAG | 19497 | 19512 | 1358 | 1373 | sssssssssssssss | 2306 |
| 1176125 | TCCCGAATGTCCGACA | 19498 | 19513 | 1359 | 1374 | sssssxsssssssss | 1337 |
| 1176126 | CCCGAATGTCCGACAG | 19497 | 19512 | 1358 | 1373 | soossxsssssssos | 2306 |
| 1176127 | CCCGAATGTCCGACAG | 19497 | 19512 | 1358 | 1373 | sssssxsssssssss | 2306 |

The modified oligonucleotides in the table below are 16 nucleosides in length. The sugar motif for the modified oligonucleotides is (from 5′ to 3′): ekkddddddddddkke; wherein each “d” represents a 2′-β-D-deoxyribosyl sugar moiety, each “e” represents a 2′-MOE sugar moiety, and each “k” represents a cEt sugar moiety. The internucleoside linkage motif for the modified oligonucleotides is (from 5′ to 3′): sssssssssssssss; wherein each “s” represents a phosphorothioate internucleoside linkage. All cytosine nucleobases are 5-methylcytosines. Further, the modified oligonucleotides in the table below are conjugated to a 6-palmitamidohexyl phosphate conjugate group (shown herein above) attached to the 5′-OH of the oligonucleotide.

TABLE 40
6-Palmitamidohexyl conjugated modified oligonucleotides with a mixed sugar
motif and uniform phosphorothioate internucleoside linkages complementary
to human DMPK

| | | SEQ | SEQ | SEQ | SEQ | |
|---|---|---|---|---|---|---|
| | | ID | ID | ID | ID | |
| | | NO: 1 | NO: 1 | NO: 2 | NO: 2 | SEQ |
| Compound | Nucleobase Sequence | Start | Stop | Start | Stop | ID |
| No. | (5′ to 3′) | Site | Site | Site | Site | NO |
| 1046948 | TGTAATGTTGTCCAGT | 21095 | 21110 | N/A | N/A | 1342 |
| 1046952 | TATGTGTAATGTTGTC | 21099 | 21114 | N/A | N/A | 1930 |
| 1207072 | GTTATATGGCTGATTC | 19920 | 19935 | N/A | N/A | 1332 |
| 1207074 | TTGTTATATGGCTGAT | 19922 | 19937 | N/A | N/A | 1637 |
| 1207075 | ATTGTTATATGGCTGA | 19923 | 19938 | N/A | N/A | 1638 |
| 1240824 | CTTTTATTCGCGAGGG | 24775 | 24790 | 2833 | 2848 | 1335 |

The modified oligonucleotides in the table below are 16 nucleosides in length. The sugar motif for the modified oligonucleotides is (from 5′ to 3′): kkeddddddddddkkk; wherein each “d” represents a 2′-β-D-deoxyribosyl sugar moiety, each “e” represents a 2′-MOE sugar moiety, and each “k” represents a cEt sugar moiety. The internucleoside linkage motif for the modified oligonucleotides is (from 5′ to 3′): sssssssssssssss; wherein each “s” represents a phosphorothioate internucleoside linkage. All cytosine nucleobases are 5-methylcytosines. Further, the modified oligonucleotides in the table below are conjugated to a 6-palmitamidohexyl phosphate conjugate group (shown herein above) attached to the 5′-OH of the oligonucleotide.

TABLE 41
6-Palmitamidohexyl conjugated modified oligonucleotides with a mixed
sugar motif and uniform phosphorothioate internucleoside linkages
complementary to human DMPK

| | | SEQ | SEQ | SEQ | SEQ | |
|---|---|---|---|---|---|---|
| | | ID | ID | ID | ID | |
| | | NO: 1 | NO: 1 | NO: 2 | NO: 2 | SEQ |
| Compound | Nucleobase Sequence | Start | Stop | Start | Stop | ID |
| No. | (5′ to 3′) | Site | Site | Site | Site | NO |
| 1273292 | GTTATATGGCTGATTC | 19920 | 19935 | N/A | N/A | 1332 |
| 1370498 | AACATGTGTCAGTACA | 20981 | 20996 | N/A | N/A | 1842 |
| 1370504 | GGAGCGGTTGTGAACT | 24123 | 24138 | 2181 | 2196 | 2296 |
| 1370510 | TGTAATGTTGTCCAGT | 21095 | 21110 | N/A | N/A | 1342 |

The modified oligonucleotides in the table below are 16 nucleosides in length. The sugar motif for the modified oligonucleotides is (from 5′ to 3′): kkkdyddddddddkkk; wherein each “d” represents a 2′-β-D-deoxyribosyl sugar moiety, each “y” represents a 2′-O-methylribosyl sugar moiety, and each “k” represents a cEt sugar moiety. The internucleoside linkage motifs for the modified oligonucleotides are presented in the column labeled “Internucleoside Linkages (5′ to 3′)” in the table below, wherein each “s” represents a phosphorothioate internucleoside linkage, and each “o” represents a phosphodiester internucleoside linkage. All cytosine nucleobases are 5-methylcytosines. Further, the modified oligonucleotides in the table below are conjugated to a 6-palmitamidohexyl phosphate conjugate group (shown herein above) attached to the 5′-OH of the oligonucleotide.

TABLE 42
6-Palmitamidohexyl conjugated modified oligonucleotides with a mixed sugar
motif and mixed internucleoside linkages complementary to human DMPK

| | | SEQ | SEQ | | |
|---|---|---|---|---|---|
| | | ID | ID | | |
| | | NO: 1 | NO: 1 | Internucleoside | SEQ |
| Compound | Nucleobase Sequence | Start | Stop | Linkages | ID |
| No. | (5′ to 3′) | Site | Site | (5′ to 3′) | NO |
| 1064823 | ATGTGTAATGTTGTCC | 21098 | 21113 | sssssssssssssss | 1333 |
| 1064824 | TATGUGTAATGTTGTC | 21099 | 21114 | sssssssssssssss | 1488 |
| 1176165 | ATGTGTAATGTTGTCC | 21098 | 21113 | soossssssssssos | 1333 |
| 1273301 | GTTAUATGGCTGATTC | 19920 | 19935 | sssssssssssssss | 2303 |

The modified oligonucleotides in the table below are 16 nucleosides in length. The sugar motifs for the modified oligonucleotides in the table below are presented in the column labeled “Sugar Motif (5′ to 3′),” wherein each “d” represents a 2′-β-D-deoxyribosyl sugar moiety, each “e” represents a 2′-MOE sugar moiety, and each “k” represents a cEt sugar moiety. The internucleoside linkage motif for the modified oligonucleotides is (from 5′ to 3′): sssssssssssssss; wherein each “s” represents a phosphorothioate internucleoside linkage. All cytosine nucleobases are 5-methylcytosines. Further, the modified oligonucleotides in the table below are conjugated to a 6-palmitamidohexyl phosphate conjugate group (shown herein above) attached to the 5′-OH of the oligonucleotide.

TABLE 43
6-Palmitamidohexyl conjugated modified oligonucleotides with mixed sugar motifs and uniform
phosphorothioate internucleoside linkages complementary to human DMPK

| | | SEQ | SEQ | SEQ | SEQ | | |
|---|---|---|---|---|---|---|---|
| | | ID | ID | ID | ID | | |
| | | NO: 1 | NO: 1 | NO: 2 | NO: 2 | | SEQ |
| Compound | Nucleobase Sequence | Start | Stop | Start | Stop | Sugar Motif | ID |
| No. | (5′ to 3′) | Site | Site | Site | Site | (5′ to 3′) | NO |
| 1207130 | GTTATATGGCTGATTC | 19920 | 19935 | N/A | N/A | ekkkddddddddkkke | 1332 |
| 1240825 | CTTTTATTCGCGAGGG | 24775 | 24790 | 2833 | 2848 | ekkddddddddddkkk | 1335 |
| 1240826 | CTTTTATTCGCGAGGG | 24775 | 24790 | 2833 | 2848 | kkkddddddddddkke | 1335 |
| 1273291 | GTTATATGGCTGATTC | 19920 | 19935 | N/A | N/A | kekddddddddddkkk | 1332 |
| 1273297 | GTTATATGGCTGATTC | 19920 | 19935 | N/A | N/A | kkkedddddddddkkk | 1332 |
| 1370496 | TGTAATGTTGTCCAGT | 21095 | 21110 | N/A | N/A | kekddddddddddkkk | 1342 |
| 1370503 | GGAGCGGTTGTGAACT | 24123 | 24138 | 2181 | 2196 | kkkedddddddddkkk | 2296 |
| 1370505 | ATGTGTAATGTTGTCC | 21098 | 21113 | N/A | N/A | kkkedddddddddkkk | 1333 |
| 1370507 | GGAGCGGTTGTGAACT | 24123 | 24138 | 2181 | 2196 | kekddddddddddkkk | 2296 |

The modified oligonucleotides in the table below are 16 nucleosides in length. The sugar motifs for the modified oligonucleotides in the table below are presented in the column labeled “Sugar Motif (5′ to 3′),” wherein each “d” represents a 2′-β-D-deoxyribosyl sugar moiety, each “e” represents a 2′-MOE sugar moiety, each “k” represents a cEt sugar moiety, each “[5′-(S)-Me-d]” represents a 5′-(S)-methyl-β-D-2′-deoxyribosyl sugar moiety, each “[5′-(R)-Me-d]” represents a 5′-(R)-methyl-β-D-2′-deoxyribosyl sugar moiety, and each “[5′-(R)-allyl-d]” represents a 5′-(R)-allyl-β-D-2′-deoxyribosyl sugar moiety. The internucleoside linkage motif for the modified oligonucleotides is (from 5′ to 3′): sssssssssssssss; wherein each “s” represents a phosphorothioate internucleoside linkage. All cytosine nucleobases are 5-methylcytosines. Further, the modified oligonucleotides in the table below are conjugated to a 6-palmitamidohexyl phosphate conjugate group (shown herein above) attached to the 5′-OH of the oligonucleotide.

TABLE 44
6-Palmitamidohexyl conjugated modified oligonucleotides with uniform phosphorothioate
internucleoside linkages complementary to human DMPK

| | | SEQ | SEQ | | |
|---|---|---|---|---|---|
| | | ID | ID | | |
| | | NO: 1 | NO: 1 | | |
| Compound | Nucleobase Sequence | Start | Stop | | SEQ ID |
| No. | (5′ to 3′) | Site | Site | Sugar Motif (5′ to 3′) | NO |
| 1176148 | TGTAATGTTGTCCAGT | 21095 | 21110 | kkkdd[5′-(S)-Me-d]dddddddkkk | 1342 |
| 1176151 | TGTAATGTTGTCCAGT | 21095 | 21110 | kkkdd[5′-(R)-Me-d]dddddddkkk | 1342 |
| 1176153 | TGTAATGTTGTCCAGT | 21095 | 21110 | kkkdd[5′-(R)-allyl-d]dddddddkkk | 1342 |
| 1176157 | ATGTGTAATGTTGTCC | 21098 | 21113 | kkkdd[5′-(S)-Me-d]dddddddkkk | 1333 |
| 1176159 | ATGTGTAATGTTGTCC | 21098 | 21113 | kkkdd[5′-(R)-Me-d]dddddddkkk | 1333 |
| 1176160 | ATGTGTAATGTTGTCC | 21098 | 21113 | kkkddd[5′-(R)-Me-d]ddddddkkk | 1333 |
| 1176161 | ATGTGTAATGTTGTCC | 21098 | 21113 | kkkdd[5′-(R)-allyl-d]dddddddkkk | 1333 |

### Example 8: Tolerability of Modified Oligonucleotides Complementary to Human DMPK in Rats, 3-Hour Study

Modified oligonucleotides described herein above were tested in rats to assess the tolerability of the oligonucleotides. Sprague Dawley rats each received a single intrathecal (IT) dose of modified oligonucleotide at 3 mg. Each treatment group consisted of 3-4 rats. A group of 3-4 rats received PBS as a negative control. Each experiment is identified in separate tables below. At 3 hours post-injection, movement in 7 different parts of the body were evaluated for each rat. The 7 body parts are (1) the rat's tail; (2) the rat's posterior posture; (3) the rat's hind limbs; (4) the rat's hind paws; (5) the rat's forepaws; (6) the rat's anterior posture; (7) the rat's head. For each of the 7 different body parts, each rat was given a sub-score of 0 if the body part was moving or 1 if the body part was paralyzed (the functional observational battery score or FOB). After each of the 7 body parts were evaluated, the sub-scores were summed for each rat and then averaged for each group. For example, if a rat's tail, head, and all other evaluated body parts were moving 3 hours after the 3 mg IT dose, it would get a summed score of 0. If another rat was not moving its tail 3 hours after the 3 mg IT dose but all other evaluated body parts were moving, it would receive a score of 1. Results are presented as the average score for each treatment group.

TABLE 45
Tolerability scores in rats

| Compound | 3 hr. |
|---|---|
| No. | FOB |
| PBS | 0.00 |
| 1052866 | 2.50 |
| 1052867 | 1.75 |
| 1052869 | 2.75 |
| 1380456 | 4.75 |
| 1380987 | 1.50 |
| 1380993 | 4.00 |
| 1381037 | 3.00 |
| 1381255 | 0.00 |
| 1381275 | 2.50 |
| 1381363 | 1.00 |
| 1381456 | 0.00 |
| 1381470 | 0.50 |
| 1381668 | 0.50 |
| 1459314 | 4.25 |
| 1459315 | 3.25 |

TABLE 46
Tolerability scores in rats

| Compound | 3 hr. |
|---|---|
| No. | FOB |
| PBS | 0.25 |
| 1459320 | 2.50 |
| 1459322 | 5.00 |
| 1459323 | 1.75 |
| 1459324 | 3.00 |
| 1459325 | 1.75 |
| 1459326 | 2.75 |
| 1459327 | 0.75 |
| 1459328 | 1.50 |
| 1459329 | 3.25 |
| 1459330 | 3.00 |

TABLE 47
Tolerability scores in rats

| Compound | 3 hr. |
|---|---|
| No. | FOB |
| PBS | 0.00 |
| 1459343 | 1.25 |
| 1459344 | 0.75 |
| 1459345 | 3.00 |
| 1459346 | 2.25 |
| 1459348 | 0.25 |
| 1459349 | 0.00 |
| 1459351 | 3.00 |
| 1459352 | 0.00 |
| 1459353 | 0.00 |
| 1459354 | 0.00 |
| 1459355 | 3.00 |

TABLE 48
Tolerability scores in rats

| Compound No. | 3 hr. FOB |
|---|---|
| PBS | 0.00 |
| 1459356 | 2.00 |
| 1459359 | 0.00 |
| 1459360 | 0.67 |
| 1459364 | 0.00 |
| 1459365 | 1.33 |
| 1459366 | 2.00 |
| 1459367 | 0.00 |
| 1459368 | 1.33 |
| 1459369 | 0.00 |
| 1459372 | 0.00 |
| 1459373 | 3.00 |
| 1459374 | 1.33 |
| 1459375 | 2.00 |

TABLE 49
Tolerability scores in rats

| Compound No. | 3 hr. FOB |
|---|---|
| PBS | 0.00 |
| 1459377 | 2.00 |
| 1459378 | 2.00 |
| 1459379 | 1.67 |
| 1459383 | 0.00 |
| 1459386 | 3.00 |
| 1459387 | 2.33 |
| 1459392 | 1.67 |
| 1459393 | 2.00 |
| 1459394 | 2.00 |
| 1459395 | 0.00 |

TABLE 50
Tolerability scores in rats

| Compound No. | 3 hr. FOB |
|---|---|
| PBS | 0.00 |
| 1459396 | 0.00 |
| 1459398 | 0.00 |
| 1459399 | 0.00 |
| 1459400 | 2.67 |
| 1459401 | 3.00 |
| 1459402 | 3.00 |

TABLE 51
Tolerability scores in rats

| Compound No. | 3 hr. FOB |
|---|---|
| PBS | 0.00 |
| 1459406 | 1.67 |
| 1459407 | 0.00 |
| 1459408 | 0.67 |
| 1459409 | 1.67 |
| 1459412 | 0.67 |
| 1459413 | 3.00 |
| 1459415 | 1.33 |
| 1459417 | 2.33 |
| 1459418 | 0.67 |
| 1459419 | 0.33 |
| 1459420 | 0.67 |
| 1459421 | 2.00 |
| 1459422 | 0.33 |
| 1459423 | 0.67 |

‡ indicates that fewer than 3 samples were available

TABLE 52
Tolerability scores in rats

| Compound No. | 3 hr. FOB |
|---|---|
| PBS | 0.00 |
| 1459424 | 0.67 |
| 1459425 | 2.00 |
| 1459426 | 1.33 |
| 1459427 | 1.33 |
| 1459428 | 0.00 |
| 1459429 | 4.33‡ |
| 1459433 | 0.00 |
| 1459434 | 0.00 |
| 1459435 | 0.00 |
| 1459436 | 2.00 |
| 1459437 | 2.00 |
| 1459438 | 5.67‡ |
| 1459439 | 0.00 |

‡indicates that fewer than 3 samples were available

TABLE 53
Tolerability scores in rats

| Compound No. | 3 hr. FOB |
|---|---|
| PBS | 0.00 |
| 1459440 | 0.33 |
| 1459441 | 0.00 |
| 1459442 | 1.67 |
| 1459443 | 2.33 |
| 1459444 | 1.00 |
| 1459445 | 1.67 |
| 1459446 | 1.67 |
| 1459449 | 0.00 |
| 1459450 | 0.00 |
| 1459451 | 0.00 |
| 1459452 | 2.67 |
| 1459453 | 2.00 |
| 1459455 | 2.00 |
| 1459456 | 0.33 |
| 1459457 | 0.00 |
| 1459458 | 0.00 |
| 1459459 | 3.00 |

TABLE 54
Tolerability scores in rats

| Compound No. | 3 hr. FOB |
|---|---|
| PBS | 0.00 |
| 1459461 | 0.00 |
| 1459462 | 3.00 |
| 1459463 | 0.00 |
| 1459464 | 3.00 |
| 1459465 | 2.67 |
| 1459973 | 0.00 |
| 1459974 | 3.33 |
| 1459975 | 4.00 |
| 1459976 | 2.33 |
| 1459977 | 2.67 |
| 1459978 | 5.33 |
| 1459979 | 3.00 |
| 1459980 | 5.00 |
| 1459981 | 3.33 |
| 1459982 | 4.67 |
| 1459983 | 4.33 |
| 1459985 | 2.00 |

TABLE 55
Tolerability scores in rats

| Compound No. | 3 hr. FOB |
|---|---|
| PBS | 0.25 |
| 1459986 | 4.50‡ |
| 1459987 | 3.33 |
| 1459988 | 4.00‡ |
| 1459989 | 4.00‡ |
| 1459990 | 4.33 |
| 1460162 | 2.33 |
| 1460163 | 3.00 |
| 1460164 | 2.00 |
| 1460166 | 1.33 |
| 1460168 | 1.25 |
| 1460170 | 0.00 |
| 1460171 | 0.67 |
| 1460174 | 6.00 |
| 1460178 | 1.33 |

‡indicates that fewer than 3 samples were available

TABLE 56
Tolerability scores in rats

| Compound No. | 3 hr. FOB |
|---|---|
| PBS | 0.00 |
| 1380378 | 3.33 |
| 1380457 | 6.00 |
| 1380571 | 3.33 |
| 1380679 | 3.00 |
| 1380686 | 5.00‡ |
| 1380815 | 2.00 |
| 1380870 | 5.33 |
| 1380962 | 5.00 |
| 1381486 | 1.50 |
| 1381507 | 1.00 |
| 1381603 | 4.00 |
| 1381621 | 2.67 |
| 1460179 | 0.67 |
| 1460184 | 2.67 |
| 1460185 | 0.33 |

‡indicates that fewer than 3 samples were available

TABLE 57
Tolerability scores in rats

| Compound No. | 3 hr. FOB |
|---|---|
| 1052879 | 5.50‡ |
| 1380380 | 4.00 |
| 1380659 | 5.67 |
| 1381513 | 1.33 |
| 1459991 | 4.00 |

‡indicates that fewer than 3 samples were available

TABLE 58
Tolerability scores in rats

| Compound No. | 3 hr. FOB |
|---|---|
| PBS | 0.00 |
| 1052868 | 2.00‡ |
| 1052877 | 5.00‡ |
| 1380469 | 3.00 |
| 1380652 | 1.00 |
| 1380821 | 3.00 |
| 1380847 | 3.67 |
| 1380868 | 3.00 |
| 1381223 | 3.00 |
| 1381285 | 0.00 |
| 1381524 | 3.00 |
| 1381657 | 1.00 |
| 1381677 | 0.00 |

‡indicates that fewer than 3 samples were available

TABLE 59
Tolerability scores in rats

| Compound No. | 3 hr. FOB |
|---|---|
| PBS | 0.25 |
| 1052877 | 3.25 |
| 1380679 | 4.25 |
| 1381668 | 1.50 |
| 1459328 | 1.25 |
| 1459329 | 1.75 |
| 1459374 | 0.50 |
| 1459375 | 0.25 |
| 1459442 | 2.50 |
| 1459443 | 2.25 |
| 1511084 | 1.25 |

### Example 9: Tolerability of Modified Oligonucleotides Complementary to Human DMPK in Wild-Type Mice, 3-Hour Study

Modified oligonucleotides described herein above were tested in wild-type female C57/B16 mice to assess the tolerability of the oligonucleotides. Wild-type female C57/B16 mice each received a single ICV dose of modified oligonucleotide at 350, 500 or 700 μg indicated in the tables below. Each treatment group consisted of 2-4 mice. A group of 3-4 mice received PBS as a negative control for each experiment. Each experiment is identified in separate tables below. At 3 hours post-injection, mice were evaluated according to seven different criteria. The criteria are (1) the mouse was bright, alert, and responsive; (2) the mouse was standing or hunched without stimuli; (3) the mouse showed any movement without stimuli; (4) the mouse demonstrated forward movement after it was lifted; (5) the mouse demonstrated any movement after it was lifted; (6) the mouse responded to tail pinching; (7) regular breathing. For each of the 7 criteria, a mouse was given a subscore of 0 if it met the criteria and 1 if it did not (the functional observational battery score or FOB). After all 7 criteria were evaluated, the scores were summed for each mouse and averaged within each treatment group. The results are presented in the tables below.

TABLE 60
Tolerability scores in mice at a dose of 500 μg

| Compound No. | 3 hr. FOB |
|---|---|
| PBS | 0.00 |
| 1052863 | 2.50 |
| 1052864 | 4.00 |
| 1052865 | 2.50 |
| 1052866 | 5.50 |
| 1052867 | 2.00 |
| 1052868 | 5.00 |
| 1052869 | 4.50 |
| 1052870 | 4.25 |
| 1052871 | 4.00 |
| 1052873 | 4.00 |
| 1052874 | 6.00 |
| 1052875 | 2.75 |
| 1052876 | 5.50 |
| 1052877 | 5.50 |
| 1052879 | 5.75 |
| 1052881 | 5.25 |
| 1052883 | 6.00 |
| 1052886 | 5.75 |
| 1052887 | 6.00 |
| 1052889 | 5.25 |

TABLE 61
Tolerability scores in mice at a dose of 700 μg

| Compound No. | 3 hr. FOB |
|---|---|
| PBS | 0.00 |
| 1052866 | 1.33 |
| 1052867 | 6.00 |
| 1052869 | 2.00 |
| 1380456 | 3.00 |
| 1380987 | 1.00 |
| 1380993 | 3.00 |
| 1381037 | 2.33 |
| 1381255 | 1.00 |
| 1381363 | 0.67 |
| 1381456 | 0.00 |
| 1381470 | 0.00 |
| 1381668 | 0.00 |
| 1459314 | 5.33 |
| 1459315 | 4.00 |
| 1459320 | 2.00 |
| 1459322 | 3.67 |
| 1459323 | 1.67 |
| 1459324 | 3.33 |
| 1459325 | 2.00 |
| 1459326 | 2.00 |
| 1459327 | 1.33 |
| 1459328 | 1.33 |
| 1459329 | 2.33 |
| 1459330 | 2.00 |
| 1459343 | 1.67 |
| 1459344 | 0.33 |
| 1459345 | 2.33 |
| 1459346 | 2.00 |
| 1459348 | 0.00 |
| 1459349 | 0.67 |
| 1459351 | 4.67 |
| 1459352 | 0.33 |
| 1459353 | 1.00 |
| 1459354 | 0.00 |
| 1459355 | 3.00 |
| 1459356 | 2.00 |
| 1459359 | 2.00 |
| 1459360 | 0.67 |

TABLE 62
Tolerability scores in mice at a dose of 700 μg

| Compound No. | 3 hr. FOB |
|---|---|
| PBS | 0.00 |
| 1459364 | 0.33 |
| 1459365 | 1.00 |
| 1459366 | 1.00 |
| 1459367 | 1.00 |
| 1459368 | 1.00 |
| 1459369 | 1.00 |
| 1459372 | 3.00 |
| 1459373 | 3.33 |
| 1459374 | 2.00 |
| 1459375 | 1.00 |
| 1459377 | 5.00 |
| 1459378 | 3.67 |
| 1459379 | 1.33 |
| 1459383 | 0.67 |
| 1459386 | 3.33 |
| 1459387 | 1.67 |
| 1459392 | 0.33 |
| 1459393 | 0.00 |
| 1459394 | 2.00 |

TABLE 63
Tolerability scores in mice at a dose of 700 μg

| Compound | |
|---|---|
| No. | 3 hr. FOB |
| PBS | 0.00 |
| 1459419 | 0.00 |
| 1459420 | 0.33 |
| 1459421 | 0.00 |
| 1459422 | 0.33 |
| 1459423 | 0.00 |
| 1459424 | 0.00 |
| 1459425 | 3.00 |
| 1459426 | 1.00 |
| 1459427 | 0.00 |
| 1459428 | 0.00 |
| 1459429 | 2.00 |
| 1459433 | 0.67 |
| 1459434 | 0.00 |
| 1459435 | 1.00 |
| 1459436 | 2.33 |
| 1459437 | 1.00 |
| 1459438 | 4.00 |
| 1459439 | 0.00 |
| 1459440 | 0.00 |
| 1459441 | 0.00 |
| 1459442 | 3.00 |
| 1459443 | 2.00 |
| 1459444 | 1.00 |
| 1459445 | 0.67 |
| 1459446 | 2.33 |
| 1459449 | 0.67 |
| 1459450 | 0.33 |
| 1459451 | 0.33 |
| 1459452 | 2.33 |
| 1459453 | 1.33 |
| 1459455 | 3.00 |
| 1459456 | 0.00 |
| 1459457 | 0.00 |
| 1459458 | 1.00 |
| 1459459 | 2.67 |
| 1459461 | 1.00 |
| 1459462 | 2.33 |
| 1459463 | 1.00 |
| 1459464 | 2.00 |

TABLE 64
Tolerability scores in mice at a dose of 700 μg

| Compound | |
|---|---|
| No. | 3 hr. FOB |
| PBS | 0.00 |
| 1380378 | 4.00 |
| 1380457 | 5.00 |
| 1380571 | 5.00 |
| 1380679 | 3.33 |
| 1380686 | 5.00 |
| 1380815 | 0.00 |
| 1380870 | 5.00 |
| 1380962 | 4.00 |
| 1381486 | 3.00 |
| 1381507 | 0.00 |
| 1381603 | 2.00 |
| 1381621 | 1.00 |
| 1459465 | 3.67 |
| 1459973 | 0.00 |
| 1459974 | 4.00 |
| 1459975 | 2.67 |
| 1459976 | 1.67 |
| 1459977 | 1.33 |
| 1459978 | 4.00 |
| 1459979 | 4.00 |
| 1459980 | 5.00 |
| 1459981 | 5.33 |
| 1459982 | 3.67 |
| 1459983 | 3.67 |
| 1459985 | 2.00 |
| 1459986 | 4.33 |
| 1459987 | 2.67 |
| 1459988 | 4.00 |
| 1459989 | 3.33 |
| 1459990 | 4.00 |
| 1459991 | 5.00 |
| 1460162 | 3.00 |
| 1460163 | 1.33 |
| 1460164 | 1.00 |
| 1460166 | 0.33 |
| 1460168 | 1.33 |
| 1460170 | 0.00 |
| 1460171 | 0.00 |
| 1460174 | 5.00 |
| 1460178 | 0.67 |
| 1460179 | 1.00 |
| 1460184 | 1.00 |
| 1460185 | 0.00 |

TABLE 65
Tolerability scores in mice at a dose of 700 μg

| Compound | |
|---|---|
| No. | 3 hr. FOB |
| PBS | 0.00 |
| 1052868 | 2.00 |
| 1052879 | 5.33 |
| 1380380 | 1.67 |
| 1380469 | 5.33 |
| 1380652 | 0.00 |
| 1380659 | 4.67 |
| 1380821 | 1.67 |
| 1380847 | 3.00 |
| 1380868 | 1.00 |
| 1381223 | 2.33 |
| 1381285 | 0.33 |
| 1381513 | 0.00 |
| 1381524 | 2.00 |
| 1381657 | 1.00 |
| 1381677 | 1.00 |
| 1459395 | 1.00 |
| 1459396 | 1.00 |
| 1459398 | 0.00 |
| 1459399 | 0.00 |
| 1459400 | 2.00 |
| 1459401 | 1.00 |
| 1459402 | 1.00 |
| 1459406 | 1.00 |
| 1459407 | 1.00 |
| 1459408 | 0.67 |
| 1459409 | 1.00 |
| 1459412 | 1.33 |
| 1459413 | 1.00 |
| 1459415 | 1.00 |
| 1459417 | 1.00 |
| 1459418 | 1.00 |

TABLE 66
Tolerability scores in mice at a dose of 700 μg

| Compound | |
|---|---|
| No. | 3 hr. FOB |
| PBS | 0.00‡ |
| 1052877 | 5.67 |

‡indicates that fewer than 3 samples were available

TABLE 67
Tolerability scores in mice at a dose of 700 μg

| Compound | 3 hr. |
|---|---|
| No. | FOB |
| PBS | 0.00 |
| 1052877 | 5.75 |
| 1380679 | 3.50 |
| 1381668 | 0.00 |
| 1459328 | 0.00 |
| 1459329 | 2.50 |
| 1459374 | 0.75 |
| 1459375 | 1.00 |
| 1459442 | 3.25 |
| 1459443 | 2.25 |
| 1511084 | 0.75 |

TABLE 68
Tolerability scores in mice at a dose of 350 μg

| Compound | |
|---|---|
| No. | 3 hr. FOB |
| PBS | 0.00 |
| 1511076 | 3.00 |
| 1511078 | 6.00 |
| 1511079 | 0.00 |
| 1511080 | 1.00 |
| 1511081 | 0.00 |
| 1511082 | 0.00 |
| 1511083 | 0.50 |
| 1511084 | 0.50 |
| 1511085 | 0.00 |
| 1511086 | 0.50 |
| 1511087 | 0.50 |
| 1511088 | 0.00 |
| 1511089 | 0.00 |
| 1511090 | 6.00 |
| 1511091 | 1.00 |
| 1511092 | 0.00 |
| 1511093 | 0.00 |
| 1511094 | 0.00 |
| 1511095 | 1.00 |
| 1511096 | 0.00 |
| 1511097 | 0.50 |
| 1511098 | 0.00 |
| 1511099 | 0.50 |
| 1511100 | 0.50 |
| 1511101 | 0.50 |
| 1511102 | 0.00 |
| 1511103 | 0.00 |
| 1511104 | 0.00 |
| 1511105 | 1.00 |
| 1511106 | 1.00 |
| 1511107 | 2.00 |
| 1380987 | 1.50 |
| 1459415 | 0.50 |
| 1459980 | 3.50 |
| 1459373 | 1.00 |
| 1459973 | 0.00 |

### Example 10: Tolerability of Modified Oligonucleotides Complementary to Human DMPK in Wild-Type Mice, 3-Hour Study

The modified oligonucleotide described herein above was tested in wild-type female C57/B16 mice to assess the tolerability of the oligonucleotide. Wild-type female C57/B16 mice each received a single ICV dose of modified oligonucleotide at various doses as indicated in the tables below. Each treatment group consisted of 4 mice. A group of 4 mice received PBS as a negative control. At 3 hours post-injection, mice were evaluated according to seven different criteria. The criteria are (1) the mouse was bright, alert, and responsive; (2) the mouse was standing or hunched without stimuli; (3) the mouse showed any movement without stimuli; (4) the mouse demonstrated forward movement after it was lifted; (5) the mouse demonstrated any movement after it was lifted; (6) the mouse responded to tail pinching; (7) regular breathing. For each of the 7 criteria, a mouse was given a subscore of 0 if it met the criteria and 1 if it did not (the functional observational battery score or FOB). After all 7 criteria were evaluated, the scores were summed for each mouse and averaged within each treatment group. The results are presented in the table below.

Compound No. 486178 is described herein above.

TABLE 69
Tolerability scores in mice

| Compound | | |
|---|---|---|
| No. | Dose (μg) | 3 hr. FOB |
| PBS | N/A | 0.00 |
| 486178 | 10 | 0.00 |
| | 50 | 3.00 |
| | 100 | 4.75 |

TABLE 70
Tolerability scores in mice

| Compound | | |
|---|---|---|
| No. | Dose (μg) | 3 hr. FOB |
| PBS | N/A | 0.00 |
| 1338115 | 100 | 5.75 |
| | 200 | 6.75 |
| 1400761 | 100 | 5.50 |

### Example 11: Activity of Modified Oligonucleotides Complementary to Human DMPK in DMSXL Transgenic Mice

Modified oligonucleotides described above were tested in DMSXL transgenic mice previously described in Huguet A, et. al, Molecular, Physiological, and Motor Performance Defects in DMSXL Mice Carrying >1,000 CTG Repeats from the Human DM1 Locus; PLOS Genetics, 2012, vol. 8, no. 11: e1003043.

### Treatment

DMSXL transgenic mice were divided into groups of 1-2 mice each. The number of animals treated in each group is indicated in the tables below in the column labeled “n”. Each mouse received a single ICV bolus of 100 or 350 μg of modified oligonucleotide as indicated in the tables below. A group of 2-4 mice received PBS as a negative control.

### RNA Analysis

Two weeks post treatment, mice were sacrificed, and RNA was extracted from cortical brain tissue, spinal cord, and hippocampus for RTPCR analysis to measure amount of DMPK RNA using human primer probe set RTS38096 (forward nucleobase sequence TTTACACCGGATTTCGAAGGT, designated herein as SEQ ID NO: 9; reverse nucleobase sequence CGAATGTCCGACAGTGTCTC, designated herein as SEQ ID NO: 10; probe nucleobase sequence TCCTCCACCAAGTCGAAGTTGCAT, designated herein as SEQ ID NO: 11). Results are presented as percent human DMPK RNA relative to PBS control, normalized to mouse GAPDH (% control). Mouse GAPDH was amplified using mouse primer probe set mGapdh_LTS00102 (forward nucleobase sequence GGCAAATTCAACGGCACAGT, designated herein as SEQ ID NO: 12; reverse nucleobase sequence GGGTCTCGCTCCTGGAAGAT, designated herein as SEQ ID NO: 13; probe nucleobase sequence AAGGCCGAGAATGGGAAGCTTGTCATC, designated herein as SEQ TD NO: 14). The values marked by the symbol “†” indicate that the modified oligonucleotide is complementary to the amplicon region of the primer probe set. In such cases, an additional human primer probe set, RTS38095 (described herein above) was run to confirm activity of the compounds.

TABLE 71
Reduction of human DMPK RNA in DMSXL
transgenic mice at a dose of 350 μg

| | | DMPK RNA (% control) | | |
|---|---|---|---|---|
| | | SPINAL | | |
| Compound | | CORD | CORTEX | HIPPOCAMPUS |
| No. | n | (RTS38096) | (RTS38096) | (RTS38096) |
| PBS | 4 | 100 | 100 | 100 |
| 1052879 | 2 | 31 | 32 | 41 |
| 1052881 | 1 | 61 | 86 | 87 |
| 1380286 | 1 | 53 | 66 | 63 |
| 1380289 | 2 | 53 | 49 | 57 |
| 1380294 | 2 | 63 | 76 | 95 |
| 1380304 | 1 | 90 | 103 | 96 |
| 1380320 | 1 | 45 | 68 | 65 |
| 1380322 | 1 | 54 | 67 | 80 |
| 1380329 | 2 | 77 | 68 | 75 |
| 1380330 | 2 | 96 | 70 | 67 |
| 1380345 | 2 | 59 | 57 | 57 |
| 1380355 | 1 | 74 | 84 | 92 |
| 1380358 | 2 | 68‡ | 68‡ | 90‡ |
| 1380380 | 1 | 32 | 41 | 33 |
| 1380405 | 1 | 78 | 51 | 48 |
| 1380431 | 2 | 66 | 63 | 62 |
| 1380432 | 1 | 41 | 60 | 59 |
| 1380446 | 2 | 75 | 51 | 56 |
| 1380447 | 1 | 44 | 57 | 49 |
| 1380453 | 2 | 73 | 63 | 66 |
| 1380454 | 2 | 68 | 70 | 78 |
| 1380460 | 1 | 49 | 64 | 78 |
| 1380469 | 1 | 44 | 39 | 43 |
| 1380489 | 2 | 38 | 41 | 47 |
| 1380502 | 1 | 79 | 75 | 78 |
| 1380516 | 2 | 79 | 74 | 76 |
| 1380517 | 2 | 58 | 54 | 57 |
| 1380522 | 1 | 52 | 66 | 75 |
| 1380539 | 1 | 62 | 65 | 86 |
| 1380545 | 1 | 70 | 77 | 98 |
| 1380550 | 1 | 31 | 46 | 44 |
| 1380564 | 1 | 55 | 68 | 70 |
| 1380574 | 1 | 77 | 83 | 82 |
| 1380598 | 2 | 56 | 59 | 53 |
| 1380605 | 2 | 64 | 81 | 88 |
| 1380630 | 1 | 55 | 44 | 41 |
| 1380634 | 2 | 43 | 50 | 53 |
| 1380647 | 1 | 50 | 40 | 51 |
| 1380652 | 2 | 35 | 39 | 33 |
| 1380656 | 1 | 70 | 70 | 82 |
| 1380659 | 2 | 37 | 30 | 35 |
| 1380665 | 2 | 79 | 76 | 77 |
| 1380668 | 2 | 72 | 66 | 61 |
| 1380679 | 2 | 39 | 27 | 46 |
| 1380683 | 2 | 40 | 41 | 51 |
| 1380684 | 1 | 56 | 46 | 50 |
| 1380686 | 2 | 36 | 34 | 30 |
| 1380693 | 2 | 47 | 59 | 64 |
| 1380700 | 1 | 86 | 78 | 89 |
| 1380701 | 2 | 51 | 55 | 61 |
| 1380721 | 1 | 84 | 62 | 63 |
| 1380725 | 2 | 33 | 46 | 49 |
| 1380740 | 1 | 50 | 44 | 58 |
| 1380765 | 2 | 54 | 55 | 79 |
| 1380777 | 2 | 65 | 68 | 81 |
| 1380778 | 2 | 65 | 59 | 62 |
| 1380782 | 1 | 56 | 54 | 62 |
| 1380785 | 2 | 65 | 56 | 57 |
| 1380791 | 1 | 47 | 63 | 69 |
| 1380798 | 1 | 68 | 71 | 83 |
| 1380821 | 1 | 29 | 37 | 51 |
| 1380828 | 2 | 54 | 50 | 75 |
| 1380835 | 2 | 98 | 88 | 105 |
| 1380847 | 1 | 46 | 36 | 39 |
| 1380857 | 1 | 44 | 61 | 73 |
| 1380862 | 1 | 32 | 47 | 64 |
| 1380864 | 1 | 102 | 104 | 99 |
| 1380868 | 2 | 40 | 38 | 51 |
| 1380870 | 1 | 67 | 63 | 76 |
| 1380872 | 1 | 46 | 58 | 65 |
| 1380892 | 2 | 61 | 49 | 74 |
| 1380995 | 2 | 76 | 66 | 64 |
| 1380905 | 1 | 61 | 64 | 89 |
| 1380975 | 1 | 83 | 94 | 88 |
| 1381015 | 2 | 89 | 90 | 75 |
| 1381021 | 1 | 60 | 56 | 62 |
| 1381059 | 2 | 72 | 75 | 75 |
| 1381061 | 1 | 32 | 50 | 44 |
| 1381062 | 2 | 56 | 44 | 55 |
| 1381079 | 1 | 57 | 61 | 75 |
| 1381083 | 2 | 78 | 79 | 77 |
| 1381132 | 1 | 47 | 51 | 83 |
| 1381153 | 1 | 67 | 49 | 67 |
| 1381156 | 1 | 83 | 78 | 95 |
| 1381159 | 2 | 40 | 50 | 61 |
| 1381170 | 1 | 63 | 48 | 60 |
| 1381176 | 2 | 45 | 46 | 55 |
| 1381178 | 1 | 59 | 56 | 78 |
| 1381183 | 1 | 64 | 58 | 63 |
| 1381200 | 1 | 34 | 47 | 57 |
| 1381203 | 1 | 70 | 76 | 78 |
| 1381214 | 1 | 46 | 52 | 60 |
| 1381215 | 2 | 74 | 61 | 57 |
| 1381217 | 2 | 72 | 66 | 70 |
| 1381223 | 1 | 36 | 40 | 39 |
| 1381285 | 2 | 36 | 39 | 45 |
| 1381320 | 1 | 45 | 45 | 39 |
| 1381332 | 2 | 54 | 53 | 53 |
| 1381407 | 1 | 38 | 61 | 48 |
| 1381435 | 1 | 61 | 64 | 65 |
| 1381456 | 2 | 32 | 29 | 24 |
| 1381490 | 2 | 61 | 68 | 60 |
| 1381507 | 2 | 29 | 43 | 30 |
| 1381513 | 2 | 27 | 39 | 36 |
| 1381524 | 1 | 32 | 38 | 39 |
| 1381554 | 2 | 53 | 48 | 34 |
| 1381603 | 2 | 34 | 47 | 39 |
| 1381614 | 2 | 70 | 74 | 63 |
| 1381619 | 2 | 61 | 64 | 71 |
| 1381657 | 1 | 25 | 39 | 33 |
| 1381668 | 2 | 34 | 28 | 28 |
| 1381671 | 2 | 45 | 52 | 49 |
| 1381677 | 1 | 30 | 34 | 43 |
| 1382666 | 2 | 86 | 75 | 81 |
| 1382683 | 2 | 101 | 74 | 80 |
| 1382684 | 2 | 88 | 93 | 101 |
| 1382695 | 2 | 80 | 66 | 62 |
| 1382701 | 2 | 68 | 62 | 60 |
| 1382703 | 2 | 98 | 82 | 78 |
| 1382715 | 2 | 99 | 92 | 97 |
| 1382732 | 2 | 95 | 85 | 83 |
| 1382734 | 1 | 94 | 111 | 99 |
| 1382741 | 2 | 91 | 87 | 83 |
| 1382751 | 2 | 94 | 86 | 75 |
| 1382753 | 2 | 88 | 78 | 76 |
| 1382755 | 1 | 79 | 76 | 73 |
| 1382760 | 2 | 87 | 81 | 85 |

‡Indicates that fewer than 2 samples were available for analysis

TABLE 72
Reduction of human DMPK RNA in
DMSXL transgenic mice at a dose of 350 μg

| | | DMPK RNA (% control) | | |
|---|---|---|---|---|
| | | SPINAL | | |
| Compound | | CORD | CORTEX | HIPPOCAMPUS |
| No. | n | (RTS38096) | (RTS38096) | (RTS38096) |
| PBS | 4 | 100 | 100 | 100 |
| 1380496 | 1 | 67 | 64 | 84 |
| 1380586 | 2 | 81 | 87 | 84 |
| 1382761 | 1 | 96 | 81 | 92 |

TABLE 73
Reduction of human DMPK RNA in DMSXL transgenic
mice at a dose of 100 or 350 μg

| | | | DMPK RNA (% control) | | | |
|---|---|---|---|---|---|---|
| Compound | Dose | | CORTEX | | SPINAL CORD | |
| No. | (ug) | n | RTS38096 | RTS38095 | RTS38096 | RTS38095 |
| PBS | N/A | 4 | 100 | 100 | 100 | 100 |
| 1381255 | 350 | 2 | 54 | 64 | 40 | 56 |
| 1381470 | 350 | 2 | 36 | 41 | 30 | 40 |
| 1381486 | 350 | 2 | 53 | 64 | 48 | 58 |
| 1459325 | 350 | 2 | 52 | 76 | 65 | 88 |
| 1459327 | 350 | 2 | 33‡ | 39‡ | 46‡ | 54‡ |
| 1459343 | 350 | 2 | 31 | 31 | 38 | 40 |
| 1459344 | 350 | 2 | 48 | 54 | 63 | 76 |
| 1459348 | 350 | 2 | 74 | 84 | 87 | 113 |
| 1459349 | 350 | 2 | 57 | 72 | 58 | 80 |
| 1459352 | 350 | 2 | 43 | 49 | 32 | 48 |
| 1459353 | 350 | 2 | 33 | 37 | 32 | 52 |
| 1459354 | 350 | 2 | 37 | 44 | 21 | 31 |
| 1459359 | 350 | 2 | 63 | 77 | 45 | 69 |
| 1459360 | 350 | 2 | 37 | 39 | 29 | 42 |
| 1459364 | 350 | 2 | 59 | 64 | 26 | 39 |
| 1459367 | 350 | 2 | 92 | 110 | 84 | 108 |
| 1459369 | 350 | 2 | 74 | 78 | 64 | 84 |
| 1459372 | 350 | 2 | 35 | 36 | 23 | 34 |
| 1459374 | 350 | 2 | 18 | 20 | 20 | 25 |
| 1459377 | 350 | 2 | 51 | 66 | 58 | 71 |
| 1459378 | 350 | 2 | 65 | 78 | 84 | 103 |
| 1459379 | 350 | 2 | 67 | 83 | 90 | 105 |
| 1459383 | 350 | 2 | 72 | 87 | 56 | 61 |
| 1459395 | 350 | 2 | 68‡ | 71‡ | 74‡ | 77‡ |
| 1459396 | 350 | 2 | 89‡ | 106‡ | 70‡ | 88‡ |
| 1459398 | 350 | 2 | 57 | 63 | 32 | 40 |
| 1459399 | 350 | 2 | 45 | 52 | 25 | 35 |
| 1459407 | 350 | 2 | 48 | 55 | 44 | 49 |
| 1459408 | 350 | 2 | 47 | 53 | 58 | 80 |
| 1459409 | 350 | 2 | 36 | 44 | 33 | 38 |
| 1459412 | 350 | 2 | 67 | 74 | 60 | 93 |
| 1459418 | 350 | 2 | 33 | 40 | 33 | 41 |
| 1459419 | 350 | 2 | 26 | 37 | 29 | 36 |
| 1459420 | 350 | 2 | 35 | 39 | 36 | 48 |
| 1459422 | 350 | 2 | 65 | 72 | 76 | 90 |
| 1459423 | 350 | 2 | 76 | 82 | 99 | 116 |
| 1459424 | 350 | 2 | 51 | 57 | 73 | 71 |
| 1459426 | 350 | 2 | 31 | 36 | 46 | 46 |
| 1459427 | 350 | 2 | 45 | 51 | 60 | 65 |
| 1459428 | 350 | 2 | 67 | 82 | 71 | 110 |
| 1459433 | 350 | 2 | 64 | 76 | 43 | 48 |
| 1459434 | 350 | 2 | 54 | 59 | 31 | 34 |
| 1459435 | 350 | 2 | 60 | 74 | 55 | 71 |
| 1459436 | 350 | 2 | 53‡ | 65‡ | 77‡ | 87‡ |
| 1459437 | 350 | 2 | 27‡ | 36‡ | 46‡ | 55‡ |
| 1459438 | 350 | 2 | 37 | 47 | 37 | 48 |
| 1459439 | 350 | 2 | 77 | 93 | 78 | 101 |
| 1459440 | 350 | 2 | 45‡ | 48‡ | 38‡ | 44‡ |
| 1459441 | 350 | 2 | 45 | 55 | 39 | 52 |
| 1459444 | 350 | 2 | 45 | 56 | 54 | 64 |
| 1459446 | 350 | 2 | 68 | 87 | 93 | 121 |
| 1459449 | 350 | 2 | 65 | 71 | 35 | 46 |
| 1459450 | 350 | 2 | 63 | 75 | 47 | 54 |
| 1459451 | 350 | 2 | 59 | 66 | 47 | 60 |
| 1459455 | 350 | 2 | 54 | 67 | 79 | 90 |
| 1459456 | 350 | 2 | 83 | 96 | 82 | 109 |
| 1459457 | 350 | 2 | 74‡ | 79‡ | 72‡ | 104‡ |
| 1459458 | 350 | 2 | 41 | 50 | 36 | 45 |
| 1459461 | 350 | 2 | 67 | 82 | 54 | 77 |
| 1459463 | 350 | 2 | 73 | 88 | 75 | 98 |
| 1459465 | 350 | 2 | 31 | 43 | 53 | 66 |
| 1459981 | 350 | 2 | 34 | 40 | 34 | 41 |
| 1459985 | 350 | 2 | 22‡ | 37 | 21‡ | 38 |
| 1459986 | 350 | 2 | 53‡ | 59 | 44‡ | 62 |
| 1459988 | 350 | 2 | 47 | 69 | 72 | 86 |
| 1459989 | 350 | 2 | 48 | 61 | 48 | 55 |
| 1460166 | 350 | 2 | 38 | 47 | 53 | 63 |
| 1460170 | 350 | 2 | 52 | 69 | 48 | 63 |
| 1460171 | 350 | 2 | 58 | 67 | 77 | 92 |
| 1460178 | 350 | 2 | 50 | 58 | 63 | 66 |
| 1460179 | 350 | 2 | 77 | 98 | 82 | 121 |
| 1460185 | 350 | 2 | 68 | 70 | 83 | 93 |
| 1052866 | 350 | 2 | 41 | 42 | 47 | 57 |
| 1052868 | 350 | 2 | 34 | 34 | 30 | 34 |
| 1052869 | 350 | 2 | 57 | 60 | 55 | 62 |
| 1052877 | 350 | 2 | 22‡ | 32‡ | 33‡ | 39‡ |
| 1052879 | 350 | 1 | 33 | 43 | 31 | 41 |
| 1273291 | 100 | 2 | 47‡ | 67‡ | 29‡ | 36‡ |
| 1338115 | 100 | 2 | 21 | 26 | 7 | 8 |
| | 350 | 2 | 13 | 16 | 5 | 4 |
| 1380378 | 350 | 2 | 30‡ | 42 | 27‡ | 41 |
| 1380456 | 350 | 1 | 99 | 134 | 88 | 107 |
| 1380457 | 350 | 1 | 59 | 73 | 43 | 60 |
| 1380571 | 350 | 1 | 29 | 39 | 44 | 54 |
| 1380815 | 350 | 2 | 59 | 63 | 71 | 78 |
| 1380870 | 350 | 1 | 62 | 78 | 58 | 69 |
| 1380962 | 350 | 1 | 57 | 70 | 55 | 62 |
| 1380993 | 350 | 2 | 71‡ | 97‡ | 80‡ | 97‡ |
| 1381037 | 350 | 2 | 55 | 60 | 55 | 67 |
| 1381363 | 350 | 2 | 78 | 69 | 44 | 52 |
| 1400744 | 100 | 2 | 27‡ | 42 | 12‡ | 21 |
| | 350 | 2 | 21‡ | 34 | 13‡ | 23 |
| 1400761 | 100 | 2 | 18 | 23 | 6 | 6 |
| 1400776 | 100 | 2 | 30 | 42 | 29 | 36 |
| 1459314 | 350 | 2 | 43‡ | 49‡ | 46‡ | 51‡ |
| 1459315 | 350 | 2 | 61 | 77 | 60 | 74 |
| 1459320 | 350 | 2 | 43 | 52 | 43 | 59 |
| 1459322 | 350 | 1 | 53 | 68 | 38 | 47 |
| 1459323 | 350 | 2 | 39 | 39 | 30 | 42 |
| 1459324 | 350 | 2 | 68 | 71 | 56 | 59 |
| 1459326 | 350 | 2 | 71 | 71 | 64 | 76 |
| 1459328 | 350 | 2 | 97 | 132 | 23 | 37 |
| 1459329 | 350 | 2 | 30 | 38 | 39 | 58 |
| 1459330 | 350 | 2 | 43 | 55 | 45 | 62 |
| 1459345 | 350 | 2 | 66 | 81 | 82 | 96 |
| 1459346 | 350 | 2 | 55‡ | 60‡ | 57‡ | 76‡ |
| 1459351 | 350 | 2 | 42 | 54 | 48 | 64 |
| 1459355 | 350 | 2 | 49 | 64 | 64 | 88 |
| 1459356 | 350 | 2 | 51 | 57 | 74 | 82 |
| 1459365 | 350 | 2 | 41‡ | 44‡ | 23‡ | 34‡ |
| 1459366 | 350 | 2 | 42 | 44 | 38 | 53 |
| 1459368 | 350 | 2 | 63 | 69 | 78 | 77 |
| 1459375 | 350 | 2 | 29 | 28 | 17 | 27 |
| 1459386 | 350 | 2 | 47 | 54 | 47 | 52 |
| 1459387 | 350 | 2 | 66 | 59 | 50 | 63 |
| 1459392 | 350 | 2 | 43 | 49 | 41 | 49 |
| 1459393 | 350 | 2 | 75‡ | 82‡ | 84‡ | 97‡ |
| 1459394 | 350 | 2 | 64‡ | 76‡ | 83‡ | 107‡ |
| 1459400 | 350 | 2 | 44 | 59 | 63 | 86 |
| 1459401 | 350 | 2 | 72 | 83 | 73 | 77 |
| 1459402 | 350 | 2 | 81 | 97 | 83 | 93 |
| 1459406 | 350 | 2 | 61 | 58 | 55 | 59 |
| 1459413 | 350 | 2 | 46‡ | 53‡ | 64‡ | 73‡ |
| 1459417 | 350 | 2 | 29 | 29 | 32 | 41 |
| 1459421 | 350 | 2 | 53 | 59 | 56 | 62 |
| 1459425 | 350 | 2 | 54 | 58 | 37 | 49 |
| 1459429 | 350 | 2 | 63 | 75 | 82 | 99 |
| 1459442 | 350 | 2 | 29 | 29 | 23 | 27 |
| 1459443 | 350 | 2 | 26 | 25 | 12 | 17 |
| 1459445 | 350 | 2 | 55‡ | 75‡ | 89‡ | 106‡ |
| 1459452 | 350 | 2 | 57‡ | 62‡ | 76‡ | 91‡ |
| 1459453 | 350 | 2 | 67 | 70 | 56 | 68 |
| 1459459 | 350 | 2 | 36 | 45 | 36 | 42 |
| 1459462 | 350 | 2 | 52 | 57 | 62 | 69 |
| 1459464 | 350 | 2 | 74 | 87 | 81 | 88 |
| 1459974 | 350 | 1 | 21† | 35 | 23† | 34 |
| 1459975 | 350 | 2 | 45† | 68 | 29† | 45 |
| 1459976 | 350 | 2 | 56† | 68 | 28† | 36 |
| 1459977 | 350 | 2 | 36† | 48 | 32† | 48 |
| 1459978 | 350 | 1 | 51† | 69 | 50† | 65 |
| 1459979 | 350 | 2 | 28† | 42 | 29† | 44 |
| 1459982 | 350 | 1 | 46 | 55 | 48 | 54 |
| 1459983 | 350 | 2 | 55‡ | 73‡ | 46‡ | 67‡ |
| 1459987 | 350 | 2 | 50† | 60 | 41† | 60 |
| 1459990 | 350 | 1 | 54 | 121 | 68 | 90 |
| 1459991 | 350 | 1 | 33 | 46 | 49 | 59 |
| 1460162 | 350 | 2 | 70 | 81 | 77 | 100 |
| 1460163 | 350 | 2 | 67 | 71 | 74 | 79 |
| 1460164 | 350 | 2 | 65 | 68 | 54 | 68 |
| 1460168 | 350 | 2 | 63 | 69 | 69 | 79 |
| 1460174 | 350 | 1 | 63 | 89 | 73 | 89 |
| 1460184 | 350 | 2 | 56 | 60 | 59 | 64 |

‡Indicates that fewer than 2 samples were available for analysis

TABLE 74
Reduction of human DMPK RNA in DMSXL
transgenic mice at a dose of 350 μg

| | | DMPK RNA (% control) | | | |
|---|---|---|---|---|---|
| Compound | | CORTEX | | SPINAL CORD | |
| No. | n | RTS38096 | RTS38095 | RTS38096 | RTS38095 |
| PBS | 3 | 100 | 100 | 100 | 100 |
| 1380987 | 2 | 81 | 92 | 65 | 85 |
| 1459373 | 2 | 23 | 20 | 26 | 27 |
| 1459415 | 2 | 49 | 66 | 55 | 72 |
| 1459973 | 2 | 88† | 84 | 72† | 84 |
| 1459980 | 2 | 75 | 86 | 69 | 77 |
| 1511076 | 2 | 75 | 82 | 69 | 73 |
| 1511077 | 2 | 73 | 81 | 67 | 79 |
| 1511078 | 2 | 84 | 95 | 60 | 67 |
| 1511079 | 2 | 85 | 87 | 71 | 83 |
| 1511080 | 2 | 73 | 85 | 59 | 65 |
| 1511081 | 2 | 75‡ | 92‡ | 63‡ | 77‡ |
| 1511082 | 2 | 84 | 95 | 84 | 98 |
| 1511083 | 2 | 62‡ | 71‡ | 44‡ | 62‡ |
| 1511084 | 2 | 52 | 60 | 42 | 56 |
| 1511085 | 2 | 60 | 69 | 59 | 75 |
| 1511086 | 2 | 57 | 69 | 52 | 60 |
| 1511087 | 2 | 67 | 94 | 54 | 72 |
| 1511088 | 2 | 56 | 68 | 57 | 77 |
| 1511089 | 2 | 66 | 98 | 68 | 98 |
| 1511090 | 2 | 61 | 91 | 49 | 71 |
| 1511091 | 2 | 70 | 94 | 60 | 93 |
| 1511092 | 2 | 72 | 92 | 59 | 73 |
| 1511093 | 2 | 70 | 89 | 67 | 94 |
| 1511094 | 2 | 66 | 84 | 70 | 106 |
| 1511095 | 2 | 59 | 82 | 56 | 92 |
| 1511096 | 2 | 76 | 103 | 66 | 104 |
| 1511097 | 2 | 84 | 110 | 71 | 104 |
| 1511098 | 2 | 90 | 114 | 89 | 112 |
| 1511099 | 2 | 70 | 93 | 74 | 101 |
| 1511100 | 2 | 50 | 64 | 57 | 81 |
| 1511101 | 2 | 62 | 78 | 64 | 98 |
| 1511102 | 2 | 56 | 74 | 49 | 72 |
| 1511103 | 2 | 89 | 118 | 87 | 117 |
| 1511104 | 2 | 78 | 90 | 58 | 85 |
| 1511105 | 2 | 85 | 110 | 87 | 106 |
| 1511106 | 2 | 67 | 79 | 52 | 64 |
| 1511107 | 2 | 62 | 68 | 61 | 88 |

‡Indicates that fewer than 2 samples were available for analysis

### Example 12: Activity of Modified Oligonucleotides Complementary to Human DMPK in DM20 Transgenic Mice

Modified oligonucleotides described above were tested in DM20 transgenic mice previously described in Seznec H, et. al, Transgenic mice carrying large human genomic sequences with expanded CTG repeat mimic closely the DM CTG repeat intergenerational and somatic instability; Human Molecular Genetics, 2000, vol. 9, no. 8: 1185-1194.

### Treatment

DM20 transgenic mice were divided into groups of 1-2 mice each. Each mouse received a single ICV bolus of 350 μg of modified oligonucleotide as indicated in the tables below. A group of 2 mice received PBS as a negative control.

### RNA Analysis

Two weeks post treatment, mice were sacrificed, and RNA was extracted from cortical brain tissue, spinal cord, hippocampus, striatum, and cerebellum for RTPCR analysis to measure amount of DMPK RNA using human primer probe set RTS3164 (forward nucleobase sequence AGCCTGAGCCGGGAGATG, designated herein as SEQ ID NO: 15; reverse nucleobase sequence GCGTAGTTGACTGGCGAAGTT, designated herein as SEQ ID NO: 16; probe nucleobase sequence AGGCCATCCGCACGGACAACCX, designated herein as SEQ ID NO: 17). Results are presented as percent human DMPK RNA relative to PBS control, normalized to mouse GAPDH (% control). Mouse GAPDH was amplified using mouse primer probe set mGapdh_LTS00102 (forward nucleobase sequence GGCAAATTCAACGGCACAGT, designated herein as SEQ ID NO: 12; reverse nucleobase sequence GGGTCTCGCTCCTGGAAGAT, designated herein as SEQ ID NO: 13; probe nucleobase sequence AAGGCCGAGAATGGGAAGCTTGTCATC, designated herein as SEQ ID NO: 14).

TABLE 75
Reduction of human DMPK RNA in DM20
transgenic mice at a dose of 350 μg

| | | DMPK RNA (% control) | | | | |
|---|---|---|---|---|---|---|
| Compound | | | Spinal | | Hippo- | Cere- |
| No. | n | Cortex | Cord | Striatum | campus | bellum |
| PBS | 2 | 100 | 100 | 100 | 100 | 100 |
| 1052865 | 2 | 67 | 66 | 105 | 106 | 86 |
| 1052866 | 2 | 41 | 65 | 92 | 57 | 77 |
| 1052867 | 2 | 38 | 77 | 62 | 39 | 41 |
| 1052868 | 2 | 40 | 45 | 47 | 43 | 49 |
| 1052871 | 2 | 107 | 63 | 55 | 73 | 78 |
| 1052874 | 2 | 47 | 67 | 64 | 96 | 68 |
| 1052876 | 2 | 61 | 66 | 86 | 116 | 75 |
| 1052877 | 2 | 28 | 35 | 75 | 56 | 46 |
| 1052883 | 1 | 69 | 74 | 64 | 99 | 65 |
| 1052887 | 2 | 92 | 84 | 79 | 81 | 71 |
| 1052889 | 2 | 72 | 114 | 67 | 92 | 78 |

### Example 13: Activity of Modified Oligonucleotides Complementary to Human DMPK in DMSXL Transgenic Mice, Multiple Dose

DMSXL transgenic mice (described herein above) were used to determine dose response activity of modified oligonucleotides complementary to human DMPK.

### Treatment

DMSXL transgenic mice were divided into groups of 2-4 mice each. Each mouse received a single ICV bolus of modified oligonucleotide at various doses indicated in the table below. A group of 4-6 mice received a single ICV bolus of PBS as a negative control.

### RNA Analysis

Two weeks post treatment, mice were sacrificed, and RNA was extracted from cortical brain tissue and spinal cord for RTPCR analysis to measure amount of DMPK RNA using human primer probe set RTS38096 (described herein above). Results are presented as percent human DMPK RNA relative to PBS control, normalized to mouse GAPDH (% control). Mouse GAPDH was amplified using mouse primer probe set mGapdh_LTS00102 (described herein above). ED50s were calculated in Prism using nonlinear fit with variable slope (four parameter), top constrained to 100% (or 1), bottom constrained to 0. Y=Bottom+(Top−Bottom)/(1+(IC50/X)\hat{ }HillSlope).

Compound No. 486178 is described herein above.

TABLE 76
Reduction of human DMPK RNA in DMSXL transgenic mice

| | | DMPK RNA (% control) | | | |
|---|---|---|---|---|---|
| | | | | SPINAL | |
| Compound | Dose | CORTEX | ED50 | CORD | ED50 |
| No. | (μg) | (RTS38096) | (μg) | (RTS38096) | (μg) |
| PBS | N/A | 100 | — | 100 | — |
| 1052877 | 10 | 100 | 220 | 92 | 68 |
| | 30 | 90 | | 64 | |
| | 100 | 74 | | 40 | |
| | 300 | 51 | | 28 | |
| | 700 | 34 | | 21 | |
| 1380679 | 10 | 98 | 226 | 78 | 95 |
| | 30 | 91 | | 59 | |
| | 100 | 87 | | 57 | |
| | 300 | 41 | | 36 | |
| | 700 | 36 | | 28 | |
| 1381668 | 10 | 100 | 221 | 90 | 89 |
| | 30 | 100 | | 77 | |
| | 100 | 76 | | 39 | |
| | 300 | 50 | | 27 | |
| | 700 | 30 | | 34 | |
| 1459328 | 10 | 110 | 261 | 84 | 152 |
| | 30 | 93 | | 70 | |
| | 100 | 74 | | 71 | |
| | 300 | 52 | | 32 | |
| | 700 | 42 | | 30 | |
| 1459329 | 10 | 117 | 384 | 94 | 226 |
| | 30 | 113 | | 89 | |
| | 100 | 84 | | 63 | |
| | 300 | 63 | | 44 | |
| | 700 | 44 | | 41 | |
| 1459374 | 10 | 84 | 139 | 65 | 26 |
| | 30 | 83 | | 43 | |
| | 100 | 71 | | 35 | |
| | 300 | 39 | | 24 | |
| | 700 | 29 | | 22 | |
| 1459375 | 10 | 102 | 175 | 60 | 16 |
| | 30 | 88 | | 35 | |
| | 100 | 68 | | 26 | |
| | 300 | 46 | | 17 | |
| | 700 | 32 | | 12 | |
| 1459442 | 10 | 82 | 109 | 75 | 32 |
| | 30 | 79 | | 46 | |
| | 100 | 64 | | 33 | |
| | 300 | 36 | | 23 | |
| | 700 | 31 | | 20 | |
| 1459443 | 10 | 78 | 149 | 71 | 25 |
| | 30 | 89 | | 43 | |
| | 100 | 73 | | 31 | |
| | 300 | 42 | | 16 | |
| | 700 | 24 | | 14 | |
| 1511084 | 10 | 91 | 289 | 114 | 205 |
| | 30 | 96 | | 91 | |
| | 100 | 81 | | 55 | |
| | 300 | 56 | | 42 | |
| | 700 | 38 | | 37 | |

TABLE 77
Reduction of human DMPK RNA in DMSXL transgenic mice

| | | DMPK RNA (% control) | | | |
|---|---|---|---|---|---|
| | | | | SPINAL | |
| Compound | Dose | CORTEX | ED50 | CORD | ED50 |
| No. | (μg) | (RTS38096) | (μg) | (RTS38096) | (μg) |
| PBS | 0 | 100 | | 100 | |
| 486178 | 3 | 108 | 69 | 67 | 5.5 |
| | 10 | 83 | | 30 | |
| | 30 | 83 | | 22 | |
| | 100 | 46 | | 24 | |
| 1400761 | 3 | 89 | 52 | 62 | 3.7 |
| | 10 | 80 | | 17 | |
| | 30 | 78 | | 15 | |
| | 100 | 41 | | 7 | |
| 1338115 | 3 | 97 | 42 | 68 | 4.4 |
| | 10 | 89 | | 21 | |
| | 30 | 75 | | 18 | |
| | 100 | 24 | | 7 | |

### Example 14: Activity of Modified Oligonucleotides Complementary to Human DMPK in DMSXL Transgenic Mice, Single Dose

Modified oligonucleotides described above were tested in DMSXL transgenic mice previously described in Huguet A, et. al, Molecular, Physiological, and Motor Performance Defects in DMSXL Mice Carrying >1,000 CTG Repeats from the Human DM1 Locus; PLOS Genetics, 2012, vol. 8, no. 11: e1003043.

### Treatment

DMSXL transgenic mice were divided into groups of 4 mice each. Each mouse received subcutaneous injections of modified oligonucleotide at a dose of 10 mg/kg once a week for two weeks (a total of 3 treatments). One group of 4 mice received subcutaneous injections of PBS once a week for two weeks (a total of 3 treatments). The PBS-injected group served as the control group to which modified oligonucleotide-treated groups were compared.

### RNA Analysis

7 days post the final treatment, mice were sacrificed and RNA was extracted from mouse quadriceps muscle, gastrocnemius muscle, and/or tibialis anterior muscle, as specified in the tables below, for real-time RTPCR analysis of DMPK RNA expression. Human DMPK primer probe set RTS3164 (forward nucleobase sequence AGCCTGAGCCGGGAGATG, designated herein as SEQ ID NO: 2360; reverse nucleobase sequence GCGTAGTTGACTGGCGAAGTT, designated herein as SEQ ID NO: 2361; probe nucleobase sequence AGGCCATCCGCACGGACAACC, designated herein as SEQ ID NO: 2362) was used to measure human DMPK RNA levels as indicated in the tables below. DMPK RNA levels were normalized either to total RNA content, as measured to mouse GAPDH. Mouse GAPDH was amplified using mouse primer probe set mGapdh_LTS00102 (forward nucleobase sequence GGCAAATTCAACGGCACAGT, designated herein as SEQ ID NO: 2363; reverse nucleobase sequence GGGTCTCGCTCCTGGAAGAT, designated herein as SEQ ID NO: 2364; probe nucleobase sequence AAGGCCGAGAATGGGAAGCTTGTCATC, designated herein as SEQ ID NO: 2365). Results are presented as percent DMPK RNA, relative to the amount in PBS treated animals (% control). In some cases, values for certain tissue types were not calculated for all ASOs. In these cases, ‘N.C.’ is used in the tables indicates that the values were not calculated. The Compound No. marked with a “†” indicates that the modified oligonucleotide is complementary to the amplicon region of the primer probe set. Additional assays may be used to measure the activity of the modified oligonucleotides complementary to the amplicon region.

Compound No. 598769 is described herein above.

TABLE 78
Reduction of human DMPK in DMSXL transgenic mice

| | DMPK RNA (% control) | | |
|---|---|---|---|
| | | | Tibialis |
| Compound No. | Quadriceps | Gastrocnemius | Anterior |
| PBS | 100 | 100 | 100 |
| 1046921 | 76 | N.C. | N.C. |
| 1046922 | 69 | N.C. | N.C. |
| 1046935 | 87 | N.C. | N.C. |
| 1046936 | 83 | N.C. | N.C. |
| 1046948 | 44 | 57 | 75 |
| 1046952 | 66 | N.C. | N.C. |

TABLE 79
Reduction of human DMPK in DMSXL transgenic mice

| | DMPK RNA |
|---|---|
| | (% control) |
| Compound No. | Quadriceps |
| PBS | 100 |
| 1059473 | 77 |
| 1059474 | 86 |
| 1059475 | 90 |
| 1059476 | 80 |
| 1059477 | 84 |
| 1059478 | 59 |
| 1059892 | 91 |
| 1059903 | 105 |

TABLE 80
Reduction of human DMPK in DMSXL transgenic mice

| | DMPK RNA (% control) | | |
|---|---|---|---|
| Compound | | | Tibialis |
| No. | Quadriceps | Gastrocnemius | Anterior |
| PBS | 100 | 100 | 100 |
| 1059889 | 33 | 57 | 53 |
| 1059890 | 49 | 66 | 62 |
| 1060866 | 105 | N.C. | N.C. |
| 1060875 | 64 | N.C. | N.C. |
| 1060883 | 60 | N.C. | N.C. |
| 1060886 | 100 | N.C. | N.C. |
| 1060889 | 82 | N.C. | N.C. |
| 1060891 | 75 | N.C. | N.C. |
| 1060897 | 79 | N.C. | N.C. |
| 1060898 | 73 | N.C. | N.C. |
| 1060899 | 62 | N.C. | N.C. |
| 1060900 | 73 | N.C. | N.C. |
| 1060906 | 80 | N.C. | N.C. |
| 1060909 | 62 | N.C. | N.C. |
| 1060914 | 89 | N.C. | N.C. |
| 1060916 | 71 | N.C. | N.C. |
| 1060917 | 86 | N.C. | N.C. |
| 1060921 | 77 | N.C. | N.C. |
| 1060923 | 40 | 64 | 58 |

TABLE 81
Reduction of human DMPK in DMSXL transgenic mice

| | DMPK RNA |
|---|---|
| Compound | (% control) |
| No. | Quadriceps |
| PBS | 100 |
| 1162310 | 66 |
| 1162311 | 65 |
| 1162628 | 79 |
| 1162632 | 104 |
| 1162633 | 97 |
| 1162634 | 101 |
| 1162636 | 96 |
| 1162638 | 126 |
| 1162643 | 62 |
| 1162645 | 105 |
| 1162647 | 126 |
| 1162654 | 77 |
| 1162655 | 60 |
| 1162658 | 84 |
| 1162635 | 126 |

TABLE 82
Reduction of human DMPK in DMSXL transgenic mice

| | DMPK RNA |
|---|---|
| Compound | (% control) |
| No. | Quadriceps |
| PBS | 100 |
| 598769 | 63 |
| 1059887 | 49 |
| 1060860 | 23 |
| 1064823 | 67 |
| 1176125 | 54 |
| 1176126 | 59 |
| 1176127 | 62 |
| 1176132 | 60 |
| 1176133 | 53 |
| 1176134 | 47 |
| 1176143 | 95 |
| 1176145 | 49 |
| 1176148 | 47 |
| 1176151 | 44 |
| 1176153 | 79 |
| 1176157 | 47 |
| 1176159 | 47 |
| 1176160 | 44 |
| 1176161 | 62 |
| 1176162 | 44 |
| 1176164 | 44 |
| 1176165 | 56 |
| 1184172 | 47 |

TABLE 83
Reduction of human DMPK in DMSXL transgenic mice

| | DMPK RNA |
|---|---|
| Compound | (% control) |
| No. | Quadriceps |
| PBS | 100 |
| 1060860 | 23 |
| 1176142 | 74 |
| 1176151 | 59 |
| 1176148 | 41 |
| 1176157 | 69 |
| 1176159 | 45 |
| 1176160 | 48 |
| 1176163 | 50 |
| 1207018 | 95 |
| 1207019 | 79 |
| 1207020 | 66 |
| 1207021 | 67 |
| 1207024 | 56 |
| 1207025 | 66 |
| 1207072 | 46 |
| 1207074 | 66 |
| 1207075 | 69 |
| 1207130 | 71 |
| 1213273 | 93 |
| 1213275 | 97 |
| 1213276 | 84 |
| 1215869 | 67 |
| 1215870 | 89 |
| 1213282 | 56 |
| 1240824 | 79 |
| 1240825 | 59 |
| 1240826 | 57 |
| 1241201 | 72 |

TABLE 84
Reduction of human DMPK in DMSXL transgenic mice

| | DMPK RNA |
|---|---|
| Compound | (% control) |
| No. | Quadriceps |
| PBS | 100 |
| 1309459 | 77 |
| 1309468 | 79 |
| 1309469 | 71 |
| 1309473 | 75 |
| 1309475 | 44 |
| 1309477† | 76 |
| 1309479 | 55 |
| 1309482 | 55 |

TABLE 85
Reduction of human DMPK in DMSXL transgenic mice

| | DMPK RNA |
|---|---|
| Compound | (% control) |
| No. | Quadriceps |
| PBS | 100 |
| 598769 | 73 |
| 1309484 | 117 |
| 1309485 | 103 |

TABLE 86
Reduction of human DMPK in DMSXL transgenic mice

| | DMPK RNA |
|---|---|
| Compound | (% control) |
| No. | Quadriceps |
| PBS | 100 |
| 1176128 | 46 |
| 1176129 | 54 |
| 1176135 | 37 |
| 1176138 | 35 |
| 1176139 | 39 |
| 1176140 | 42 |
| 1243719 | 31 |
| 1370496 | 49 |
| 1370498 | 103 |
| 1370503 | 73 |
| 1370504 | 82 |
| 1370505 | 37 |
| 1370507 | 80 |
| 1370510 | 50 |

TABLE 87
Reduction of human DMPK in DMSXL transgenic mice

| | DMPK RNA (% control) | | |
|---|---|---|---|
| Compound | | | Tibialis |
| No. | Gastrocnemius | Quadriceps | Anterior |
| PBS | 100 | 100 | 100 |
| 570052 | 73 | 70 | 45 |
| 598769 | 83 | 75 | 71 |
| 1338115 | 52 | 48 | 51 |
| 1400737 | 70 | 66 | 65 |
| 1400739 | 71 | 64 | 51 |
| 1400742 | 88 | 89 | 86 |
| 1400743 | 92 | 82 | 71 |
| 1400744 | 79 | 67 | 63 |
| 1400748 | 69 | 77 | 97 |
| 1400750 | 86 | 79 | 57 |
| 1400752 | 70 | 68 | 62 |
| 1400755 | 87 | 85 | 62 |
| 1400760 | 95 | 96 | 59 |
| 1400761 | 67 | 63 | 31 |
| 1400769 | 69 | 76 | 69 |
| 1400772 | 80 | 79 | 69 |
| 1400775 | 82 | 72 | 35 |
| 1400776 | 73 | 58 | 35 |
| 1400777 | 81 | 83 | 79 |
| 1400778 | 73 | 77 | 62 |

### Example 15: Activity of Modified Oligonucleotides Complementary to Human DMPK in DM20 Transgenic Mice, Single Dose

Modified oligonucleotides described above were tested in DM20 transgenic mice previously described in Seznec H, et. al, Transgenic mice carrying large human genomic sequences with expanded CTG repeat mimic closely the DM CTG repeat intergenerational and somatic instability; Human Molecular Genetics, 2000, vol. 9, no. 8: 1185-1194.

### Treatment

DM20 transgenic mice were divided into groups of 4 mice each. Each mouse received subcutaneous injections of modified oligonucleotide at a dose of 10 mg/kg once a week for two weeks (a total of 3 treatments). One group of 4 mice received subcutaneous injections of PBS once a week for two weeks (a total of 3 treatments). The PBS-injected group served as the control group to which modified oligonucleotide-treated groups were compared.

### RNA Analysis

7 days after the final treatment, mice were sacrificed and RNA was extracted from mouse quadriceps muscle, gastrocnemius muscle, and/or tibialis anterior muscle for real-time RTPCR analysis of DMPK RNA expression. Human DMPK primer probe set RTS3164 (described herein above) was used to measure human DMPK RNA levels as indicated in the tables below. DMPK RNA levels were normalized total RNA content, as measured to mouse GAPDH.

Mouse GAPDH was amplified using mouse primer probe set mGapdh_LTS00102 (described herein above) Results are presented as percent DMPK RNA, relative to the amount in PBS treated animals (% control).

TABLE 88
Reduction of human DMPK in DM20 transgenic mice

| | DMPK RNA |
|---|---|
| Compound | (% control) |
| No. | Quadriceps |
| PBS | 100 |
| 1046924 | 83 |
| 1046933 | 90 |
| 1046934 | 75 |
| 1046937 | 72 |
| 1060860 | 21 |
| 1060864 | 79 |
| 1060869 | 113 |
| 1060872 | 48 |
| 1060873 | 51 |
| 1060880 | 125 |
| 1060884 | 163 |
| 1060887 | 85 |
| 1060892 | 99 |
| 1060896 | 115 |
| 1060901 | 130 |
| 1060903 | 95 |
| 1060904 | 157 |
| 1060908 | 85 |
| 1162627 | 104 |
| 1162641 | 42 |

TABLE 89
Reduction of human DMPK in DM20 transgenic mice

| | DMPK RNA (% control) | | |
|---|---|---|---|
| Compound | | | Tibialis |
| No. | Quadriceps | Gastrocnemius | Anterior |
| PBS | 100 | 100 | 100 |
| 1046919 | 57 | 48 | 53 |
| 1046926 | 66 | N.C. | N.C. |
| 1059502 | 63 | N.C. | N.C. |
| 1059503 | 89 | N.C. | N.C. |
| 1059506 | 96 | N.C. | N.C. |
| 1059894 | 81 | N.C. | N.C. |

TABLE 90
Reduction of human DMPK in DM20 transgenic mice

| | DMPK RNA (% control) | | |
|---|---|---|---|
| Compound | | | Tibialis |
| No. | Quadriceps | Gastrocnemius | Anterior |
| PBS | 100 | 100 | 100 |
| 1060867 | 55 | 71 | 83 |
| 1060870 | 59 | 56 | 76 |
| 1060876 | 112 | N.C. | N.C. |
| 1060877 | 108 | N.C. | N.C. |
| 1060878 | 59 | 64 | 83 |
| 1060882 | 93 | N.C. | N.C. |
| 1060893 | 57 | 70 | 86 |
| 1060905 | 61 | N.C. | N.C. |
| 1060911 | 89 | N.C. | N.C. |
| 1060915 | 61 | N.C. | N.C. |
| 1060918 | 91 | N.C. | N.C. |
| 1064824 | 72 | N.C. | N.C. |

### Example 16: Activity of Modified Oligonucleotides Complementary to Human DMPK in DMSXL Transgenic Mice, Multiple Dose

DMPK DMSXL transgenic mice (described herein above) were used to determine activity of modified oligonucleotides complementary to human DMPK.

### Treatment

DMSXL transgenic mice were divided into groups of 3-5 mice each. Each mouse received subcutaneous injections of modified oligonucleotide at various doses as indicated in the tables below at once a week for two weeks (a total of 3 treatments). One group of 4-6 mice received subcutaneous injections of PBS once a week for two weeks (a total of 3 treatments). The PBS-injected group served as the control group to which oligonucleotide-treated groups were compared.

### RNA Analysis

7 days post the final treatment, mice were sacrificed and RNA was extracted from mouse quadricep muscle, gastrocnemius muscle, heart, tibialis anterior muscle, and triceps muscle for real-time RTPCR analysis of DMPK RNA expression. Human DMPK primer probe sets RTS3164 (described herein above) was used to measure human DMPK RNA levels. DMPK RNA levels were normalized either to total RNA content, as measured to mouse GAPDH. Mouse GAPDH was amplified using mouse primer probe set mGapdh_LTS00102 (described herein above). Results are presented as percent DMPK RNA, relative to the amount in PBS treated animals (% control). ED50s were calculated in GraphPad Prism using nonlinear fit with variable slope (four parameter), top constrained to 100% (or 1), bottom constrained to 0. Y=Bottom+(Top−Bottom)/(1+(IC50/X)\hat{ }HillSlope).

Compound No. 598769 is described herein above.

Compound No. 877864 was previously described in WO 2017/053995 A1 and consists of the nucleobase sequence (from 5′ to 3′): ACAATAAATACCGAGG (SEQ ID NO: 1336). The sugar motif for Compound No. 877864 is (from 5′ to 3′): kkkddddddddddkkk; wherein each “d” represents a 2′-β-D-deoxyribosyl sugar moiety, and each “k” represents a cEt sugar moiety. The internucleoside linkage motif for Compound No. 877864 is (from 5′ to 3′): sssssssssssssss; wherein each “s” represents a phosphorothioate internucleoside linkage. Each cytosine residue in Compound No. 877864 is a 5-methylcytosine. Compound No. 877864 is conjugated to a 6-palmitamidohexyl phosphate conjugate group attached to the 5′-OH of the oligonucleotide. The structure for the conjugate group is:

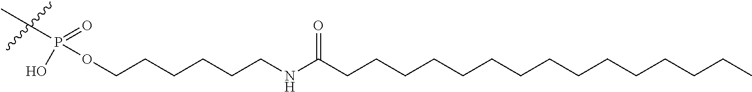 [SMILES: *P(=O)(O)OCCCCCCNC(=O)CCCCCCCCCCCCCCC]

TABLE 91
Reduction of human DMPK in DMSXL transgenic mice

Quadriceps

| | | DMPK RNA | |
|---|---|---|---|
| Compound | Dose | (% control) | ED50 |
| No. | (mg/kg) | RTS3164 | (mg/kg) |
| PBS | N/A | 100 | — |
| 570052 | 9 | 58 | — |
| 598769 | 1 | 82 | 12.5 |
| | 3 | 71 | |
| | 9 | 62 | |
| | 27 | 41 | |
| | 81 | 26 | |
| 1059889 | 1 | 80 | 8.1 |
| | 3 | 82 | |
| | 9 | 49 | |
| | 27 | 20 | |
| | 81 | 5 | |
| 1060923 | 1 | 80 | 5.0 |
| | 3 | 67 | |
| | 9 | 36 | |
| | 27 | 22 | |
| | 81 | 11 | |
| 1064823 | 9 | 55 | — |
| 1273291 | 10 | 24 | — |
| 1273292 | 10 | 41 | — |
| 1273297 | 10 | 23 | — |
| 1273301 | 10 | 38 | — |
| 1273313 | 1 | 91 | 22.5 |
| | 3 | 86 | |
| | 9 | 74 | |
| | 27 | 51 | |
| | 81 | 22 | |

TABLE 92
Reduction of human DMPK in DMSXL transgenic mice

Quadriceps

| Compound | Dose | DMPK RNA | ED50 |
|---|---|---|---|
| No. | (mg/kg) | (% control) | (mg/kg) |
| PBS | N/A | 100 | — |
| 598769 | 12.5 | 50 | — |
| 1003033 | 1 | 94 | 5.5 |
| | 3 | 73 | |
| | 9 | 35 | |
| | 27 | 14 | |
| 1273291 | 1 | 96 | 6.1 |
| | 3 | 86 | |
| | 9 | 30 | |
| | 27 | 6 | |

TABLE 93
Reduction of human DMPK in DMSXL transgenic mice

| | | Quadriceps | | | | Gastrocnemius | | Heart | |
|---|---|---|---|---|---|---|---|---|---|
| | | DMPK | | DMPK | | DMPK | | DMPK | |
| | | RNA (% | | RNA (% | | RNA (% | | RNA (% | |
| Compound | Dose | control) | ED50 | control) | ED50 | control) | ED50 | control) | ED50 |
| No. | (mg/kg) | RTS3164 | (mg/kg) | RTS38095 | (mg/kg) | RTS3164 | (mg/kg) | RTS3164 | (mg/kg) |
| PBS | N/A | 100 | — | 100 | — | 100 | — | 100 | — |
| 598769 | 3 | 71 | 15 | 77 | 21 | 73 | 28 | 88 | 73 |
| | 10 | 56 | | 73 | | 67 | | 91 | |
| | 30 | 39‡ | | 35‡ | | 41‡ | | 65‡ | |
| | 60 | 30 | | 32 | | 52 | | 59 | |
| 1400737 | 3 | 76 | 23 | 78 | 25 | 61 | 15 | 96 | 70 |
| | 10 | 73 | | 71 | | 60 | | 107 | |
| | 30 | 42‡ | | 48‡ | | 47‡ | | 62‡ | |
| | 60 | 32 | | 33 | | 26 | | 60 | |
| 1400739 | 3 | 84 | 15 | 82 | 18 | 93 | 21 | 106 | 29 |
| | 10 | 57 | | 63 | | 65 | | 63 | |
| | 30 | 34 | | 37 | | 41 | | 46 | |
| | 60 | 24‡ | | 21 | | 26 | | 43 | |
| 1400743 | 3 | 75 | 24 | 85 | 36 | 71 | 20 | 76 | 42 |
| | 10 | 63 | | 75 | | 58 | | 73 | |
| | 30 | 48 | | 57 | | 51 | | 58 | |
| | 60 | 39 | | 40 | | 33 | | 51 | |
| 1400744 | 3 | 71 | 18 | 88 | 26 | 71 | 14 | 82 | 48 |
| | 10 | 59 | | 69 | | 54 | | 77 | |
| | 30 | 46 | | 46 | | 38 | | 63 | |
| | 60 | 29 | | 34 | | 27 | | 49 | |
| 1400752 | 3 | 78 | 23 | 93 | 29 | 61 | 11 | 83 | 56 |
| | 10 | 62 | | 64 | | 51 | | 65 | |
| | 30 | 44 | | 47 | | 35 | | 73 | |
| | 60 | 39 | | 43 | | 29 | | 53 | |
| 1400769 | 3 | 90 | 18 | 93 | 15 | 71 | 11 | 98 | 52 |
| | 10 | 62 | | 51 | | 53 | | 93 | |
| | 30 | 37 | | 37 | | 31 | | 61 | |
| | 60 | 23‡ | | 21‡ | | 20‡ | | 38‡ | |
| 1400775 | 3 | 96 | 115 | 91 | 68 | 81 | 40 | 100 | 142 |
| | 10 | 77 | | 85 | | 72 | | 96 | |
| | 30 | 92 | | 72 | | 54 | | 80 | |
| | 60 | 61 | | 53 | | 51 | | 73 | |
| 1400776 | 3 | 80 | 23 | 88‡ | 33 | 76 | 23 | 92 | 67 |
| | 10 | 63‡ | | 70‡ | | 48‡ | | 59‡ | |
| | 30 | 41 | | 46 | | 43 | | 62 | |
| | 60 | 40 | | 47 | | 41 | | 62 | |

‡indicates that fewer than 4 samples are available

TABLE 94
Reduction of human DMPK in DMSXL transgenic mice

Quadriceps

| Compound | Dose | DMPK RNA | ED50 |
|---|---|---|---|
| No. | (mg/kg) | (% control) | (mg/kg) |
| PBS | N/A | 100 | — |
| 877864 | 3 | 88 | 17.1 |
| | 9 | 63 | |
| | 27 | 40 | |
| 1046939 | 3 | 69 | 8.4 |
| | 9 | 48 | |
| | 27 | 29 | |

### Example 17: Activity of Modified Oligonucleotides Complementary to Human DMPK DM20 Transgenic Mice, Multiple Dose

DM20 transgenic mice (described herein above) were used to determine activity of modified oligonucleotides complementary to human DMPK.

### Treatment

DM20 transgenic mice were divided into groups of 4 mice each. Each mouse received subcutaneous injections of modified oligonucleotide at various doses indicated in the table below at once a week for two weeks (a total of 3 treatments). One group of 4 mice received subcutaneous injections of PBS once a week for two weeks (a total of 3 treatments). The PBS-injected group served as the control group to which oligonucleotide-treated groups were compared.

### RNA Analysis

7 days after the final treatment, mice were sacrificed and RNA was extracted from mouse quadricep muscle for real-time RTPCR analysis of DMPK RNA expression. Human DMPK primer probe sets RTS3164 (described herein above) was used to measure human DMPK RNA levels. DMPK RNA levels were normalized either to total RNA content, as measured to mouse GAPDH. Mouse GAPDH was amplified using mouse primer probe set mGapdh_LTS00102 (described herein above). Results are presented as percent DMPK RNA, relative to the amount in PBS treated animals (% control). ED50s were calculated in GraphPad Prism using nonlinear fit with variable slope (four parameter), top constrained to 100% (or 1), bottom constrained to 0. Y=Bottom+(Top−Bottom)/(1+(IC50/X)\hat{ }HillSlope).

Compound No. 877864 is described herein above.

TABLE 95
Reduction of human DMPK in DM20 transgenic mice

Quadriceps

| Compound | Dose | DMPK RNA | ED50 |
|---|---|---|---|
| No. | (mg/kg) | (% control) | (mg/kg) |
| PBS | N/A | 100 | — |
| 877864 | 3 | 91 | 17.8 |
| | 9 | 75 | |
| | 27 | 34‡ | |

‡indicates that there are fewer than 4 samples available

### Example 18: Dose-Dependent Inhibition of Human DMPK in A431 Cells by Modified Oligonucleotides

Modified oligonucleotides selected from the examples above were tested at various doses in A431 cells. The modified oligonucleotides were tested in a series of experiments using the same culture conditions. The results for each experiment are presented in separate tables shown below. Cells were plated at a density of 11,000 cells per well and were treated using free uptake with modified oligonucleotides at various doses, as specified in the tables below. After a treatment period of approximately 48 hours, DMPK RNA levels were measured as previously described using the human DMPK primer-probe set RTS38095 (described herein above). DMPK RNA levels were normalized to total RNA, as measured by RIBOGREEN®. Results are presented as percent DMPK RNA, relative to the amount in untreated control cells (% UTC).

The half maximal inhibitory concentration (IC₅₀) of each modified oligonucleotide was calculated in GraphPad prism using a log(inhibitor) vs. normalized response—Variable slope formula.

TABLE 96
Dose-dependent reduction of human DMPK RNA
in A431 cells by modified oligonucleotides

| | DMPK RNA (% UTC) | | | | | | | | | | |
|---|---|---|---|---|---|---|---|---|---|---|---|
| Compound | 10000 | 3333 | 1111 | 370 | 123 | 41 | 14 | 5 | 2 | 1 | IC₅₀ |
| No. | nM | nM | nM | nM | nM | nM | nM | nM | nM | nM | (μM) |
| 570052 | 5 | 8 | 8 | 14 | 31 | 64 | 77 | 94 | 98 | 90 | 0.06 |
| 598769 | 13 | 17 | 24 | 36 | 51 | 69 | 77 | 81 | 85 | 76 | 0.12 |
| 1338115 | 24 | 18 | 20 | 19 | 19 | 36 | 58 | 67 | 78 | 76 | 0.02 |
| 1400737 | 3 | 3 | 5 | 13 | 28 | 58 | 84 | 81 | 80 | 82 | 0.05 |
| 1400739 | 1 | 1 | 3 | 8 | 26 | 58 | 84 | 84 | 97 | 97 | 0.05 |
| 1400742 | 7 | 12 | 25 | 42 | 67 | 83 | 110 | 118 | 105 | 111 | 0.30 |

TABLE 97
Dose-dependent reduction of human DMPK RNA
in A431 cells by modified oligonucleotides

| | DMPK RNA (% UTC) | | | | | | | | | | |
|---|---|---|---|---|---|---|---|---|---|---|---|
| Compound | 10000 | 3333 | 1111 | 370 | 123 | 41 | 14 | 5 | 2 | 1 | IC₅₀ |
| No. | nM | nM | nM | nM | nM | nM | nM | nM | nM | nM | (μM) |
| 1400743 | 18 | 23 | 31 | 48 | 63 | 72 | 82 | 89 | 91 | 83 | 0.29 |
| 1400744 | 9 | 10 | 17 | 28 | 52 | 71 | 80 | 81 | 89 | 88 | 0.11 |
| 1400748 | 11 | 15 | 24 | 36 | 57 | 82 | 84 | 86 | 100 | 88 | 0.20 |
| 1400750 | 12 | 12 | 23 | 37 | 55 | 69 | 85 | 89 | 105 | 84 | 0.17 |
| 1400752 | 25 | 25 | 31 | 46 | 66 | 78 | 87 | 98 | 98 | 103 | 0.40 |
| 1400755 | 0 | 0 | 1 | 3 | 15 | 54 | 95 | 97 | 104 | 118 | 0.05 |
| 1400760 | 21 | 20 | 23 | 26 | 44 | 82 | 125 | 128 | 121 | 136 | 0.16 |

1. (canceled)

2. (canceled)

3. An oligomeric compound comprising a modified oligonucleotide consisting of 12 to 30 linked nucleosides, wherein the nucleobase sequence of the modified oligonucleotide is at least 80% complementary to an equal length portion of a DMPK nucleic acid, wherein the modified oligonucleotide has at least one modification selected from a modified sugar moiety and a modified internucleoside linkage, and wherein the nucleobase sequence of the modified oligonucleotide is at least 80% complementary to an equal length portion within:
nucleobases 9052-9103 of SEQ ID NO: 1;
nucleobases 9228-9256 of SEQ ID NO: 1;
nucleobases 9574-9610 of SEQ ID NO: 1;
nucleobases 10010-10043 of SEQ ID NO: 1;
nucleobases 10271-10298 of SEQ ID NO: 1;
nucleobases 10364-10391 of SEQ ID NO: 1;
nucleobases 10683-10707 of SEQ ID NO: 1;
nucleobases 10709-10734 of SEQ ID NO: 1;
nucleobases 10812-10857 of SEQ ID NO: 1;
nucleobases 11853-11879 of SEQ ID NO: 1;
nucleobases 13310-13350 of SEQ ID NO: 1;
nucleobases 13999-14046 of SEQ ID NO: 1;
nucleobases 14090-14118 of SEQ ID NO: 1;
nucleobases 14232-14258 of SEQ ID NO: 1;
nucleobases 17565-17594 of SEQ ID NO: 1;
nucleobases 17731-17761 of SEQ ID NO: 1;
nucleobases 19719-19753 of SEQ ID NO: 1;
nucleobases 19795-19869 of SEQ ID NO: 1;
nucleobases 19888-19942 of SEQ ID NO: 1;
nucleobases 19915-19942 of SEQ ID NO: 1;
nucleobases 20871-20905 of SEQ ID NO: 1;
nucleobases 21117-21153 of SEQ ID NO: 1; or
nucleobases 22118-22143 of SEQ ID NO: 1.

4. The oligomeric compound of claim 3, wherein the modified oligonucleotide has a nucleobase sequence comprising at least 12, 13, 14, 15, or 16 contiguous nucleobases of a nucleobase sequence selected from:
SEQ ID NOs: 132, 186, 256, 327, 446, 1374, 1596, 1667, 1747, 1818, 1895, 1964, 2038, 2121, 2191;
SEQ ID NOs: 510, 1173, 1668, 1748, 1819, 1896;
SEQ ID NOs: 1376, 1448, 1526, 1599, 1670;
SEQ ID NOs: 1823, 1900, 1969, 2043;
SEQ ID NOs: 1380, 1452, 1530, 1901, 1970, 2044, 2127, 2197;
SEQ ID NOs: 1206, 1381, 1453, 1531, 1604, 1971, 2045, 2128, 2198;
SEQ ID NOs: 640, 714, 821, 1172, 1677, 1757, 1828;
SEQ ID NOs: 43, 115, 202, 900, 960, 1027, 1195, 1905;
SEQ ID NOs: 1384, 1456, 1534, 1607, 1678, 1758;
SEQ ID NOs: 1387, 1977, 2051, 2134, 2204;
SEQ ID NOs: 1296, 1351, 1425, 1501, 1793, 1867, 1979, 2052, 2083, 2092, 2206;
SEQ ID NOs: 49, 159, 208, 293, 402, 471, 556, 618, 676, 692, 754, 817, 901, 971, 1038, 1744, 1791, 1863, 1960, 2016, 2119, 2163;
SEQ ID NOs: 1718, 1814, 1891, 1941;
SEQ ID NOs: 41, 140, 888, 981, 1033, 2081, 2154;
SEQ ID NOs: 444, 508, 573, 1874, 1949, 2060, 2103;
SEQ ID NOs: 274, 337, 410, 526, 575, 665, 712, 829, 897, 1397, 1467, 2138, 2210, 2270;
SEQ ID NOs: 1432, 1509, 1580, 1654, 1729, 1801;
SEQ ID NOs: 355, 412, 506, 567, 673, 747, 832, 904, 956, 1399, 1469, 1545, 1581, 1655, 1730, 1841, 1916, 1988, 1989, 2027, 2106, 2177;
SEQ ID NOs: 160, 249, 313, 371, 424, 503, 588, 647, 755, 789, 882, 1248-1254, 1263-1264, 1266-1273, 1284-1285, 1332, 1400, 1489, 1619, 1637, 1638, 1639, 1656, 1709, 2006, 2079, 2082, 2085, 2153, 2303;
SEQ ID NOs: 503, 588, 647, 755, 789, 882, 1263, 1264, 1332, 1400, 1619, 1637, 1638, 1639, 1656, 1709, 2006, 2079, 2082, 2085, 2153, 2303;
SEQ ID NOs: 144, 233, 291, 328, 435, 482, 564, 642, 748, 808, 874, 955, 1339, 1340, 1341, 1492, 1732, 1803, 2321, 2322, 2323;
SEQ ID NOs: 576, 652, 724, 811, 870, 1359, 1433, 1510, 1583, 1692; or
SEQ ID NOs: 696, 1255-1259, 1265, 1274-1277, 1283, 1330, and 1331.

5. (canceled)

6. An oligomeric compound comprising a modified oligonucleotide consisting of 12 to 30 linked nucleosides, wherein the nucleobase sequence of the modified oligonucleotide comprises at least 8, at least 9, at least 10, at least 11, at least 12, at least 13, at least 14, at least 15, or at least 16 contiguous nucleobases of the nucleobase sequence of any one of SEQ ID NOs: 18-2334, and wherein the modified oligonucleotide has at least one modification selected from a modified sugar moiety and a modified internucleoside linkage.

7. (canceled)

8. The oligomeric compound of claim 6, wherein the modified oligonucleotide has a nucleobase sequence comprising the nucleobase sequence of any one of SEQ ID NOs: 18-2334.

9. (canceled)

10. (canceled)

11. (canceled)

12. The oligomeric compound of claim 6, wherein the modified oligonucleotide consists of 16, 18, or 20 linked nucleosides.

13. (canceled)

14. (canceled)

15. The oligomeric compound of claim 6, wherein at least one nucleoside of the modified oligonucleotide comprises a modified sugar moiety, wherein the modified sugar moiety comprises a bicyclic sugar moiety, a non-bicyclic modified sugar moiety, or a sugar surrogate.

16. (canceled)

17. The oligomeric compound of claim 15, wherein the modified sugar moiety comprises a bicyclic sugar moiety, wherein the bicyclic sugar moiety comprises a 2′-4′ bridge selected from —O—CH₂—; and —O—CH(CH₃)—.

18. (canceled)

19. The oligomeric compound of claim 15, wherein the modified sugar moiety comprises a non-bicyclic sugar moiety, wherein the non-bicyclic modified sugar moiety is a 2′-MOE sugar moiety or a 2′-OMe sugar moiety.

20. (canceled)

21. The oligomeric compound of claim 6, wherein the modified oligonucleotide comprises at least one modified internucleoside linkage, wherein the at least one modified internucleoside linkage is a phosphorothioate internucleoside linkage, a mesyl phosphoramidate internucleoside linkage, or a methoxypropyl phosphonate internucleoside linkage.

22. (canceled)

23. (canceled)

24. (canceled)

25. (canceled)

26. The oligomeric compound of claim 6, wherein at least one internucleoside linkage of the modified oligonucleotide is a phosphodiester internucleoside linkage.

27. (canceled)

28. The oligomeric compound of claim 6, wherein each internucleoside linkage of the modified oligonucleotide is independently selected from a phosphodiester internucleoside linkage, a phosphorothioate internucleoside linkage, a mesyl phosphoramidate internucleoside linkage, or a methoxypropyl phosphonate internucleoside linkage.

29. (canceled)

30. (canceled)

31. The oligomeric compound of claim 21, wherein the internucleoside linkage motif of the modified oligonucleotide is selected from soooossssssssssooss, sssssssssssssss, sooossssssssssoooss, soosssssssssoooss, sooosssssssssooss, sooooossssssssssoss, soooosssssssssoss, ssssxssssssssss, sssssssssssss, soossssssssssos, sosssssssssssos, soosxssssssssos, ooooxoooooooooo, sssssxsssssssss, soossxsssssssos, wherein each “s” represents a phosphorothioate internucleoside linkage, each “o” represents a phosphodiester internucleoside linkage, and each “x” represents a methoxypropyl phosphonate internucleoside linkage.

32. The oligomeric compound of claim 6, wherein the modified oligonucleotide comprises at least one modified nucleobase, wherein the modified nucleobase is 5-methylcytosine.

33. (canceled)

34. (canceled)

35. The oligomeric compound of claim 6, wherein the modified oligonucleotide comprises a deoxy region.

36. (canceled)

37. (canceled)

38. (canceled)

39. The oligomeric compound of claim 35, wherein the deoxy region is flanked on the 5′-side by a 5′-external region consisting of 1-6 linked 5′-external region nucleosides and is flanked on the 3′-side by a 3′-external region consisting of 1-6 linked 3′-external region nucleosides; wherein:
the 3′-most nucleoside of the 5′ external region comprises a modified sugar moiety; and
the 5′-most nucleoside of the 3′ external region comprises a modified sugar moiety.

40. (canceled)

41. (canceled)

42. The oligomeric compound of claim 39, wherein the modified oligonucleotide has:
(a) a 5′ external region consisting of 5 linked nucleosides;
a deoxy region consisting of 10 linked nucleosides; and
a 3′ external region consisting of 5 linked nucleosides;
(b) a 5′ external region consisting of 6 linked nucleosides;
a deoxy region consisting of 10 linked nucleosides; and
a 3′ external region consisting of 4 linked nucleosides; or
(c) a 5′ external region consisting of 4 linked nucleosides;
a deoxy region consisting of 10 linked nucleosides; and
a 3′ external region consisting of 6 linked nucleosides,
wherein each of the 5′ external region nucleosides and each of the 3′ external region nucleosides is a 2′-MOE nucleoside.

43. (canceled)

44. (canceled)

45. The oligomeric compound of claim 39, wherein the modified oligonucleotide has:
a 5′ external region consisting of 3 linked nucleosides;
a deoxy region consisting of 10 linked nucleosides; and
a 3′ external region consisting of 3 linked nucleosides;
wherein each of the 5′ external region nucleosides and each of the 3′ external region nucleosides is a cEt nucleoside.

46. The oligomeric compound of claim 39, wherein the modified oligonucleotide has:
a 5′ external region consisting of 1-6 linked nucleosides;
a deoxy region consisting of 6-10 linked nucleosides; and
a 3′ external region consisting of 1-6 linked nucleosides;
wherein each of the 5′ external region nucleosides and each of the 3′ external region nucleosides is independently selected from a cEt nucleoside or a 2′-MOE nucleoside, and each of the deoxy region nucleosides is a 2′-β-D-deoxynucleoside.

47. The oligomeric compound of claim 39, wherein the modified oligonucleotide has a sugar motif comprising:
a 5′ external region consisting of 3-6 linked nucleosides;
a deoxy region consisting of 7-8 linked nucleosides; and
a 3′ external region consisting of 3-6 linked nucleosides;
wherein each of the 3′ external region nucleosides is independently selected from a 2′-MOE nucleoside and a cEt nucleoside, and the 5′ external region has the following formula:
(Nk)n(Nd)(Nx)
wherein each Nk is a bicyclic nucleoside, Nx is 2′-OMe nucleoside, Nd is a 2′-β-D-deoxynucleoside, and n is from 1-4.

48. The oligomeric compound of claim 6, wherein the modified oligonucleotide has a sugar motif (5′ to 3′) selected from: eeeeeddddddddddeeeee, kkkddddddddddkkk, eekkddddddddkkee, eeeeddddddddddeeeeee, eeeeddddddddeeeeee, eeeeeddddddddeeeee, eeeeeeddddddddddeeee, eeeeeeddddddddeeee, kkkedddddddddkkk, kkkdyddddddddkkk, kkeddddddddddkkk, kekddddddddddkkk, ekkddddddddddkke, kkddddddddddkk, ekkkddddddddkkke, ekkddddddddddkkk, kkkddddddddddkke, kkkdd[5′-(S)-Me-d]dddddddkkk, kkkdd[5′-(R)-Me-d]dddddddkkk, kkkdd[5′-(R)-allyl-d]dddddddkkk, kkkddd[5′-(R)-Me-d]ddddddkkk, wherein each “d” represents a 2′-ββ-D-deoxyribosyl sugar moiety, each “e” represents a 2′-MOE sugar moiety, each “y” represents a 2′-OMe sugar moiety, each “[5′-(S)-Me-d]” represents a 5′-(S)-methyl-β-D-2′-deoxyribosyl sugar moiety, each “[5′-(R)-Me-d]” represents a 5′-(R)-methyl-β-D-2′-deoxyribosyl sugar moiety, and each “[5′-(R)-allyl-d]” represents a 5′-(R)-allyl-β-D-2′-deoxyribosyl sugar moiety, and each “k” represents a cEt sugar moiety.

49. The oligomeric compound of claim 6, wherein the oligomeric compound comprises a conjugate group, wherein the conjugate group comprises a conjugate linker and a conjugate moiety.

50. (canceled)

51. (canceled)

52. (canceled)

53. The oligomeric compound of claim 49, wherein the conjugate moiety is a 6-palmitamidohexyl conjugate moiety.

54. The oligomeric compound of claim 49, wherein the conjugate linker is a phosphodiester linker.

55. The oligomeric compound of claim 49, wherein the conjugate group has the following structure:

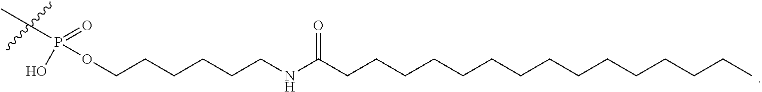 [SMILES: *P(=O)(O)OCCCCCCNC(=O)CCCCCCCCCCCCCCC]

56. The oligomeric compound of claim 49, wherein the conjugate linker consists of a single bond.

57. The oligomeric compound of claim 49, wherein the conjugate linker is cleavable.

58. (canceled)

59. (canceled)

60. The oligomeric compound of claim 49, wherein the conjugate group is attached to the modified oligonucleotide at the 5′-end of the modified oligonucleotide or at the 3′-end of the modified oligonucleotide.

61.-67. (canceled)

68. A population of oligomeric compounds of claim 6, wherein all of the phosphorothioate internucleoside linkages of the modified oligonucleotide are stereorandom.

69.-73. (canceled)

74. A pharmaceutical composition comprising the oligomeric compound of claim 6, and a pharmaceutically acceptable diluent.

75. The pharmaceutical composition of claim 74, wherein the pharmaceutically acceptable diluent is phosphate-buffered saline or artificial cerebrospinal fluid.

76. The pharmaceutical composition of claim 75, wherein the pharmaceutical composition consists essentially of the oligomeric compound and phosphate-buffered saline or artificial cerebrospinal fluid.

77. (canceled)

78. A method of treating a disease associated with DMPK, comprising administering to a subject having the disease associated with DMPK a therapeutically effective amount of the oligomeric compound of claim 6, thereby treating the disease associated with DMPK.

79. The method of claim 78, wherein the disease associated with DMPK is type 1 myotonic dystrophy, wherein administering the oligomeric compound reduces one or more of muscle stiffness, myotonia, disabling distal weakness, weakness in face and jaw muscles, difficulty in swallowing, drooping of the eyelids (ptosis), weakness of neck muscles, weakness in arm and leg muscles, persistent muscle pain, hypersomnia, muscle wasting, dysphagia, respiratory insufficiency, irregular heartbeat, heart muscle damage, apathy, insulin resistance, and cataracts, and wherein the subject is human.

80.-87. (canceled)
